(19) United States
(12) Patent Application Publication
(10) Pub. No.: US 2009/0227591 A1
(43) Pub. Date: Sep. 10, 2009

(54) Cyclopentene compounds

(76) Inventors: Gerard Martin Paul Giblin, Essex (GB); Adrian Hall, Essex (GB); David Nigel Hurst, Essex (GB); Ian Reginald Kilford, Essex (GB); Xiao Qing Lewell, Hertfordshire (GB); Alan Naylor, Essex (GB); Riccardo Novelli, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 10/574,901

(22) PCT Filed: Oct. 6, 2004
(86) PCT No.: PCT/EP04/11314

§ 371 Date: May 22, 2006

(30) Foreign Application Priority Data
Oct. 8, 2003 (GB) 0323581.9

(51) Int. Cl. A61K 31/501; A61K 31/44; C07D 401/02; C07D 213/62

(52) U.S. Cl. 514/252.03; 514/345; 546/290; 544/238

(57) ABSTRACT

Compounds of formula (I) or a pharmaceutically acceptable derivative thereof:

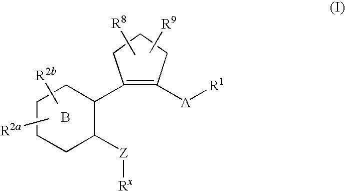

wherein A, B, Z, R¹, R^{2a}, R^{2b}, R⁸, R⁹, and R^{x} are as defined in the specification, a process for the preparation of such compounds, pharmaceutical compositions comprising such compounds and the use of such compounds in medicine.

This invention relates to cyclopentene compounds, to processes for their preparation, to pharmaceutical compositions containing them and to their use in medicine, in particular their use in the treatment of conditions mediated by the action of PGE₂ at EP₁ receptors.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Madouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289-R-294), studies suggest that PGE₂-induced hyperthermia in the rat is mediated predominantly through the EP₁ receptor.

WO 96/06822 (Mar. 7, 1996), WO 96/11902 (Apr. 25, 1996), EP 752421-A1 (Jan. 8, 1997), WO 01/19814 (22 Mar. 2001), WO 03/084917 (16 Oct. 2003), WO 03/101959 (11 Dec. 2003) and WO 2004/039753 (13 May 2004) disclose compounds as being useful in the treatment of prostaglandin mediated diseases.

It is now suggested that a novel group of cyclopentene derivatives surprisingly are selective for the EP₁ receptor over the EP₃ receptor, and are therefore indicated to be useful in treating conditions mediated by the action of PGE₂ at EP₁ receptors. Such conditions include pain, or inflammatory, immunological, bone, neurodegenerative or renal disorders.

Accordingly the present invention provides cyclopentene compounds of formula (I):

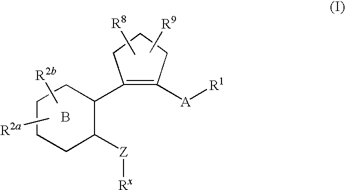

wherein:
A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂H, CN, CONR⁵R⁶, CH₂CO₂H, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2; optionally substituted alkenyl; or optionally substituted alkynyl: or R^{x} represents optionally substituted alkenyl, optionally substituted CQ^{a}Q^{b}-heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ and R⁹ each independently represents hydrogen, chloro, fluoro, CF₃, C₁₋₃alkoxy or C₁₋₃alkyl;
Q^{a} and Q^{b} are each independently selected from hydrogen and CH₃;
wherein when A is a 6-membered ring the R¹ substituent and cyclopentene ring are attached to carbon atoms 1,2-, 1,3- or 1,4-relative to each other, and when A is a five-membered ring or bicyclic heterocyclyl group the R¹ substituent and cyclopentene ring are attached to substitutable carbon atoms 1,2- or 1,3-relative to each other; and derivatives thereof.

When A is a six membered ring, preferably R¹ is attached to the group A in the 3 position relative to the bond attaching A to the cyclopentene ring.

Suitable examples of A include phenyl, pyridyl, pyridazinyl, pyrimidinyl and pyrazinyl, all of which may be optionally substituted.

Optional substituents for A include up to four substituents, preferably 0 or 1 substituent, independently selected from halogen, optionally substituted C₁₋₄alkyl e.g. CF₃, CH₃, and C₂H₅, NH₂, NHC₁₋₄alkyl, NHCOC₁₋₄alkyl, and SCH₃.

When B is pyridyl, in one aspect the pyridine N atom is situated adjacent to the ring carbon carrying the Z substituent.

Preferably Z is O,

Suitably R¹ includes CO₂H and CONHSO₂phenyl.

Particular examples of R^{2a} and R^{2b} include hydrogen, halogen, optionally substituted C₁₋₆alkyl e.g. CF₃ or CH₃, and optionally substituted C₁₋₆alkoxy.

Preferably R^{2a} is hydrogen or CH₃. More preferably R^{2a} is hydrogen.

Preferably R^{2b} represents hydrogen, halogen, CF₃, or CH₃.

Preferably R^{2b} is positioned 1,4-relative to the Z substituent and 1,3-relative to the cyclopentene ring.

Suitably R⁴ includes hydrogen and C₁₋₄alkyl.

Suitably R⁵ includes hydrogen or C₁₋₄alkyl.

Suitably R⁶ includes hydrogen, C₁₋₄alkyl or SO₂phenyl.

Suitably R⁷ include hydrogen or C₁₋₄alkyl.

Suitably R⁸ include CH₃ or hydrogen, in one aspect R⁸ represents hydrogen.

An example of R⁹ is hydrogen.

An example of Q^{a} is hydrogen.

An example of Q^{b} is hydrogen.

Suitably R^{x} includes optionally substituted C₁₋₈alkyl, optionally substituted C₂₋₈alkenyl and CH₂₋₈phenyl optionally substituted by one, two or three substituents, selected from Cl, Br, F, CF₃, OCF₃, C₁₋₄alkyl, and OC₁₋₄alkyl.

In one aspect R^{x} is optionally substituted C₃₋₈alkyl, optionally substituted C₃₋₈alkenyl and CH₂phenyl optionally substituted by one, two or three substituents, selected from Cl, Br, F, CF₃, OCF₃, C₁₋₄alkyl, and OC₁₋₄alkyl.

Suitably R^{x} when an optionally substituted C₃₋₈alkyl includes e.g. isobutyl, CH₂cyclopentene and CH₂cyclohexene.

Suitably R^{x} when an optionally substituted C₂₋₈alkenyl include e.g. CH₂CH═CH₂ and CH₂CH═CH-phenyl.

A certain group of compounds of formula (I) are compounds of formula (IA):

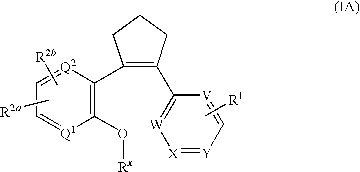

wherein:
W, X, and Y each represent CR¹² or N;
V represents CR¹, CR¹² or N;
wherein at least two of W, X, Y and V is CR¹², and R¹² is independently selected from hydrogen, halogen, CF₃, CH₃, NH₂, NHC₁₋₆alkyl, NHCOC₁₋₆alkyl, and SCH₃;
Q¹ and Q² each represents CH, or one of Q¹ and Q² is N and the other is CH;
R¹ is CO₂H, CONR⁵R⁶, CH₂CO₂H, SO₂C₁₋₆alkyl, SO₂NR⁵R⁶, NR⁵CONR⁶R⁶, tetrazolyl or COSO₂NR⁵R⁶;
R^{2a} and R^{2b} are selected from hydrogen, halogen, optionally substituted C₁₋₆alkyl, and optionally substituted C₁₋₆alkoxy;
R^{x} represents optionally substituted C₁₋₆alkyl, optionally substituted C₃₋₈alkenyl, and optionally substituted CH₂phenyl;
R⁵ is hydrogen or C₁₋₄alkyl;
R⁶ is hydrogen, C₁₋₄alkyl or SO₂phenyl;
R¹² is selected from hydrogen, halogen, NR⁵R⁶, NR⁵COC₁₋₆alkyl, NR⁵SO₂C₁₋₆alkyl, OR⁵, SR⁵, and optionally substituted C₁₋₆alkyl;
or derivatives thereof.

Suitably R¹ includes CO₂H and CONHSO₂phenyl.

Suitably R^{x} includes optionally substituted C₃₋₈alkyl, optionally substituted C₃₋₈alkenyl, and CH₂phenyl optionally substituted by one, two or three substituents, selected from Cl, Br, F, CF₃, OCF₃, C₁₋₄alkyl, and OC₁₋₄alkyl.

In one aspect R¹ is positioned 1,3-relative to the cyclopentene ring.

In another aspect one or two of W, X, Y and V is N.

In yet another aspect one of Q¹ and Q² is N and the other is CH.

A particular set of compounds are those wherein one or two of W, X, Y and V is N and Q¹ and Q² are both CH. A further set of compounds are those where one of Q¹ and Q² is N and W, X, Y, and V are each CR¹².

In one aspect Q¹ is N or CH and Q² is CH.

Suitably R^{2a} is hydrogen.

Suitably R^{2b} is positioned 1,4-relative to OR^{x} and 1,3-relative to the cyclopentene ring.

Suitably R^{2b}is selected from hydrogen, F, Br, Cl, CH₃ and CF₃.

Suitably R¹² includes hydrogen, halogen e.g. F or C₁, CF₃, NH₂, NHCOC₁₋₄alkyl, SCH₃, and C₁₋₄alkyl, e.g. CH₃ and C₂H₅;

Compounds of formula (I) include the compounds of Examples 1 to 417 and derivatives thereof.

A particular group of compounds of formula (I) include the compounds of Examples 145-148, 213-241, 342-368, and 388-417 and derivatives thereof.

The compounds of the invention are selective for EP₁ over EP₃. The compounds of the examples are at least 20 fold selective. Preferred compounds are at least 100 fold selective for EP₁ over EP₃.

Derivatives of the compounds of formula (I) include pharmaceutically acceptable derivatives. The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I).

It will be appreciated by those skilled in the art that the compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts referred to above will be pharmaceutically acceptable salts, but other salts may find use, for example in the preparation of compounds of formula (I) and the pharmaceutically acceptable salts thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. A particular salt is the sodium salt. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropyl amine, tromethamine, and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and if crystalline, may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine.

The term “alkyl” as a group or part of a group means a straight, branched or cyclic chain alkyl group or combinations thereof. Unless hereinbefore defined, examples of alkyl include C₁₋₈alkyl, for example methyl, ethyl, n-propyl, iso-propyl, n-butyl, sec-butyl, iso-butyl, t-butyl, pentyl, hexyl, 1,1-dimethylethyl, cyclopentyl or cyclohexyl or combinations thereof such as cyclohexylmethyl and cyclopentylmethyl.

The term “alkoxy” as a group or as part of a group means a straight, branched or cyclic chain alkoxy group. Unless hereinbefore defined “alkoxy” includes C₁₋₈alkoxy, e.g. methoxy, ethoxy, n-propoxy, iso-propoxy, n-butoxy, sec-butoxy, iso-butoxy, tert-butoxy, pentoxy, hexyloxy, cyclopentoxy or cyclohexyloxy. In one aspect “alkoxy” is C₁₋₆ alkoxy.

The term “alkenyl” means linear or branched structures and combinations thereof, of the indicated number of carbon atoms, having at least one carbon-to-carbon double bond, wherein hydrogen may be replaced by an additional carbon to carbon double bond. In one aspect “alkenyl” is C₂₋₆alkenyl, for example ethenyl, propenyl, 1-methylethenyl, butenyl and the like.

The term “alkynyl” means linear or branched structures and combinations thereof, of the indicated number of carbon atoms, having at least one carbon-to-carbon triple bond. C₂₋₈alkynyl, for example, includes ethynyl, propynyl, butynyl and the like.

The term “heterocyclyl” as a group or as part of a group means an aromatic or non-aromatic five or six membered ring which contains from 1 to 4 heteroatoms selected from nitrogen, oxygen or sulfur and is unsubstituted or substituted by, for example, up to three substituents. Examples of 5-membered heterocyclyl groups include furyl, dioxalanyl, pyrrolyl, oxazolyl, thiazolyl, imidazolyl, oxadiazolyl, thiadiazolyl, triazolyl, triazinyl, isothiazolyl, isoxazolyl, thiophenyl, pyrazolyl or tetrazolyl. Examples of 6-membered heterocyclyl groups are pyridyl, pyridazinyl, pyrimidinyl, pyrazinyl, triazinyl or tetrazinyl.

The term “aryl” as a group or part of a group means a 5- or 6-membered aromatic ring, for example phenyl, or a 7 to 12 membered bicyclic ring system where at least one of the rings is aromatic, for example naphthyl. An aryl group may be optionally substituted by one or more substituents, for example up to 4, 3 or 2 substituents. Preferably the aryl group is phenyl.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substituents, for example up to 3 or up to 2 substituents. Examples of “heteroaryl” used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl.

When the heteroatom nitrogen replaces a carbon atom in an alkyl group, or when nitrogen is present in a heteroaryl, heterocyclyl or bicyclic heterocyclyl group, the nitrogen atom will, where appropriate, be substituted by one or two substituents selected from hydrogen and C₁₋₈alkyl, preferably hydrogen and C₁₋₆alkyl, more preferably hydrogen.

Optional substituents for alkyl or alkenyl groups unless hereinbefore defined include phenyl or halo e.g. Cl, Br or F. An alkyl or alkenyl group may be substituted by one or more optional substituents, for example up to 5, 4, 3, or 2 optional substituents. Particular substituted alkyl groups include those substituted by one or more fluorines e.g. CH₂F, CHF₂, CF₃, C₂F₅ etc, especially CF₃.

Optional substituents for alkoxy groups unless hereinbefore defined include halo e.g. Cl, Br or F. An alkoxy group may be substituted by one or more optional substituents, for example up to 5, 4, 3, or 2 optional substituents. Particular substituted alkoxy groups include those substituted by one or more fluorines e.g. OCH₂F, OCHF₂, OCF₃, OC₂F₅etc.

Unless otherwise defined, optional substituents for aryl, heteroaryl or heterocycyl moieties as a group or part of a group are selected from C₁₋₆alkyl, C₁₋₆alkoxy and halogen.

Compounds of formula (I) can be prepared as set forth in the following schemes and in the examples. The following processes form another aspect of the present invention.

For example, compounds of formula (I) may be prepared by the general route below:

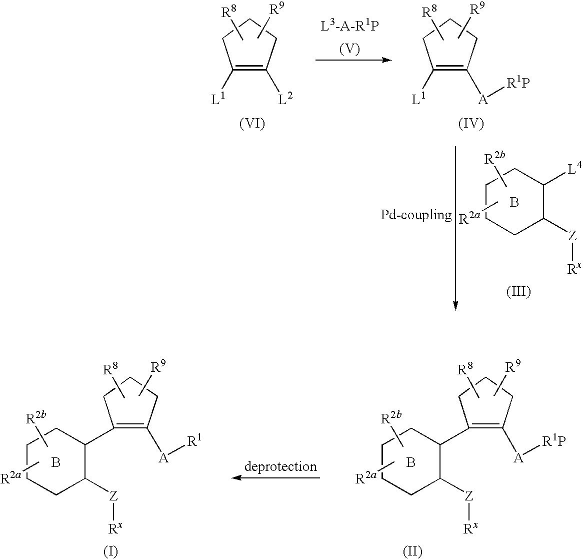

wherein L¹ and L² each represent a leaving group for example halo, or triflate; L³ and L⁴ each represent an activating group, for example boronic acid; P is an optional protecting group; and A, B, R¹, R^{2a}, R^{2b}, R⁸, R⁹, Z and R^{x} are as defined for compounds of formula (I). L¹ can be converted to L^{1a}, and L² can be converted to L^{2a} wherein L^{1a} and L^{2a} each represent an activating group for example a boronic acid, and in this situation L³ and L⁴ can be halo or triflate.

When R¹ is CO₂H examples of P include methyl, ethyl or optionally substituted benzyl esters.

Suitable reaction conditions for the deprotection of a compound of formula (II) include heating in aqueous ethanolic sodium hydroxide solution.

Suitable reaction conditions for the reaction of a compound of formula (VI) with a boronic acid of formula (V) (wherein L³ is —B(OH)₂) or a compound of formula (IV) with a boronic acid of formula (III) (wherein L⁴ is —B(OH)₂) include heating with tetrakis(triphenylphosphine)palladium (0) and an inorganic base, for example potassium carbonate, in a solvent, e.g. ethylene glycol dimethyl ether (DME), toluene and ethanol, preferably in a ratio of 1:1.

Accordingly the present invention also provides a process for the preparation of a compound of formula (I) or a derivative thereof:

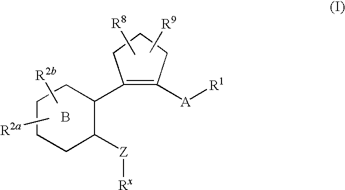

wherein:
A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂H, CN, CONR⁵R⁶, CH₂CO₂H, optionally substituted SO₂alkyl, SO₂NR⁵R⁶, NR⁵CONR⁶R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2; optionally substituted alkenyl; or optionally substituted alkynyl: or R^{x} represents optionally substituted alkenyl, optionally substituted CQ^{a}Q^{b}-heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ and R⁹ each independently represents hydrogen, chloro, fluoro, CF₃, C₁₋₃alkoxy or C₁₋₃alkyl;
Q^{a} and Q^{b} are each independently selected from hydrogen and CH₃;
wherein when A is a 6-membered ring the R¹ substituent and cyclopentene ring are attached to carbon atoms 1,2-, 1,3- or 1,4-relative to each other, and when A is a five-membered ring or bicyclic heterocyclyl group the R¹ substituent and cyclopentene ring are attached to substitutable carbon atoms 1,2- or 1,3-relative to each other; comprising:
reacting a compound of formula (IV):

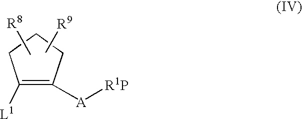

wherein R⁸, R⁹, A, and R¹ are as hereinbefore defined above for a compound of formula (I),
L¹ is a leaving group and P is an optional protecting group;
with a compound of formula (III):

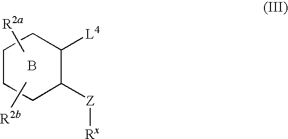

wherein R^{2a}, R^{2b}, B, Z, and R^{x} are as hereinbefore defined above for a compound of formula (I) and L⁴ is an activating group;
and where required converting:
one group A to another group A, and/or
one group R^{x} to another group R^{x};
and where required carrying out the following optional steps in any order: effecting deprotection; and/or
converting one group R¹ to another group R¹; and/or
forming a derivative of the compound of formula (I) so formed.

Alternatively compounds of formula (I) may be prepared according to the route described below:

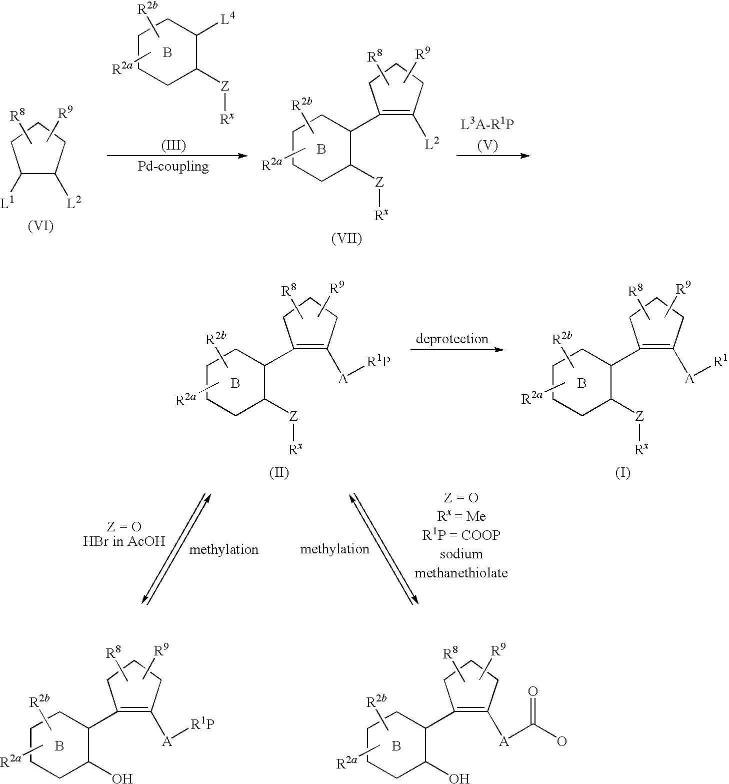

wherein L¹, L², L³, L⁴ and P are as defined above, and A, B, R¹, R^{2a}, R^{2b}, R⁸, R⁹, Z, and R^{x} are as defined for compounds of formula (I). L¹ can be converted to L^{1a}, and L² can be converted to L^{2a} wherein L^{1a} and L^{2a} each represent an activating group for example a boronic acid, and in this situation L³ and L⁴ can represent halo or triflate.

Accordingly the present invention also provides a process for the preparation of a compound of formula (I) or a derivative thereof:

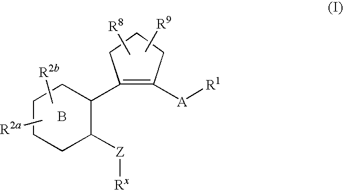

wherein:
A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂H, CN, CONR⁵R⁶, CH₂CO₂H, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group Independently selected from NR⁴, O and SOₙ, wherein n is 0, 1 or 2; optionally substituted alkenyl; or optionally substituted alkynyl: or R^{x} represents optionally substituted alkenyl, optionally substituted CQ^{a}Q^{b}-heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ and R⁹ each independently represents hydrogen, chloro, fluoro, CF₃, C₁₋₃alkoxy or C₁₋₃alkyl;
Q^{a} and Q^{b} are each independently selected from hydrogen and CH₃;
wherein when A is a 6-membered ring the R¹ substituent and cyclopentene ring are attached to carbon atoms 1,2-, 1,3- or 1,4-relative to each other, and when A is a five-membered ring or bicyclic heterocyclyl group the R¹ substituent and cyclopentene ring are attached to substitutable carbon atoms 1,2- or 1,3-relative to each other; comprising:
reacting a compound of formula (VII):

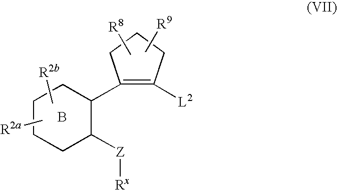

wherein R^{2a}, R^{2b}, R⁸, R⁹, A, B, R^{x} and R¹ are as hereinbefore defined above for a compound of formula (I), and L² is a leaving group;
with a compound of formula (V):

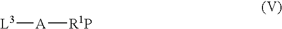

wherein R¹, and A are as hereinbefore defined above for a compound of formula (I); L³ is an activating group and P is an optional protecting group;
and where required converting:
one group A to another group A, and/or
one group R^{x} to another group R^{x};
and where required carrying out the following optional steps in any order:
effecting deprotection; and/or
converting one group R¹ to another group R¹; and/or
forming a derivative of the compound of formula (I) so formed.

It will be appreciated that certain substituents in intermediates and compounds of formula (I) may be converted to other substituents by conventional methods known to those skilled in the art.

A group R¹ may be converted to another group R¹ by use of conventional organic transformations known to those skilled in the art. For example R¹═CO₂H may be converted to an amide, e.g. CONHCQ^{a}Q^{b}aryl or CONHCQ^{a}Q^{b}heteroaryl wherein Q^{a} and Q^{b} are selected from hydrogen and CH₃, by conventional methods for the preparation of amides as described in, for example, Richard Larock, Comprehensive Organic Transformations, 2nd edition, Wiley-VCH, ISBN 0-71-19031-4.

Cyclopentene derivatives of formula (VI), boronic acids of formula (III) and (V), and tetrakis(triphenylphosphine)palladium (0) are commercially available, or readily prepared by methods known to those skilled in the art.

The preparation and reactions of boronic acids of formula (III) and formula (V) is reviewed in Suzuki et al, Synth. Commun., 1981, 11, 513; Martin et al, Acta. Chim. Scand., 1993, 47, 221; and Miyaura et al, Chem. Rev., 1995, 95, 2457. For example, 2-benzyloxy-5-chlorophenylboronic acid may be prepared from 2-benzyloxy-5-chloro-iodobenzene. 2-Benzyloxy-5-chloro-iodobenzene may be prepared from 4-chloro-2-iodoanisole by demethylation followed by benzylation according to known methods.

Certain substituents in any of the reaction intermediates and compounds of formula (I) may be converted to other substituents by conventional methods known to those skilled in the art. Examples of substituents which may be converted include one group R^{x} to another group R^{x}; and one substituent on a group A to another substituent on a group A. Examples of such transformations include the reduction of a nitro group to give an amino group; alkylation and amidation of amino groups; hydrolysis of esters, alkylation of hydroxy and amino groups; and amidation and esterification of carboxylic acids. Such transformations are well known to those skilled in the art and are described in for example, Richard Larock, Comprehensive Organic Transformations, 2nd edition, Wiley-VCH, ISBN 0-471-19031-4.

For example, when R^{x} is p-methoxybenzyl, cleavage of the ether to give the phenol or pyridinol is carried out using, for example, using acid e.g. HCl/dioxane or using sodium methanethiolate. When R^{x} is methyl, cleavage of the ether to give the phenol is carried out using, for example, sodium methanethiolate. Cleavage of the ether to give a pyridinol is carried out in the presence of, for example, trifluoroacetic acid. Conversion to another R^{x} group, for example a substituted benzyl group, may be effected by reaction of the phenol or pyridinol with a suitable substituted benzyl bromide. The skilled person will appreciate that conversion of the protecting group P to another protecting group P may also occur under the reaction conditions used. When R^{x} is benzyl, cleavage of the ether to give the phenol or pyridinol may be carried out by hydrogenation according to known methods e.g. H₂—Pd/C or NH₄CO₂H—Pd/C. The resulting phenol or pyridinol can then be converted to another group R^{x} as described above.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

Cyclopentene intermediates of the formula (VI):

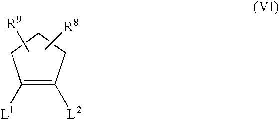

wherein L¹, L² are as defined above, and R⁸ and R⁹ are as hereinbefore defined for compounds of formula (I) are commercially available or may be readily prepared according to known methods.

Compounds of the formula (III):

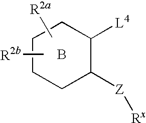

wherein L⁴ is as hereinbefore defined, R^{2a}, R^{2b}, Z, B and R^{x} and are as defined for compounds of formula (I) are commercially available, or may readily be prepared by methods known to those skilled in the art, for example from suitable commercially available pyridinols, anisoles or phenols using methods as described in the examples.

Compounds of the formula (V):

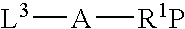

wherein L³ and P are as defined above and R¹ and A are as hereinbefore defined for compounds of formula (I) are commercially available or may readily be prepared, for example, from suitable halobenzoic acid esters according to known methods, for example using methods as described in the examples.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The compounds of the invention bind to the EP₁ receptor and they are therefore considered to be useful in treating conditions mediated by the action of PGE₂ at EP₁ receptors.

Conditions mediated by the action of PGE₂ at EP₁ receptors include pain; fever; inflammation; immunological diseases; abnormal platelet function diseases; impotence or erectile dysfunction; bone disease; hemodynamic side effects of non-steroidal anti-inflammatory drugs; cardiovascular diseases; neurodegenerative diseases and neurodegeneration; neurodegeneration following trauma; tinnitus; dependence on a dependence-inducing agent; complications of Type I diabetes; and kidney dysfunction.

The compounds of formula (I) are considered to be useful as analgesics. They are therefore considered useful in the treatment or prevention of pain.

The compounds of formula (I) are considered useful as analgesics to treat acute pain, chronic pain, neuropatic pain, inflammatory pain, visceral pain, pain associated with cancer and fibromyalgia, pain associated with migraine, tension headache and cluster headaches, and pain associated with functional bowel disorders, non-cardiac chest pain and non-ulcer dispepsia.

The compounds of formula (I) are considered useful in the treatment of chronic articular pain (e.g. rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever, pain associated with functional bowel disorders such as non-ulcer dyspepsia, non-cardiac chest pain and irritable bowel syndrome; pain associated with myocardial ischemia; post operative pain; headache; toothache; and dysmenorrhea. The compounds of this invention may also be useful in the treatment of visceral pain.

The compounds of the invention are considered to be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, or an inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, or an inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof adapted for use in human or veterinary medicine.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular Injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as diclofenac, indomethacin, nabumetone or ibuprofen; leukotriene receptor antagonists; removal of the stimulation (hyperpathia) or an absence of or deficit in selective sensory pathways (hypoalgesia).

The compounds of formula (I) are also considered useful in the treatment of fever.

The compounds of formula (I) are also considered useful in the treatment of inflammation, for example in the treatment of skin conditions (e.g. sunburn, burns, eczema, dermatitis, psoriasis); ophthalmic diseases such as glaucoma, retinitis, refinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation; other conditions with an inflammatory component such as vascular disease, migraine, periarteritis nodosa, thyroiditis, aplastic anaemia, Hodgkin's disease, scierodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

The compounds of formula (I) are also considered useful in the treatment of immunological diseases such as autoimmune diseases, immunological deficiency diseases or organ transplantation. The compounds of formula (I) are also effective in increasing the latency of HIV infection.

The compounds of formula (I) are also considered useful in the treatment of diseases relating to abnormal platelet function (e.g. occlusive vascular diseases).

The compounds of formula (I) are also considered useful for the preparation of a drug with diuretic action.

The compounds of formula (I) are also considered useful in the treatment of impotence or erectile dysfunction.

The compounds of formula (I) are also considered useful in the treatment of bone disease characterised by abnormal bone metabolism or resorbtion such as osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

The compounds of formula (I) are also considered useful for attenuating the hemodynamic side effects of non-steroidal anti-inflammatory drugs (NSAID's) and cyclooxygenase-2 (COX-2) inhibitors.

The compounds of formula (I) are also considered useful in the treatment of cardiovascular diseases such as hypertension or myocardiac ischemia; functional or organic venous insufficiency; varicose therapy; haemorrhoids; and shock states associated with a marked drop in arterial pressure (e.g. septic shock).

The compounds of formula (I) are also considered useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

The compounds of formula (I) are also considered useful in the treatment of tinnitus.

The compounds of formula (I) are also considered useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

The compounds of formula (I) are also considered useful in the treatment of complications of Type 1 diabetes (e.g. diabetic microangiopathy, diabetic retinopathy, diabetic nephropathy, macular degeneration, glaucoma), nephrotic syndrome, aplastic anaemia, uveitis, Kawasaki disease and sarcoidosis.

The compounds of formula (I) are also considered useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

The compounds of formula (I) and pharmaceutically acceptable derivatives thereof are also useful in the treatment of overactive bladder and urge incontenance.

DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ antagonists; EP₂ antagonists and EP₃ antagonists; cannabanoid receptor ligands; bradykinin receptor ligands and vanilloid receptor ligand. When the compounds are used in combination with other therapeutic agents, the compounds may be administered either sequentially or simultaneously by any convenient route.

Additional COX-2 inhibitors are disclosed in U.S. Pat. Nos. 5,474,995 5,633,272; 5,466,823, 6,310,099 and 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A proposed daily dosage of compounds of formula (I) or their pharmaceutically acceptable derivatives for the treatment of man is from 0.01 to 30 mg/kg body weight per day and more particularly 0.1 to 10 mg/kg body weight per day, which may be administered as a single or divided dose, for example one to four times per day The dose range for adult human beings is generally from 8 to 2000 mg/day, such as from 20 to 1000 mg/day, preferably 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

No unacceptable toxicological effects are expected with compounds of the invention when administered in accordance with the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

### Abbreviations

Bn (benzyl), Bu, Pr, Me, Et (butyl, propyl, methyl ethyl), DMSO (dimethyl sulfoxide), DCM (dichloromethane), DME (ethylene glycol dimethyl ether), DMF (N,N-dimethylformamide), EDC (1-(3-dimethylaminopropyl)-3-ethylcarbodiimide), EDTA (ethylenediamine tetraacetic acid), EtOAc (ethyl acetate), EtOH (ethanol), HPLC (High pressure liquid chromatography), LCMS (Liquid chromatography/Mass spectroscopy), MDAP (Mass Directed Purification), MeCN (acetonitrile), MeOH (methanol), NMR (Nuclear Magnetic Resonance (spectrum)), Ph (phenyl), pTSA (para-toluene sulphonic acid), SPE (Solid Phase Extraction), TBAF (tetrabutylammonium fluoride), THF (tetrahydrofuran), s, d, t, q, m, br (singlet, doublet, triplet, quartet, multiplet, broad.)

### LCMS

Column: 3.3 cm×4.6 mm ID, 3 um ABZ+PLUS
Flow Rate: 3 ml/min
Injection Volume: 5 μl
Temp: RT
UV Detection Range: 215 to 330 nm

| Solvents: | A: 0.1% Formic Acid + 10 mMolar Ammonium Acetate. | | |
|---|---|---|---|
| | B: 95% Acetonitrile + 0.05% Formic Acid | | |
| Gradient: | Time | A % | B % |
| | 0.00 | 100 | 0 |
| | 0.70 | 100 | 0 |
| | 4.20 | 0 | 100 |
| | 5.30 | 0 | 100 |
| | 5.50 | 100 | 0 |

### Mass Directed Autopreparation

### Hardware:

Waters 600 gradient pump
Waters 2767 inject/collector

### Waters Reagent Manager

Micromass ZMD mass spectrometer
Gilson Aspec—waste collector
Gilson 115 post-fraction UV detector

### Software:

Micromass Masslynx version 4.0

### Column

The column used is typically a Supelco LCABZ++ column whose dimensions are 20 mm internal diameter by 100 mm in length. The stationary phase particle size is 5 μm.

### Solvents:

A: Aqueous solvent=Water+0.1% Formic Acid
B: Organic solvent=MeCN:Water 95:5+0.05% Formic Acid
Make up solvent=MeOH:Water 80:20+50 mMol Ammonium Acetate
Needle rinse solvent=MeOH:Water:DMSO 80:10:10

The method used depends on the analytical retention time of the compound of interest. 15-minute runtime, which comprises a 10-minute gradient followed by a 5-minute column flush and re-equilibration step.

### MDP 1.5-2.2=0-30% B

### MDP 2.0-2.8=5-30% B

### MDP 2.5-3.0=15-55% B

### MDP 2.84.0=30-80% B

### MDP 3.8-5.5=50-90% B

Flow rate:
flow rate 20 ml/min.

### Preparation of Intermediates

### 1-[(Phenylmethyl)oxy]-4-(trifluoromethyl)benzene

A solution of 4-(trifluoromethyl)phenol (8.55 g, 52.78 mmol) in acetone (200 ml) was treated with benzyl bromide (9.87 g, 6.86 ml, 58.05 mmol) and potassium carbonate (10.94 g, 79.16 mmol). The mixture was stirred and heated to reflux under nitrogen for 3 h. After cooling, diethyl ether (400 ml) and water (400 ml) were added and the aqueous phase re-extracted with diethyl ether (100 ml). The combined organic layers were washed with water, dried (MgSO₄) and the solvent removed in vacuo to leave the title compound as a white solid. (12.71 g, 95%)

¹H NMR (CDCl₃) δ: 5.11 (2H, s), 7.03 (2H, d), 7.34-7.44 (5H, m), 7.55 (2H, d).

### 2-Iodo-1-[(phenylmethyl)oxy]-4-(trifluoromethyl)benzene

A solution of 1-[(phenylmethyl)oxy]-4-(trifluoromethyl)benzene (12.71 g, 50.4 mmol) In acetonitrile (300 ml) was stirred under nitrogen and 1-(chloromethyl)-4-fluoro-1,4 diazoniabicyclo[2.2.2]octane bis(tetrafluoroborate) (17.75 g, 50.4 mmol) and iodine (6.4 g, 25.2 mmol) added. The mixture was stirred at room temperature for 88 h. The solvent was evaporated and the residue partitioned between ethyl acetate (400 ml) and water (400 ml). The organic layer was washed with water, dried (MgSO₄) and evaporated to an orange oil which was purified by flash chromatography (silica gel, 5% ethyl acetate: isohexane) to give the title compound as an orange oil (15.07 g, 79%)

¹H NMR (CDCl₃) δ: 5.21 (2H, s), 6.89 (1H, d J), 7.32-7.55 (6H, m), 8.04 (1H, d).

### 1-Chloro-5-iodo-2-methyl-4(methyloxy)benzene

A mixture of 1-chloro-5-iodo-2-methyl-4-(methyloxy)benzene (5.0 g, 32 mmol), 1-(chloromethyl)-4-fluoro-1,4-diazoniabicyclo[2.2.2]octane bis(tetrafluoroborate) (11.3 g, 32 mmol), and iodine (4.06 g, 16 mmol) in dry acetonitrile (100 ml) was stirred at room temperature for 6 hours. The solvent was evaporated at <30° C. The residue was partitioned between ethyl acetate (50 ml) and water (50 ml). The organic phase was dried (MgSO₄) and evaporated to leave the the compound as a yellow gum (9.0 g).

¹H NMR (CDCl₃) δ: 2.31(3H, s), 3.83 (3H, s), 6.65 (1H, s), 7.68 (1H, s).

### 4,5-dichloro-2-iodophenyl methyl ether

The title compound was prepared in a similar manner to 1-Chloro-5iodo-2-methyl-4-(methyloxy)benzene using 4,5-dichlorophenyl methyl ether.

¹H NMR (CDCl₃) δ: 3.87(3H, s), 6.87 (1H, s), 7.82 (1H, s).

### Ethyl 5-iodo-2-methylbenzoate

A solution of 5-amino-2-methylbenzoic acid ethyl ester (500 mg, 2.8 mmol) and iodine (425 mg, 1.68 mmol) in toluene (20 ml) was cooled to 0° C. and treated with t-butyl nitrite (303 mg, 2.94 mmol). The reaction mixture was stirred at 0° C. for 1 hour then at room temperature for 72 hours. The reaction mixture was washed with 10% aqueous sodium thiosulphate (20 ml), and brine (20 ml), dried (MgSO₄) and evaporated. Flash chromatography [silica, iso-hexane/EtOAc, 9:1] gave ethyl 5-iodo-2-methylbenzoate as a brown oil (510 mg).

¹H NMR (CDCl₃) δ: 1.39 (3H, t), 2.53 (3H, s), 4.36 (2H, q), 6.97 (1H, d), 7.37 (1H, d), 8.20 (1H, s).

### Ethyl 2-fluoro-5-iodobenzoate

Ethyl 2-fluoro-5-aminobenzoate (6.5 g, 35.48 mmol) was stirred in 5N hydrochloric acid (60 ml) and cooled to 0° C. Sodium nitrite (2.7 g, 39.03 mmol) in water (5 ml) was added at 0-5° C. The resulting mixture was added to a solution of potassium iodide (7.07 g, 42.58 mmol) in water (50 ml) over 5 minutes. The reaction was stirred at room temperature for 1 hour, then extracted with diethyl ether. The organic solution was washed with water and 5% sodium thiosulphate solution, dried (MgSO₄) and evaporated. The residue was purified by flash chromatography, eluting with 5% ethyl acetate/isohexane to give the title compound as a colourless oil (7.8 g).

¹H NMR (CDCl₃) δ: 1.40 (3H, t), 4.39 (2H, q), 6.91 (1H, dd), 7.79 (1H, td), 8.22 (1H, dd).

### Ethyl 3-fluoro-5-nitrobenzoate

5-Fluoro-3-nitrobenzoic acid (4.8 g, 25.92 mmol) was dissolved in ethanol (50 ml) and sulphuric acid (0.5 ml) added carefully. The mixture was heated to reflux for 16 hours. The solvent was evaporated and the residue dissolved in ethyl acetate and washed with water, 5% sodium bicarbonate solution and brine, dried (MgSO₄) and evaporated. The residue was purified by flash chromatography, eluting with 10% ethyl acetate/isohexane to give the title compound as a yellow oil (2.04 g)

¹H NMR (CDCl₃) δ: 1.44 (3H, t), 4.46 (2H, q), 8.07-8.14(2H, m), 8.69 (1H, s).

### Ethyl 3-amino-5-fluorobenzoate

Ethyl 3-fluoro-5-nitrobenzoate (5.0 g, 23.46 mmol) was dissolved in ethanol (150 ml) and tin(II)chloride (44.24 g, 0.234 mol) added portionwise with stirring. The mixture was stirred at 80° C. for 1 hour. The solvent was evaporated and the residue partitioned between ethyl acetate and 2M sodium hydroxide solution. The resulting glutinous mixture was slowly filtered through a Kieselguhr bed, which was washed copiously with ethyl acetate. The organic phase was washed with water, dried (MgSO₄) and evaporated to give the title compound as a cream solid (3.98 g).

¹H NMR (CDCl₃) δ: 1.38 (3H, t), 3.94 (2H, br s), 4.35 (2H, q), 6.53 (1H, dd), 7.08 (1H, dd), 7.14 (1H, d).

### Ethyl 3-fluoro-5iodobenzoate

Ethyl 3-fluoro-5-aminobenzoate (3.98 g, 21.73 mmol) was stirred in 5N hydrochloric acid (45 ml) and cooled to 0° C. Sodium nitrite (1.65 g, 23.91 mmol) in water (2 ml) was added at 0-5° C. The resulting mixture was added dropwise to a solution of potassium iodide (4.33 g, 26.09 mmol) in water (30 ml) over 20 minutes. The reaction was stirred at room temperature for 1 hour, then extracted with diethyl ether (×2). The organic solution was washed with water and 5% sodium-thiosulphate solution, dried (MgSO₄) and evaporated to give the title compound as an orange oil (5.0 g).

¹H NMR (CDCl₃) δ: 1.40 (3H, t), 4.39 (2H, q), 7.62 (1H, dd), 7.69 (1H, td), 8.17 (1H, s).

### Ethyl 3-amino-5-nitrobenzoate

3-Amino-5-nitrobenzoic acid (10.0 g, 54.9 mmol) was dissolved in ethanol (100 ml) and treated with conc. sulphuric acid (5 ml). The mixture was heated at reflux overnight. After cooling the ethanol was removed in vacuo and the residue was dissolved in diethyl ether. The solution was basified with saturated aqueous sodium bicarbonate and the layers separated. The aqueous layer was further extracted with diethyl ether (×3) and the combined organic extracts were dried (Na₂SO₄) and concentrated in vacuo to give the ester (6.5 g).

¹H NMR (CDCl₃) δ: 1.41 (3H, t), 4.15 (2H, br s), 4.40 (2H, q), 7.60-7.68 (2H, m), 8.20 (1H, s).

### Ethyl 3-iodo-5-nitrobenzoate

Ethyl 3-amino-5-nitrobenzoate (6.5 g, 30.9mmol) was suspended in 5M aqueous HCl (50 ml), cooled to 0° C. and sodium nitrite (2.34 g, 33.9 mmol) in water (4 ml) was added slowly. The resulting solution of the diazonium salt was added slowly to a solution of potassium iodide (6.16 g, 37.1 mmol) in water (40 ml), and the resulting mixture was stirred at room temperature for 1 hour. The mixture was extracted with diethyl ether, and the extract was washed with water and aqueous sodium thiosulphate solution, dried (Na₂SO₄) and concentrated in vacuo. The residue was purified by flash chromatography on silica (gradient elution, 10-20% ethyl acetate/cyclohexane) to give the title compound (5.46 g).

¹H NMR (CDCl₃) δ: 1.44 (3H, t), 4.46 (2H, q), 8.68 (1H, t), 8.73 (1H, t), 8.81 (1H, t).

### Ethyl 3-amino-5-iodobenzoate

Ethyl 3-iodo-5-nitrobenzoate (4.45 g, 13.9 mmol) was dissolved in ethanol and tin (II) chloride (27 g, 146 mmol) was added. The mixture was heated to reflux for 2 hours. After cooling, the reaction was concentrated in vacuo. The residue was partitioned between ethyl acetate and aqueous sodium hydroxide solution, and the aqueous extracted with further ethyl acetate. The combined extracts were washed with water, dried (Na₂SO₄) and concentrated in vacuo to give the title compound as a yellow oil which slowly crystallised (3.56 g). LC/MS Rt=3.23 min [MH⁺] 292.

¹H NMR (CDCl₃) δ: 1.38 (3H, t), 3.80 (2H, br s), 4.45 (2H, q), 7.20 (1H, t), 7.30 (1H, t), 7.74 (1H, t).

### 3-Bromo-5-chloro-2(1H)-pyridinone

5-Chloro-2-pyridinol (5.18 g, 40 mmol) was dissolved in glacial acetic acid (50 ml) and bromine (7.51 g, 2.41 ml, 47 mmol) added dropwise. The mixture was stirred at room temperature for 48 hours. Ethyl acetate and water were added and the organic layer washed with water (×3), dried (MgSO₄) and evaporated. The residue was triturated with diethyl ether and the buff solid filtered and dried (5.59 g).

¹H NMR (CDCl₃) δ: 7.52 (1H, d), 7.87 (1H, d).

### 3-Bromo-5-chloro-2-[(phenylmethyl)oxy]pyridine

3-Bromo-5-chloro-2-pyridinol (7.0 g, 33.6 mmol) was stirred in toluene (160 ml) and silver carbonate (10.23 g, 36.9 mmol) added, followed by benzyl bromide (6.32 g, 4.39 ml, 36.9 mmol). The mixture was heated to reflux for 1 hour. After cooling, the mixture was filtered, washed with water (×2), dried (MgSO₄) and evaporated. The residue was triturated with isohexane and the pale yellow solid filtered and dried. (8.36 g).

¹H NMR (CDCl₃) δ: 5.43 (2H, s), 7.32-7.48 (5H, m), 7.82 (1H, d), 8.04 (1H, d).

### 5-Chloro-3-iodo-2-[(phenylmethyl)oxy]pyridine

5-Chloro-3-iodo-2(1H)-pyridinone (6.69 g, 26.18 mmol) was dissolved in toluene (125 ml) and silver carbonate (7.97 g, 28.8 mmol) added, followed by benzyl bromide (3.43 ml, 28.8 mmol). The mixture was stirred and heated to reflux for 2 hours. The mixture was cooled, filtered through a Kieselguhr pad and the solvent evaporated. The residue was triturated with isohexane containing a trace of diethyl ether and the title compound filtered and dried in vacuo (6.8 g).

¹H NMR (CDCl₃) δ: 5.41 (2H, s), 7.32-7.49 (5H, m), 8.03 (1H, d), 8.06 (1H, d).

### 3-Iodo-2-[(phenylmethyl)oxy]-5-(trifluoromethyl)pyridine

The title compound was prepared in a similar manner to 5-chloro-3-iodo-2-[(phenylmethyl)oxy]pyridine using 3-iodo-5-(trifluoromethyl)-2(1H)-pyridinone.

¹H NMR (CDCl₃) δ: 5.49 (2H, s), 7.33-7.50 (5H, m), 8.23 (1H, d), 8.39 (1H, d).

### Ethyl 3-bromo-5-fluorobenzoate

3-Bromo-5-fluorobenzoic acid (ex. Fluorochem) (6.0 g, 22.8 mmol) was dissolved in ethanol (50 ml) and treated with conc. sulphuric acid (2.5 ml). The mixture was heated at reflux overnight. After cooling the ethanol was removed in vacuo and the residue was dissolved in diethyl ether. The solution was basified with saturated aqueous sodium bicarbonate, and the layers separated. The aqueous layer was further extracted with diethyl ether (×3), and the combined organic extracts were dried (Na₂SO₄) and concentrated in vacuo to give the ester (6.17 g).

¹H NMR (CDCl₃) δ: 1.41 (3H, t), 4.40 (2H, q), 7.44 (1H, dt), 7.68 (1H, ddd), 7.99 (1H, s).

### Ethyl 3-amino-5-nitrobenzoate

3-Amino-5-nitrobenzoic acid (ex Lancaster) (10.0 g, 54.9 mmol) was dissolved in ethanol (100 ml) and treated with conc. sulphuric acid (5 ml). The mixture was heated at reflux overnight. After cooling the ethanol was removed in vacuo and the residue was dissolved in diethyl ether. The solution was basified with saturated aqueous sodium bicarbonate, and the layers separated. The aqueous layer was further extracted with diethyl ether (×3), and the combined organic extracts were dried (Na₂SO₄) and concentrated in vacuo to give the ester (6.5 g).

¹H NMR (CDCl₃) δ: 1.41 (3H, t), 4.15 (2H, br s), 4.40 (2H, q), 7.60-7.68 (2H, m), 8.20 (1H, s).

### Ethyl 3-iodo-5-nitrobenzoate

Ethyl 3-amino-5-nitrobenzoate (6.5 g, 30.9 mmol) was suspended in 5M aqueous HCl (50 ml), cooled to 0° C., and treated with aqueous sodium nitrite (2.34 g 33.9 mmol in 4 ml water) added slowly. The resulting solution of the diazonium salt was added slowly to a solution of potassium iodide (6.16 g, 37.1 mmol) in water (40 ml), and the resulting mixture was stirred at room temperature for 1 hour. The mixture was extracted with diethyl ether, and the extract was washed with water, aqueous sodium thiosulphate solution, dried (Na₂SO₄) and concentrated in vacuo. The residue was purified by flash chromatography on silica (gradient elution, 10-20% ethyl acetate/cyclohexane) to give the title compound (5.46 g).

¹H NMR (CDCl₃) δ: 1.44 (3H, t), 4.46 (2H, q), 8.68 (1H, t), 8.73 (1H, t), 8.81 (1H, t).

### Ethyl 3-amino-5-iodobenzoate

Ethyl 3-iodo-5-nitrobenzoate (4.45 g, 13.9 mmol) was dissolved in ethanol and tin (II) chloride (27 g, 146 mmol) was added. The mixture was heated to reflux for 2 hours, by which time LC/MS analysis showed that reaction was complete. After cooling, the reaction was concentrated in vacuo. The residue was partitioned between ethyl acetate and aqueous sodium hydroxide solution, and the aqueous extracted with further ethyl acetate. The combined extracts were washed with water, dried (Na₂SO₄) and concentrated in vacuo to give the title compound as a yellow oil which slowly crystallised (3.56 g).

¹H NMR (CDCl₃) δ: 1.38 (3H, t), 3.80 (2H, br s), 4.45 (2H, q), 7.20 (1H, t), 7.30 (1H, t), 7.74 (1H, t). LC/MS Rt=3.23 min [MH⁺] 292.

### Ethyl 3,6-dichloro-2-pyridinecarboxylate

3,6-Dichloro-2-pyridinecarboxylic acid (530 mg, 2.76 mmol) was dissolved in a mixture of ethanol (20 ml) and sulphuric acid (0.25 ml) and refluxed for 2 hours then left at room temperature for 3 days. The resulting solution was evaporated and the residue dissolved in diethyl ether/water and basified with potassium carbonate. The organic layer was dried (magnesium sulphate) and evaporated to give a colourless oil (602 mg).

LC/MS t=2.56, [MH+] 220.3

### Ethyl 3-methyl-2-pyridinecarboxylate 1-oxide

A solution of ethyl 3-methyl-2-pyridinecarboxylate (12.1 g, 73 mmol) and 3-chloroperbenzoic acid (28 g, 50-55%, 80 mmol) in dichloromethane (200 ml) was left at room temperature for 16 hours then washed with sodium thiosulphate solution and sodium bicarbonate solution. The organic solution was dried (magnesium sulphate) and evaporated to give a light coloured oil (12.2 g). LC/MS Rt=1.39, [MH+] 182.3

### Ethyl 6-chloro-3-methyl-2-pyridinecarboxylate

Ethyl 3-methyl-2-pyridinecarboxylate 1-oxide (12.1 g, 66.85 mmol) was added in portions with water bath cooling to phosphorus oxychloride (50 ml) and the resulting mixture stirred for 30 minutes and evaporated to dryness. The residue was dissolved in diethyl ether/water and basified with 2M sodium hydroxide solution. The organic layer was separated, dried (magnesium sulphate), evaporated and purified by chromatography on silica eluting with ethyl acetate/iso-hexane (1:9) to give a colourless oil (2.4 g).

LC/MS Rt=2.52, [MH+] 200.3, 202.3

¹H NMR (CDCl₃) δ: 1.43 (3H, t), 2.54 (3H, s), 4.44 (2H, q), 7.35 (1H, d), 7.57 (1H, d).

### Methyl 5-chloro-2-ethyl-3-pyridinecarboxylate

Potassium-tert-butoxide (1.176 g, 10.5 mmol) was added slowly to a stirring solution of methyl 3-oxopentanoate (1.30 g, 10 mmol) in tetrahydrofuran (33 ml) and stirred for 45 minutes before adding 2-chloro-1,3-bis(dimethylamino)trimethinium hexafluorophosphate (4.6 g, 15.00 mmol) and 1,4-diazabicyclo(2.2.2) octane (1.12 g, 10 mmol) and stirring at 45° C. for 3 hours. Ammonium acetate (1.54 g, 20 mmols) was added and the reaction mixture was refluxed for 6 hours. The reaction mixture was cooled to room temperature and diluted with diethyl ether and water. The ether layer was separated, dried over magnesium sulphate and evaporated to dryness to give the title compound as a yellow oil. 1.24 g, 62%. LC/MS: Rt=2.65 min, [M+H] 200.

### Methyl 5-bromo-2-(trifluoromethyl)-3-pyridinecarboxylate

(Trimethylsilyl)diazomethane (2M solution in hexanes, 5 ml, 10 mmol) was added to a solution of 5-bromo-2-(trifluoromethyl)-3-pyridinecarboxylic acid (Eur. J. Org. Chem. 2002, 327-330) (2.05 g, 7.59 mmol) in tetrahydrofuran (10 ml). The resulting solution was evaporated to dryness and the residue purified by chromatography on silica eluting with ethyl acetate/iso-hexane (1:19) to give 950 mg of pale coloured oil.

¹H NMR (CDCl₃) δ: 3.84 (3H, s), 8.12 (1H, d), 8.71 (1H, d).

### Ethyl 6-chloro-4-(trifluoromethyl)-2-pyridinecarboxylate

A mixture of 6-chloro-4-(trifluoromethyl)-2-pyridinecarboxylic acid (5 g, 22.16 mmol) sulphuric acid (5 ml) and ethanol (80 ml) was stirred and refluxed for 14 hours then cooled and evaporated. The residue was dissolved in ether/water and basified with aqueous ammonia. The organic layer was dried (magnesium sulphate) and evaporated to give the title compound as a colourless oil (4.88 g).

LC/MS: [M+H] 254.3, 256.4, Rt=2.98 min

### (2-Bromo-1-cyclopenten-1-yl)boronic acid

1,2-Dibromocyclopentene (10.1 g, 0.044 mol) was dissolved in 100 mL of tetrahydrofuran, cooled to −78° C. and n-butyllithium (1.6 M solution in hexanes; 28 mL, 0.044 mol), was added dropwise over 20 minutes under nitrogen. The mixture was stirred at −78° C. for 20 minutes, then triisopropylborate (20.8 mL, 0.089 mol) was added dropwise. The cooling bath was then removed and the reaction mixture was allowed to reach room temperature. The reaction mixture was then quenched with 1M HCl (40 mL) and stirred vigorously at room temperature for 15 minutes. The organic layer was separated, dried over magnesium sulphate and evaporated down. The residue was triturated with dichloromethane to yield the title compound as a white solid (2.2 g, 26%).

¹H NMR (CDCl₃) δ: 1.92-1.98 (2H, m), 2.50-2.55 (2H, m), 2.73-2.78 (2H, m), 5.02 (2H, s).

### [2-(Methyloxy)-5-(trifluoromethyl)phenyl]boronic acid

2-Bromo-1-methoxy-4-(trifluoromethyl)benzene (20 g, 78 mmol) was dissolved in dry Et₂O (300 ml) and cooled to −70° C., n-butyllithium (1.6M solution in hexanes; 53.4 ml, 86 mmol) was added slowly keeping the temperature at about −70° C. and the reaction stirred for 30 minutes. Tri-isopropyl borate (36.2 ml, 0.16 mol) was added slowly keeping the temperature at about −70° C. and the reaction allowed to warm to RT and stirred under nitrogen for 16 hours. 2N HCl (300 ml) was added and the reaction stirred vigorously for 3 hours. The reaction was diluted with EtOAc and the organics separated, the aqueous washed with 3×EtOAc. The combined organics were then washed with brine, dried over MgSO4, filtered and concentrated in vacuo to yield a yellow oil, this was triturated in iso-hexane to yield a white solid (14.6 g, 85%). LC/MS Rt=2.57.

### {5-Chloro-2-[(Phenylmethyl)oxy]-3-pyridinyl}boronic acid

a) 3-Bromo-5-chloro-2-[(phenylmethyl)oxy]pyridine (3.65 g, 12.21 mmol) was dissolved in diethyl ether (80 ml) and added dropwise to a stirring solution of 1.6M n-butyllithium in hexanes (9.16 ml, 14.6 mmol) in diethyl ether (20 ml) at −78° C. under nitrogen over 30 minutes. The mixture was stirred at −78° C. for 1 hour. Triisopropyl borate (3.37 ml, 14.6 mmol) in diethyl ether (10 ml) was added dropwise over 10 minutes at −78° C. The reaction was allowed to warm to room temperature then stirred for 1 hour. 2M sodium hydroxide solution (100 ml) was added and the mixture stirred for 15 minutes. The layers were separated and the organic layer re-extracted with 2M sodium hydroxide solution (50 ml). The combined aqueous layers were acidified to pH6 with 2M hydrochloric acid solution at <10° C. and extracted with ethyl acetate (×2). The combined organic phases were washed with water, dried (MgSO₄) and evaporated to a white solid (1.83 g).

¹H NMR (CDCl₃) δ: 5.45 (2H, s), 5.71 (2H, s), 7.36-7.45 (5H, m), 8.09 (1H, d), 8.20 (1H, d).

b) 5-Chloro-3-iodo-2-[(phenylmethyl)oxy]pyridine (3.35 g, 9.7 mmol) was dissolved in tetrahydrofuran (50 ml) under nitrogen and cooled to −40° C. 2M isopropyl magnesium chloride solution in diethyl ether (9.7 ml, 19.4 mmol) was added dropwise at 40° C. and the mixture stirred at 40° C. for 15 minutes, then cooled to −78° C. Trimethyl borate (2.02 g, 2.23 ml, 19.4 mmol) was added dropwise at −78° C. and the reaction was stirred and allowed to warm to room temperature over 2 hours. 2M sodium hydroxide solution (50 ml) was added and the mixture stirred for 15 minutes. The organic layer was re-extracted with 2M sodium hydroxide solution (20 ml) and the combined aqueous layers acidified with glacial acetic acid and extracted with diethyl ether (×2). The combined organic phases were washed with water, dried (MgSO₄) and evaporated. The residue was triturated with isohexane, filtered and dried in vacuo to give the title compound (2.13 g).

¹H NMR (CDCl₃) δ: 5.45 (2H, s), 5.71 (2H, s), 7.36-7.45 (5H, m), 8.09 (1H, d), 8.20 (1H, d).

### [5-Bromo-2-(methyloxy)-3-pyridinyl]boronic acid

The title compound was prepared in a similar manner to {5-chloro-2-[(phenylmethyl)oxy]-3-pyridinyl}boronic acid using 3,5-dibromo-2-(methyloxy)pyridine.

¹H NMR (DMSOd₆) δ: 3.85 (3H, s), 7.92 (1H, d), 8.11 (2H, s), 8.29 (1H, d).

### [2-[(Phenylmethyl)oxy]-5-(trifluoromethyl)-3-pyridinyl]boronic acid

3-Iodo-2-(phenylmethoxy)-5-(trifluoromethyl)pyridine (15.0 g, 39.5 mmol) was dissolved in tetrahydrofuran (90 mL) under nitrogen and cooled to −40° C. Isopropyl magnesium chloride solution in diethyl ether (2.0M, 39.5 mL, 79 mmol) was added dropwise at −40° C. and the mixture stirred at 40° C. for 15 minutes, then cooled to −78° C. Trimethyl borate (8.9 mL, 8.25 g, 79.4 mmol) was added dropwise at −78° C. and the reaction was stirred and allowed to warm to room temperature over 18 hours. 2M aqueous sodium hydroxide solution was added and the layers were separated. The organic phase was dried (MgSO₄) and concentrated in vacuo. The residue was triturated with dichloromethane, and the solid material was collected by filtration and dried in vacuo to give the title compound (10.53 g).

LC/MS Rt=3.45 min [MH⁺] 298.

### 2-(2-Bromo-1-cyclopenten-1-yl)-4-chloro-1-(methyloxy)benzene

4-Chloro-2-iodoanisole (16.8 g, 0.062 mol), (2-bromo-1-cyclopenten-1-yl)boronic acid (12 g, 0.062 mol), potassium carbonate (35 g, 0.25 mol) and tetrakis(triphenylphosphine)palladium(0) (3.6 g, 0.003 mol) were dissolved in toluene-ethanol (1:1300 mL) and stirred at 90° C., under nitrogen, for 2 hrs. Upon cooling, the reaction mixture was poured into water and extracted with ethyl acetate (150 mL×3). The organic layers were dried (MgSO₄), filtered and concentrated. The residue was purified by flash chromatography using 2% ethyl acetate/iso-hexane to give a clear oil that was recrystallized from iso-hexane at 0-4° C. to give the required product as a white solid (7.55 g).

¹H NMR (CDCl₃) δ: 12.01-2.09 (2H, m), 2.65-2.69 (2H, m), 2.77-2.81 (2H, m), 3.79 (3H, s), 6.79-6.82 (1H, m), 7.2-7.25 (2H, m).

The following intermediates were prepared by a similar route to 2-(2-bromo-1-cyclopenten-1-yl)-4-chloro-1-(methoxy)benzene from the appropriate intermediates.

| Name | Data |
|---|---|
| 1-(2-Bromo-1-cyclopenten-1-yl)-5-chloro-4- | ¹H NMR: CDCl₃ 2.00-2.08(2H, m), |
| methyl-2-(methyloxy)benzene | 2.34(3H, s), 2.65(2H, t), 2.78(2H, t), |
| | 3.78(3H, s), 6.74(1H, s), 7.21(1H, s). |
| 3-(2-Bromo-1-cyclopenten-1-yl)-2- | ¹H NMR: (CDCl₃) δ: 2.03-2.11(2H, m), |
| (methyloxy)pyridine | 2.69-2.74(2H, m), 2.78-2.83(2H, m), |
| | 3.95(3H, s), 6.90(1H, dd), 7.58(1H, |
| | dd), 8.12(1H, dd). |
| 2-(2-Bromo-1-cyclopenten-1-yl)-1- | ¹H NMR: CDCl₃ 2.02-2.09(2H, m), |
| [(phenylmethyl)oxy]-4- | 2.70-2.75(2H, t), 2.78-2.82(2H, t), |
| (trifluoromethyl)benzene | 5.14(2H, s), 6.98(1H, d), 7.33- |
| | 7.40(5H, m), 7.48(1H, dd), 7.54(1H,d). |
| 2-(2-Bromo-1-cyclopenten-1-yl)-4-chloro-1- | ¹H NMR: CDCl₃ 1.99-2.07(2H, m), |
| [(phenylmethyl)oxy]benzene | 2.67-2.72(2H, t), 2.76-2.81(2H, t), |
| | 5.06(2H, s), 6.84(1H, d), 7.18(1H, dd), |
| | 7.24-7.38(6H, m). |
| 2-(2-bromo-1-cydopenten-1-yl)-4,5- | ¹H NMR: CDCl₃ 2.01-2.09(2H, m), |
| dichlorophenyl methyl ether | 2.63-2.65(2H, m), 2.77-2.80(2H, m), |
| | 3.79(3H, s), 6.95(1H, s), 7.31(1H, s). |

### 2-(2-Bromo-1-cyclopenten-1-yl)-1-(methyloxy)-4-(trifluoromethyl)benzene

[2-Methoxy-5-(trifluoromethyl)phenyl]boronic acid (20 g, 90.9 mmol), 1,2-dibromocyclopentene (32.5 ml, 0.27 mol), potassium carbonate (62.8 g, 0.45 mol) and tetrakis(triphenylphosphine)palladium(0) were refluxed in 1:1 ethanol/toluene (900 ml), in the dark, under a nitrogen atmosphere, for 2 hours. After cooling the reaction was filtered over celite and the solvent removed in vacuo, the residue was taken up in ethyl acetate and washed with water and brine, dried over MgSO4, filtered and concentrated in vacuo to yield a dark oil. This was purified by column chromatography eluting with isohexane. This yielded the title compound as a yellow oil (26.79, 61%). LC/MS Rt=3.88.

### 2-(2-Bromo-1-cyclopenten-1-yl)-4-fluoro-1-(methyloxy)benzene

Procedure as for 2-(2-bromo-1-cyclopenten-1-yl)-1-(methyloxy)-4-(trifluoromethyl)benzene starting from [5-fluoro-2-(methyloxy)phenyl]boronic acid. LC/MS Rt=3.70, [MH] 270.

### {2-[2-(Methyloxy)-5-(trifluoromethyl)phenyl]-1-cyclopenten-1-yl}boronic acid

2-(2-Bromo-1-cyclopenten-1-yl)-1-(methyloxy)-4-(trifluoromethyl)benzene (26 g, 81.3 mmol) was dissolved in dry THF (350 ml) and the solution cooled to −70° C. n-butyllithium (1.6M solution in hexanes; 101.6 ml, 0.16 mol) was added slowly keeping the temperature below −65° C. and the reaction allowed to stir for 45 minutes. Tri-isopropyl borate (37.5 ml, 0.16 mol) was added slowly keeping the temperature below 60° C. and the cooling removed and the reaction stirred under nitrogen at RT for a further 15 hours. 2N HCl (300 ml) was added and the reaction stirred at RT for a further 2 hours. The reaction was diluted with ethyl acetate and the organics separated, the aqueous washed with ethyl acetate (×3). The combined organics were then washed with brine, dried over MgSO4, filtered and concentrated in vacuo to yield a yellow oil. This was purified by column chromatography on a 75 L Biotage column eluting in 40% ethyl acetate/isohexane. This yielded the title compound as a white solid. LC/MS Rt=2.96.

The following intermediates were prepared by a similar route to {2-[2-(methyloxy)-5-(trifluoromethyl)phenyl]-1-cyclopenten-1-yl}boronic acid from the appropriate intermediates.

| Name | Data |
|---|---|
| {2-[2-(Methyloxy)phenyl]-1-cyclopenten-1- | LC/MS Rt = 3.69 min. |
| yl}boronic acid | [2MH⁺] 417.2 |
| {2-[5-Fluoro-2-(methyloxy)phenyl]-1- | LC/MS Rt = 2.52 min. |
| cyclopenten-1-yl}boronic acid | |
| {2-[5-Chloro-2-(methyloxy)phenyl]-1- | ¹H NMR (CDCl₃) δ: 1.91-1.98 (2H, |
| cyclopenten-1-yl}boronic acid | m), 2.66-2.73 (4H, m), 3.80 (3H, |
| | s), 4.30 (2H, s), 6.85 (1H, s), 7.16 |
| | (1H, s), 7.21 (1H, dd). |
| {2-[2-[(Phenylmethyl)oxy]-5- | LC/MS: Rt = 3.44 min, [M + H₂O] |
| (trifluoromethyl)phenyl]-1-cyclopenten-1-yl} | 380, [2M] 724 |
| boronic acid | |
| {2-[5-Chloro-4-methyl-2-(methyloxy)phenyl]-1- | ¹H NMR (CDCl₃) δ: 1.90-1.97(2H, |
| cyclopenten-1-yl}boronic acid | m), 2.36(3H, s), 2.65-2.71(4H, m), |
| | 3.79(3H, s), 4.41(2H, s), 6.78(1H, |
| | s), 7.14(1H, s). |
| (2-{5-Chloro-2-[(phenylmethyl)oxy]phenyl}-1- | LC/MS: Rt = 3.39 min, [2MH] 637 |
| cyclopenten-1-yl)boronic acid | |
| {2-[4,5-dichloro-2-(methyloxy)phenyl]-1- | LCMS: Rt = 3.13 min |
| cyclopenten-1-yl}boronic acid | |

### 5-(2-Bromo-1-cyclopenten-1-yl)-N-(1,1-dimethylethyl)-3-pyridazinecarboxamide

(2-Bromo-1-cyclopenten-1-yl)boronic acid (0.6 g, 3.2 mmol), N-(1,1-dimethylethyl)-5-iodo-3-pyridazinecarboxamide (1.0 g, 3.2 mmol), tetrakis(triphenylphosphine)palladium(0) (200 mg, 0.172 mmol) and potassium carbonate (1.1 g, 8 mmol) in toluene/ethanol (1:1, 10 ml) were refluxed overnight under nitrogen in the dark. The reaction mixture was then filtered through celite, and chromatographed with diethyl ether/iso-hexane gradient giving (0.78 g, 71% yield).

LC/MS Rt=3.13 min [MH⁺] 326, 327

### Ethyl 6-(2bromo-1-cyclopenten-1-yl)-3-chloro-2pyridinecarboxylate

A mixture of ethyl 3,6-dichloro-2-pyridinecarboxylate (220 mg, 1 mmol), (2-bromo-1-cyclopenten-1-yl)boronic acid (191 mg, 1 mmol), potassium carbonate (552 mg, 4 mmol) and tetrakis(triphenylphosphine)palladium(0) (58 mg, 0.05 mmol) in 1:1 ethanol/toluene (4 ml) was stirred and heated at 90° C. under nitrogen for 2 hours after cooling the mixture was dissolved in diethyl ether/water and the organic phase dried (magnesium sulphate) evaporated and the residue purified by chromatography on silica eluting with ethyl acetate/iso-hexane (1:19) to give 110 mg of colourless oil.

LC/MS t=3.81, [MH+] 332.3.

The following compounds were prepared by a similar route to ethyl 6-(2-bromo-1-cyclopenten-1-yl)-3-chloro-2-pyridinecarboxylate from the appropriate intermediates.

| Name | Data |
|---|---|
| Ethyl 6-(2-bromo-1-cyclopenten-1-yl)-2- | LC/MS: Rt = 3.07 min. [M + H] = 297, |
| pyrazinecarboxylate | 299. |
| Ethyl 6-(2-bromo-1-cyclopenten-1-yl)-2- | LC/MS: Rt = 3.27 min. [M + H] = |
| pyridinecarboxylate | 296, 298. |
| Ethyl 3-(2-bromo-1-cyclopenten-1- | Rt = 3.98 min. [MH⁺] 295, 297. |
| yl)benzoate | |
| Ethyl 5-(2-bromo-1-cyclopenten-1-yl)-2- | ¹H NMR (CDCl₃) δ: 1.39(3H, t), 2.01- |
| methylbenzoate | 2.08(2H, m), 2.59(3H, s), |
| | 2.77(2H, m), 2.85(2H, m), 4.36(2H, |
| | q), 7.24(1H, t), 7.65(1H, d), 8.12(1H, |
| | s). |
| Ethyl 5-(2-bromo-1-cyclopenten-1-yl)-2- | Rt = 3.82 min. [MH⁺] 313, 315. |
| fluorobenzoate | |
| Ethyl 3-(2-bromo-1-cyclopenten-1-yl)-5- | Rt = 3.91 min. [MH⁺] 313, 315. |
| fluorobenzoate | |
| Ethyl 3-amino-5-(2-bromocyclopent-1- | LC/MS Rt = 3.51 min [MH⁺] 310, 312. |
| enyl)benzoate | |
| Ethyl 2-amino-5-(2-bromo-1-cyclopenten-1- | ¹H NMR (CDCl₃) δ: 1.39 (3H, t, |
| yl)benzoate | J = 7 Hz), 1.98-2.06 (2H, m), 2.71-2.76 |
| | (2H, m), 2.81-2.86 (2H, m) 4.33 (2H, |
| | q, J = 7 Hz), 5.80 (2H, br s), 6.65 (1H, |
| | d, J = 9 Hz), 7.65 (1H, dd, J = 9 Hz, |
| | 2 Hz), 8.14 (1H, d, J = 2 Hz). |

### Ethyl 5-(2-bromocyclopent-1-enyl)-2-fluorobenzoate

Ethyl 2-fluoro-5-iodobenzoate (4.7 g, 16.0 mmol), 2-bromo-cyclopent-1-enylboronic acid (3.06 g, 16.0 mmol), potassium carbonate (15.5 g, 112 mmol) and Pd(PPh₃)₄ (0.925 g, 0.8 mmol) were dissolved in toluene-ethanol (1:1, 110 mL) and stirred at 100° C. under nitrogen for 1.5 hours. Upon cooling, the reaction mixture was diluted with diethyl ether, and washed with water. The aqueous layer was extracted with further diethyl ether, and the combined organic extracts were dried (MgSO₄), and concentrated in vacuo. The residue was purified by flash chromatography on silica (gradient elution, 1-5% ethyl acetate/cyclohexane) to give the required product as a yellow oil (3.84 g).

LC/MS Rt=3.80 min [MH⁺] 313, 315.

### Ethyl 3-(2-bromocyclopent-1-enyl)-5-fluorobenzoate

Ethyl 3-bromo-5-fluorobenzoate (5.17 g, 20.9 mmol), 2-bromo-cyclopent-1-enylboronic acid (3.99 g, 20.9 mmol), potassium carbonate (23 g, 167 mmol) and Pd(PPh₃)₄ (1.1 g, 1.0 mmol) were dissolved in toluene-ethanol (1:1, 150 mL) and heated to reflux for 1.5 hours under a nitrogen atmosphere. Upon cooling, the reaction mixture was diluted with diethyl ether, and washed with water. The aqueous layer was extracted with further diethyl ether, and the combined organic extracts were dried (Na₂SO₄), and concentrated in vacuo. The residue was purified by flash chromatography on silica (gradient elution, 0-5% ethyl acetate/cyclohexane) to give the required product as a yellow oil (5.93 g).

LC/MS Rt=3.93 min [MH⁺] 313, 315.

### Ethyl 3-amino-5-(2bromocyclopent-1-enyl)benzoate

Ethyl 3-amino-5-iodobenzoate (3.66 g, 12.6 mmol), 2-bromo-cyclopent-1-enylboronic acid (2.41 g, 12.6 mmol), potassium carbonate (12.2 g, 88.2 mmol) and Pd(PPh₃)₄ (0.73 g, 0.63 mmol) were dissolved in toluene-ethanol (1:1, 50 mL) and heated to reflux for 1.75 hours under a nitrogen atmosphere. After cooling, the reaction mixture was diluted with diethyl ether, and washed with water. The aqueous layer was extracted with further diethyl ether, and the combined organic extracts were dried (Na₂SO₄), and concentrated in vacuo. The residue was purified by flash chromatography on silica (gradient elution, 0-5% ethyl acetate/cyclohexane) to give the required product (4.21 g).

LC/MS Rt=3.51 min [MH⁺] 310, 312.

### Ethyl 6-{2-[5-chloro-2-(methyloxy)phenyl]-1-cyclopenten-1-yl}-2-pyridinecarboxylate

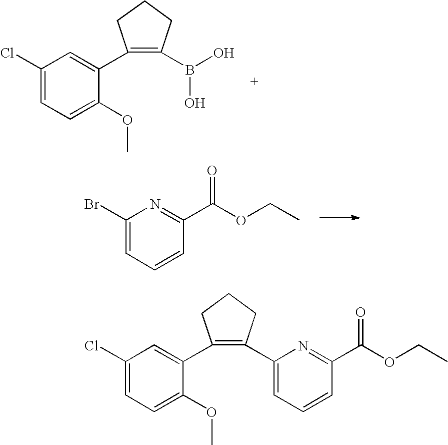

A mixture of ethyl 6-bromo-2-pyridinecarboxylate (4.1 g, 17.8 mmol), {2-[5-chloro-2-(methyloxy)phenyl]-1-cyclopenten-1-yl}boronic acid (4.1 g, 16 mmol), potassium carbonate (11.2 g, 81 mmol) and tetrakis(triphenylphosphine)palladium(0) (1.88 g, 1.6 mmol) was stirred and heated in 1:1 toluene/ethanol (50 ml) at 90° C. under nitrogen for 2 hours. After cooling the mixture was diluted with ethyl acetate/water and the organic phase dried (magnesium sulphate), evaporated to dryness and the residue purified by chromatography (12% ethyl acetate in iso-hexane) to yield the title compound as a clear oil (4 g).

LC/MS: Rt 3.8 [MH+] 358, 361

The following compounds were prepared by a similar route to ethyl 6-{2-[5-chloro-2-(methyloxy)phenyl]-1-cyclopenten-1-yl}-2-pyridinecarboxylate from the appropriate intermediates.

| | Name | Data |
|---|---|---|
| 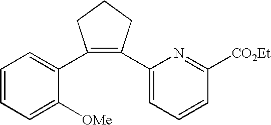 | Ethyl 6-{2-[2-(methyloxy)phenyl]-1- cyclopenten-1-yl}-2- pyridinecarboxylate | LC/MS: Rt = 3.53 min, [M + H] 324. |
| 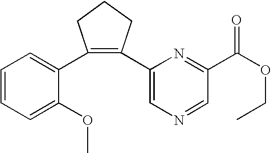 | Ethyl 6-{2-[2-(methyloxy)phenyl]-1- cyclopenten-1-yl}-2- pyrazinecarboxylate | LC/MS: Rt = 3.47 min, [M + H] 325. |
| 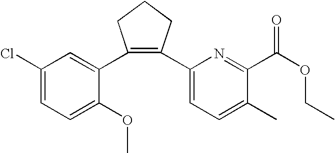 | Ethyl 6-{2-[5-chloro-2- (methyloxy)phenyl]-1- cyclopenten-1- yl}-3-methyl-2-pyridinecarboxylate | LC/MS: Rt = 3.98, [MH+] 372.4, 374.5 |
| 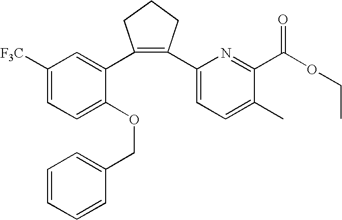 | Ethyl 3-methyl-6-{2-[2- [(phenylmethyl)oxy]-5- (trifluoromethyl)phenyl]-1- cyclopenten-1-yl}-2- pyridinecarboxylate | LC/MS: Rt = 4.39 min, [M + H] 482. |
| 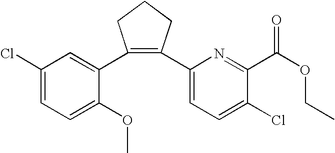 | Ethyl 3-chloro-6-{2-[5-chloro-2- (methyloxy)phenyl]-1-cyclopenten-1- yl}-2-pyridinecarboxylate | LC/MS Rt = 3.95, [MH+] 392.4 |
| 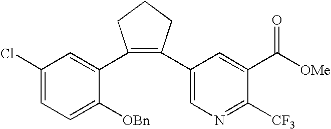 | Methyl 5-(2-{5-chloro-2- [(phenylmethyl)oxy]phenyl}-1- cyclopenten-1-yl)-2-(trifluoromethyl)- 3-pyridinecarboxylate | LC/MS Rt = 4.20, [MH+] 488.4 |
| 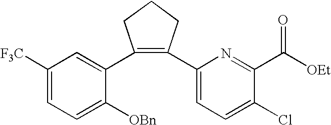 | Ethyl 3-chloro-6-{2-[2- [(phenylmethyl)oxy]-5- (trifluoromethyl)phenyl]-1- cyclopenten-1-yl}-2- pyridinecarboxylate | LC/MS Rt = 4.15, [MH+] 502.4, 504.4 |
| 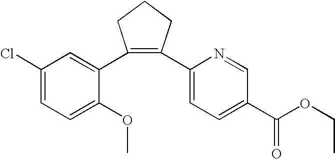 | Ethyl 6-{2-[5-chloro-2- (methyloxy)phenyl]-1-cyclopenten-1- yl}-3-pyridinecarboxylate | LC/MS Rt = 3.84, [MH+] 358.3 |
| 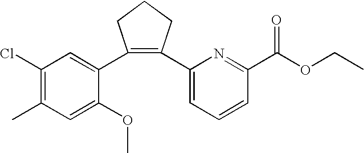 | Ethyl 6-{2-[5-chloro-4-methyl-2- methyloxy)phenyl]-1-cyclopenten-1- yl}-2-pyridinecarboxylate | LC/MS: Rt = 3.77 min. [M + H] = 372. |
| 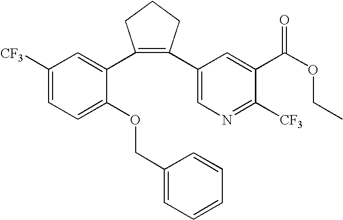 | Ethyl 5-{2-[2-[(phenylmethyl)oxy]-5- (trifluoromethyl)phenyl]-1- cyclopenten-1-yl}-2-(trifluoromethyl)- 3-pyridinecarboxylate | LC/MS: Rt = 3.91 min. [M + H] = 536 |
| 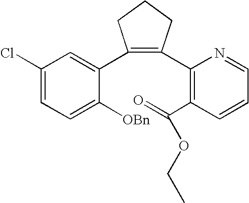 | Ethyl 2-(2-{5-chloro-2- [(phenylmethyl)oxy]phenyl}-1- cyclopenten-1-yl)-3- pyridinecarboxylate | LC/MS: Rt = 3.89 min. [M + H] = 434, 436 |
| 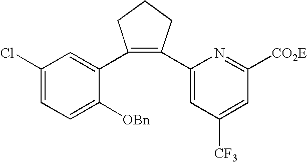 | Ethyl 6-(2-{5-chloro-2- [(phenylmethyl)oxy]phenyl}-1- cyclopenten-1-yl)-4-(trifluoromethyl)- 2-pyridinecarboxylate | LC/MS: [M + H] 502.4, 504.4, Rt = 4.58 min |
| 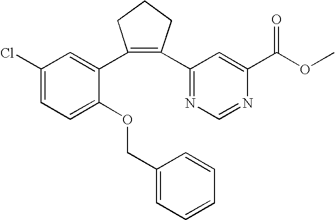 | Methyl 6-(2-{5-chloro-2- [(phenylmethyl)oxy]phenyl}-1- cyclopenten-1-yl)-4- pyrimidinecarboxylate | LC/MS: Rt = 3.74 min [M + H] 421, 423 |
| 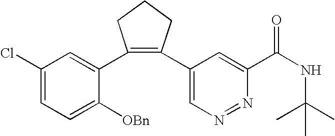 | 5-(2-{5-Chloro-2- [(phenylmethyl)oxy]phenyl}-l- cyclopenten-1-yl)-N-(1,1- dimethylethyl)-3- pyridazinecarboxamide | LC/MS: Rt = 3.98 min [M + H] 462, 464 |
| 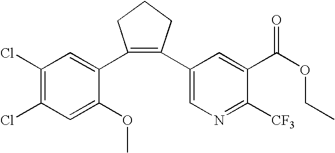 | Ethyl 5-{2-[4,5-dichloro-2- (methyloxy)phenyl]-1- cyclopenten-1- yl}-2-(trifluoromethyl)-3- pyridinecarboxylate | LC/MS: Rt = 3.68 min [M + H] 392 |

### Methyl 5-{2-[5-chloro-2-(methyloxy)phenyl]-1-cyclopenten-1-yl}-2-ethyl-3-pyridinecarboxylate

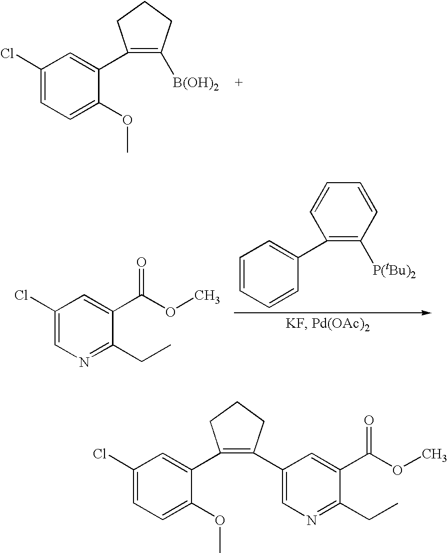

A mixture of {2-[5-chloro-2-(methyloxy)phenyl]-cyclopenten-1-yl}boronic acid (2.53 g, 10 mmol), methyl 5-chloro-2-ethyl-3-pyridinecarboxylate (1.995 g, 10 mmol), palladium acetate (22 mg, 0.0909 mmol), potassium fluoride on alumina (40%) (4.35 g, 30 mmol) and (di-tert-butylphosphino)biphenyl (60 mg, 0.20 mmol) In anhydrous tetrahydrofuran (25 ml) was heated at 50° C. under an atmosphere of nitrogen for 3 hours. The reaction mixture was cooled to room temperature and diluted with diethyl ether and water. The ether layer was separated, dried over magnesium sulphate and evaporated to dryness. The residue was purified using flash chromatography eluting with ethyl acetate/iso-hexane (15%) to give the title compound as a yellow oil. 1.93 g, 52%.

¹H NMR (CDCl₃) δ: 1.24 (3H, t), 2.06-2.14 (2H, m), 2.81-2.56 (2H, m), 2.91-2.95 (2H, m), 3.08 (2H, q), 3.63 (3H, s), 3.85 (3H, s), 6.79 (1H, d), 7.00 (1H, d), 7.18 (1H, dd), 7.91 (1H, d), 8.34 (1H, d). LC/MS: Rt=3.83 min, [M+H] 372.

The following intermediates were prepared by a similar route to methyl 5-{2-[5-chloro-2-(methyloxy)phenyl]-1-cyclopenten-1-yl}-2-ethyl-3-pyridinecarboxylate from the appropriate intermediates.

| | Name | Data |
|---|---|---|
| 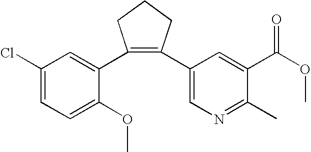 | Methyl 5-{2-[5-chloro-2- (methyloxy)phenyl]-1- cyclopenten-1-yl)-2-methyl-3- pyridinecarboxylate | LC/MS Rt = 3.64, [MH+] 358.4, 360.4 |
| 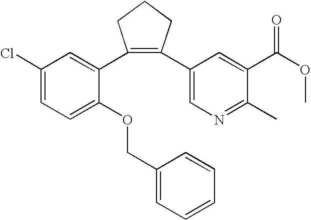 | Methyl 5-(2-{5-chloro-2- [(phenylmethyl)oxy]phenyl}-1- cyclopenten-1-yl)-2-methyl-3- pyridinecarboxylate | LC/MS Rt = 4.06, [MH+] 334.4, 436.4 |
| 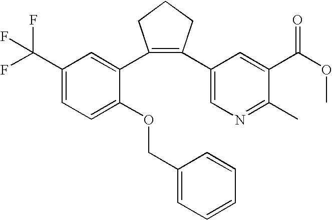 | Methyl 2-methyl-5-{2-[2- [(phenylmethyl)oxy]-5- (trifluoromethyl)phenyl]-1- cyclopenten-1-yl}-3- pyridinecarboxylate | LC/MS Rt = 4.04, [MH+] 468.4 |
| 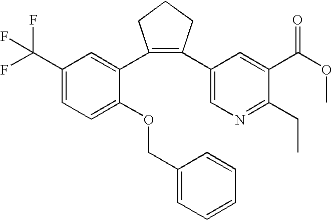 | Methyl 2-ethyl-5-{2-[2- [(phenylmethyl)oxy]-5- (trifluoromethyl)phenyl]-1- cyclopenten-1-yl}-3- pyridinecarboxylate | LC/MS Rt = 4.17, [MH+] 482.5 |

### Ethyl 6-{2-[2-(methyloxy)-3-pyridinyl]-1-cyclopenten-1-yl}-pyridinecarboxylate

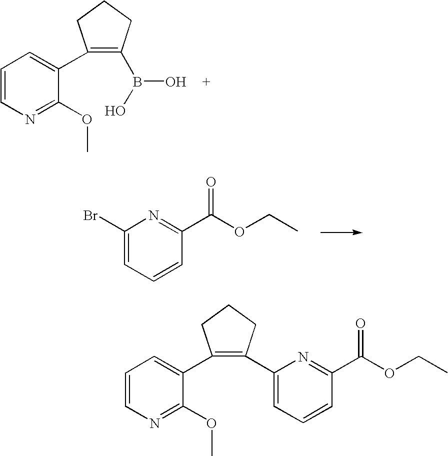

{2-[2-(Methyloxy)-3-pyridinyl]-1-cyclopenten-1-yl}boronic acid (219 mg, 1 mmol) and ethyl 6-bromo-2-pyridinecarboxylate (230 mg, 1 mmol) were dissolved in toluene/ethanol (1:1, 10 ml) under nitrogen and tetrakis(triphenylphosphine)palladium(0) (58 mg, 0.05 mmol) and potassium carbonate (1.104 g, 8 mmol) added. The mixture was heated at 80° C. in a Smithcreator® microwave for 20 minutes. Diethyl ether and water were added and the organic layer washed with water, dried (MgSO₄) and evaporated. The brown oil was purified by flash chromatography, eluting with 5-20% ethyl acetate/isohexane to give the title compound (120 mg).

¹H NMR (CDCl₃) δ: 1.38 (3H, t), 2.07-2.15 (2H, m), 2.87-2.92 (2H, m), 3.09-3.14 (2H, m), 3.78 (3H, s), 4.38 (2H, q), 6.78 (1H, dd), 7.08 (1H, d), 7.33 (1H, dd), 7.54 (1H, t), 7.84 (1H, d), 8.07 (1H, dd).

### 6-{2-[2-(Methyloxy)-5-(trifluoromethyl)phenyl]-1-cyclopenten-1-yl}-2-pyridinecarboxylic acid

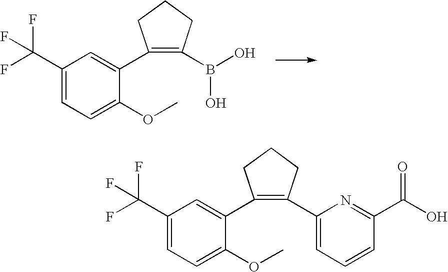

{2-[2-(Methyloxy)-5-(trifluoromethyl)phenyl]-1-cyclopenten-1-yl}boronic acid (5.5 g, 19.2 mmol), ethyl 6-bromopyridine-2-carboxylate (4.42 g, 19.2 mmol), potassium carbonate (13.29 g, 96.2 mmol) and tetrakis(triphenylphosphine)palladium(0) were refluxed in 1:1 ethanol/toluene (200 ml) under nitrogen in the dark for 16 hours. After cooling the reaction was filtered over celite and the solvent removed in vacuo, the residue was taken up in ethyl acetate and washed with water and brine, dried over MgSO₄, filtered and concentrated in vacuo to yield a yellow solid. This was purified by column chromatography eluting in 50% ethyl acetate/isohexane. This yielded the title compound as a yellow solid (5.51 g, 79%). LC/MS Rt=3.22, [MH⁺] 364.

The following compounds were prepared by a similar route to 6-{2-[2-(methyloxy)-5-(trifluoromethyl)phenyl]-1-cyclopenten-1-yl}-2-pyridinecarboxylic acid from the appropriate intermediates.

| | Name | LC/MS |
|---|---|---|
| 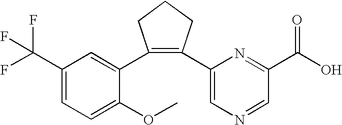 | 6-{2-[2-(Methyloxy)-5- (trifluoromethyl)phenyl]-1- cyclopenten-1-yl}-2- pyrazinecarboxylic acid | t = 3.66, [MH⁺] 365 |
| 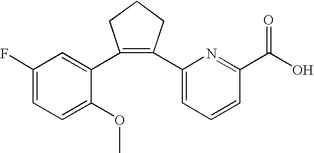 | 6-{2-[5-Fluoro-2- (methyloxy)phenyl]-1- cyclopenten-1-yl}-2- pyridinecarboxylic acid | t = 2.70 min, [MH⁺] 314 |
| 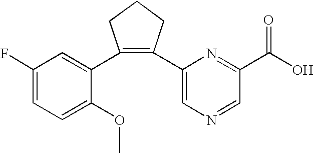 | 6-{2-[5-Fluoro-2- (methyloxy)phenyl]1- cyclopenten-1-yl}-2- pyrazinecarboxylic acid | t = 3.59 min, [MH⁺] 315 |

### 5-{2-[5-Bromo-2-(methyloxy)phenyl]-1-cyclopenten-1-yl}-N-(1,1-dimethylethyl)-3-pyridazinecarboxamide

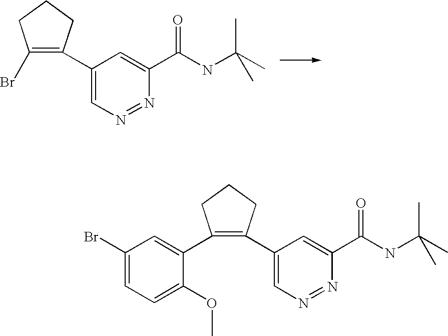

5-(2-Bromo-1-cyclopenten-1-yl)-N-(1,1-dimethylethyl)-3-pyridazinecarboxamide (8.3 g, 25.5 mmol), 5-bromo-2-(methyloxy)phenylboronic acid (6.9 g, 30 mmol), tetrakis(triphenylphosphine)palladium(0) (1.51 g, 1.3 mmol) and potassium carbonate (8.0 g, 57.97 mmol) in dimethoxyethane (60 ml) were refluxed overnight under nitrogen, in the dark. The reaction mixture was then filtered through celite and chromatographed giving the title compound (7.0 g, 65% yield). LC/MS Rt=3.71mins [MH⁺] 432, 433.

### Ethyl 6-{2-[5-bromo-2-(methyloxy)phenyl]-1-cyclopenten-1-yl}-chloro-2-pyridinecarboxylate

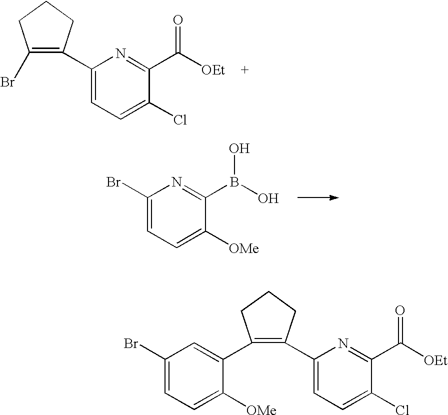

A mixture of ethyl 6-(2-bromo-1-cyclopenten-1-yl)-3-chloro-2-pyridinecarboxylate (110 mg, 0.33 mmol), 5-bromo-2-(methyloxy)phenylboronic acid (77 mg, 0.33 mmol), potassium carbonate (276 mg, 2 mmol) and tetrakis(triphenylphosphine)palladium(0) (38 mg, 0.033 mmol) in 1,2-dimethoxyethane (4 ml) was stirred and heated at 70° C. under nitrogen for 2 hours when a further portion of 5-bromo-2-methoxyphenylboronic acid (77 mg, 0.33 mmol) was added. After heating for a further 2 hours the mixture was cooled, dissolved in diethyl ether/water and the organic phase dried (magnesium sulphate) evaporated and the residue purified by chromatography on silica eluting with ethyl acetate/iso-hexane (7:93) to give 110 mg of colourless oil.

LC/MS Rt=4.14, [MH+] 438.3.

The following compounds were prepared by a similar route to ethyl 6-{2-[5-bromo-2-(methyloxy)phenyl]-1-cyclopenten-1-yl}-3-chloro-2-pyridinecarboxylate from the appropriate intermediates.

| | Name | Data |
|---|---|---|
| 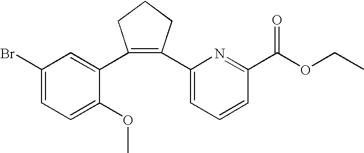 | Ethyl 6-{2-[5-bromo-2- (methyloxy)phenyl]-1- cyclopenten-1-yl}-2- pyridinecarboxylate | LC/MS: Rt = 3.80 min. [M + H] = 402, 404. |
| 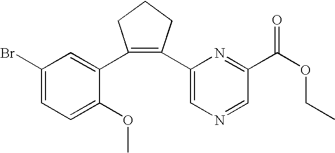 | Ethyl 6-{2-[5-bromo-2- (methyloxy)phenyl]-1- cyclopenten-1-yl}-2- pyrazinecarboxylate | LC/MS: Rt = 3.66 min. [M + H] = 403, 405. |

### Ethyl 5-(2-{5-chloro-2-[(phenylmethyl)oxy]-3-Pyridinyl}-cyclopenten-1-yl)-2-methylbenzoate

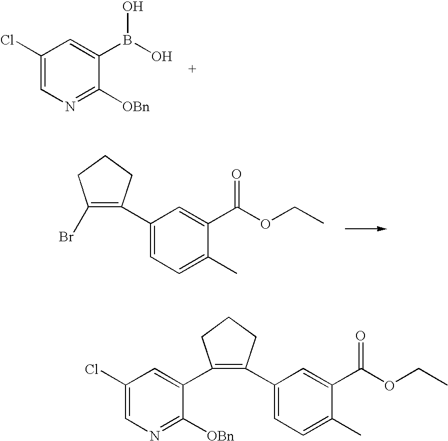

{5-Chloro-2-[(phenylmethyl)oxy]-3-pyridinyl}boronic acid (247 mg, 0.938 mmol) and ethyl 5-(2-bromo-1-cyclopenten-1-yl)-2-methylbenzoate (290 mg, 0.938 mmol) were dissolved in toluene/ethanol (1:1, 4 ml) under nitrogen and tetrakis(triphenylphosphine)palladium(0) (54 mg, 0.047 mmol) and potassium carbonate (1.04 g, 7.5 mmol) added. The mixture was heated at 80° C. in a Smithcreator® microwave for 10 minutes. Diethyl ether and water were added and the organic layer washed with water, dried (MgSO₄) and evaporated. The brown oil was purified by flash chromatography, eluting with 3% ethyl acetate/isohexane to give the title compound (262 mg). LC/MS Rt=4.47 min [MH⁺] 448, 450.

The following compounds were prepared by a similar route to ethyl 5-(2-{5-chloro-2-[(phenylmethyl)oxy]-3-pyridinyl}-1-cyclopenten-1-yl)-2-methylbenzoate from the appropriate intermediates.

| | COMPOUND NAME | ¹H NMR/LCMS |
|---|---|---|
| 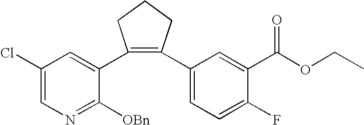 | Ethyl 5-(2-{5-chloro-2- [(phenylmethyl)oxy]-3- pyridinyl)-1-cyclopenten-1-yl}- 2-fluorobenzoate | (CDCl₃) δ: 1.33 (3 H, t), 2.05-2.08 (2 H, m), 2.82- 2.91 (4 H, m), 4.31 (2 H, q), 5.27 (2 H, s), 6.84 (1 H, dd), 7.11 (1 H, m), 7.22- 7.29 (6 H, m), 7.66 (1 H, dd), 8.00 (1 H, d) |
| 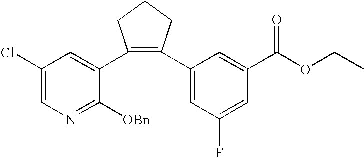 | Ethyl 5-(2-{5-chloro-2- [(phenylmethyl)oxy]-3- pyridinyl)-1-cyclopenten-1-yl}- 3-fluorobenzoate | (CDCl₃) δ: 1.32 (3 H, t), 2.04-2.11 (2 H, m), 2.83- 2.92 (4 H, m), 4.29 (2 H, q), 5.26 (2 H, s), 6.89 (1 H, dd), 7.21-7.56 (8 H, m), 8.01 (1 H, d) |
| 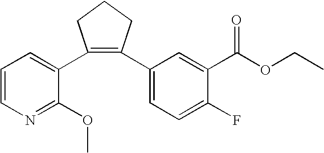 | Ethyl 2-fluoro-5-{2-[2- (methyloxy)-3-pyridinyl]-1- cyclopenten-1-yl}benzoate | (CDCl₃) δ: 1.34 (3 H, t), 2.05-2.12 (2 H, m), 2.83- 2.87 (2 H, m), 2.89- 2.94 (2 H, m), 3.85 (3 H, s), 4.32 (2 H, q), 6.77 (1 H, dd), 6.88 (1 H, dd), 7.15 (1 H, td), 7.24 (1 H, dd), 7.69 (1 H, dd), 8.07 (1 H, dd). |
| 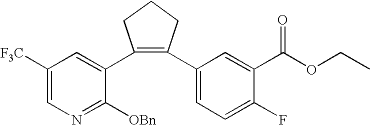 | Ethyl 2-fluoro-5-{2-[2- [(phenylmethyl)oxy]-5- (trifluoromethyl)-3-pyridinyl]-1- cyclopenten-1-yl}benzoate | Rt = 4.42 min. [MH⁺] 486 |
| 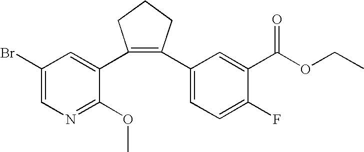 | Ethyl 5-{2-[5-bromo-2- (methyloxy)-3-pyridinyl]-1- cyclopenten-1-yl}-2- fluorobenzoate | Rt = 4.10 min. [MH⁺] 420, 422. |

### Ethyl 6-{2-[2-[(phenylmethyl)oxy]-5-(trifluoromethyl)-3-pyridinyl]-1-Cyclopenten-1-yl}-2-pyridinecarboxylate

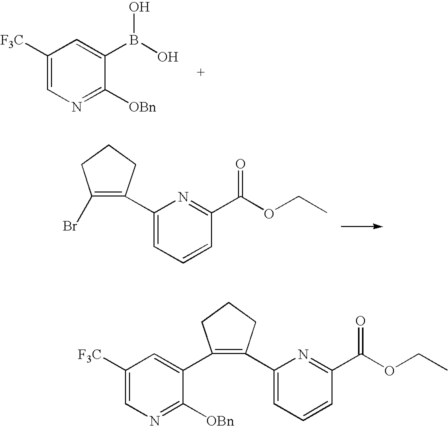

[2-[(Phenylmethyl)oxy]-5-(trifluoromethyl)-3-pyridinyl]boronic acid (3.71 g, 12.5 mmol) and 6-(2-bromocyclopent-1-enyl)-pyridine-2-carboxylic acid ethyl ester (1.85 g, 6.25 mmol) were dissolved in dioxane (75 mL) under nitrogen together with tris(dibenzylideneacetone)dipalladium(0) (86 mg, 0.094 mmol), tri(t-butyl)phosphonium tetrafluoroborate (82 mg, 0.28 mmol) and potassium fluoride (1.19 g, 20.5 mmol). The mixture was heated at 100° C. for 3 hours. After cooling, the dioxane was removed in vacuo and the residue was partitioned between diethyl ether and water. The aqueous was extracted with further ether, dried (Na₂SO₄) and concentrated in vacuo. The resulting brown oil was purified by flash chromatography on silica (gradient elution, 0-3% ethyl acetate/cyclohexane) to give the title compound (871 mg).

LC/MS Rt=4.09 min [MH⁺] 469.

### Ethyl 3-amino-5-{2-[2-(phenylmethoxy)-5-(trifluoromethyl)pyridin-3-yl]cyclopent-1-en-1-yl}benzoate

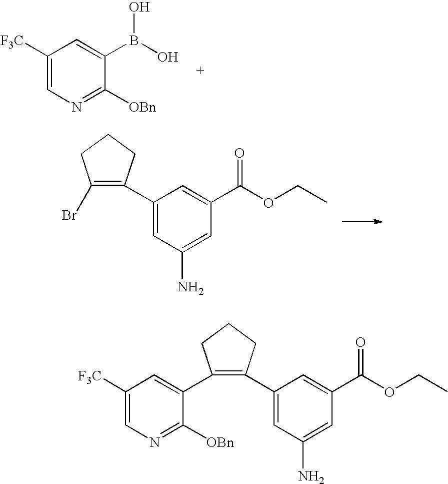

2-(Phenylmethoxy)-5-(trifluoromethyl)pyridine-3-boronic acid (6.0 g, 20.2 mmol) and ethyl 3-amino-5-(2-bromocyclopent-1-enyl)benzoate (3.16 g, 10.1 mmol) were dissolved in dimethoxyethane (50 mL) under nitrogen, and tetrakis(triphenylphosphine)palladium(0) (0.58 g, 0.5 mmol) and 2N aqueous sodium carbonate solution (10 ml) were added. The mixture was heated at 80° C. for 18 hours. After cooling, the solvents were removed in vacuo, and the residue was partitioned between diethyl ether and water. The aqueous was extracted with further ether (×2), and the combined organic layers were dried (Na₂SO₄) and concentrated in vacuo. The resulting dark brown oil was purified using an acidic solid phase cartridge (Isolute® Flash SCX-2, 50 g), loading the crude material as a methanol solution and eluting with 10% aqueous ammonia in methanol. Concentration of the relevant fractions in vacuo gave the title compound (4.01 g).

LC/MS Rt=4.01 min [MH⁺] 483.

### Ethyl 3-{2-[5-bromo-2-(methyloxy)-3-pyridinyl]-1-cyclopenten-1-yl}benzoate

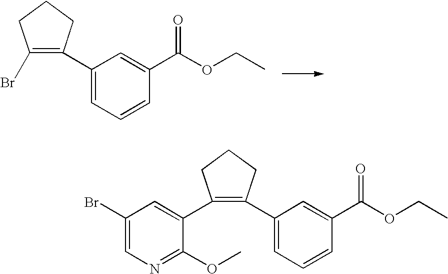

Ethyl 3-(2-bromo-1cyclopenten-1-yl)benzoate (0.5 g, 1.7 mmol), [5-bromo-2-(methyloxy)-3-pyridinyl]boronic acid (0.45 g, 1.7 mmol), potassium carbonate (1.2 g, 8.5 mmol) and 1,2-dimethoxyethane (10 ml) were combined and degassed for 15 minutes tetrakis(triphenylphosphine)palladium(0) (0.2 g, 0.17 mmol) was added and the reaction stirred under a nitrogen atmosphere in the dark at 80° C. for 3 hours. A further equivalent of [5-bromo-2-(methyloxy)-3-pyridinyl]boronic acid (0.45 g, 1.7 mmol) was added and the reaction continued under the above conditions for a further 14 hours. A further equivalent of [5-bromo-2-(methyloxy)-3-pyridinyl]boronic acid (0.45 g, 1.7 mmol) and a further equivalent of tetrakis(triphenylphosphine)palladium(0) (0.2 g, 0.17 mmol) was added and the reaction continued under the above conditions for a further 24 hours. The reaction was then filtered through celite and the solvent removed in vacuo. The residue was purified by column chromatography eluting with 10% diethyl ether/isohexane. This yielded the title compound as a clear oil (0.201 g, 30%). LC/MS Rt=4.30 [MH⁺] 402/404.

### 6-[2-(5-Chloro-2-hydroxyphenyl)-1-cyclopenten-1-yl]-2-pyridinecarboxylic acid

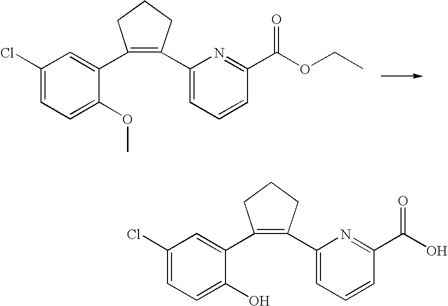

Ethyl 6-{2-[5-chloro-2-(methyloxy)phenyl]-1-cyclopenten-1-yl}-2-pyridinecarboxylate (3.9 g, 0.011 mol) and sodium methanethiolate (49, 0.055 mol) in dry DMF (40 ml) were heated at 100° C. under nitrogen for 5 h. After cooling the mixture was poured into water and washed with diethyl ether. The aqueous phase was then acidified with acetic acid and extracted with ethyl acetate (50 ml×3). The combined organic layers were dried (magnesium sulphate) and evaporated. The residue was redissolved in toluene and evaporated again to give a yellow solid that was triturated with diethyl ether to give the title compound as a yellow oil (2.6 g, 76%). LC/MS: Rt 2.85 [MH+] 316, 318.

The following intermediates were prepared by a similar route to 6-[2-(5-chloro-2-hydroxyphenyl)-1-cyclopenten-1-yl]-2-pyridinecarboxylic acid from the appropriate intermediates.

| Structure | Name | Data |
|---|---|---|
| 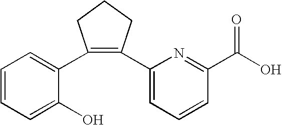 | 6-[2-(2-Hydroxyphenyl)-1- cyclopenten-1-yl]-2- pyridinecarboxylic acid | LC/MS: Rt = 2.20 min, [M + H] 282. |
| 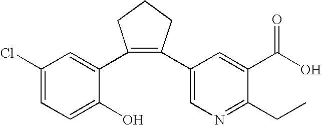 | 5-[2-(5-Chloro-2- hydroxyphenyl)-1- cyclopenten-1-yl]-2-ethyl-3- pyridinecarboxylic acid | LC/MS: Rt = 2.99 min, [M + H] 344. |
| 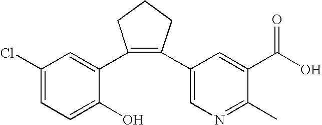 | 5-[2-(5-Chloro-2- hydroxyphenyl)-1- cyclopenten-1-yl]-2-methyl- 3-pyridinecarboxylic acid | LC/MS Rt = 2.80 min, [MH+] 330.4, 332.4 |
| 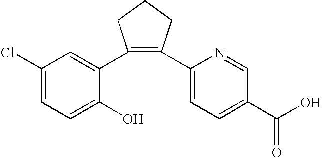 | 6-[2-(5-Chloro-2- hydroxyphenyl)-1- cyclopenten-1-yl]-3- pyridinecarboxylic acid | LC/MS Rt = 3.70 min, [MH+] 316.3, 318.4 |
| 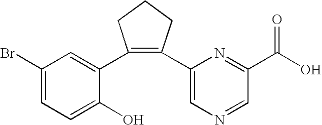 | 6-[2-(5-Bromo-2- hydroxyphenyl)-1- cyclopenten-1-yl]-2- pyrazinecarboxylic acid | LC/MS: Rt = 4.37 min. [M + H] = 361, 363. |
| 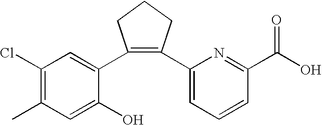 | 6-[2-(5-Chloro-2-hydroxy-4- methylphenyl)-1- cyclopenten-1-yl]-2- pyridinecarboxylic acid | LC/MS: Rt = 3.02 min. [M + H] = 330 |
| 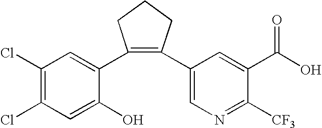 | 5-[2-(4,5-dichloro-2- hydroxyphenyl)-1 cyclopenten-1-yl]-2- (trifluoromethyl)-3- pyridinecarboxylic acid | LC/MS: Rt = 3.17 min. [M + H] = 418 |

### 6-{2-[2-Hydroxy-5-(trifluoromethyl)phenyl]-1-cyclopenten-1-yl}-2-pyrazinecarboxylic acid

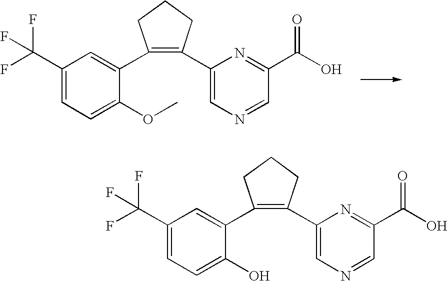

6-{2-[2-(Methyloxy)-5-(trifluoromethyl)phenyl]-1-cyclopenten-1-yl}-2-pyrazinecarboxylic acid (1.92 g, 5.27 mmol), sodium methanethiolate (1.87 g, 26.4 mmol) and DMF (40 ml) were heated to 75° C. for 4.5 hours. After cooling the reaction was diluted with ethyl acetate washed with water and brine. The organic layer was dried over MgSO₄, filtered and concentrated in vacuo to yield a yellow solid (1.66 g). LC/MS Rt=3.57, [MH⁺] 351.

### 6-[2-(5-Chloro-2-hydroxyphenyl)-1-cyclopenten-1-yl]-(methylthio)-2-pyridinecarboxylic acid

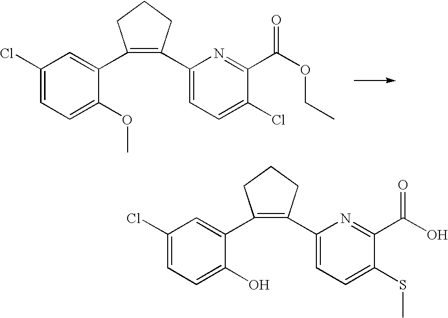

A mixture of ethyl 3-chloro-6-{2-[5-chloro-2-(methyloxy)phenyl]-1-cyclopenten-1-yl}-2-pyridinecarboxylate (960 mg, 2.49 mmol) and sodium methanethiolate (857 mg, 12.25 mmol) in dimethylformamide (10 ml) was stirred and heated at 10° C. under nitrogen for 4 hours. After cooling the mixture was diluted with diethyl ether/water and the aqueous separated, acidified with acetic acid and extracted with ether which was washed three times with water then dried (magnesium sulphate) evaporated and triturated with ether to give an orange solid (695 mg). LC/MS Rt=3.46, [MH+] 362.4, 364.4.

### 6-{2-[2-Hydroxy-5-(trifluoromethyl)phenyl]cyclopent-1-en-1-yl}pyridine-2-carboxylic acid

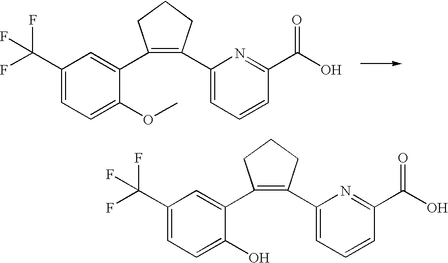

6-{2-[2-Methoxy-5-(trifluoromethyl)phenyl]cyclopent-1-en-1-yl}pyridine-2-carboxylic acid (2 g, 5.5 mmol) was dissolved in anhydrous dichloromethane (80 ml) and cooled to −70° C. Boron tribromide (5 ml, 55 mmol) was added slowly and the reaction allowed to warm to −3° C. and stirred under nitrogen for 19 hours. The reaction was quenched with ice and then water and stirred vigorously for 30 minutes. The aqueous layer was washed with dichloromethane (×2), the combined organic layers were washed with brine, dried over MgSO4, filtered and concentrated in vacuo to yield the title compound as a dark solid (2.13 g). LC/MS Rt=3.07 min, [MH⁺] 350.

The following intermediates were prepared by a similar route to 6-{2-[2-hydroxy-5-(trifluoromethyl)phenyl]cyclopent-1-en-1-yl}pyridine-2-carboxylic acid from the appropriate intermediates.

| | Name | LC/MS |
|---|---|---|
| 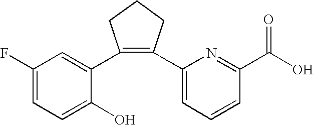 | 6-{2-[5-Fluoro-2-hydroxyphenyl]- 1-cyclopenten-1-yl}2- pyridinecarboxylic acid | Rt = 2.50 min [MH⁺] 300. |
| 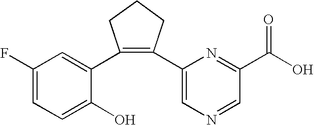 | 6-{2-[5-Fluoro-2-hydroxyphenyl]- 1-cyclopenten-1-yl}2- pyrazinecarboxylic acid | Rt = 3.53 min [MH⁺] 301. |

### 3-Chloro-6-[2-(5-chloro-2-hydroxyphenyl)-1-cyclopenten-1-yl]-2-pyridinecarboxylic acid

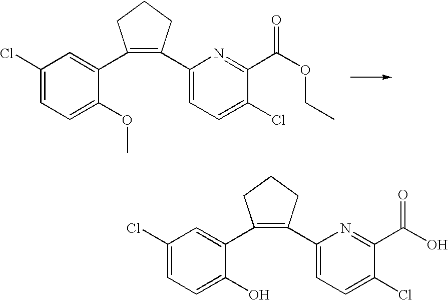

A solution of ethyl 3-chloro-{2-[5-chloro-2-(methyloxy)phenyl]-1-cyclopenten-1-yl}-2-pyridinecarboxylate (2.35 g, 6 mmol) in dichloromethane (15 ml) was cooled to −50° C. and 1M boron tribromide in dichloromethane (20 ml) was added.

The mixture was allowed to warm to room temperature and after 3 hours was poured onto ice and basified with 2M sodium hydroxide solution then acidified with acetic acid. The organic layer was separated, dried (magnesium sulphate), toluene (30 ml) added and evaporated to give a yellow gum (2.16 g). LC/MS t=4.09, [MH+] 350.4

### 6-[2-(5-Chloro-2-hydroxyphenyl)-1-cyclopenten-1-yl]-3-methyl-2-pyridinecarboxylic acid

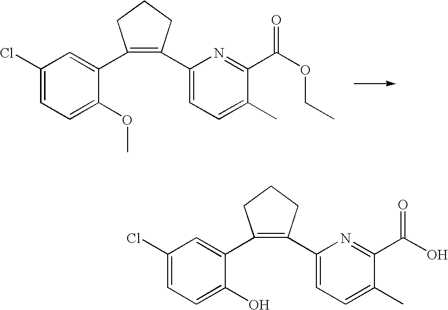

Procedure as for 3-chloro-6-[2-(5-chloro-2-hydroxyphenyl)-1-cyclopenten-1-yl]-2-pyridinecarboxylic acid. LC/MS t=3.05, [MH+] 330.4

### Ethyl 6-[2-(5-bromo-2-hydroxyphenyl)-1-cyclopenten-1-yl]-2-pyridinecarboxylate

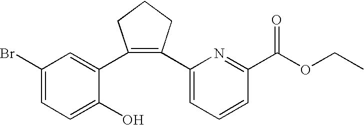

1.0M boron tribromide in dichloromethane (9.95 ml, 9.95 mmol) was added to a solution of ethyl 6-[2-(5-bromo-2-hydroxyphenyl)-1-cyclopenten-1-yl]-2-pyridinecarboxylate (2.0 g, 4.98 mmol) in dry dichloromethane (50 ml) at −78° C. The reaction mixture was allowed to warm to room temperature over 4 hours. The mixture was quenched with water (50 ml). The organic phase was separated, dried and evaporated to give the title compound as a yellow solid 2.0 g 100%. LC/MS: Rt=3.64 min. [M+H]=388, 390 (1 Br).

### Ethyl 6-[2-(5-bromo-2-hydroxyphenyl)-cyclopenten-1-yl]-3-chloro-2-pyridinecarboxylate

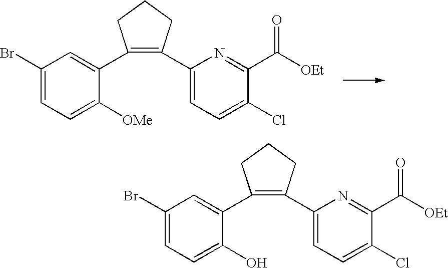

A solution of ethyl 6-{2-[5-bromo-2-(methyloxy)phenyl]-1-cyclopenten-1-yl}-chloro-2-pyridinecarboxylate (501 mg, 1.15 mmol) in dichloromethane (5 ml) was cooled to −50° C. and 1M boron tribromide in dichloromethane (5 ml) was added.

The mixture was allowed to warm to room temperature and after 2 hours was poured onto ice and basified with 2M sodium hydroxide solution then acidified with acetic acid. The organic layer was separated, dried (magnesium sulphate), toluene (10 ml) added and evaporated to dryness. The residue was dissolved in ethanol (25 ml) and sulphuric acid (2 ml) and refluxed for 5 hours then left at room temperature for 15 hours. After evaporation the residue was dissolved in ether/water, basified with potassium carbonate and the organic phase dried (magnesium sulphate), evaporated and purified by chromatography on silica eluting with ethyl acetate/iso-hexane (18:82) to give 415 mg of colourless gum. LC/MS t=3.97, [MH+] 424.3

### Ethyl 5-[2(5-chloro-2-oxo-1,2-dihydro-3-pyridinyl)-1-cyclopenten-1-yl]-2-methylbenzoate

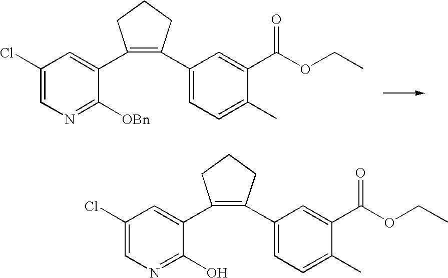

Ethyl 5-(2-{5-chloro-2-[(phenylmethyl)oxy]-3-pyridinyl}-1-cyclopenten-1-yl)-2-methylbenzoate (200 mg, 0.447 mmol) was dissolved in glacial acetic acid (0.5 ml) and 45% hydrogen bromide in acetic acid (1 ml) added. The mixture was stirred at room temperature for 1.5 hours. 5% sodium bicarbonate solution was added carefully, followed by diethyl ether. The aqueous layer was reextracted with diethyl ether and the combined extracts washed with 5% sodium bicarbonate solution, dried (MgSO₄) and evaporated to leave the title compound (76 mg). LC/MS Rt=3.51 min [MH⁻] 356, 358.

The following intermediates were prepared by a similar route to ethyl 5-[2-(5-chloro-2-oxo-1,2-dihydro-3-pyridinyl)-1-cyclopenten-1-yl]-2-methylbenzoate from the appropriate intermediates.

| | Name | Data |
|---|---|---|
| 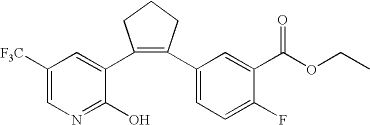 | Ethyl 2-fluoro-5-{2-[2-oxo-5- (trifluoromethyl)-1,2-dihydro-3- pyridinyl]-1-cyclopenten-1- yl}benzoate | LC/MS Rt = 3.54 min [MH⁺] 396. |
| 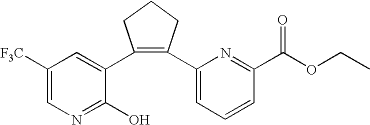 | Ethyl 6-{2-[2-oxo-5- (trifluoromethyl)-1,2-dihydro-3- pyridinyl]-1-cyclopenten-1-yl}-2- pyridinecarboxylate | LC/MS Rt = 3.12 min [MH⁺] 379. |

### 5-[2-(5-Bromo-2-oxo-1,2-dihydro-3-pyridinyl)-1-cyclopenten-1-yl]-2-fluorobenzoic acid

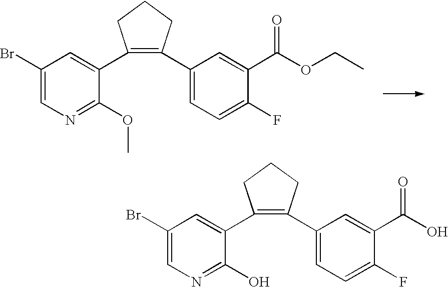

Ethyl 5-{2-[5bromo-2-(methyloxy)-3-pyridinyl]-1-cyclopenten-1-yl}-fluorobenzoate (127 mg, 0.302 mmol), was dissolved in glacial acetic acid (1 ml) and 48% aqueous hydrogen bromide (1 ml) added. The mixture was heated to reflux for 45 minutes. 5% Sodium bicarbonate solution was added carefully and the mixture extracted with ethyl acetate. The organic layer was washed with water, dried (MgSO₄) and evaporated to give the title compound (96 mg). LC/MS Rt=3.19 min [MH⁺] 378, 380.

The following intermediates were prepared by a similar route to 5-[2-(5-bromo-2-oxo-1,2-dihydro-3pyridinyl)-cyclopenten-1-yl]-2-fluorobenzoic acid from the appropriate intermediates.

| | Name | LC/MS |
|---|---|---|
| 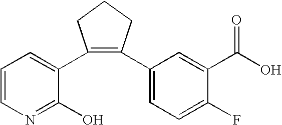 | 2-Fluoro-5-[2-(2-oxo-1,2-dihydro- 3-pyridinyl)-1-cyclopenten-1- yl]benzoic acid | Rt = 2.66 min [MH⁺] 300. |
| 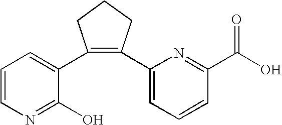 | 6-[2-(2-Oxo-1,2-dihydro-3- pyridinyl)-1-cyclopenten-1-yl]-2- pyridinecarboxylic acid | Rt = 1.48 min [MH⁺] 283 |
| 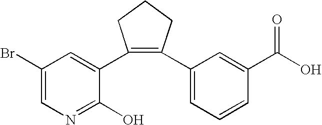 | 3-[2-(5-Bromo-2-oxo-1,2-dihydro- 3-pyridinyl)-1-cyclopenten-1- yl]benzoic acid | Rt = 2.89 min [MH⁺] 358, 360 |

### Ethyl 5-[2-(5-chloro-2-oxo-1,2-dihydro-3-pyridinyl)-1-cyclopenten-1-yl]-2-fluorobenzoate

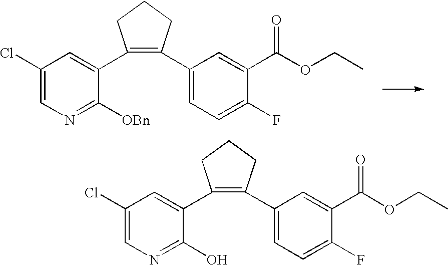

Ethyl 5-(2-{5-chloro-2-[(phenylmethyl)oxy]-3pyridnyl}-cyclopenten-1-yl)-2-fluorobenzoate (1.32 g, 2.93 mmol) was stirred in trifluoracetic acid (1 ml) at room temperature for 20 hours and at 50° C. for 12 hours. The mixture was poured carefully into 5% sodium bicarbonate solution and diethyl ether added. The organic layer was washed with 5% sodium bicarbonate solution, dried (MgSO₄) and evaporated to give the title compound as an orange oil which crystallised (1.06 g).

LC/MS Rt=3.25 min [MH⁺] 362.5, 364.5.

### Ethyl 6-[2-(5-chloro-2-hydroxyphenyl)-1-cyclopenten-1-yl]-2-pyridine carboxylate

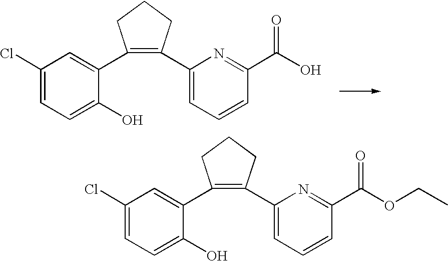

6-[2-(5-chloro-2-hydroxyphenyl)-1-cyclopenten-1-yl]-2-pyridinecarboxylic acid (2.6 g, 0.0082 mol) and concentrated sulphuric acid (1 ml) in 100 ml of ethanol were refluxed overnight. After cooling the mixture was quenched with ammonia, diluted with water and extracted with ethyl acetate (30 ml×3). The combined organic layers were washed with a saturated solution of sodium bicarbonate, dried (magnesium sulphate) and evaporated to dryness to give the title compound as a light yellow oil (2.5 g, 89%).

LC/MS: Rt 3.65 [MH⁺] 344, 346 [MH−] 342, 344

The following intermediates were prepared by a similar route to ethyl 6-[2-(5-chloro-2-hydroxyphenyl)-1-cyclopenten-1-yl]-2-pyridine carboxylate from the appropriate intermediates.

| | Name | Data |
|---|---|---|
| 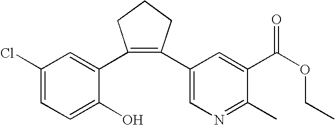 | Ethyl 5-[2-(5-chloro-2- hydroxyphenyl)-1-cyclopenten-1- yl]-2-methyl-3-pyridinecarboxylate | LC/MS t = 3.62, [MH+] 358.4, 360.4 |
| 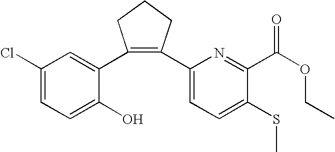 | Ethyl 6-[2-(5-chloro-2- hydroxyphenyl)-1-cyclopenten-1- yl]-3-(methylthio)-2- pyridinecarboxylate | LC/MS t = 3.75, [MH+] 390.4, 392.4 |
| 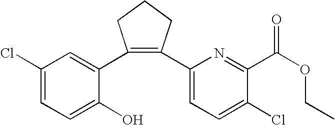 | Ethyl 3-chloro-6-[2-(5-chloro-2- hydroxyphenyl)-1-cyclopenten-1- yl]-2-pyridinecarboxylate | LC/MS t = 3.93, [MH+] 378.4 |
| 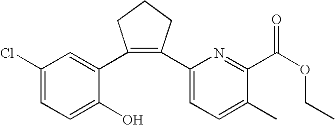 | Ethyl 6-[2-(5-chloro-2- hydroxyphenyl)-1-cyclopenten-1- yl]-3-methyl-2-pyridinecarboxylate | LC/MS t = 3.82, [MH+] 358.4, 360.4 |
| 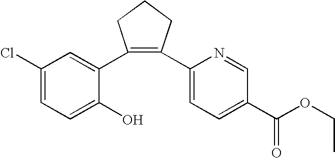 | Ethyl 6-[2-(5-chloro-2- hydroxyphenyl)-1-cyclopenten-1- yl]-3-pyridinecarboxylate | LC/MS t = 3.67, [MH+] 344.3, 346.3 |
| 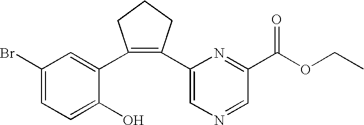 | Ethyl 6-[2-(5-bromo-2- hydroxyphenyl)-1-cyclopenten-1- yl]-2-pyrazinecarboxylate | LC/MS: Rt = 3.48 min. [M + H] = 389, 391. |
| 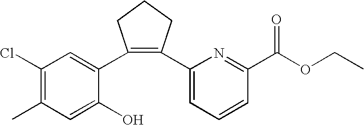 | Ethyl 6-[2-(5-chloro-2-hydroxy-4- methylphenyl)-1-cyclopenten-1-yl]- 2-pyridinecarboxylate | LC/MS: Rt = 3.74 min. [M + H] = 358. |
| 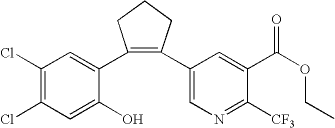 | Ethyl 5-[2-(4,5-dichloro-2- hydroxyphenyl)-1-cyclopenten-1- yl]-2-(trifluoromethyl)-3- pyridinecarboxylate | LC/MS: Rt = 3.86 min. [M + H] = 378, 380. |

### Ethyl 6-{2-[2-hydroxy-5-(trifluoromethyl)phenyl]-1-cyclopenten-1-yl}-3-methyl-2-pyridinecarboxylate

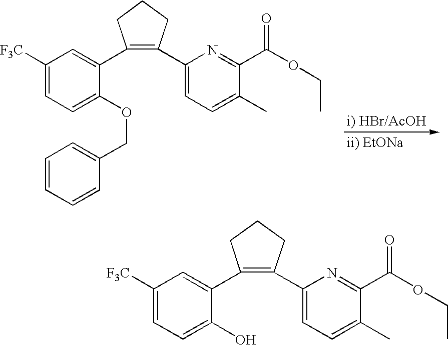

A mixture of ethyl 3-methyl-6-{2-[2-[(phenylmethyl)oxy]-5-(trifluoromethyl)phenyl]-1-cyclopenten-1-yl}-2-pyridinecarboxylate (2.21 g, 4.59 mmol) dissolved in acetic acid (5 ml) and 45% hydrogen bromide in acetic acid (10 ml) was stirred at room temperature for 3 hours. The reaction mixture was diluted with diethyl ether and water and basified with potassium carbonate. The ether layer was separated, dried over magnesium sulphate and evaporated to dryness. The residue was purified using flash chromatography eluting with ethyl acetate/iso-hexane (15%) to give 1.44 g of yellow solid. Sodium hydride (2 mg) was added to the product dissolved in ethanol and left at room temperature for 12 hours. The reaction mixture was diluted with diethyl ether and water, then acidified with acetic acid. The ether layer was washed with sodium hydrogen carbonate solution, dried over magnesium sulphate and evaporated to dryness. The residue was purified using flash chromatography eluting with 20% ethyl acetate/iso-hexane to give the title compound as a light coloured solid. 1.11 g, 62%.

¹H NMR (CDCl₃) δ: 1.48 (3H, t), 2.08-2.15 (2H, m), 2.50 (3H, s), 2.86-3.90 (2H, m), 3.01-3.05 (2H, m), 4.45 (2H, q), 7.08 (1H, d), 7.33-7.37 (2H, m), 7.39 (1H, dd), 7.61 (1H, d).

The following intermediates were prepared by a similar route to ethyl 6-{2-[2-hydroxy-5-(trifluoromethyl)phenyl]-1-cyclopenten-1-yl}-3-methyl-2-pyridinecarboxylate from the appropriate intermediates.

| | Name | Data |
|---|---|---|
| 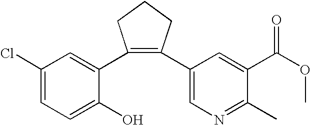 | Methyl 5-[2-(5-chloro-2- hydroxyphenyl)-1-cyclopenten- 1-yl]-2-methyl-3- pyridinecarboxylate | LC/MS Rt = 3.45, [MH+] 344.3, 346.3 |
| 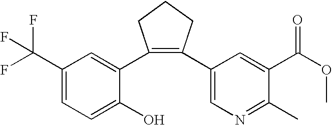 | Methyl 5-{2-[2-hydroxy-5- (trifluoromethyl)phenyl]-1- cyclopenten-1-yl}-2-methyl-3- pyridinecarboxylate | LC/MS Rt = 3.49, [MH+] 378.5 |
| 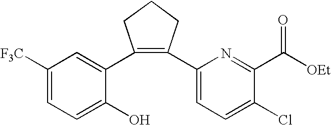 | Ethyl 3-chloro-6-{2-[2-hydroxy-5- (trifluoromethyl)phenyl]-1- cyclopenten-1-yl)-2- pyridinecarboxylate | LC/MS Rt = 3.90, [MH+] 412.5, 414.4 |
| 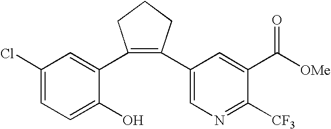 | Methyl 5-[2-(5-chloro-2- hydroxyphenyl)-1-cyclopenten- 1-yl]-2-(trifluoromethyl)-3- pyridinecarboxylate | LC/MS Rt = 3.75, [MH+] 398.4, 400.4 |
| 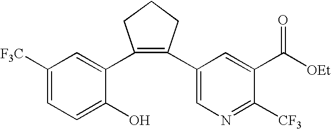 | Methyl 5-{2-[2-hydroxy-5- (trifluoromethyl)phenyl]-1- cyclopenten-1-yl}-2- (trifluoromethyl)-3- pyridinecarboxylate | LC/MS Rt = 4.02, [MH+] 446. |
| 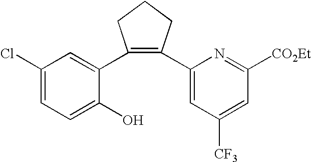 | Ethyl 6-[2-(5-chloro-2- hydroxyphenyl)-1-cyclopenten- 1-yl]-4-(trifluoromethyl)-2- pyridinecarboxylate | LC/MS Rt = 3.45, [MH+] 412. |
| 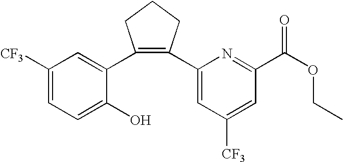 | Ethyl 6-{2-[2-hydroxy-5- trifluoromethyl)phenyl]-1- cyclopenten-1-yl}-4- (trifluoromethyl)-2- pyridinecarboxylate | LC/MS Rt = 4.09, [MH+] 446. |
| 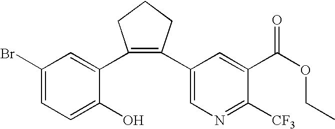 | Ethyl 5-[2-(5-bromo-2- hydroxyphenyl)-1-cyclopenten- 1-yl]-2-(trifluoromethyl)-3- pyridinecarboxylate | LC/MS Rt = 3.17, [MH+] 457 |
| 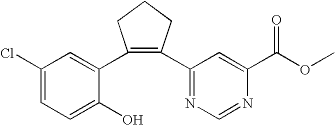 | Methyl 6-[2-(5-chloro-2- hydroxyphenyl)-1-cyclopenten- 1-yl]-4-pyrimidinecarboxylate | LC/MS Rt = 3.07, [MH+] 331, 333 |
| 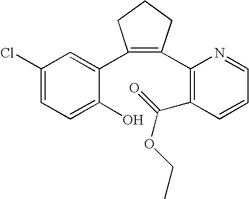 | Ethyl 2-[2-(5-chloro-2- hydroxyphenyl)-1-cyclopenten- 1-yl]-3-pyridinecarboxylate | LC/MS Rt = 3.26, [MW+] 344.4, 346.3 |

### 5-[2-(5-Bromo-2-hydroxyphenyl)-1-cyclopenten-1-yl]-N-(1,1-dimethylethyl)-3-pyridazinecarboxamide

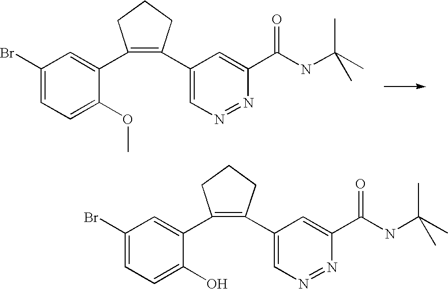

5-{2-[5-Bromo-2-(methyloxy)phenyl]-1-cyclopenten-1-yl}-N-(1,1-dimethylethyl)-3pyridazinecarboxamide (5.3 g, 12.3 mmol) in dry dichloromethane (200 ml) was cooled to −75° C. under nitrogen and was treated slowly with boron tribromide (8.0 ml, 84.8 mmol). The reaction mixture was then heated to reflux for 1.5 hour. The reaction mixture was then quenched in ice-water (400 ml) and after stirring at room temperature for 2 hours the organic layer was dried and evaporated to a dark brown solid (6.0 g).

LC/MS Rt=3.55 min [MH⁺] 418, 419.

### Ethyl 5-[2-(5-bromo-2-hydroxyphenyl)-1-cyclopenten-1-yl]-3-pyridazinecarboxylate

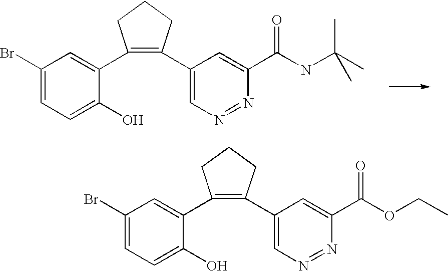

5-[2-(5-Bromo-2-hydroxyphenyl)-1-cyclopenten-1-yl]-N-(1,1-dimethylethyl)-3-pyridazinecarboxamide (6.0 g, 13.95 mmol) in ethanol (75 ml) was treated with concentrated sulphuric acid/water (24/10 ml) and refluxed for two hours. The reaction was poured into water (200 ml) and extracted with ethyl acetate(3×30 ml). After drying the product was purified by chromatography giving the title compound (1.8 g, 32% yield).

LC/MS Rt=3.2 min [MH⁺] 391, 392.

### Ethyl 5-[2-(5-chloro-2-hydroxyphenyl)-1-cyclopenten-1-yl]-3-pyridazinecarboxylate

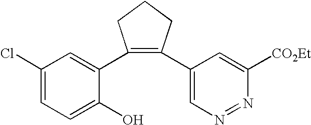

5-(2-{5-Chloro-2-[(phenylmethyl)oxy]phenyl}-1-cyclopenten-1-yl)-N-(1,1-dimethylethyl)-3-pyridazinecarboxamide (970 mg, 2.1 mmol) in ethanol/sulphuric acid/water 2:2:1 (20 ml) was heated at 90° C. for 2 hours. After cooling the solution was diluted with water/ether and the organic layer dried (magnesium sulphate) evaporated and purified by chromatography on silica eluting with ethyl acetate/iso-hexane (1:1) to give the title compound as a white solid (260 mg).

LC/MS: [M+H] 345.3, 347.3, Rt=3.15 min

### Methyl 6-{2-[2-hydroxy-5-trifluoromethyl)phenyl]-1-cyclopenten-1-yl}-2-pyridinecarboxylate

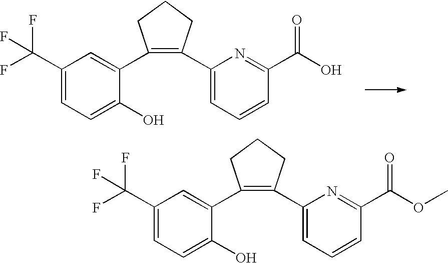

6-{2-[2-hydroxy-5-(trifluoromethyl)phenyl]cyclopent-1-en-1-yl}pyridine-2-carboxylic acid (2.49 g, 7.1 3 mmol) was dissolved in anhydrous methanol (100 ml) and cooled in an ice bath. 2M Trimethylsilyldiazomethane in hexanes (25 ml) was added slowly. Bubbles of nitrogen were observed after the addition of 5 ml; the addition was continued until no more bubbling was observed. The solvent was then removed in vacuo to yield a dark oil. This was purified by column chromatography eluting with 30% ethyl acetate/isohexane. This yielded the title compound as a yellow solid. LC/MS Rt=3.47 [MH⁺] 364.

### Methyl 6-{2-[2-hydroxy-5-(trifluoromethyl)phenyl]-1-cyclopenten-1-yl}-2-pyrazinecarboxylate

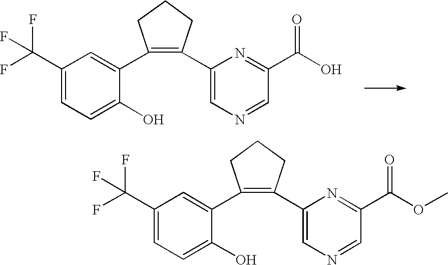

Procedure as for methyl 6-{2-[2-hydroxy-5-trifluoromethyl)phenyl]-1-cyclopenten-1-yl}-2-pyridinecarboxylate. LC/MS t=3.47, [MH⁺] 365.

### Methyl 6-[2-(5-fluoro-2-hydroxyphenyl)-1-cyclopenten-1-yl]-2-pyridinecarboxylate

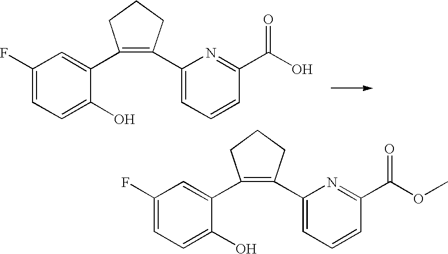

6-[2-(5-Fluoro-2-hydroxyphenyl]-cyclopenten-1-yl-2-pyridinecarboxylic acid (5.0 g, 16.72 mmol) in methanol (200 ml) and concentrated sulphuric acid (4 ml) were refluxed overnight under nitrogen. The reaction mixture was then cooled and treated with 0.880 ammonia (8 ml) and evaporated to an oil under reduced pressure. After partitioning between ethyl acetate and water, the resulting product was purified by flash chromatography with a gradient of diethyl ether/iso-hexane(10-30%) giving the title compound (3.5 g, 83%). LC/MS Rt=3.16 min. [MH⁺] 314

### Methyl 6-[2-(5-fluoro-2-hydroxyphenyl)-1-cyclopenten-1-yl]-2-pyrazinecarboxylate

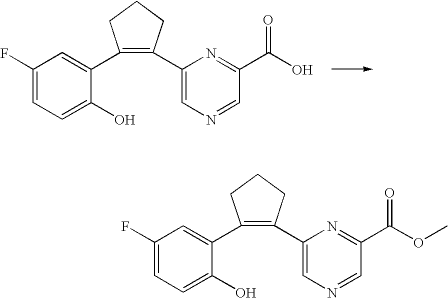

Procedure as for methyl 6-[2-(5-fluoro-2-hydroxyphenyl)-1-cyclopenten-1-yl]-2-pyridinecarboxylate.

LC/MS Rt=2.98min, [MH⁺] 315.

### Ethyl 2-fluoro-5-{2-[2-hydroxy-5-(trifluoromethyl)pyridin-3-yl]cyclopent-1-en-1-yl}benzoate

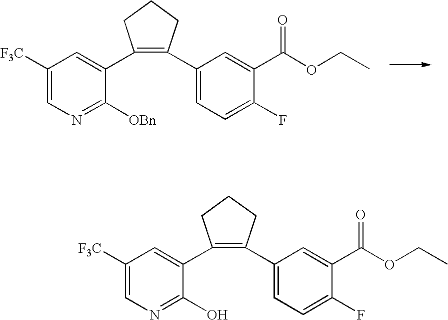

Ethyl 2-fluoro-5{2-[2-(phenylmethoxy)-5-(trifluoromethyl)pyridin-3-yl]cyclopent-1-en-1-yl}benzoate (6.77 g, 14.0 mmol) was dissolved in trifluoroacetic acid (50 ml). The solution was stirred at room temperature for 36 hours. The mixture was treated with 5% aqueous sodium bicarbonate solution, and extracted with diethyl ether (×2). The combined extracts were dried (Na₂SO₄) and concentrated in vacuo. The residue was purified by flash chromatography on silica (gradient elution, 0-30% ethyl acetate/cyclohexane) to give the title compound (3.35 g). LC/MS Rt=3.46 min [MH⁺] 396.

### Ethyl 3-fluoro-5-{2-[2-hydroxy-5-(trifluoromethyl)pyridin-3-yl]cyclopent-1-en-1yl}benzoate

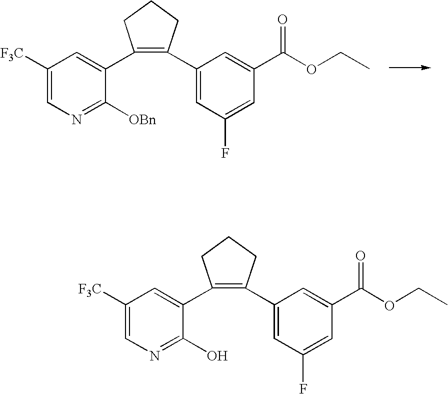

Ethyl 3-fluoro-5-{2-[2-(phenylmethoxy)-5-(trifluoromethyl)pyridin-3-yl]cyclopent-1-en-1-yl}benzoate (7.42 g, 15.3 mmol) was dissolved in trifluoroacetic acid (50 ml). The solution was stirred at room temperature for 18 hours. The mixture was treated with 5% aqueous sodium bicarbonate solution, and extracted with diethyl ether (×2). The combined extracts were dried (Na₂SO₄) and concentrated in vacuo. The residue was purified by flash chromatography on silica (gradient elution, 0-30% ethyl acetate/cyclohexane) to give the title compound (3.3 g). LC/MS Rt=3.56 min [MH⁺] 396.

### Ethyl 5-{2-[2-(hydroxy)-5-(trifluoromethyl)pyridin-3-yl]cyclopent-1-en-1-yl}-3-(trifluoroacetamido)benzoate

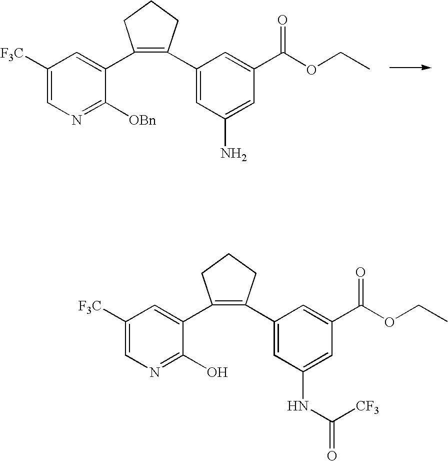

Ethyl 3-amino-5-{2-[2-(phenylmethoxy)-5-(trifluoromethyl)pyridin-3-yl]cyclopent-1-en-1-yl}benzoate (4.5 g, 7.79 mmol) was dissolve in trifluoroacetic acid (50 ml) and stirred at room temperature for 20 hours. The mixture was neutralised with 5% aqueous sodium hydrogen carbonate, and extracted with water. The organic extracts were washed with further water, dried (Na₂SO₄), and concentrated in vacuo. The residue was purified by flash chromatography on silica (gradient elution, 0-65% ethyl acetate/cyclohexane) to give the required product (1.99 g). LC/MS Rt=3.52 min [MH⁺] 489.

### (2,4-Dichlorophenyl)methyl 6-{2-[2-{[(2,4-dichlorophenyl)methyl]oxy}-5-(trifluoromethyl)phenyl]-1-cyclopenten-1-yl}-2-pyridinecarboxylate

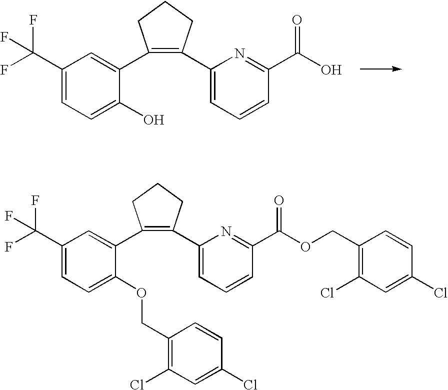

6-{2-[2-Hydroxy-5-(trifluoromethyl)phenyl]cyclopent-1-en-1-yl}-pyridine-2-carboxylic acid (0.067 g, 0.19 mmol), potassium carbonate (0.079 g, 0.57 mmol), 2,4-dichlorobenzyl bromide (0.082 g, 0.42 mmol) and DMF (2 ml) were heated at 55° C. for 3 hours under a nitrogen atmosphere. After cooling the reaction was diluted with ethyl acetate and washed with water (×2). The aqueous layers were washed with ethyl acetate (×2). The combined organic layers were then washed with brine, dried over MgSO₄, filtered and concentrated in vacuo to give a dark oil. This was purified by column chromatography eluting with 10% ethyl acetate/isohexane to yield the title compound as a brown oil (0.095 g, 74%).

LC/MS t=4.97, [MH⁺] 668

### (2,6-Difluorophenyl)methyl 6-{2-[2-{[(2,6-difluorophenyl)methyl]oxy}-5-(trifluoromethyl)phenyl]-1-cyclopenten-1-yl}-2-pyridinecarboxylate

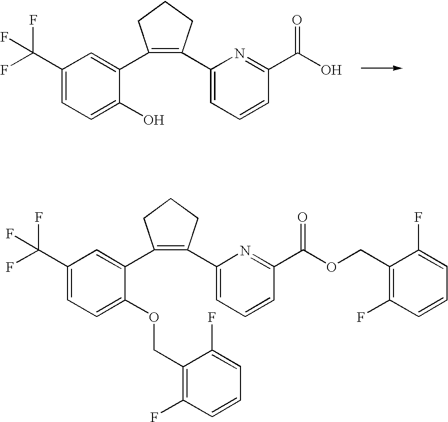

Procedure as for (2,4-dichlorophenyl)methyl 6-{2-[2-{[(2,4-dichlorophenyl)methyl]oxy}-5-(trifluoromethyl)phenyl]-1-cyclopenten-1-yl}-2-pyridinecarboxylate.

LC/MS t=4.34, [MH⁺] 602

### Methyl 5-[2-(5-chloro-2-{[(4-fluorophenyl)methyl]oxy}phenyl)-1-cyclopenten-1-yl]-2-methyl-3-pyridinecarboxylate

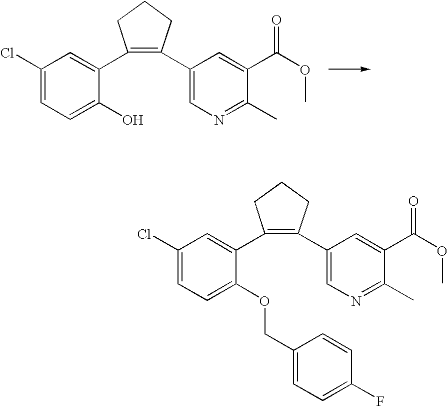

A mixture of methyl 5-[2-(5-chloro-2-hydroxyphenyl)-1-cyclopenten-1-yl]-2-methyl-3-pyridinecarboxylate (150 mg, 0.44 mmol), 4-fluorobenzyl bromide (95 mg, 0.50 mmol) and potassium carbonate (138 mg, 1 mmol) in acetone (5 ml) was stirred and refluxed for 3 hours then cooled, filtered, evaporated and purified by chromatography on silica eluting with ethyl acetate/iso-hexane (15:85) to give a colourless gum (191 mg).

LC/MS t=4.07, [MH+] 452.3

The following intermediates were prepared by a similar route to methyl 5-[2-(5-chloro-2-{[(4-fluorophenyl)methyl]oxy}phenyl)-cyclopenten-1-yl]-2-methyl-3-pyridinecarboxylate from the appropriate intermediates.

| | Name | LC/MS |
|---|---|---|
| 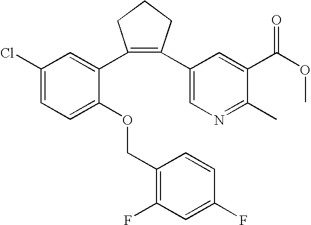 | Methyl 5-[2-(5-chloro-2-{[(2,4- difluorophenyl)methyl]oxy}phenyl)-1- cyclopenten-1-yl]-2-methyl-3- pyridinecarboxylate | Rt = 4.09, [MH+] 470.4, 472.3 |
| 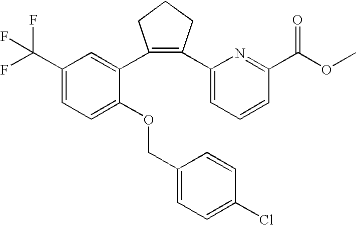 | Methyl 6-{2-[2-{[(4- chlorophenyl)methyl}oxy)-5- (trifluoromethyl)phenyl]-1- cyclopenten-1-yl}-2- pyridinecarboxylate | Rt = 4.16, [MH⁺] 488 |
| 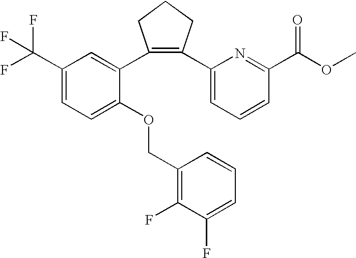 | Methyl 6-{2-[2-{[(2,3- difluorophenyl)methyl]oxy}-5- (trifluoromethyl)phenyl]-1- cyclopenten-1-yl}-2- pyridinecarboxylate | Rt = 4.08 [MH⁺] 490 |
| 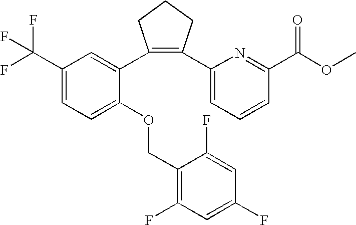 | Methyl 6-[2-(5-(trifluoromethyl)-2- {[(2,4,6- trifluorophenyl)methyl]oxy}phenyl)-1- cyclopenten-1-yl]-2- pyridinecarboxylate | Rt = 4.02, [MH⁺] 508 |
| 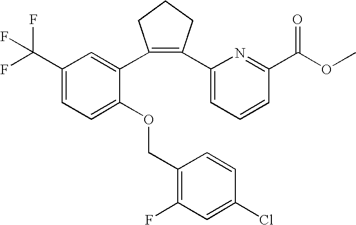 | Methyl 6-{2-[2-{[(4-chloro-2- fluorophenyl)methyl]oxy}-5- (trifluoromethyl)phenyl]-1- cyclopenten-1-yl}-2- pyridinecarboxylate | Rt = 4.20, [MH⁺] 506 |
| 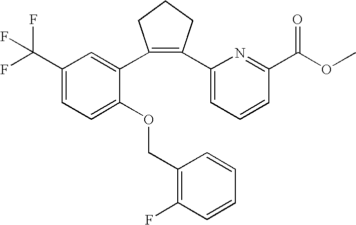 | Methyl 6-{2-[2-{[(2- fluorophenyl)methyloxy}-5- (trifluoromethyl)phenyl]-1- cyclopenten-1-yl}-2- pyridinecarboxylate | Rt = 4.02, [MH⁺] 472 |
| 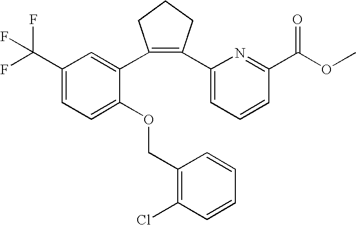 | Methyl 6-{2-[2-{[(2- chlorophenyl)methyl]oxy}-5- (trifluoromethyl)phenyl]-1- cyclopenten-1-yl}-2- pyridinecarboxylate | Rt = 4.19, [MH⁺] 488. |
| 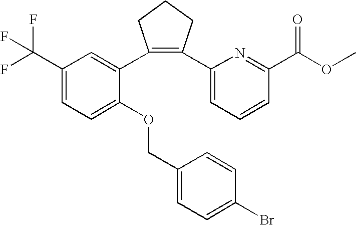 | Methyl 6-{2-[2-{[(4- bromophenyl)methyl]oxy)-5- (trifluoromethyl)phenyl]-1- cyclopenten-1-yl}-2- pyridinecarboxylate | Rt = 4.22, [MH⁺] 532, 534 |
| 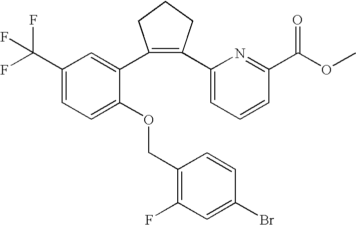 | Methyl 6-{2-[2-{[(4-bromo-2- fluorophenyl)methyl]oxy}-5- (trifluoromethyl)phenyl]-1- cyclopenten-1-yl}-2- pyridinecarboxylate | Rt = 4.24, [MH⁺] 550, 552 |
| 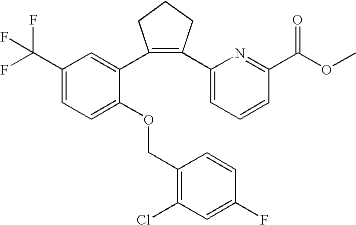 | Methyl 6-{2-[2-{[(2-chloro-4- fluorophenyl)methyl]oxy}-5- (trifluoromethyl)phenyl]-1- cyclopenten-1-yl}-2- pyridinecarboxylate | Rt = 4.21, [MH⁺] 506 |
| 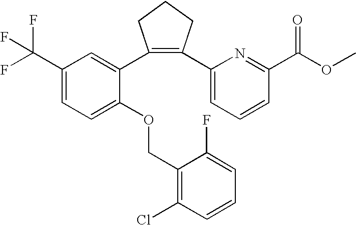 | Methyl 6-{2-[2-{[(2-chloro-6- fluorophenyl)methyl]oxy}-5- (trifluoromethyl)phenyl]-1- cyclopenten-1-yl}-2- pyridinecarboxylate | Rt = 4.09, [MH⁺] 506 |
| 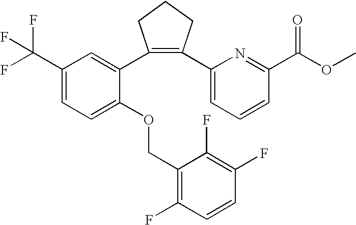 | Methyl 6-[2-(5-(trifluoromethyl)-2- {[(2,3,6- trifluorophenyl)methyl]oxy}phenyl)-1- cyclopenten-1-yl]-2- pyridinecarboxylate | Rt = 3.99, [MH⁺] 508 |
| 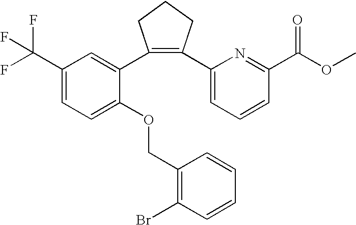 | Methyl 6-{2-[2-{[(2- bromophenyl)methyl]oxy}-5- (trifluoromethyl)phenyl]-1- cyclopenten-1-yl}-2- pyridinecarboxylate | Rt = 4.22, [MH⁺] 534, 534 |
| 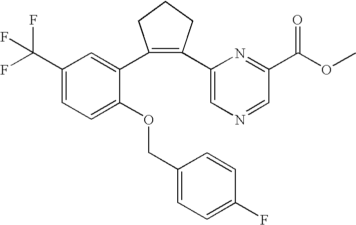 | Methyl 6-{2-(2-{[(4- fluorophenyl)methyl]oxy}-5- (trifluoromethyl)phenyl]-1- cyclopenten-1-yl}-2- pyrazinecarboxylate | Rt = 3.92, [MH⁺] 473 |
| 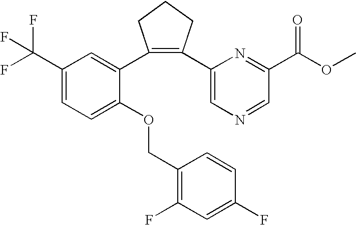 | Methyl 6-{2-[2-{[(2,4- difluorophenyl)methyl]oxy}-5- (trifluoromethyl)phenyl]-1- cyclopenten-1-yl}-2- pyrazinecarboxylate | Rt = 3.95, [MH⁺] 491 |
| 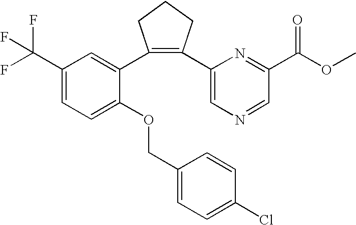 | Methyl 6-{2-[2-{[(4- chlorophenyl)methyl]oxy}-5- (trifluoromethyl)phenyl]-1- cyclopenten-1-yl}-2- pyrazinecarboxylate | Rt = 4.21, [MH⁺] 489 |
| 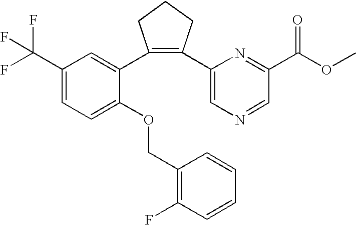 | Methyl 6-{2-[2-{[(2- fluorophenyl)methyl]oxy}-5- (trifluoromethyl)phenyl]-1- cyclopenten-1-yl}-2- pyrazinecarboxylate | Rt = 3.95, [MH⁺] 473 |
| 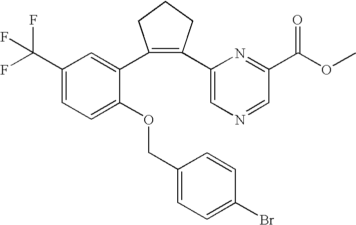 | Methyl 6-{2-[2-{[(4- bromophenyl)methyl]oxy}-5- (trifluoromethyl)phenyl]-1- cyclopenten-1-yl}-2- pyrazinecarboxylate | Rt = 4.15, [MH⁺] 533, 535 |
| 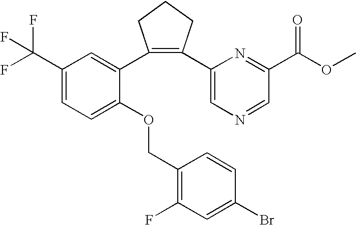 | Methyl 6-{2-[2-{[(4-bromo-2- fluorophenyl)methyl]oxy}-5- (trifluoromethyl)phenyl]-1- cyclopenten-1-yl}-2- pyrazinecarboxylate | Rt = 4.26, [MH⁺] 551, 553 |
| 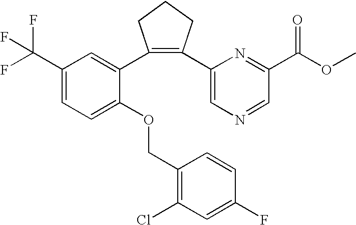 | Methyl 6-{2-[2-{[(2-chloro-4- fluorophenyl)methyl]oxy}-5- (trifluoromethyl)phenyl]-1- cyclopenten-1-yl}-2- pyazinecarboxylate | Rt = 4.23, [MH⁺] 507 |
| 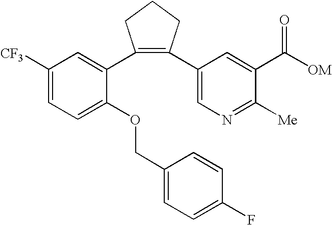 | Methyl 5-{2-[2-{[(4- fluorophenyl)methyl]oxy}-5- (trifluoromethyl)phenyl]-1- cyclopenten-1-yl}-2-methyl-3- pyridinecarboxylate | Rt = 3.98, [MH+] 486.5 |
| 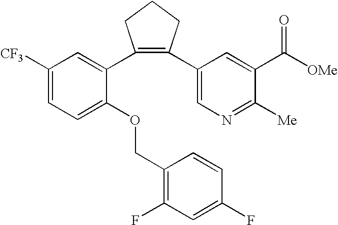 | Methyl 5-{2-[2-{[(2,4- difluorophenyl)methyl]oxy}-5- (trifluoromethyl)phenyl]-1- cyclopenten-1-yl}-2-methyl-3- pyridinecarboxylate | Rt = 4.03 [MH+] 504.4 |
| 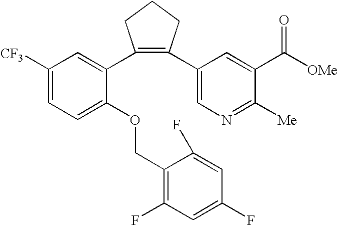 | Methyl 5-{2-[2-{[(2,4,6- trifluorophenyl)methyl]oxy}-5- (trifluoromethyl)phenyl]-1- cyclopenten-1-yl}-2-methyl-3- pyridinecarboxylate | Rt = 4.06 [MH+] 522.4 |
| 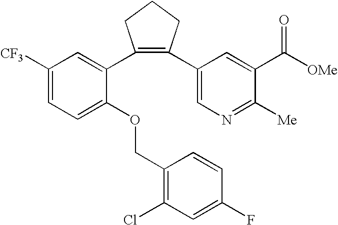 | Methyl 5-{2-[2-{[(2-chloro-4- fluorophenyl)methyl]oxy}-5- (trifluoromethyl)phenyl]-1 cyclopenten-1-yl}-2-methyl-3- pyridinecarboxylate | Rt = 4.22 [MH+] 520.4, 522.4 |
| 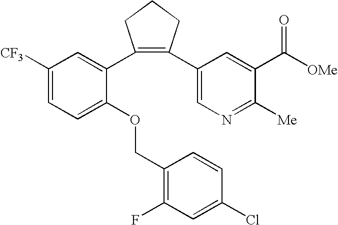 | Methyl 5-{2-[2-{[(4-chloro-2- fluorophenyl)methyl]oxy}-5- (trifluoromethyl)phenyl]-1- cyclopenten-1-yl}-2-methyl-3- pyridinecarboxylate | Rt = 4.23 [MH+] 520.4, 522.4 |
| 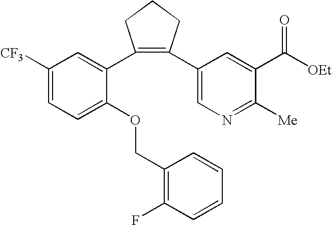 | Ethyl 5-{2-[2-{[(2- fluorophenyl)methyl]oxy}-5- (trifluoromethyl)phenyl]-1- cyclopenten-1-yl}-2-methyl-3- pyridinecarboxylate | Rt = 4.41 [MH+] 500.4 |
| 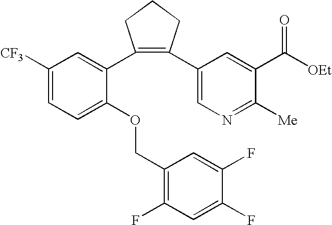 | Ethyl 5-{2-[2-{[(2,4,5- trifluorophenyl)methyl]oxy}-5- (trifluoromethyl)phenyl]-1- cyclopenten-1-yl}-2-methyl-3- pyridinecarboxylate | Rt = 4.57 [MH+] 536.4 |
| 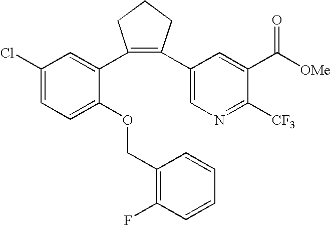 | Methyl 5-[2-(5-chloro-2-{[(2- fluorophenyl)methyl]oxy}phenyl)-1- cyclopenten-1-yl]-2-(trifluoromethyl)- 3-pyridinecarboxylate | Rt = 3.95, [MH+] 506.4 |
| 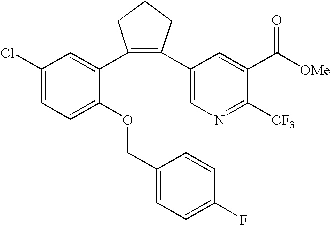 | Methyl 5-[2-(5-chloro-2-{[(4- fluorophenyl)methyl]oxy}phenyl)-1- cyclopenten-1-yl]-2-(trifluoromethyl)- 3-pyridinecarboxylate | Rt = 3.96 [MH+] 506.4 |
| 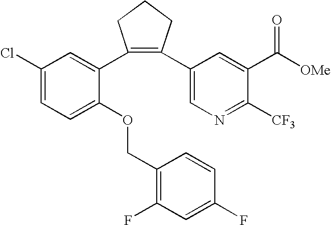 | Methyl 5-[2-(5-chloro-2-{[(2,4- difluorophenyl)methyl]oxy}phenyl)-1- cyclopenten-1-yl]-2-(trifluoromethyl)- 3-pyridinecarboxylate | Rt = 3.95 [MH+] 524.4 |
| 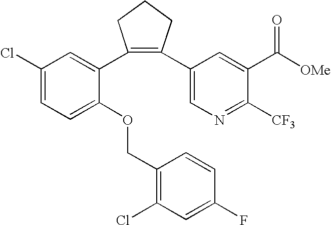 | Methyl 5-[2-(5-chloro-2-{[(2-chloro-4- fluorophenyl)methyl]oxy}phenyl)-1- cyclopenten-1-yl]-2-(trifluoromethyl)- 3-pyridinecarboxylate | Rt = 4.06 [MH+] 540.3 |
| 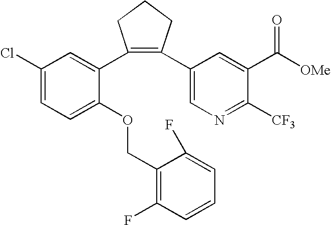 | Methyl 5-[2-(5-chloro-2-{[(2,6- difluorophenyl)methyl]oxy}phenyl)-1- cyclopenten-1-yl]-2-(trifluoromethyl)- 3-pyridinecarboxylate | Rt = 3.93 [MH+] 524.4 |
| 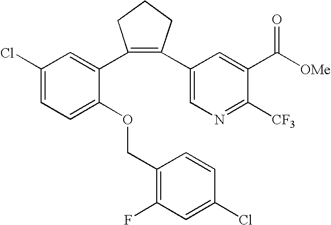 | Methyl 5-[2-(5-chloro-2-{[(4-chloro-2- fluorophenyl)methyl]oxy}phenyl)-1- cyclopenten-1-yl]-2-(trifluoromethyl)- 3-pyridinecarboxylate | Rt = 4.11 [MH+] 540.3 |
| 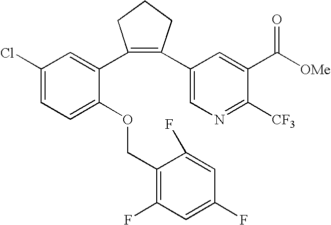 | Methyl 5-[2-(5-chloro-2-{[(2,4,6- trifluorophenyl)methyl]oxy}phenyl)-1- cyclopenten-1-yl]-2-(trifluoromethyl)- 3-pyridinecarboxylate | Rt = 4.02 [MH+] 542.3 |
| 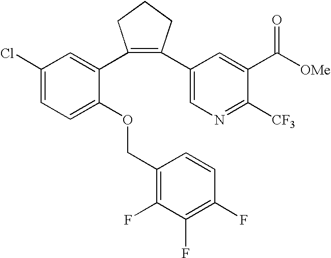 | Methyl 5-[2-(5-chloro-2-{[(2,3,4- trifluorophenyl)methyl]oxylphenyl)-1- cyclopenten-1-yl]-2-(trifluoromethyl)- 3-pyridinecarboxylate | Rt = 4.01 [MH+] 542.3 |
| 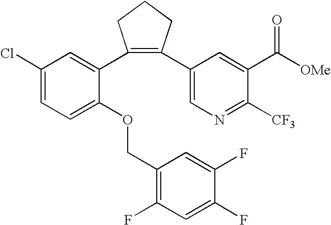 | Methyl 5-[2-(5-chloro-2-{[2,4,5- trifluorophenyl)methyl]oxy}phenyl)-1- cyclopenten-1-yl]-2-(trifluoromethyl)- 3-pyridinecarboxylate | Rt = 4.04 [MH+] 542.3 |
| 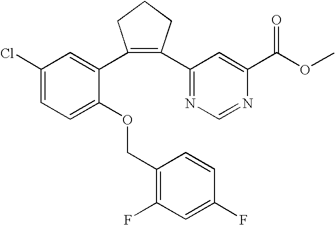 | Methyl 6-[2-(5-chloro-2-{[(2,4- difluorophenyl)methyl]oxy}phenyl)-1- cyclopenten-1-yl-4- pyrimidinecarboxylate | Rt = 4.10 [MH+] 457, 459 |

### Ethyl 6-[2-(5-chloro-2-{[(2-fluorophenyl)methyl]oxy}phenyl)-1-cyclopenten-1-yl]-2-pyridinecarboxylate

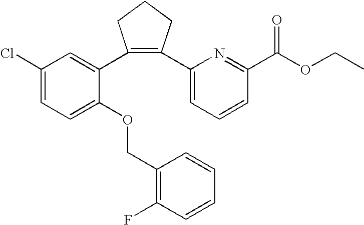

Ethyl 6-[2-(5-chloro-2-hydroxyphenyl)-1-cyclopenten-1-yl]-2-pyridinecarboxylate (100 mg, 0.29 mmol), 2-fluorobenzyl bromide (0.035 ml, 0.32 mmol) and potassium carbonate (100 mg, 0.73 mmol) in acetone (3 ml) were refluxed overnight under nitrogen. The reaction mixture was then filtered through hiflo and evaporated to give the title compound.

LC/MS: Rt=4.1 [MH⁺] 452, 455

The following intermediates were prepared by a similar route to ethyl 6-[2-(5-chloro-2-{[(2-fluorophenyl)methyl]oxy}phenyl)-1-cyclopenten-1-yl]-2-pyridinecarboxylate from the appropriate intermediates.

| | Name | LC/MS |
|---|---|---|
| 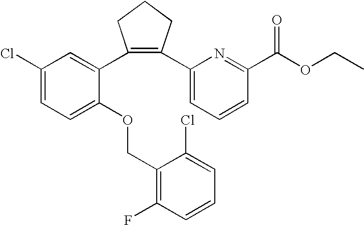 | Ethyl 6-[2-(5-chloro-2-{[(2-chloro- 6-fluorophenyl)methyl]oxy}phenyl)- 1-cyclopenten-1-yl]-2- pyridinecarboxylate | Rt = 4.20, [MH+] 486, 489 |
| 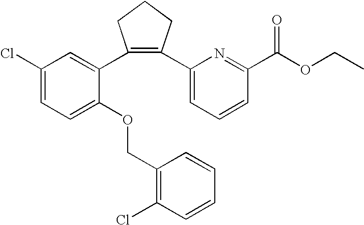 | Ethyl 6-[2-(5-chloro-2-{[(2- chlorophenyl)methyl]oxy}phenyl)- 1-cyclopenten-1-yl]-2- pyridinecarboxylate | Rt = 4.28, [MH+] 468, 471 |
| 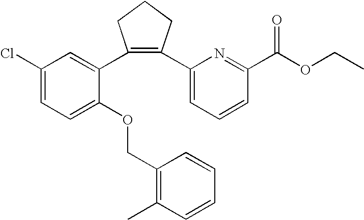 | Ethyl 6-[2-(5-chloro-2-{[(2- methylphenyl)methyl]oxy}phenyl)- 1-cyclopenten-1-yl]-2- pyridinecarboxylate | Rt = 3.99, [MH+] 448, 451 |
| 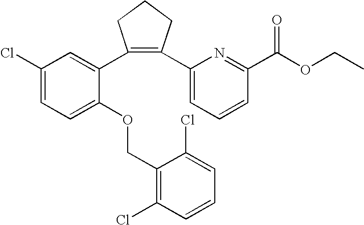 | Ethyl 6-[2-(5-chloro-2-{[(2,6- dichlorophenyl)methyl]oxy}phenyl)- 1-cyclopenten-1-yl-2- pyridinecarboxylate | Rt = 4.08, [MH+] 504, 506 |
| 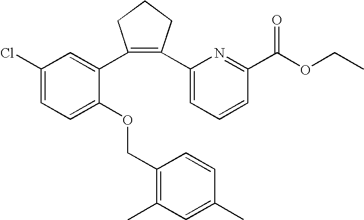 | Ethyl 6-[2-(5-chloro-2-{[(2,4- dimethylphenyl)methyl]oxy}phenyl)- cyclopenten-1-yl]-2- pyridinecarboxylate | Rt = 4.09, [MH+] 462, 464 |
| 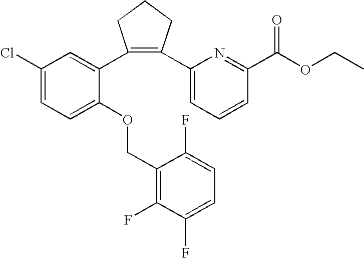 | Ethyl 6-[2-(5-chloro-2-{[(2,3,6- trifluorophenyl)methyl]oxy}phenyl)- 1-cyclopenten-1-yl]-2- pyridinecarboxylate | Rt = 4.11, [MH+] 488, 490 |
| 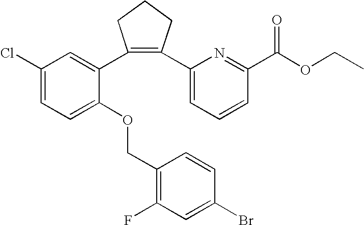 | Ethyl 6-[2-(2-{[(4-bromo-2- fluorophenyl)methyl]oxy}-5- chlorophenyl)-1-cyclopenten-1-yl]- pyridinecarboxylate | Rt = 4.11, [MH+] 532, 534 |
| 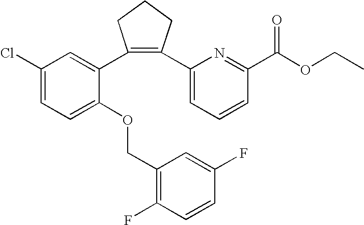 | Ethyl 6-[2-(5-chloro-2-{[(2,5- difluorophenyl)methyl]oxy}phenyl)- 1-cyclopenten-1-yl]-2- pyridinecarboxylate | Rt = 3.95, [MH+] 470, 473 |
| 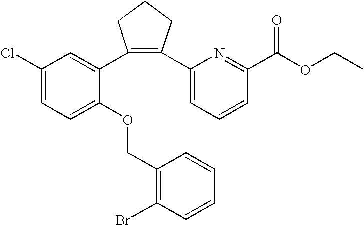 | Ethyl 6-[2-(2-{[(2- bromophenyl)methyl]oxy}-5- chlorophenyl)-1-cyclopenten-1-yl]- 2-pyridinecarboxylate | Rt = 4.32, [MH+] 514, 516 |
| 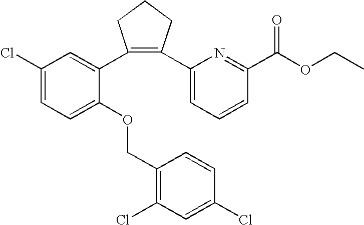 | Ethyl 6-[2-(5-chloro-2-{[(2,4- dichlorophenyl)methyl]oxy}phenyl)- 1-cyclopenten-1-yl]-2- pyridinecarboxylate | Rt = 4.45, [MH+] 504, 506 |
| 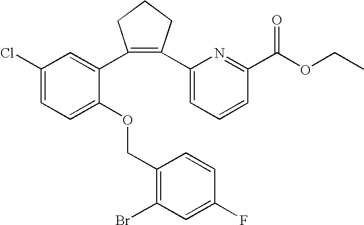 | Ethyl 6-[2-(2-{[(2-bromo-4- fluorophenyl)methyl]oxy}-5- chlorophenyl)-1-cyclopenten-1-yl]- 2-pyridinecarboxylate | Rt = 4.33, [MH+] 532, 534 |
| 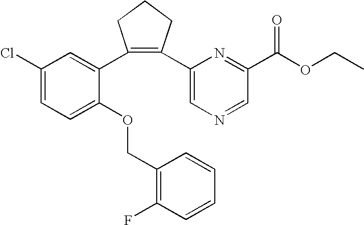 | Ethyl 6-[2-(5-chloro-2-{[(2- fluorophenyl)methyl]oxy}phenyl)-1- cyclopenten-1-yl]-2- pyrazinecarboxylate | Rt = 3.81, [MH+] 453, 456 |
| 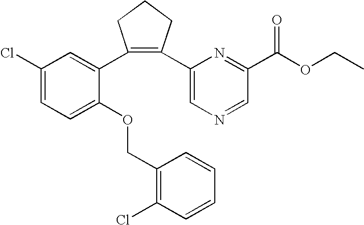 | Ethyl 6-[2-(5-chloro-2-{[(2- chlorophenyl)methyl]oxy}phenyl)- 1-cyclopenten-1-yl]-2- pyrazinecarboxylate | Rt = 3.96, [MH+] 469, 472 |
| 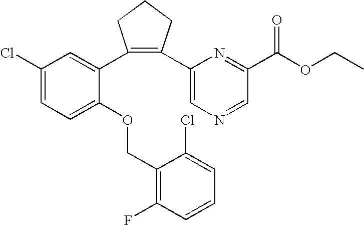 | Ethyl 6-[2-(5-chloro-2-{[(2-chloro- 6-fluorophenyl)methyl]oxy}phenyl)- 1-cyclopenten-1-yl]-2- pyrazinecarboxylate | Rt = 3.88, [MH+] 487, 490 |
| 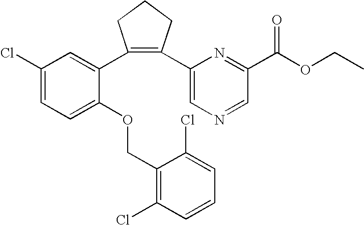 | Ethyl 6-[2-(5-chloro-2-{[(2,6- dichlorophenyl)methyl]oxy}phenyl)- 1-cyclopenten-1-yl]-2- pyrazinecarboxylate | Rt = 4.19, [MH+] 505, 507 |
| 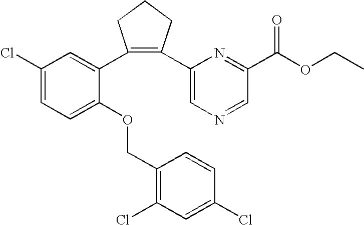 | Ethyl 6-[2-(5-chloro-2-{[(2,4- dichlorophenyl)methyl]oxy}phenyl)- 1-cyclopenten-1-yl]-2- pyrazinecarboxylate | Rt = 4.32, [MH+] 505, 507 |
| 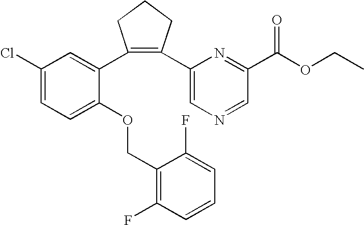 | Ethyl 6-[2-(5-chloro-2-{[(2,6- difluorophenyl)methyl]oxy}phenyl)- 1-cyclopenten-1-yl]-2- pyrazinecarboxylate | Rt = 3.97, [MH+] 473, 474 |
| 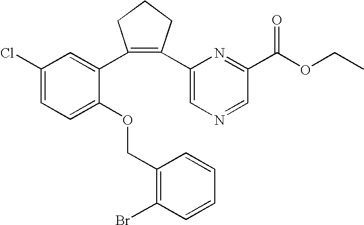 | Ethyl 6-[2-(2-{[(2- bromophenyl)methyl]oxy}-5- chlorophenyl)-1-cyclopenten-1-yl]- 2-pyrazinecarboxylate | Rt = 3.99, [MH+] 515, 517 |
| 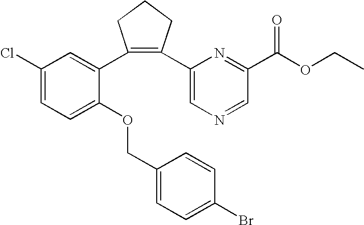 | Ethyl 6-[2-(2-{[(4- bromophenyl)methyl]oxy}-5- chlorophenyl)-1-cyclopenten-1-yl]- 2-pyrazinecarboxylate | Rt = 3.98, [MH+] 515, 517 |
| 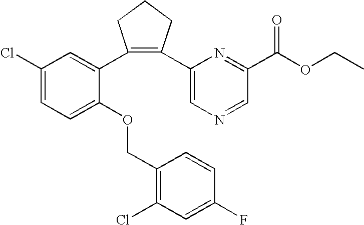 | Ethyl 6-[2-(5-chloro-2-{[(2-chloro- 4-fluorophenyl)methyl]oxy}phenyl)- 1-cyclopenten-1-yl]-2- pyrazinecarboxylate | Rt = 3.97, [MH+] 487, 490 |
| 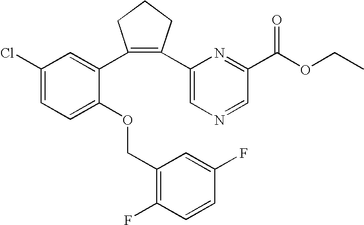 | Ethyl 6-[2-(5-chloro-2-{[(2,5- difluorophenyl)methyl]oxy}phenyl)- 1-cyclopenten-1-yl]-2- pyrazinecarboxylate | Rt = 3.83, [MH+] 471, 473 |
| 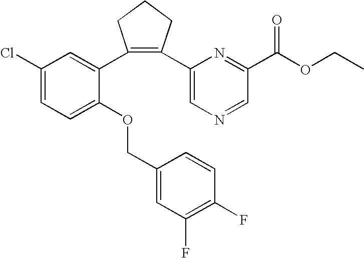 | Ethyl 6-[2-(5-chloro-2-{[(3,4- difluorophenyl)methyl]oxy}phenyl)- 1-cyclopenten-1-yl]-2- pyrazinecarboxylate | Rt = 3.84, [MH+] 471, 473 |
| 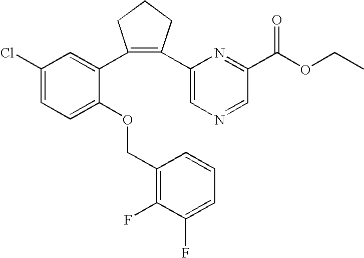 | Ethyl 6-[2-(5-chloro-2-{[(2,3- difluorophenyl)methyl]oxy}phenyl)- 1-cyclopenten-1-yl]-2- pyrazinecarboxylate | Rt = 3.82, [MH+] 471, 473 |
| 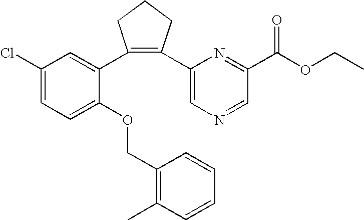 | Ethyl 6-[2-(5-chloro-2-{[(2- methylphenyl)methyl]oxy}phenyl)- 1-cyclopenten-1-yl]-2- pyrazinecarboxylate | Rt = 4.34, [MH+] 449 |
| 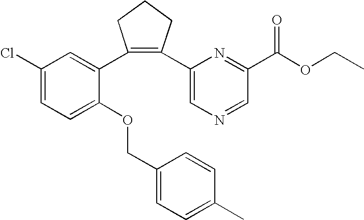 | Ethyl 6-(2-(5-chloro-2-{[(4- methylphenyl)methyl]oxy}phenyl)- 1-cyclopenten-1-yl}-2- pyrazinecarboxylate | Rt = 4.37, [MH+] 449, 451 |
| 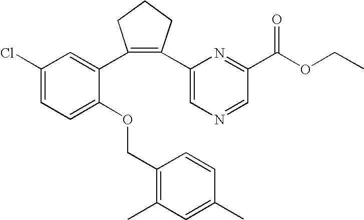 | Ethyl 6-[2-(5-chloro-2-{[(2,4- dimethylphenyl)methyl]oxy}phenyl)- 1-cyclopenten-1-yl]-2- pyrazinecarboxylate | Rt = 4.48, [MH+] 463 |
| 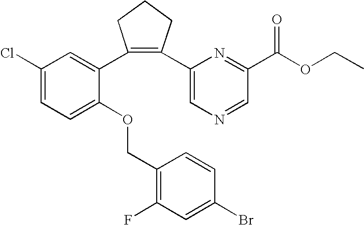 | Ethyl 6-[2-(2-{[(4-bromo-2- fluorophenyl)methyl]oxy}-5- chlorophenyl)-1-cyclopenten-1-yl]- 2-pyrazinecarboxylate | Rt = 4.47, [MH+] 533, 535 |
| 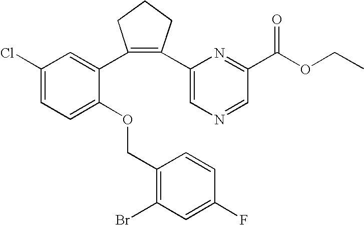 | Ethyl 6-[2-(2-{[(2-bromo-4- fluorophenyl)methyl]oxy}-5- chlorophenyl)-1-cyclopenten-1-yl]- 2-pyrazinecarboxylate | Rt = 4.48, [MH+] 533 [MH−] 531 |
| 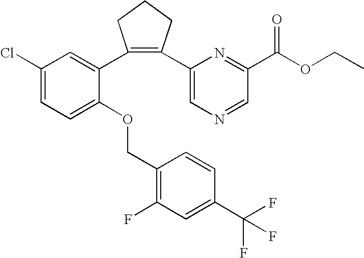 | Ethyl 6-{2-[5-chloro-2-({[2-fluoro-4- (trifluoromethyl)phenyl]methyl}oxy) phenyl]-1-cyclopenten-1-yl}-2- pyrazinecarboxylate | Rt = 4.46, [MH+] 521, 523 |
| 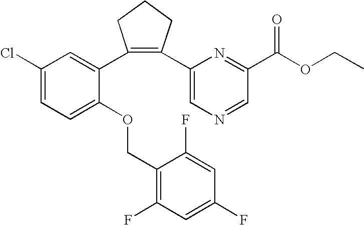 | Ethyl 6-[2-(5-chloro-2-{[(2,4,6- trifluorophenyl)methyl]oxy}phenyl)- 1-cyclopenten-1-yl]-2- pyrazinecarboxylate | Rt = 4.23, [MH+] 489, 491 |
| 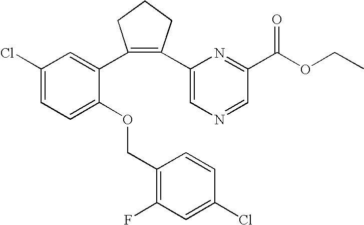 | Ethyl 6-[2-(5-chloro-2-{[(4-chloro- 2-fluorophenyl)methyl]oxy}phenyl)- 1-cyclopenten-1-yl]-2- pyrazinecarboxylate | Rt = 4.23, [MH+] 487 |
| 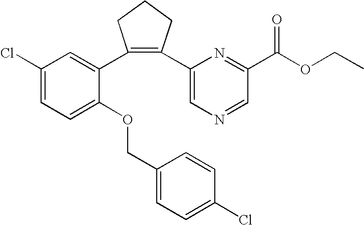 | Ethyl 6-[2-(5-chloro-2-{[(4- chlorophenyl)methyl]oxy}phenyl)- 1-cyclopenten-1-yl]-2- pyrazinecarboxylate | Rt = 4.23, [MH+] 469, 471 |
| 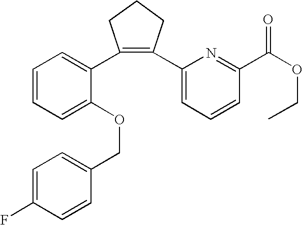 | Ethyl 6-(2-(2-{[(4- fluorophenyl)methyl]oxy}phenyl)- 1-cyclopenten-1-yl]-2- pyridinecarboxylate | Rt = 3.99 min, [M + H] 418. |
| 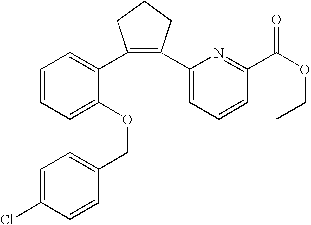 | Ethyl 6-[2-(2-{[(4- chlorophenyl)methyl]oxy}phenyl)- 1-cyclopenten-1-yl]-2- pyridinecarboxylate | Rt = 4.16 min, [M + H] 434. |
| 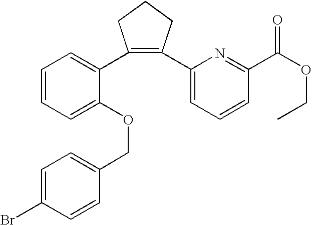 | Ethyl 6-[2-(2-{[(4- bromophenyl)methyl]oxy}phenyl)- 1-cyclopenten-1-yl]-2- pyridinecarboxylate | Rt = 4.21 min, [M + H] 480. |
| 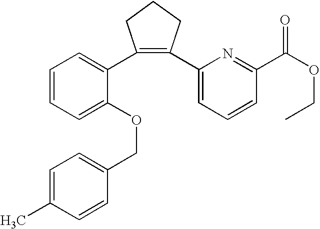 | Ethyl 6-[2-(2-{[(4- methylphenyl)methyl]oxy}phenyl)- 1-cyclopenten-1-yl]-2- pyridinecarboxylate | Rt = 4.11 min, [M + H] 414. |
| 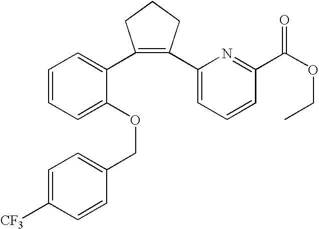 | Ethyl 6-{2-[2-({[4- (trifluoromethyl)phenyl]methyl}oxy) phenyl]-1-cyclopenten-1-yl}-2- pyridinecarboxylate | Rt = 4.17 min, [M + H] 486. |
| 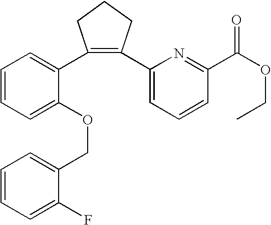 | Ethyl 6-[2-(2-{[(2- fluorophenyl)methyl]oxy}phenyl)- 1-cyclopenten-1-yl]-2- pyridinecarboxylate | Rt = 3.99 min, [M + H] 418. |
| 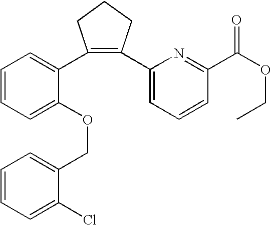 | Ethyl 6-[2-(2-{[(2- chlorophenyl)methyl]oxy}phenyl)- 1-cyclopenten-1-yl]-2- pyridinecarboxylate | Rt = 4.18 min, [M + H] 434. |
| 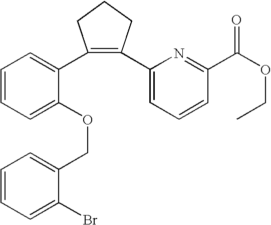 | Ethyl 6-[2-(2-{[(2- bromophenyl)methyl]oxy}phenyl)- 1-cyclopenten-1-yl]-2- pyridinecarboxylate | Rt = 4.18 min, [M + H] 480. |
| 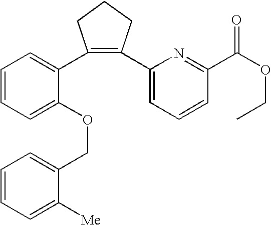 | Ethyl 6-[2-(2-{[(2- methylphenyl)methyl]oxy}phenyl)- 1-cyclopenten-1-yl]-2- pyridinecarboxylate | Rt = 4.06 min, [M + H] 414. |
| 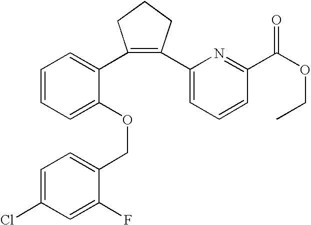 | Ethyl 6-[2-(2-{[(4-chloro-2- fluorophenyl)methyl]oxy}phenyl)- 1-cyclopenten-1-yl]-2- pyridinecarboxylate | Rt = 4.18 min, [M + H] 452. |
| 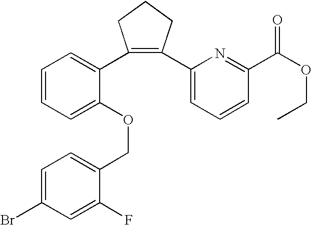 | Ethyl 6-[2-(2-{[(4-bromo-2- fluorophenyl)methyl]oxy}phenyl)- 1-cyclopenten-1-yl-2- pyridinecarboxylate | Rt = 4.21 min, [M + H] 498. |
| 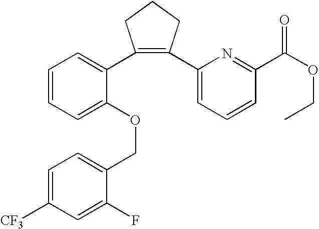 | Ethyl 6-{2-[2-({[2-fluoro-4- trifluoromethyl)phenyl]methyl}oxy) phenyl]-1-cyclopenten-1-yl}-2- pyridinecarboxylate | Rt = 4.22 min, [M + H] 486. |
| 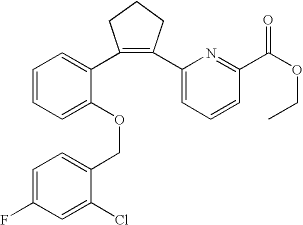 | Ethyl 6-[2-(2-{[(2-chloro-4- fluorophenyl)methyl]oxy}phenyl)- 1-cyclopenten-1-yl]-2- pyridinecarboxylate | Rt = 4.21 min, [M + H] 452. |
| 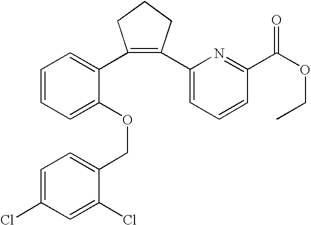 | Ethyl 6-[2-(2-{[(2,4- dichlorophenyl)methyl]oxy}phenyl)- 1-cyclopenten-1-yl]-2- pyridinecarboxylate | Rt = 4.43 min, [M + H] 468. |
| 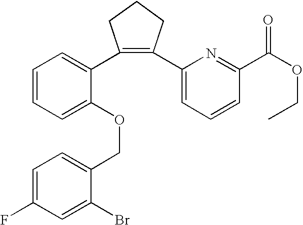 | Ethyl 6-[2-(2-{[(2-bromo-4- fluorophenyl)methyl]oxy}phenyl)- 1-cyclopenten-1-yl]-2- pyridinecarboxylate | Rt = 4.26 min, [M + H] 498. |
| 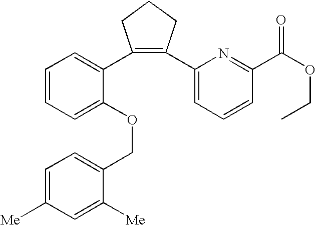 | Ethyl 6-[2-(2-{[(2,4- dimethylphenyl)methyl]oxy}phenyl)- 1-cyclopenten-1-yl]-2- pyridinecarboxylate | Rt = 4.22 min, [M + H] 428. |
| 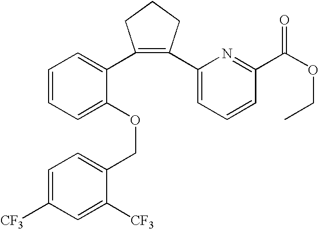 | Ethyl 6-{2-[2-({[2,4- bis(trifluoromethyl)phenyl]methyl} oxy)phenyl]-1-cyclopenten-1-yl}- 2-pyridinecarboxylate | Rt = 4.43 min, [M + H] 536. |
| 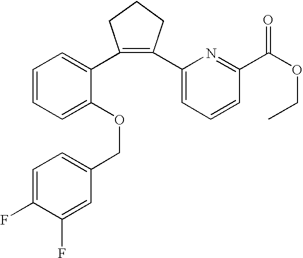 | Ethyl 6-[2-(2-{[(3,4- difluorophenyl)methyl]oxy}phenyl)- 1-cyclopenten-1-yl]-2- pyridinecarboxylate | Rt = 4.03 min, [M + H] 436. |
| 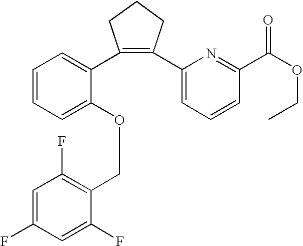 | Ethyl 6-[2-(2-{[(2,4,6- trifluorophenyl)methyl]oxy}phenyl)- 1-cyclopenten-1-yl]-2- pyridinecarboxylate | Rt = 3.98 min, [M + H] 454. |
| 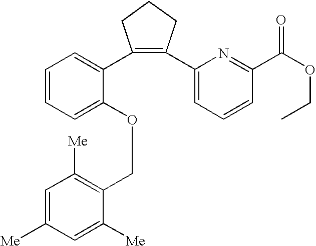 | Ethyl 6-[2-(2-{[(2,4,6- trimethylphenyl)methyl]oxy}phenyl)- 1-cyclopenten-1-yl]-2- pyridinecarboxylate | Rt = 4.31 min, [M + H] 442. |
| 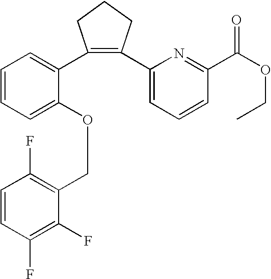 | Ethyl 6-[2-(2-{[(2,3,6- trifluorophenyl)methyl]oxy}phenyl)- 1-cyclopenten-1-yl]-2- pyridinecarboxylate | Rt = 3.95 min, [M + H] 454. |
| 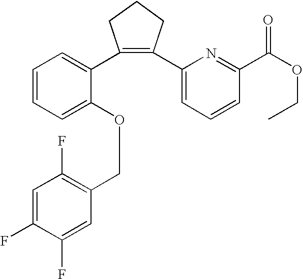 | Ethyl 6-[2-(2-{[(2,4,5- trifluorophenyl)methyl]oxy}phenyl)- 1-cyclopenten-1-yl]-2- pyridinecarboxylate | Rt = 4.08 min, [M + H] 454. |
| 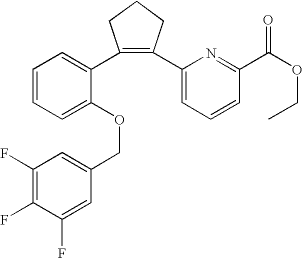 | Ethyl 6-[2-(2-{[(3,4-5- trifluorophenyl)methyl]oxy}phenyl)- 1-cyclopenten-1-yl]-2- pyridinecarboxylate | Rt = 4.12 min, [M + H] 454. |
| 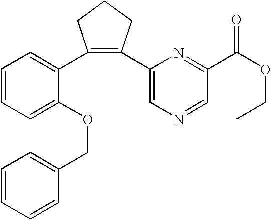 | Ethyl 6-(2-{2- [(phenylmethyl)oxy]phenyl}-1- cyclopenten-1-yl)-2- pyrazinecarboxylate | Rt = 3.83 min, [M + H] 401. |
| 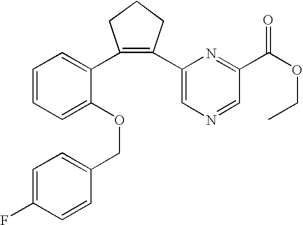 | Ethyl 6-[2-(2-{[(4- fluorophenyl)methyl]oxy}phenyl)- 1-cyclopenten-1-yl-2- pyrazinecarboxylate | Rt = 3.86 min, [M + H] 419. |
| 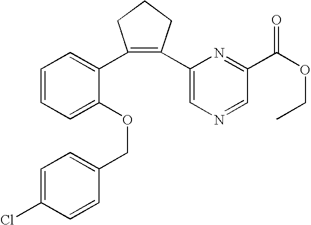 | Ethyl 6-[2-(2-{[(4- chlorophenyl)methyl]oxy}phenyl)- 1-cyclopenten-1-yl]-2- pyrazinecarboxylate | Rt = 3.75 min, [M + H] 437. |
| 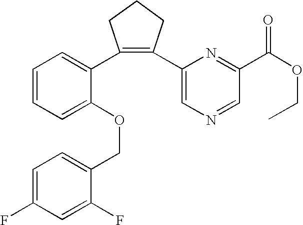 | Ethyl 6-[2-(2-{[(2,4- difluorophenyl)methyl]oxy}phenyl)- 1-cyclopenten-1-yl]-2- pyrazinecarboxylate | Rt = 4.03 min, [M + H] 435. |
| 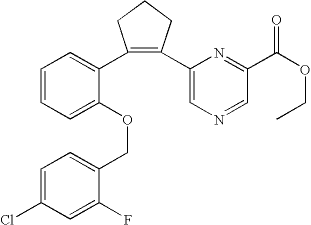 | Ethyl 6-[2-(2-{[(4-chloro-2- fluorophenyl)methyl]oxy}phenyl)- 1-cyclopenten-1-yl]-2- pyrazinecarboxylate | Rt = 4.07 min, [M + H] 453. |
| 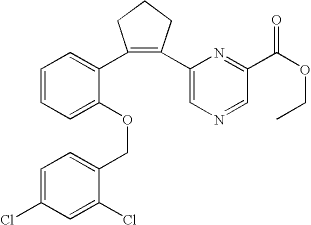 | Ethyl 6-[2-(2-{[(2,4- dichlorophenyl)methyl]oxy}phenyl)- 1-cyclopenten-1-yl]-2- pyrazinecarboxylate | Rt = 4.21 min, [M + H] 469. |
| 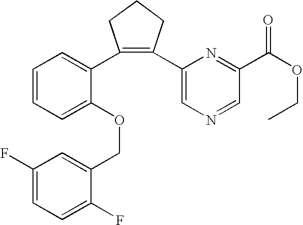 | Ethyl 6-[2-(2-{[(2,5- difluorophenyl)methyl]oxy}phenyl)- 1-cyclopenten-1-yl]-2- pyrazinecarboxylate | Rt = 3.89 min, [M + H] 437. |
| 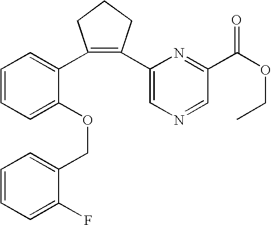 | Ethyl 6-[2-(2-{[(2- fluorophenyl)methyl]oxy}phenyl)- 1-cyclopenten-1-yl]-2- pyrazinecarboxylate | Rt = 3.85 min, [M + H] 419. |
| 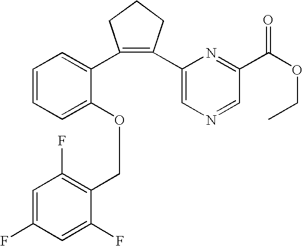 | Ethyl 6-[2-(2-{[(2,4,6- trifluorophenyl)methyl]oxy}phenyl)- 1-cyclopenten-1-yl]-2- pyrazinecarboxylate | Rt = 3.84 min, [M + H] 455. |
| 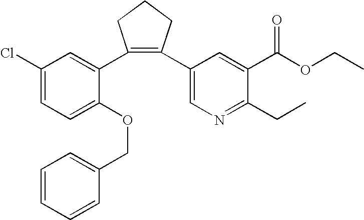 | Ethyl 5-(2-{5-chloro-2- [(phenylmethyl)oxy]phenyl}-1- cyclopenten-1-yl)-2-ethyl-3- pyridinecarboxylate | Rt = 4.35 min, [M + H] 462. |
| 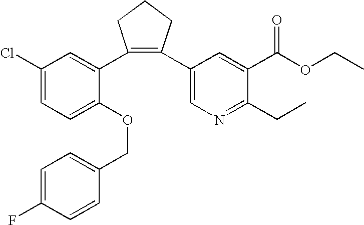 | Ethyl 5-[2-(5-chloro-2-{[(4- fluorophenyl)methyl]oxy}phenyl)- 1-cyclopenten-1-yl]-2-ethyl-3- pyridinecarboxylate | Rt = 4.34 min, [M + H] 480. |
| 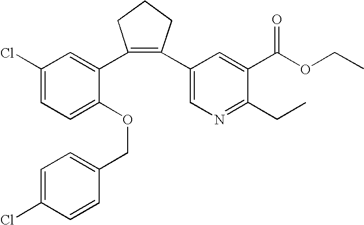 | Ethyl 5-[2-(5-chloro-2-{[(4- chlorophenyl)methyl]oxy}phenyl)- 1-cyclopenten-1-yl]-2-ethyl-3- pyridinecarboxylate | Rt = 4.51 min, [M + H] 496. |
| 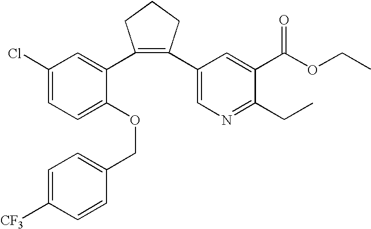 | Ethyl 5-{2-[5-chloro-2-({[4- (trifluoromethyl)phenyl]methyl}oxy) phenyl]-1-cyclopenten-1-yl}-2- ethyl-3-pyridinecarboxylate | Rt = 4.51 min, [M + H] 530. |
| 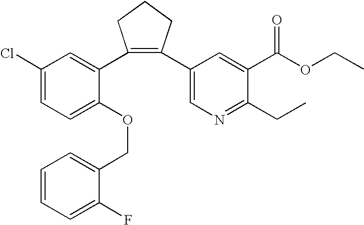 | Ethyl 5[2-(5-chloro-2-{[(2- fluorophenyl)methyl]oxy}phenyl)- 1-cyclopenten-1-yl]-2-ethyl-3- pyridinecarboxylate | Rt = 4.35 min, [M + H] 480. |
| 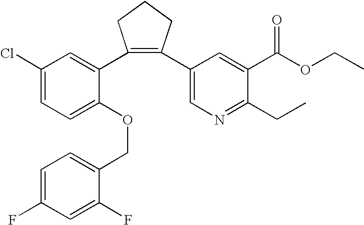 | Ethyl 5-[2-(5-chloro-2-{[(2,4- difluorophenyl)methyl]oxy}phenyl)- 1-cyclopenten-1-yl]-2-ethyl-3- pyridinecarboxylate | Rt = 4.37 min, [M + H] 498. |
| 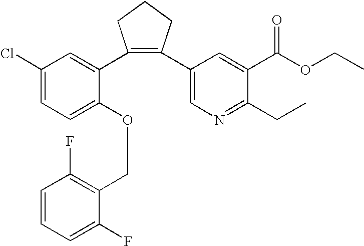 | Ethyl 5-[2-(5-chloro-2-{[(2,6- difluorophenyl)methyl]oxy}phenyl)- 1-cyclopenten-1-yl]-2-ethyl-3- pyridinecarboxylate | Rt = 4.29 min, [M + H] 498. |
| 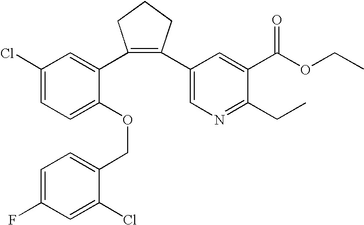 | Ethyl 5-[2-(5-chloro-2-{[(2- chloro-4- fluorophenyl)methyl]oxy}phenyl)- 1-cyclopenten-1-yl]-2-ethyl-3- pyridinecarboxylate | Rt = 4.54 min, [M + H] 514. |
| 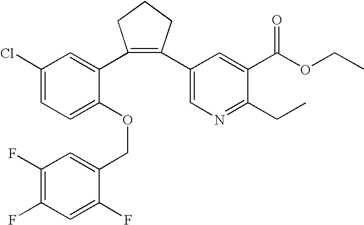 | Ethyl 5-[2-(5-chloro-2-{[(2,4,5- trifluorophenyl)methyl]oxy}pheny)- 1-cyclopenten-1-yl]-2-ethyl-3- pyridinecarboxylate | Rt = 4.40 min, [M + H] 516. |
| 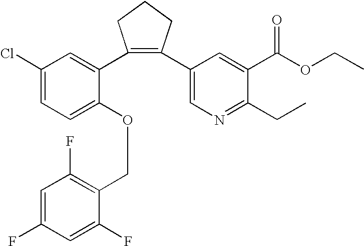 | Ethyl 5-[2-(5-chloro-2-{[(2,4,6- trifluorophenyl)methyl]oxy}phenyl)- 1-cyclopenten-1-yl]-2-ethyl-3- pyridinecarboxylate | Rt = 4.33 min, [M + H] 516. |
| 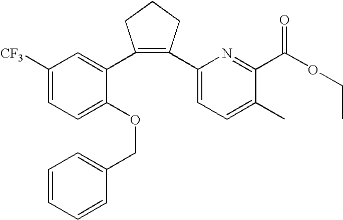 | Ethyl 3-methyl-6-{2-[2- [(phenylmethyl)oxy]-5- (trifluoromethyl)phenyl]-1- cyclopenten-1-yl}-2- pyridinecarboxylate | Rt = 4.35 min, [M + H] 482. |
| 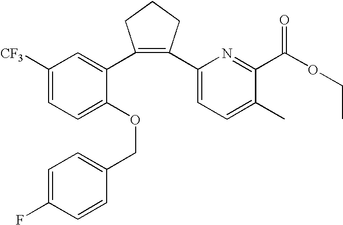 | Ethyl 6-{2-[2-{(4- fluorophenyl)methyl]oxy}-5- (trifluoromethyl)phenyl]-1- cyclopenten-1-yl}-3-methyl-2- pyridinecarboxylate | Rt = 4.36 min, [M + H] 500. |
| 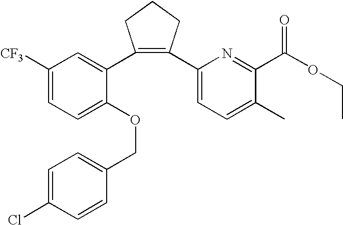 | Ethyl 6-{2-[2-{[(4- chlorophenyl)methyl]oxy}-5- (trifluoromethyl)phenyl]-1- cyclopenten-1-yl}-3-methyl-2- pyridinecarboxylate | Rt = 4.49 min, [M + H] 516. |
| 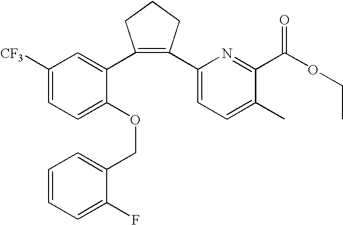 | Ethyl 6-{2-[2-{[(2- fluorophenyl)methyl)oxy}-5- (trifluoromethyl)phenyl]-1- cyclopenten-1-yl}-3-methyl-2- pyridinecarboxylate | Rt = 4.39 min, [M + H] 500. |
| 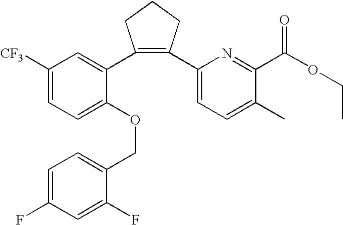 | Ethyl 6-{2-[2-{[(2,4- difluorophenyl)methyl]oxy}-5- (trifluoromethyl)phenyl]-1- cyclopenten-1-yl}-3-methyl-2- pyridinecarboxylate | Rt = 4.43 min, [M + H] 518. |
| 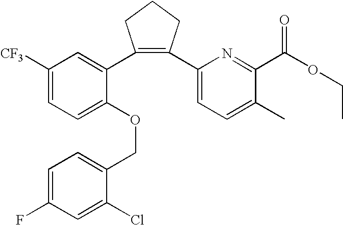 | Ethyl 6-{2-[2-{[(2-chloro-4- fluorophenyl)methyl]oxy]-5- (trifluoromethyl)phenyl]-1- cyclopenten-1-yl)-3-methyl-2- pyridinecarboxylate | Rt = 4.58 min, [M + H] 534. |
| 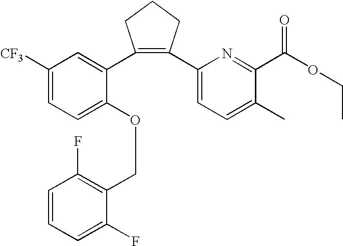 | Ethyl 6-{2-[2-{[(2,6- difluorophenyl)methyl]oxy}-5- (trifluoromethyl)phenyl]-1- cyclopenten-1-yl}-3-methyl-2- pyridinecarboxylate | Rt = 4.34 min, [M + H] 518. |
| 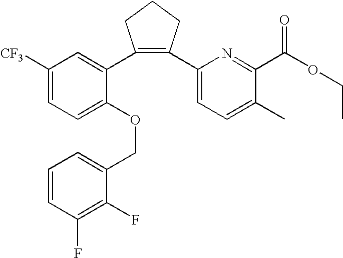 | Ethyl 6-{2-[2-{[(2,3- difluorophenyl)methyl]oxy)-5- (trifluoromethyl)phenyl]-1- cyclopenten-1-yl}-3-methyl-2- pyridinecarboxylate | Rt = 4.39 min, [M + H] 518. |
| 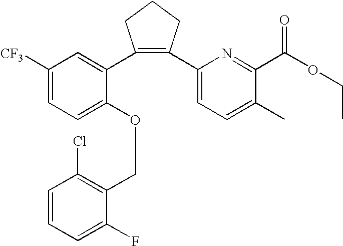 | Ethyl 6-{2-[2-{[(2-chloro-6- fluorophenyl)methyl]oxy}-5- (trifluoromethyl)phenyl]-1- cyclopenten-1-yl}-3-methyl-2- pyridinecarboxylate | Rt = 4.44 min, [M + H] 534. |
| 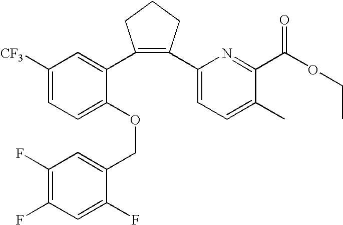 | Ethyl 3-methyl-6-[2-(5- (trifluoromethyl)-2-{[(2,4,5- trifluorophenyl)methyl]oxy}phenyl)- 1-cyclopenten-1-yl]-2- pyridinecarboxylate | Rt = 4.30 min, [M + H] 536. |
| 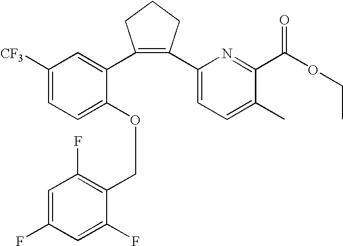 | Ethyl 3-methyl-6-[2-(5- (trifluoromethyl)-2-{[(2,4,6- trifluorophenyl)methyl]oxy}phenyl)- 1-cyclopenten-1-yl]-2- pyridinecarboxylate | Rt = 4.30 min, [M + H] 536. |
| 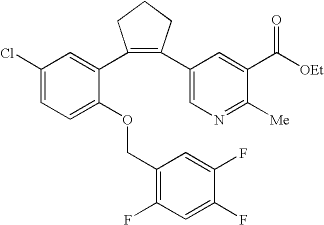 | Ethyl 5-[2-(5-chloro-2-{[(2,4,5- trifluorophenyl)methyl]oxy}phenyl)- 1-cyclopenten-1-yl]-2- methyl-3-pyridinecarboxylate | Rt = 4.24, [MH+] 502.4 |
| 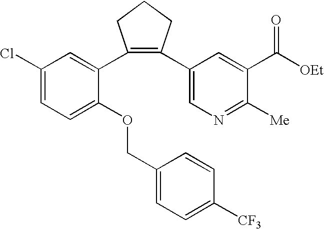 | Ethyl 5-{2-[5-chloro-2-({[4- (trifluoromethyl)phenyl]methyl} oxy)phenyl]-1-cyclopenten-1- yl}-2-methyl-3- pyridinecarboxylate | Rt = 4.35 [MH+] 516.5, 518.4 |
| 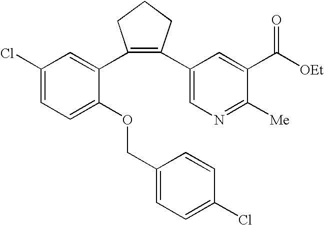 | Ethyl 5-[2-(5-chloro-2-{[(4- chlorophenyl)methyl]oxy}phenyl)- 1-cyclopenten-1-yl]-2- methyl-3-pyridinecarboxylate | Rt = 4.35 [MH+] 482.4 |
| 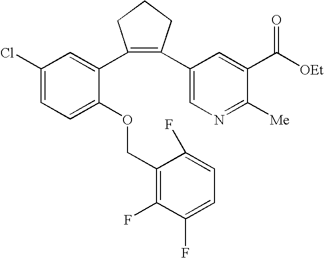 | Ethyl 5-[2-(5-chloro-2-{[(2,3,6- trifluorophenyl)methyl]oxy}phenyl)- 1-cyclopenten-1-yl]-2- methyl-3-pyridinecarboxylate | Rt = 4.13 [MH+] 502.4, 504.4 |
| 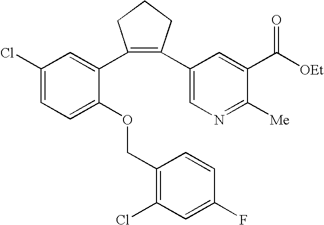 | Ethyl 5-[2-(5-chloro-2-{[(2- chloro-4- fluorophenyl)methyl]oxy}phenyl)- 1-cyclopenten-1-yl]-2- methyl-3-pyridinecarboxylate | Rt = 4.38 [MH+] 500.4 |
| 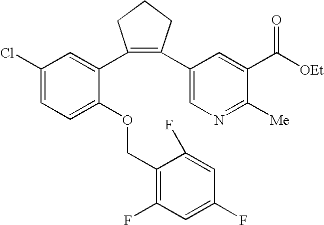 | Ethyl 5-[2-(5-chloro-2-{[(2,4,6- trifluorophenyl)methyl]oxy}phenyl)- 1-cyclopenten-1-yl]-2- methyl-3-pyridinecarboxylate | Rt = 4.16 [MH+] 502.4, 504.4 |
| 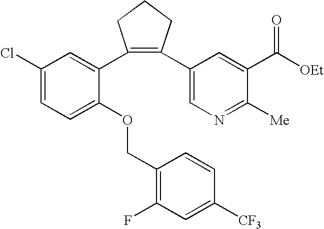 | Ethyl 5-{2-[5-chloro-2-({[2- fluoro-4- (trifluoromethyl)pheny]methyl} oxy)phenyl]-1-cyclopenten-1- yl}-2-methyl-3- pyridinecarboxylate | Rt = 4.38 [MH+] 534.5, 536.5 |
| 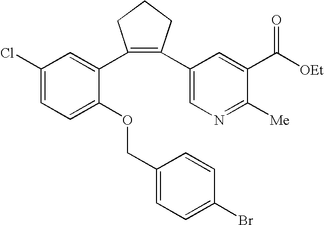 | Ethyl 5-[2-(2-{[(4- bromophenyl)methyl]oxy}-5- chlorophenyl)-1-cyclopenten- 1-yl]-2-methyl-3- pyridinecarboxylate | Rt = 4.40 [MH+] 528.4, 530.4 |
| 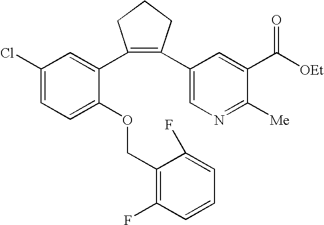 | Ethyl 5-[2-(5-chloro-2-{[(2,6- difluorophenyl)methyl]oxy}phenyl)- 1-cyclopenten-1-yl]-2- methyl-3-pyridinecarboxylate | Rt = 4.13 [MH+] 484.4, 486.5 |
| 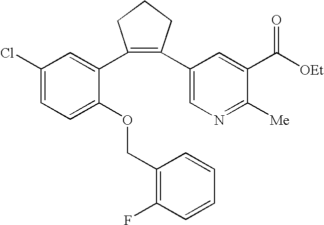 | Ethyl 5-[2-(5-chloro-2-{[(2- fluorophenyl)methyl]oxy}phenyl)- 1-cyclopenten-1-yl]-2- methyl-3-pyridinecarboxylate | Rt = 4.18 [MH+] 466.5, 468.5 |
| 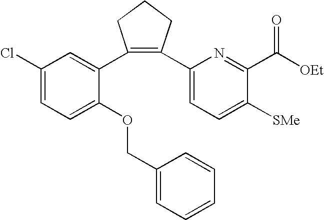 | Ethyl 6-(2-{5-chloro-2- [(phenylmethyl)oxy]phenyl}-1- cyclopenten-1-yl)-3- (methylthio)-2- pyridinecarboxylate | Rt = 4.24, [MH+] 480.4, 482.4 |
| 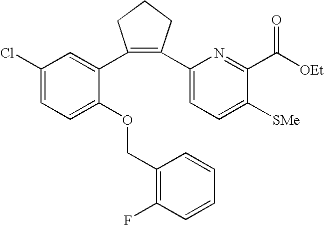 | Ethyl 6-[2-(5-chloro-2-{[(2- fluorophenyl)methyl]oxy}phenyl)- 1-cyclopenten-1-yl]-3- (methylthio)-2- pyridinecarboxylate | Rt = 4.27 [MH+] 498.4, 500.4 |
| 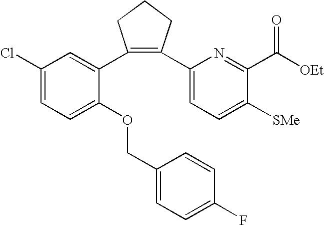 | Ethyl 6-[2-(5-chloro-2-{[(4- fluorophenyl)methyl]oxy}phenyl)- 1-cyclopenten-1-yl]-3- (methylthio)-2- pyridinecarboxylate | Rt = 4.27 [MH+] 498.4, 500.4 |
| 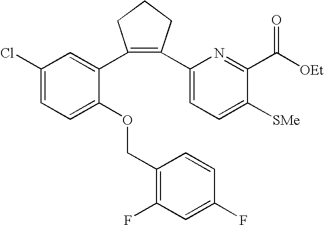 | Ethyl 6-[2-(5-chloro-2-{[(2,4- difluorophenyl)methyl]oxy}phenyl)- 1-cyclopenten-1-yl]-3- (methylthio)-2- pyridinecarboxylate | Rt = 4.31 [MH+] 516.4, 518.4 |
| 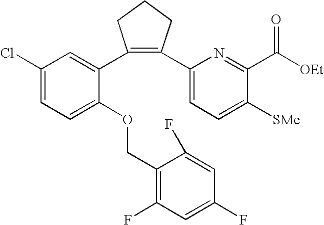 | Ethyl 6-[2-(5-chloro-2-{[(2,4,6- trifluorophenyl)methyl]oxy}phenyl)- 1-cyclopenten-1-yl]-3- (methylthio)-2- pyridinecarboxylate | Rt = 4.27 [MH+] 534.4, 536.4 |
| 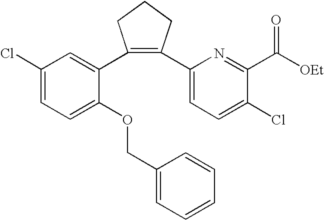 | Ethyl 3-chloro-6-(2-{5-chloro- 2-[(phenylmethyl)oxy]phenyl}- 1-cyclopenten-1-yl)-2- pyridinecarboxylate | Rt = 4.50, [MH+] 468.4 |
| 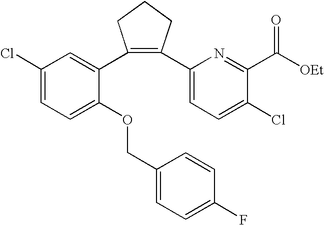 | Ethyl 3-chloro-6-[2-(5-chloro- 2-{[(4- fluorophenyl)methyl]oxy}phenyl)- 1-cyclopenten-1-yl]-2- pyridinecarboxylate | Rt = 4.49 [MH+] 486.4 |
| 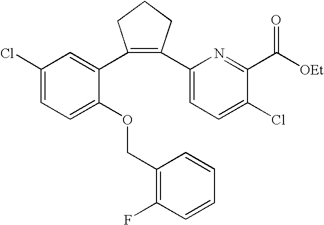 | Ethyl 3-chloro-6-[2-(5-chloro- 2-{[(2- fluorophenyl)methyl]oxy}phenyl)- 1-cyclopenten-1-yl]-2- pyridinecarboxylate | Rt = 4.45 [MH+] 486.4 |
| 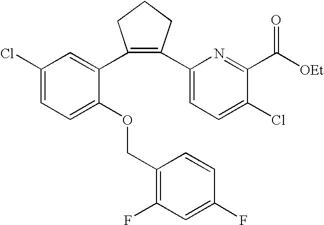 | Ethyl 3-chloro-6-[2-(5-chloro- 2-{[(2,4- difluorophenyl)methyl]oxy}phenyl)- 1-cyclopenten-1-yl]-2- pyridinecarboxylate | Rt = 4.52 [MH+] 504.4 |
| 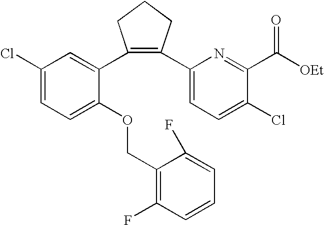 | Ethyl 3-chloro-6-[2-(5-chloro- 2-{[(2,6- difluorophenyl)methyl]oxy}phenyl)- 1-cyclopenten-1-yl]-2- pyridinecarboxylate | Rt = 4.46 [MH+] 504.4 |
| 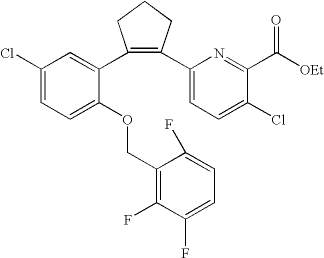 | Ethyl 3-chloro-6-[2-(5-chloro- 2-{[(2,3,6- trifluorophenyl)methyl]oxy}phenyl)- 1-cyclopenten-1-yl]-2- pyridinecarboxylate | Rt = 4.46 [MH+] 522.4 |
| 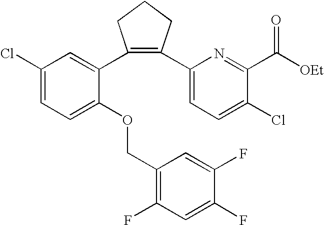 | Ethyl 3-chloro-6-[2-(5-chloro- 2-{[(2,4,5- trifluorophenyl)methyl]oxy}phenyl)- 1-cyclopenten-1-yl]-2- pyridinecarboxylate | Rt = 4.55 [MH+] 522.4 |
| 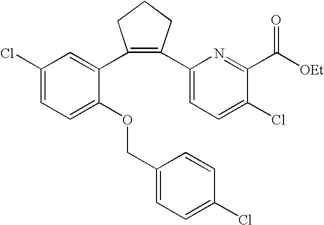 | Ethyl 3-chloro-6-[2-(5-chloro- 2-{[(4- chlorophenyl)methyl]oxy}phenyl)- 1-cyclopenten-1-yl]-2- pyridinecarboxylate | Rt = 4.65 [MH+] 502.4 |
| 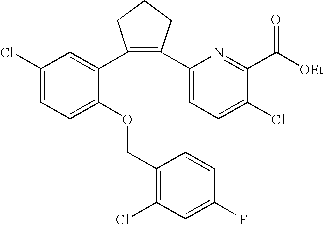 | Ethyl 3-chloro-6-[2-(5-chloro- 2-{[(2-chloro-4- fluorophenyl)methyl]oxy}phenyl)- 1-cyclopenten-1-yl]-2- pyridinecarboxylate | Rt = 4.70 [MH+] 522.3 |
| 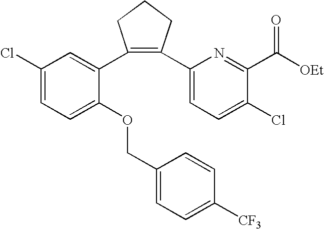 | Ethyl 3-chloro-6-{2-[5-chloro- 2-({[4- (trifluoromethyl)phenyl]methyl} oxy)phenyl]-1-cyclopenten-1- yl}-2-pyridinecarboxylate | Rt = 4.66 [MH+] 536.4 |
| 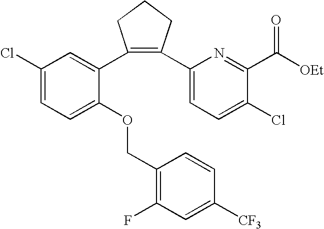 | Ethyl 3-chloro-6-{2-[5-chloro- 2-({[2-fluoro-4- (trifluoromethyl)pheny]methyl} oxy)phenyl]-1-cyclopenten-1- yl}-2-pyridinecarboxylate | Rt = 4.69 [MH+] 554.4 |
| 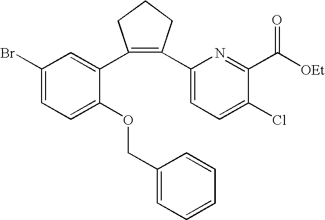 | Ethyl 6-(2-{5-bromo-2- [(phenylmethyl)oxy]phenyl}-1- cyclopenten-1-yl)-3-chloro-2- pyridinecarboxylate | Rt = 4.54, [MH+] 514.4 |
| 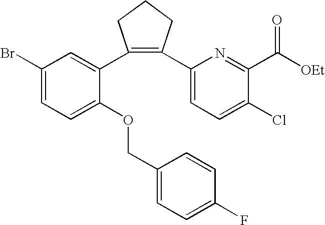 | Ethyl 6-[2-(5-bromo-2-{[(4- fluorophenyl)methyl]oxy}phenyl)- 1-cyclopenten-1-yl]-3-chloro- 2-pyridinecarboxylate | Rt = 4.10 [MH+] 532.3 |
| 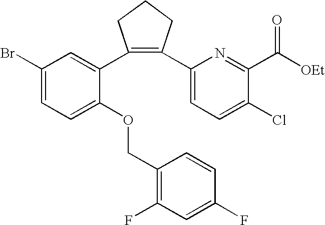 | Ethyl 6-[2-(5-bromo-2-{[(2,4- difluorophenyl)methyl]oxy}phenyl)- 1-cyclopenten-1-yl]-3- chloro-2-pyridinecarboxylate | Rt = 4.57 [MH+] 550.3 |
| 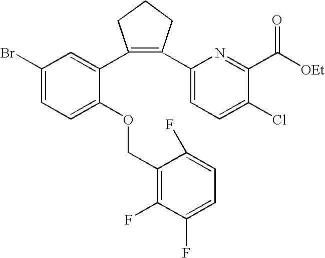 | Ethyl 6-[2-(5-bromo-2-{[(2,3,6- trifluorophenyl)methyl]oxy}phenyl)- 1-cyclopenten-1-yl]-3- chloro-2-pyridinecarboxylate | Rt = 4.35 [MH+] 568.3 |
| 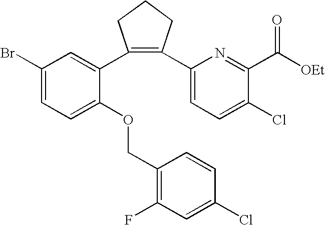 | Ethyl 6-[2-(5-bromo-2-{[(4- chloro-2- fluorophenyl)methyl]oxy}phenyl)- 1-cyclopenten-1-yl]-3-chloro- 2-pyridinecarboxylate | Rt = 4.60 [MH+] 566.3, 568.3 |
| 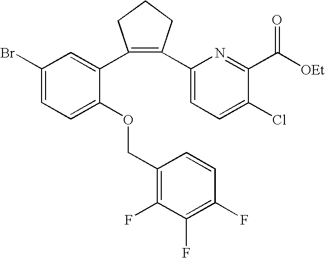 | Ethyl 6-[2-(5-bromo-2-{[(2,3,4- trifluorophenyl)methyl]oxy}phenyl)- 1-cyclopenten-1-yl]-3- chloro-2-pyridinecarboxylate | Rt = 4.52 [MH+] 568.3 |
| 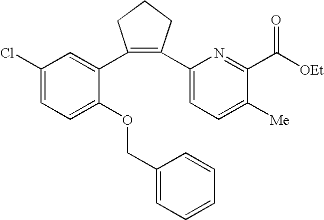 | Ethyl 6-(2-{5-chloro-2- [(phenylmethyl)oxy]phenyl}-1- cyclopenten-1-yl)-3-methyl-2- pyridinecarboxylate | Rt = 4.40, [MH+] 448.5, 450.4 |
| 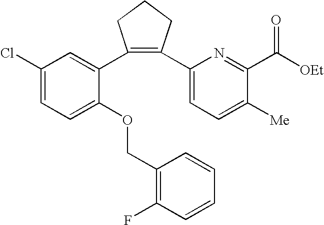 | Ethyl 6-[2-(5-chloro-2-{[(2- fluorophenyl)methyl]oxy}phenyl)- 1-cyclopenten-1-yl]-3- methyl-2-pyridinecarboxylate | Rt = 4.42 [MH+] 466.5, 468.4 |
| 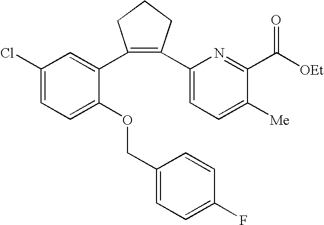 | Ethyl 6-[2-(5-chloro-2-{[(4- fluorophenyl)methyl]oxy}phenyl)- 1-cyclopenten-1-yl]-3- methyl-2-pyridinecarboxylate | Rt = 4.26 [MH+] 466.4, 468.4 |
| 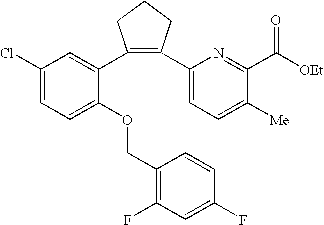 | Ethyl 6-[2-(5-chloro-2-{[(2,4- difluorophenyl)methyl]oxy}phenyl)- 1-cyclopenten-1-yl]-3- methyl-2-pyridinecarboxylate | Rt = 4.09 [MH+] 484.4, 486.4 |
| 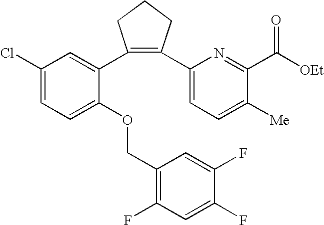 | Ethyl 6-[2-(5-chloro-2-{[(2,4,5- trifluorophenyl)methyl]oxy}phenyl)- 1-cyclopenten-1-yl]-3- methyl-2-pyridinecarboxylate | Rt = 4.49 [MH+] 502.4, 504.4 |
| 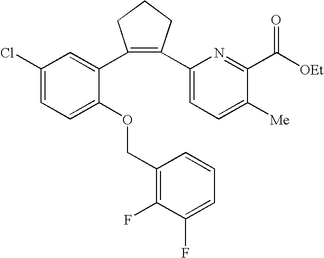 | Ethyl 6-[2-(5-chloro-2-{[(2,3- difluorophenyl)methyl]oxy}phenyl)- 1-cyclopenten-1-yl]-3- methyl-2-pyridinecarboxylate | Rt = 4.44 [MH+] 484.4, 486.4 |
| 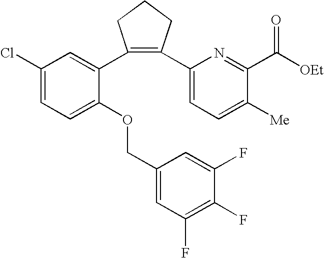 | Ethyl 6-[2-(5-chloro-2-{[(3,4,5- trifluorophenyl)methyl]-oxy}phenyl)- 1-cyclopenten-1-yl]-3- methyl-2-pyridinecarboxylate | Rt = 4.31 [MH+] 502.4, 504.4 |
| 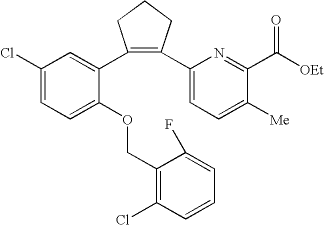 | Ethyl 6-[2-(5-chloro-2-{[(2- chloro-6- fluorophenyl)methyl]oxy}phenyl)- 1-cyclopenten-1-yl-3- methyl-2-pyridinecarboxylate | Rt = 4.18 [MH+] 500.4 |
| 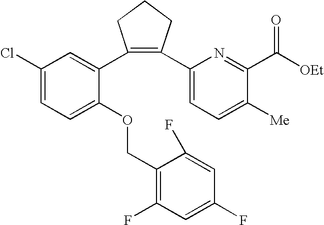 | Ethyl 6-[2-(5-chloro-2-{[(2,4,6- trifluorophenyl)methyl]oxy}phenyl)- 1-cyctopenten-1-yl]-3- methyl-2-pyridinecarboxylate | Rt = 4.10 [MH+] 502.4, 504.4 |
| 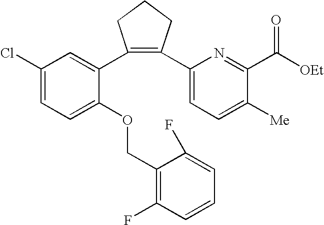 | Ethyl 6-[2-(5-chloro-2-{[(2,6- difluorophenyl)methyl]oxy}phenyl)- 1-cyclopenten-1-yl]-3- methyl-2-pyridinecarboxylate | Rt = 4.15 [MH+] 484.4, 486.4 |
| 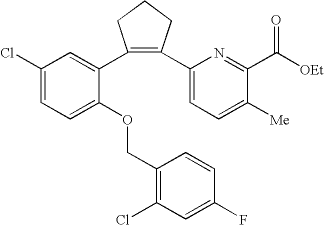 | Ethyl 6-[2-(5-chloro-2-{[(2- chloro-4- fluorophenyl)methyl]oxy}phenyl)- 1-cyclopenten-1-yl]-3- methyl-2-pyridinecarboxylate | Rt = 4.35 [MH+] 500.4 |
| 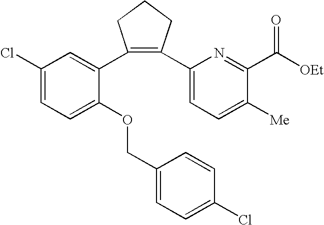 | Ethyl 6-[2-(5-chloro-2-{[(4- chlorophenyl)methyl]oxy}phenyl)- 1-cyclopenten-1-yl]-3- methyl-2-pyridinecarboxylate | Rt = 4.33 [MH+] 482.4 |
| 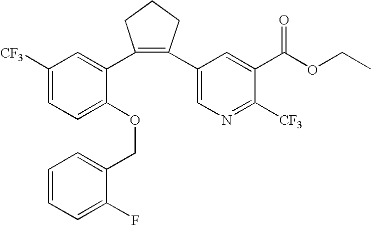 | Ethyl 5-{2-[2-{[(2- fluorophenyl)methyl]oxy}-5- (trifluoromethyl)phenyl]-1- cyclopenten-1-yl}-2- (trifluoromethyl)-3- pyridinecarboxylate | Rt = 4.49 min [M + H] 554 |
| 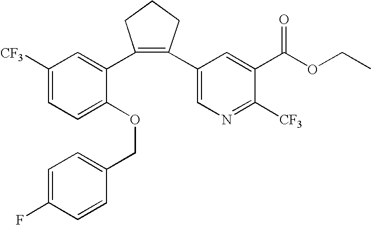 | Ethyl 5-{2-[2-{[(4- fluorophenyl)methyl]oxy}-5- (trifluoromethyl)phenyl]-1- cyclopenten-1-yl}-2- (trifluoromethyl)-3- pyridinecarboxylate | Rt = 4.49 min [MH] 554 |
| 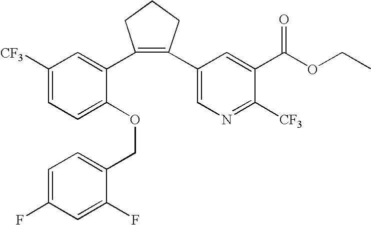 | ethyl 5-{2-[2-{[(2,4- difluorophenyl)methyl]oxy}-5- (trifluoromethyl)phenyl]-1- cyclopenten-1-yl}-2- (trifluoromethyl)-3- pyridinecarboxylate | Rt = 3.88 min [M + H] 572 |
| 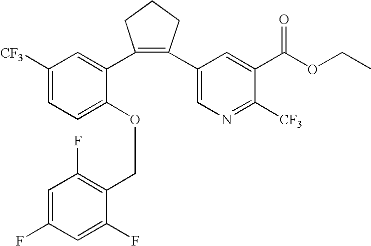 | ethyl 2-(trifluoromethyl)-5- [2-(5-trifluoromethyl)-2- {[(2,4,6- trifluorophenyl)methyl]oxy}phenyl)- 1-cyclopenten-1-yl]- 3-pyridinecarboxylate | Rt = 4.49 min [M + H] 590 |
| 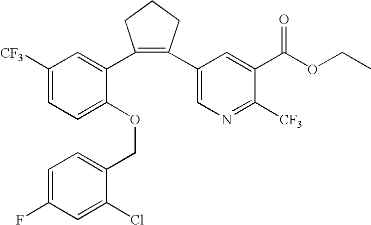 | ethyl 5-{2-[2-{[(2-chloro-4- fluorophenyl)methyl]oxy}-5- (trifluoromethyl)phenyl]-1- cyclopenten-1-yl}-2- (trifluoromethyl)-3- pyridinecarboxylate | Rt = 4.62 min [M + H] 588 (1 Cl) |
| 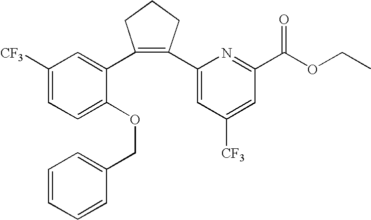 | ethyl 6-{2-[2- [(phenylmethyl)oxy]-5- (trifluoromethyl)phenyl]-1- cyclopenten-1-yl}-4- (trifluoromethyl)-2- pyridinecarboxylate | Rt = 4.57 min [M + H] 536 |
| 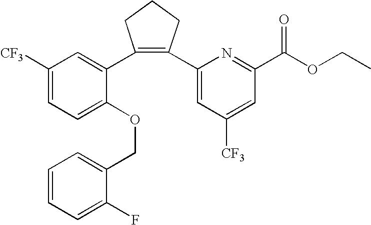 | Ethyl 6-{2-[2-{[(2- fluorophenyl)methyl]oxy}-5- (trifluoromethyl)phenyl]-1- cyclopenten-1-yl}-4- (trifluoromethyl)-2- pyridinecarboxylate | Rt = 4.54 min [M + H] 554 |
| 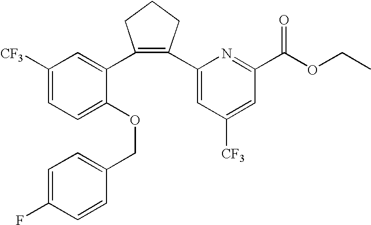 | Ethyl 6-{2-[2-{[(4- fluorophenyl)methyl]oxy}-5- (trifluoromethyl)phenyl]-1- cyclopenten-1-y[}-4- (trifluoromethyl)-2- pyridinecarboxylate | Rt = 4.26 min [M + H] 554 |
| 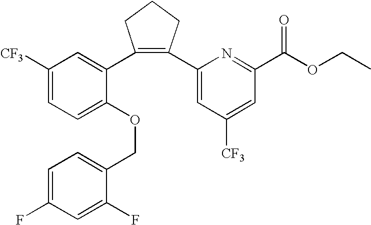 | Ethyl 6-{2-[2-{[(2,4- difluorophenyl)methyl]oxy}-5- (trifluoromethyl)phenyl]-1- cyclopenten-1-yl}-4- (trifluoromethyl)-2- pyridinecarboxylate | Rt = 4.58 min [M + H] 572 |
| 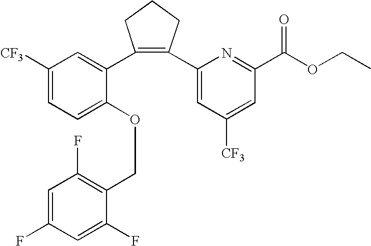 | Ethyl 4-(trifluoromethyl)-6-[2- (5-(trifluoromethyl)-2-{[(2,4,6- trifluorophenyl)methyl]oxy}phenyl)- 1-cyclopenten-1-yl]-2- pyridinecarboxylate | Rt = 4.56 min [M + H] 590 |
| 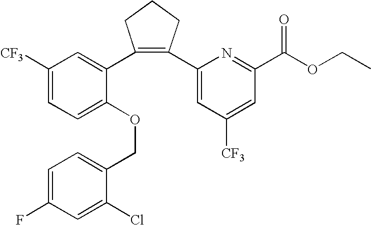 | Ethyl 6-{2-[2-{[(2-chloro-4- fluorophenyl)methyl]oxy}-5- (trifluoromethyl)phenyl]-1- cyclopenten-1-yl}-4- (trifluoromethyl)-2- pyridinecarboxylate | Rt = 4.71 min [M + H] 588 (1 Cl) |
| 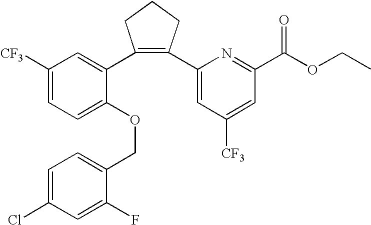 | Ethyl 6-{2-[2-{[(4-chloro-2- fluorophenyl)methyl]oxy}-5- (trifluoromethyl)phenyl]-1- cyclopenten-1-yl}-4- (trifluoromethyl)-2- pyridinecarboxylate | Rt = 4.18 min [M + H] 588 (1 Cl) |
| 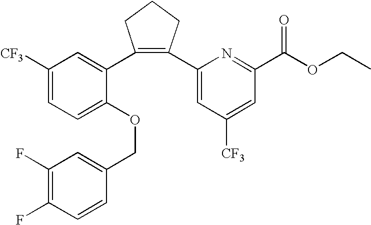 | Ethyl 6-{2-[2-{[(3,4- difluorophenyl)methyl]oxy}-5- (trifluoromethyl)phenyl]-1- cyclopenten-1-yl}-4- (trifluoromethyl)-2- pyridinecarboxylate | Rt = 4.58 min [M + H] 572 |
| 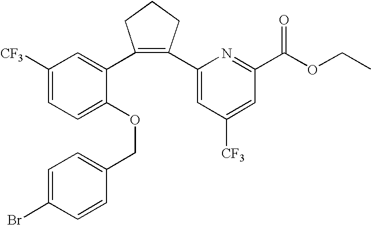 | Ethyl 6-{2-(2-{[(4- bromophenyl)methyl]oxy}-5- (trifluoromethyl)phenyl]-1- cyclopenten-1-yl}-4- (trifluoromethyl)-2- pyridinecarboxylate | Rt = 4.73 min [M + H] 614, 616 (1 Br) |
| 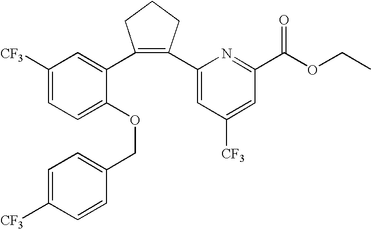 | Ethyl 4-(trifluoromethyl)-6-{2- [5-(trifluoromethyl)-2-({[4- (trifluoromethyl)phenyl]methyl} oxy)phenyl]-1-cyclopenten-1- yl}-2-pyridinecarboxylate | Rt = 4.60 min [M + H] 604 |
| 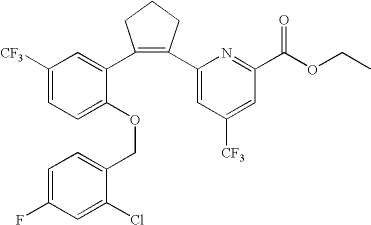 | Ethyl 6-{2-[2-{[(2-chloro-4- fluorophenyl)methyl]oxy}-5- (trifluoromethyl)phenyl]-1- cyclopenten-1-yl}-4- (trifluoromethyl)-2- pyridinecarboxylate | [M + H] 599 Rt = 4.34 min |
| 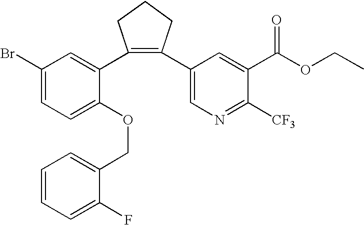 | Ethyl 5-[2-(5-bromo-2-{[(2- fluorophenyl)methyl]oxy}phenyl)- 1-cyclopenten-1-yl]-2- (trifluoromethyl)-3- pyridinecarboxylate | [M + H] 565 Rt = 4.21 min |
| 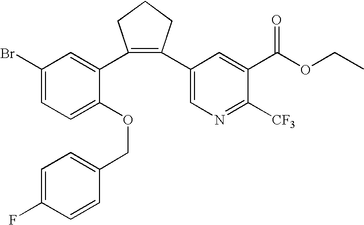 | Ethyl 5-[2-(5-bromo-2-{[(4- fluorophenyl)methyl]oxy}phenyl)- 1-cyclopenten-1-yl]-2- (trifluoromethyl)-3- pyridinecarboxylate | [M + H] 565 Rt = 4.21 min |
| 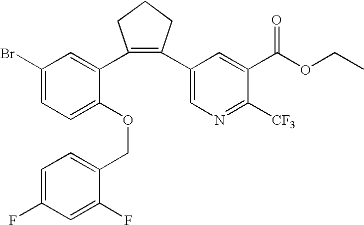 | ethyl 5-[2-(5-bromo-2-{[(2,4- difluorophenyl)methyl]oxy} phenyl)-1-cyclopenten-1-yl]- 2-(trifluoromethyl)-3- pyridinecarboxylate | [M + H] 583 Rt = 4.37 min |
| 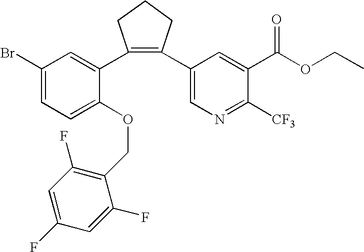 | ethyl 5-[2-(5-bromo-2- {[(2,4,6- trifluorophenyl)methyl]oxy} phenyl)-1-cyclopenten-1-yl]- 2-(trifluoromethyl)-3- pyridinecarboxylate | [M + H] 601 Rt = 4.39 min |
| 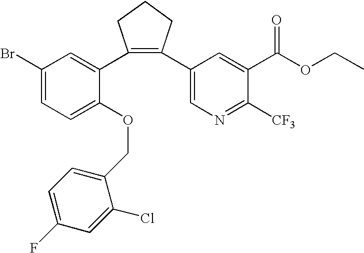 | ethyl 5-[2-(5-bromo-2-{[(2- chloro-4- fluorophenyl)methyl]oxy} phenyl)-1-cyclopenten-1-yl]-2- (trifluoromethyl)-3- pyridinecarboxylate | [M + H] 599 Rt = 4.31 min |
| 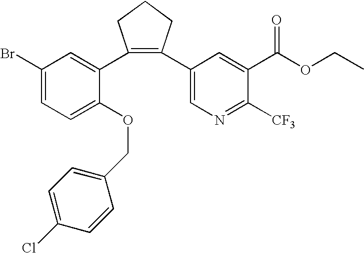 | ethyl 5-[2-(5-bromo-2-{[(4- chlorophenyl)methyl]oxy} phenyl)-1-cyclopenten-1-yl]-2- (trifluoromethyl)-3- pyridinecarboxylate | [M + H] 581 Rt = 4.39 min |
| 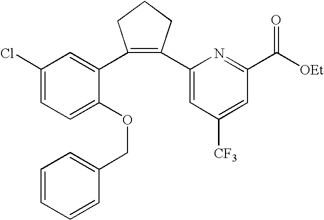 | Ethyl 6-(2-{5-chloro-2- [(phenylmethyl)oxy]phenyl}-1- cyclopenten-1-yl)-4- (trifluoromethyl)-2- pyridinecarboxylate | [M + H] 502.4, 504.4 Rt = 4.58 min |
| 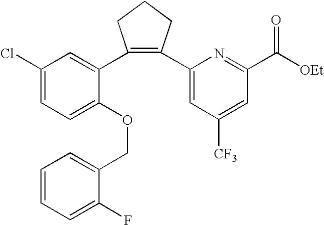 | Ethyl 6-[2-(5-chloro-2-{[(2- fluorophenyl)methyl]oxy}phenyl)- 1-cyclopenten-1-yl]-4- (trifluoromethyl)-2- pyridinecarboxylate | [M + H] 520.4, 522.4 Rt = 4.55 min |
| 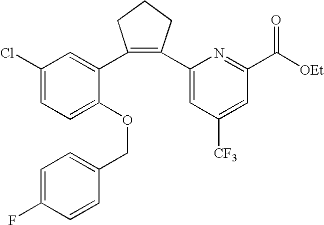 | Ethyl 6-[2-(5-chloro-2-{[(4- fluorophenyl)methyl]oxy}phenyl)- 1-cyclopenten-1-yl]-4- (trifluoromethyl)-2- pyridinecarboxylate | [M + H] 520.4, 522.4 Rt = 4.57 min |
| 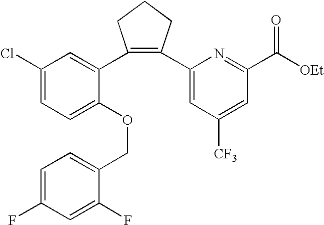 | Ethyl 6-[2-(5-chloro-2-{[(2,4- difluorophenyl)methyl]oxy}phenyl)- 1-cyclopenten-1-yl]-4- (trifluoromethyl)-2- pyridinecarboxylate | [M + H] 538.4, 540.4 Rt = 4.59 min |
| 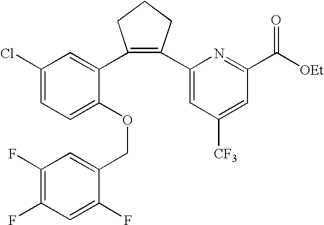 | Ethyl 6-[2-(5-chloro-2-{[(2,4,5- trifluorophenyl)methyl]oxy}phenyl)- 1-cyclopenten-1-yl]-4- (trifluoromethyl)-2- pyridinecarboxylate | [M + H] 556.3, 558.4 Rt = 4.61 min |
| 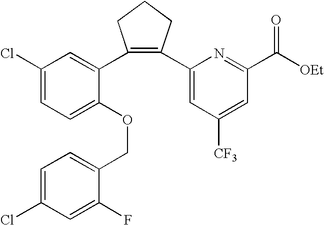 | Ethyl 6-[2-(5-chloro-2-{[(4- chloro-2- fluorophenyl)methyl]oxy}phenyl)- 1-cyclopenten-1-yl]-4- (trifluoromethyl)-2- pyridinecarboxylate | [M = H] 554.3, 556.4 Rt = 4.73 min |
| 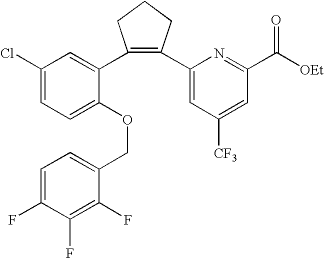 | Ethyl 6-[2-(5-chloro-2-{[(2,3,4- trifluorophenyl)methyl]oxy}phenyl)- 1-cyclopenten-1-yl]-4- (trifluoromethyl)-2- pyridinecarboxylate | [M + H] 556.3, 558.4 Rt = 4.61 min |
| 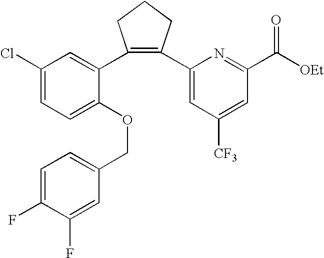 | Ethyl 6-[2-(5-chloro-2-{[(3,4- difluorophenyl)methyl]oxy}phenyl)- 1-cyclopenten-1-yl]-4- (trifluoromethyl)-2- pyridinecarboxylate | [M + H] 538.4, 540.4 Rt = 4.59 min |
| 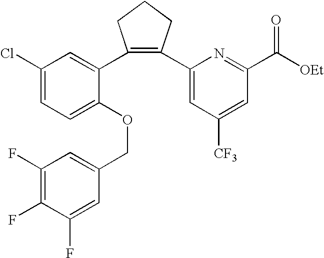 | Ethyl 6-{2-(5-chloro-2-{[(3,4,5- trifluorophenyl)methyl]oxy}phenyl)- 1-cyclopenten-1-yl]-4- (trifluoromethyl)-2- pyridinecarboxylate | [M + H] 556.3, 558.4 Rt = 4.65 min |
| 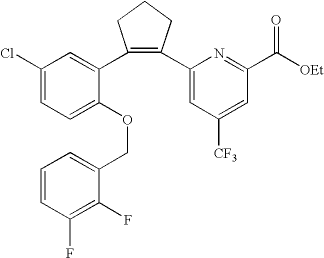 | Ethyl 6-[2-(5-chloro-2-{[(2,3- difluorophenyl)methyl]oxy}phenyl)- 1-cyclopenten-1-yl]-4- (trifluoromethyl)-2- pyridinecarboxylate | [M + H] 538.4, 540.4 Rt = 4.57 min |
| 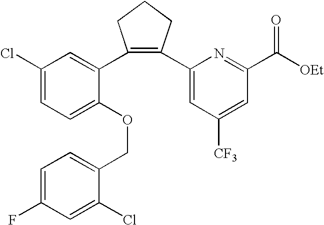 | Ethyl 6-[2-(5-chloro-2-{[(2- chloro-4- fluorophenyl)methyl]oxy}phenyl)- 1-cyclopenten-1-yl]-4- (trifluoromethyl)-2- pyridinecarboxylate | [M + H] 554.3, 556.3 Rt = 4.75 min |
| 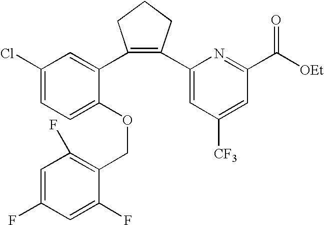 | Ethyl 6-[2-(5-chloro-2-{[(2,4,6- trifluorophenyl)methyl]oxy}phenyl)- 1-cyclopenten-1-yl]-4- (trifluoromethyl)-2- pyridinecarboxylate | [M + H] 556.3, 558.4 Rt = 4.56 min |
| 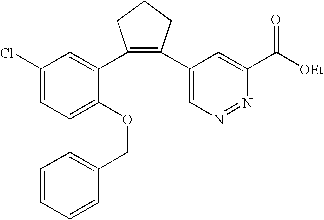 | Ethyl 5-(2-{5-chloro-2- [(phenylmethyl)oxy]phenyl}-1- cyclopenten-1-yl)-3- pyridazinecarboxylate | [M + H] 435.2, 437.2, Rt = 3.76 min |
| 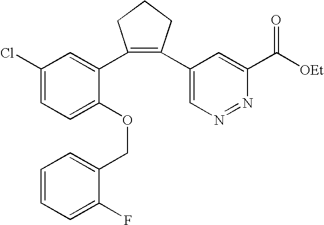 | Ethyl 5-[2-(5-chloro-2-{[(4- fluorophenyl)methyl]oxy}phenyl)- 1-cyclopenten-1-yl]-3- pyridazinecarboxylate | [M + H] 453.3, 455.3, Rt = 3.60 min |
| 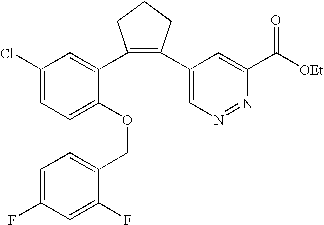 | Ethyl 5-[2-(5-chloro-2-{[(4- fluorophenyl)methyl]oxy}phenyl)- 1-cyclopenten-1-yl]-3- pyridazinecarboxylate | [M + H] 471.3, 473.3, Rt = 3.61 min |
| 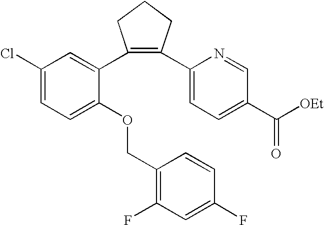 | Ethyl 6-[2-(5-chloro-2-{[(2,4- difluorophenyl)methyl]oxy}phenyl)- 1-cyclopenten-1-yl]-3- pyridinecarboxylate | Rt = 4.27, [MH+] 470.3, 472.3 |
| 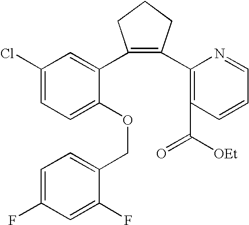 | Ethyl 2-[2-(5-chloro-2-{[(2,4- difluorophenyl)methyl]oxy}phenyl)- 1-cyclopenten-1-yl]-3- pyridinecarboxylate | Rt = 3.94, [MH+] 470.3, 472.3 |
| 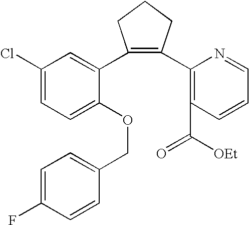 | Ethyl 2-[2-(5-chloro-2-{[(4- fluorophenyl)methyl]oxy}phenyl)- 1-cyclopenten-1-yl]-3- pyridinecarboxylate | Rt = 3.90, [MH+] 452.3, 454.3 |
| 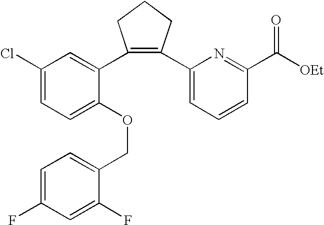 | Ethyl 6-[2-(5-chloro-2-{[(2,4- difluorophenyl)methyl]oxy}phenyl)- 1-cyclopenten-1-yl]-2- pyridinecarboxylate | Rt = 4.20 [MH+] 470.3, 472.3 |
| 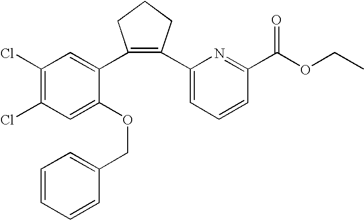 | Ethyl 6-[2-{4,5-dichloro-2- [(phenylmethyl)oxy]phenyl}-1- cyclopenten-1-yl)-2- pyridinecarboxylate | Rt = 4.33 [MH+] 468.4, 470.4 |
| 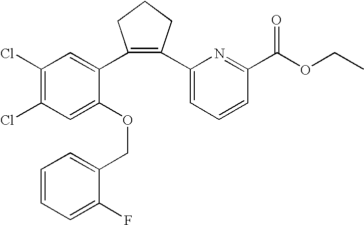 | Ethyl 6-[2-(4,5-dichloro-2-{[(2- fluorophenyl)methyl]oxy}phenyl)- 1-cyclopenten-1-yl]-2- pyridinecarboxylate | Rt = 4.35 [MH+] 486.4, 488.4 |
| 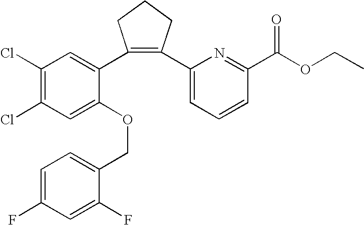 | Ethyl 6-[2-(4,5-dichloro-2- {[(2,4- difluorophenyl)methyl]oxy}phenyl)- 1-cyclopenten-1-yl]-2- pyridinecarboxylate | Rt = 4.36 [MH+] 504.4, 506.4 |
| 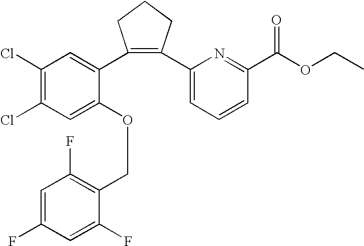 | Ethyl 6-[2-(4,5-dichloro-2- {[(2,4,6- trifluorophenyl)methyl]oxy}phenyl)- 1-cyclopenten-1-yl]-2- pyridinecarboxylate | Rt = 4.34 [MH+] 522.4, 524.4 |

### Ethyl 6-(2-{5-chloro-2-[(cyclopentylmethyl)oxy]phenyl}-1-cyclopenten-1-yl)-2-pyridinecarboxylate

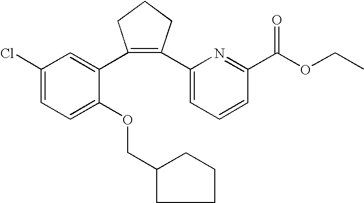

A mixture of 6-[2-(5-chloro-2-hydroxyphenyl)-1-cyclopenten-1-yl]-2-pyridinecarboxylic acid (100 mg, 0.29 mmol), potassium carbonate (200 mg, 1.45 mmol) and cyclopentylmethyl 4-methylbenzenesulfonate (90 mg, 0.35 mmol) in DMF (3 ml) was heated at 90° C. under nitrogen for 2 hours. More cyclopentylmethyl 4-methylbenzenesulfonate (40 mg, 0.16 mmol) was added and the mixture heated for another 2 hours. After cooling the solution was diluted with water and extracted with ethyl acetate (3×10 ml). The combined extracts were dried (MgSO₄) and evaporated. Purification was carried out by flash chromatography (10% ethyl acetate:iso-hexane) to yield the title compound as a clear oil.

LC/MS: Rt=4.68, [MH+] 426, 428

### Ethyl 6-(2-{5-chloro-2-[(2-methylpropyl)oxy]phenyl}-1-cyclopenten-1-yl)-2-pyridinecarboxylate

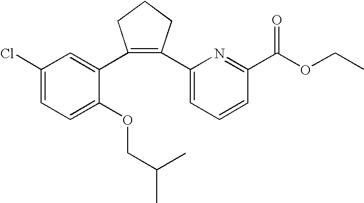

Prepared in a similar manner to ethyl 6-(2-{5-chloro-2-[(cyclopentylmethyl)oxy]phenyl}-1-cyclopenten-1-yl)-2-pyridinecarboxylate using 1-bromo-2-methylpropane instead of cyclopentylmethyl 4-methylbenzenesulfonate. LC/MS: Rt=4.49 [MH+] 400, 402

### Ethyl 6-(2-{5-bromo-2-[(1-methylethyl)oxy]phenyl}-1-cyclopenten-1-yl)-2-pyridinecarboxylate

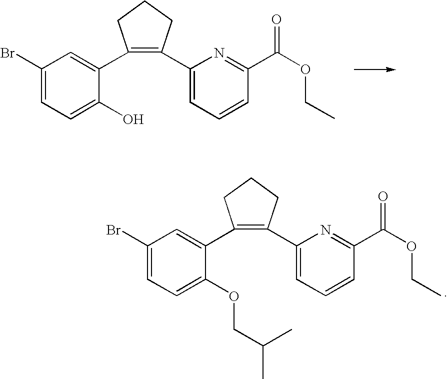

A solution of ethyl 6-[2-(5bromo-2-hydroxyphenyl)-1-cyclopenten-1-yl]-2-pyridinecarboxylate (125 mg, 0.32 mmol) in dry THF (2 ml) was treated with diethyl azodicarboxylate (65 mg, 67 μl, 0.35 mmol), triphenylphosphine (84 mg, 0.35 mmol) and iso-butyl alcohol (22 mg, 27 μl, 0.3 mmol). The reaction mixture was stirred at room temperature overnight. The solvent was evaporated and the residue chromatographed using hexane/ethyl acetate 95:5 to give the title compound as a colourless oil.

LCMS: Rt=4.32 min. [M+H]=444, 446.

The following intermediates were prepared by a similar route to Ethyl 6-(2-{5-bromo-2-[(1-methylethyl)oxy]phenyl}-cyclopenten-1-yl)-2-pyridinecarboxylate from the appropriate intermediates.

| Structure | Name | LC/MS |
|---|---|---|
| 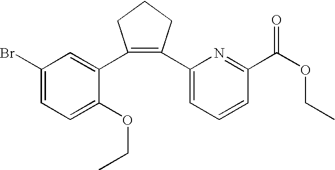 | Ethyl 6-{2-[5-bromo-2- (ethyloxy)phenyl]-1-cyclopenten- 1-yl}-2-pyridinecarboxylate | Rt = 3.96 min. [M + H]= 416, 418 |
| 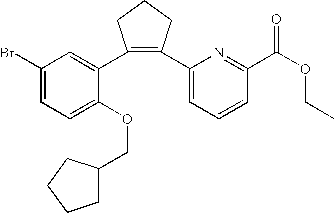 | Ethyl 6-(2-{5-bromo-2- [(cyclopentylmethyl)oxy]phenyl}- 1-cyclopenten-1-yl)-2- pyridinecarboxylate | Rt = 4.52 min. [M + H] = 470, 472 |
| 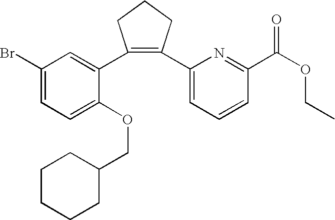 | Ethyl 6-(2-{5-bromo-2- [(cyclohexylmethyl)oxy]phenyl}- 1-cyclopenten-1-yl)-2- pyridinecarboxylate | Rt = 4.64 min [M + H] = 484, 486 |

### Ethyl 2-(acetylamino)-5-(2-{5-chloro-2-[(phenylmethyl)oxy]phenyl}-1-cyclopenten-1-yl)benzoate

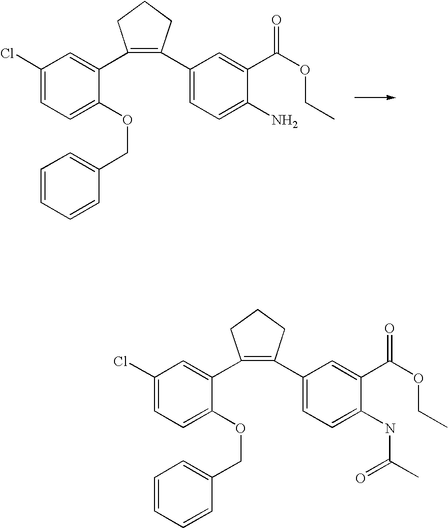

A mixture of ethyl 2-amino-5-(2-{5-chloro-2-[(phenylmethyl)oxy]phenyl}-1-cyclopenten-1-yl)benzoate (75 mg, 0.17 mmol), acetyl chloride (21 mg, 0.3 mmol), and triethylamine (30 g, 42 μl, 0.3 mmol) in dichloromethane (3 ml) was stirred at room temperature for 30 mins. The solvent was evaporated and the residue was chromatographed eluting with ethyl acetate/hexane 1:4 to give the title compound as colourless glass.

Rt=4.08 min. [M+H]=490

The following intermediates were prepared by a similar route to ethyl 2-(acetylamino)-5-(2-{5-chloro-2-[(phenylmethyl)oxy]phenyl}-1-cyclopenten-1-yl)benzoate from the appropriate intermediates.

| Structure | Name | LC/MS |
|---|---|---|
| 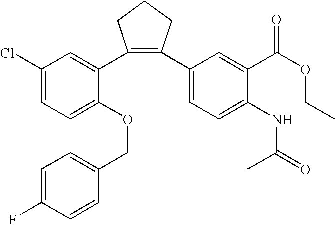 | Ethyl 2-(acetylamino)-5-[2-(5-chloro-2- {[(4-fluorophenyl)methyl]oxy}phenyl)- 1-cyclopenten-1-yl]benzoate | Rt = 4.09 min. [M + H] = 508 |
| 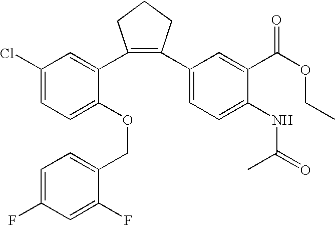 | Ethyl 2-(acetylamino)-5-[2-(5-chloro-2- {[(2,4-difluorophenyl)methyl]oxy} phenyl)-1-cyclopenten-1-yl)benzoate | Rt = 4.11 min. [M + H] = 526 |
| 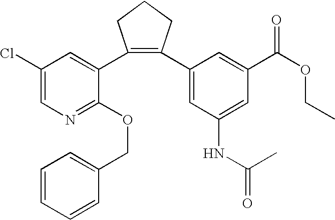 | Ethyl 3-(acetylamino)-5-(2-{5-chloro-2- [(phenylmethyl)oxy]-3-pyridinyl}-1- cyclopenten-1-yl)benzoate | Rt = 4.04 min [M + H] = 491 |
| 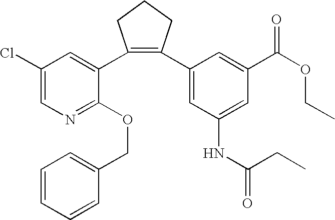 | Ethyl 3-(2-{5-chloro-2- [(phenylmethyl)oxy]-3-pyridinyl}-1- cyclopenten-1-yl)-5- (propanoylamino)benzoate | Rt = 4.03 min. [M + H] = 505 |
| 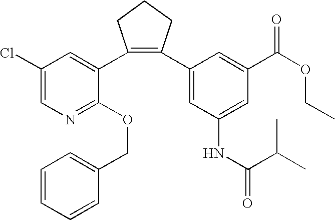 | Ethyl 3-(2-{5-chloro-2- [(phenylmethyl)oxy]-3-pyridinyl}-1- cyclopenten-1-yl)-5-[(2- methylpropanoyl)amino]benzoate | Rt = 4.25 min. [M + H} = 519 |

### Ethyl 5-[2-(5-chloro-2-{[(4-fluorophenyl)methyl]oxy}-3-pyridinyl)-1-cyclopenten-1-yl]-2-methylbenzoate

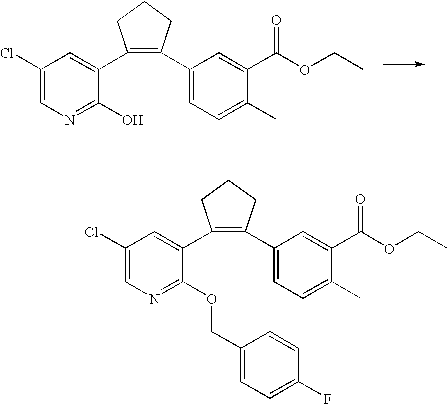

Ethyl 5-[2-(5-chloro-2-oxo-1,2-dihydro-3-pyridinyl)-1-cyclopenten-1-yl]-2-methylbenzoate (76 mg, 0.213 mmol) was dissolved in toluene (3 ml) and silver carbonate (65 mg, 0.234 mmol) and 4-fluorobenzyl bromide (29 □l, 0.234 mmol) added. The mixture was heated to reflux for 1 hour then stirred at room temperature for 16 hours. After filtration, the solution was washed with water, dried (MgSO₄) and evaporated. The residue was flash chromatographed eluting with 2% ethyl acetate/isohexane to give the title compound (47 mg). LC/MS Rt=4.47 min [MH⁺] 466, 468.

The following intermediates were prepared by a similar route to ethyl 5-[2-(5-chloro-2-{[(4-fluorophenyl)methyl]oxy}-3-pyridinyl)-1-cyclopenten-1-yl]-2-methylbenzoate from the appropriate intermediates.

| | COMPOUND NAME | LC/MS |
|---|---|---|
| 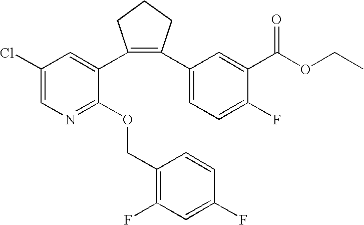 | Ethyl 5-[2-(5-chloro-2-{[(2,4- difluorophenyl) methyl]oxy}-3- pyridinyl)-1-cyclopenten-1-yl]-2- fluorobenzoate | Rt = 4.46 min. [MH⁺] 488, 490. |
| 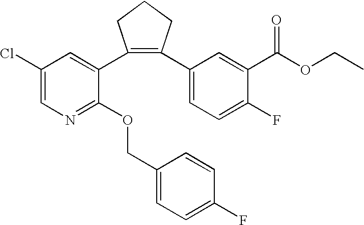 | Ethyl 5-[2-(5-chloro-2-{[(4- fluorophenyl) methyl]oxy}-3-pyridinyl)- 1-cyolopenten-1-yl]-2-fluorobenzoate | Rt = 4.42 min. [MH⁺] 470, 472. |
| 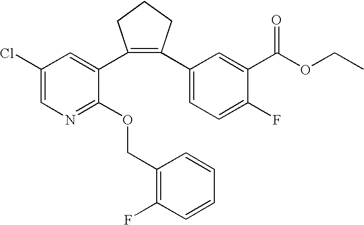 | Ethyl 5-[2-(5-chloro-2-{[(2- fluorophenyl) methyl]oxy}-3-pyridinyl)- 1-cyclopenten-1-yl]-2-fluorobenzoate | Rt = 4.50 min. [MH⁺] 470, 472. |
| 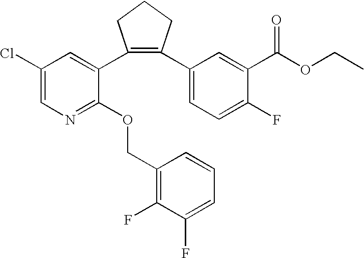 | Ethyl 5-[2-(5-chloro-2-{[(2,3- difluorophenyl)methyl]oxy}-3- pyridinyl)-1-cyclopenten-1-yl]-2- fluorobenzoate | Rt = 4.40 min. [MH⁺] 488, 490. |
| 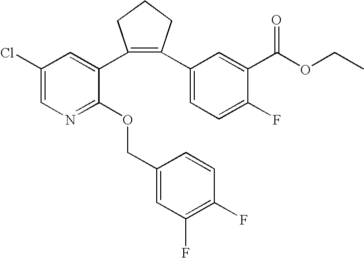 | Ethyl 5-[2-(5-chloro-2-{[(3,4- difluorophenyl) methyl]oxy}-3- pyridinyl)-1-cyclopenten-1-yl]-2- fluorobenzoate | Rt = 4.44 min. [MH⁺] 488, 490. |
| 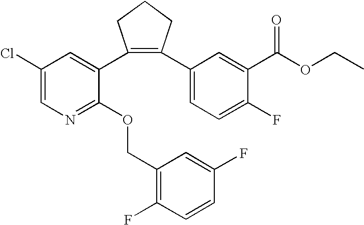 | Ethyl 5-[2-(5-chloro-2-{[(2,5- difluorophenyl)methyl]oxy}-3- pyridinyl)-1-cyclopenten-1-yl]-2- fluorobenzoate | Rt = 4.43 min. [MH⁺] 488, 490. |
| 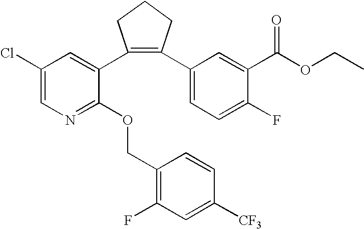 | ethyl 5-{2-[5-chloro-2-({[2-fluoro-4- (trifluoromethyl)phenyl] methyl}oxy)- 3-pyridinyl]-1-cyclopenten-1-yl}-2- fluorobenzoate | Rt = 4.32 min. [MH⁺] 538, 540. |
| 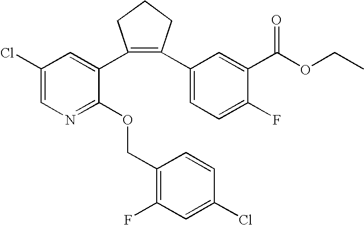 | Ethyl 5-[2-(5-chloro-2-{[(4-chloro-2- fluorophenyl)methyl]oxy}-3-pyridinyl)- 1-cyclopenten-1-yl]-2-fluorobenzoate | Rt = 4.32 min. [MH⁺] 504, 506. |
| 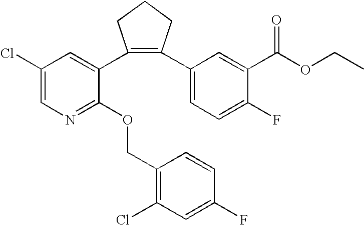 | Ethyl 5-[2-(5-chloro-2-{[(2-chloro-4- fluorophenyl)methyl]oxy}-3-pyridinyl)- 1-cyclopenten-1-yl]-2-fluorobenzoate | Rt = 4.50 min. [MH⁺] 504, 506. |
| 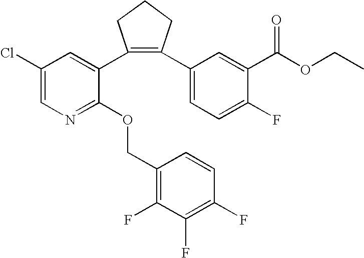 | Ethyl 5-[2-(5-chloro-2-{[(2,3,4- trifluorophenyl)methyl]oxy}-3- pyridinyl)-1-cyclopenten-1-yl]-2- fluorobenzoate | Rt = 4.43 min. [MH⁺] 506, 508. |
| 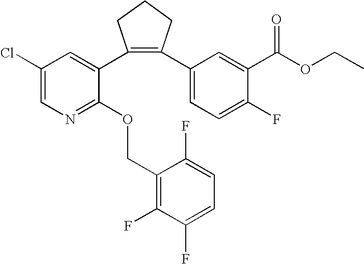 | Ethyl 5-[2-(5-chloro-2-{[(2,3,6- trifluorophenyl) methyl]oxy}-3- pyridinyl)-1-cyclopenten-1-yl]-2- fluorobenzoate | Rt = 4.55 min. [MH⁺] 506, 508. |
| 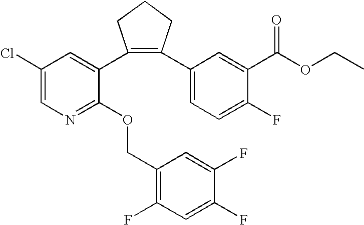 | Ethyl 5-[2-(5-chloro-2-{[(2,4,5- trifluorophenyl) methyl]oxy}-3- pyridinyl)-1-cyclopenten-1-yl]-2- fluorobenzoate | Rt = 4.62 min. [MH⁺] 506, 508. |
| 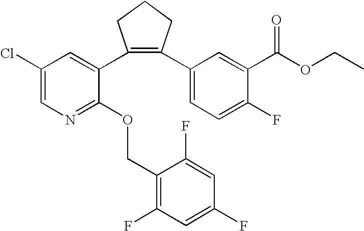 | Ethyl 5-[2-(5-chloro-2-{[(2,4,6- trifluorophenyl) methyl]oxy}-3- pyridinyl)-1-cyclopenten-1-yl]-2- fluorobenzoate | Rt = 4.40 min. [MH⁺] 506, 508. |
| 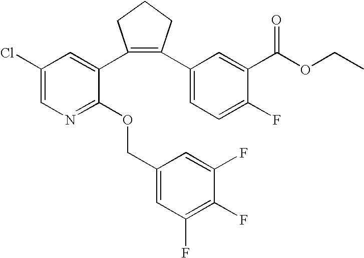 | Ethyl 5-[2-(5-chloro-2-{[(3,4,5- trifluorophenyl) methyl]oxy}-3- pyridinyl)-1-cyclopenten-1-yl]-2- fluorobenzoate | Rt = 4.49 min. [MH⁺] 506, 508. |
| 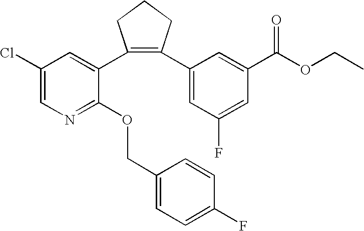 | Ethyl 3-[2-(5-chloro-2-{[(4- fluorophenyl) methyl]oxy}-3-pyridinyl)- 1-cyclopenten-1-yl]-5-fluorobenzoate | Rt = 4.36 min. [MH⁺] 470, 472. |
| 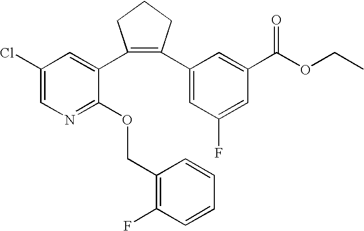 | Ethyl 3-[2-(5-chloro-2-{[(2- fluorophenyl) methyl]oxy}-3-pyridinyl)- 1-cyclopenten-1-yl]-5-fluorobenzoate | Rt = 4.38 min. [MH⁺] 470, 472. |
| 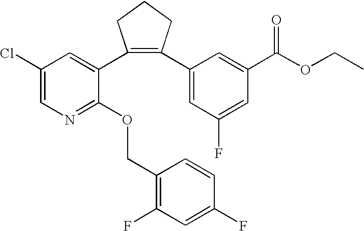 | Ethyl 3-[2-(5-chloro-2-{[(2,4- difluorophenyl) methyl]oxy}-3- fluorobenzoate | Rt = 4.50 min. [MH⁺] 488, 490. |
| 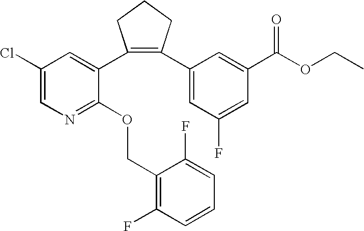 | Ethyl 3-[2-(5-chloro-2-{[(2,6- difluorophenyl) methyl]oxy}-3- pyridinyl)-1-cyclopenten-1-yl]-5- fluorobenzoate | Rt = 4.50 min. [MH⁺] 488, 490. |
| 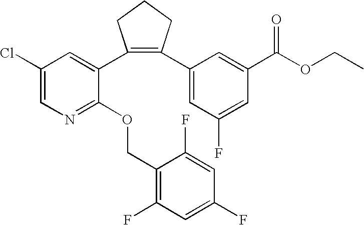 | Ethyl 3-[2-(5-chloro-2-{[(2,4,6- trifluorophenyl) methyl]oxy}-3- pyridinyl)-1-cyclopenten-1-yl]-5- fluorobenzoate | Rt = 4.53 min. [MH⁺] 506, 508. |
| 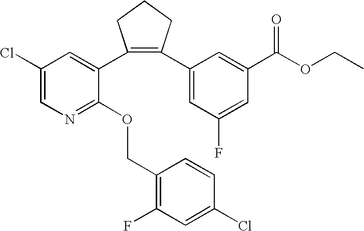 | Ethyl 3-[2-(5-chloro-2-{[(4-chloro-2- fluoro phenyl)methyl}oxy}-3-pyridinyl)- 1-cyclopenten-1-yl}-5-fluorobenzoate | Rt = 4.72 min. [MH⁺] 504, 506. |
| 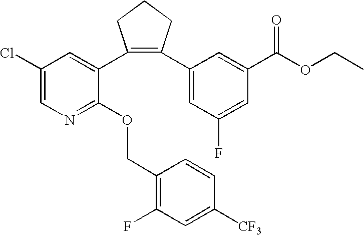 | Ethyl 3-{2-[5-chloro-2-({[2-fluoro-4- (trifluoromethyl)phenyl]methyl)oxy)-3- pyridinyl]-1-cyclopenten-1-yl)-5- fluorobenzoate | Rt = 4.72 min. [MH⁺] 538, 540. |
| 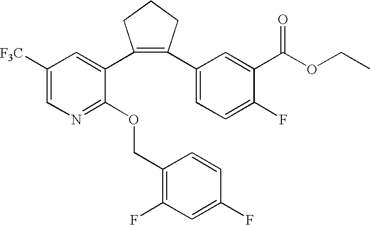 | Ethyl 5-{2-[2-{[(2,4- difluorophenyl)methyl]oxy}-5- (trifluoromethyl)-3-pyridinyl]-1- cyclopenten-1-yl}-2-fluorobenzoate | Rt = 4.44 min. [MH⁺] 522. |
| 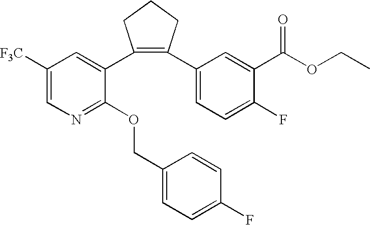 | Ethyl 2-fluoro-5-{2-[2-{[(4- fluorophenyl)methyl]oxy}-5- (trifluoromethyl)-3-pyridinyl]-1- cyclopenten-1-yl}benzoate | Rt = 4.41 min. [MH⁺] 504. |
| 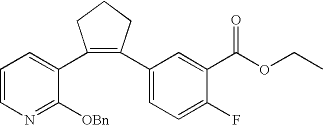 | Ethyl 2-fluoro-5-(2-{2- [(phenylmethyl)oxy]-3-pyridinyl}-1- cyclopenten-1-yl)benzoate | Rt = 4.14 min. [MH⁺] 418. |
| 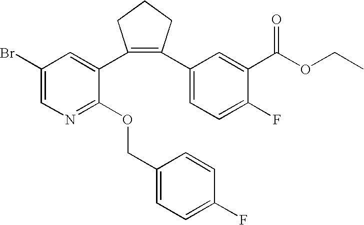 | Ethyl 5-[2-(5-bromo-2-{[(4- fluorophenyl) methyl]oxy}-3-pyridinyl)- 1-cyclopenten-1-yl]-2-fluorobenzoate | Rt = 4.36 min. [MH⁺] 514, 516. |
| 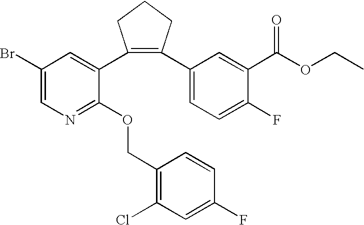 | Ethyl 5-[2-(5-bromo-2-{[(2-Chloro-4- fluoro phenyl)methyl]oxy}-3-pyridinyl)- 1-cyclopenten-1-yl]-2-fluorobenzoate | Rt = 4.64 min. [MH⁺] 548, 550. |
| 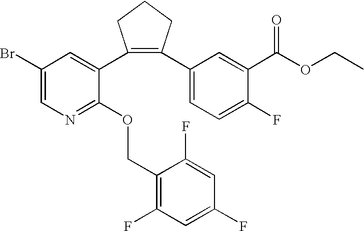 | Ethyl 5-[2-(5-bromo-2-{[(2,4,6- trifluorophenyl) methyl]oxy}-3- pyridinyl)-1-cyclopenten-1-yl]-2- fluorobenzoate | Rt = 4.44 min. [MH⁺] 550, 552. |
| 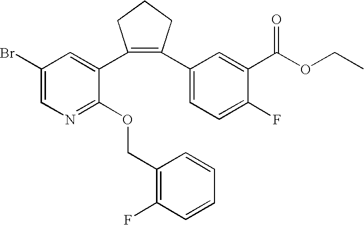 | Ethyl 5-[2-(5-bromo-2-{[(2- fluorophenyl) methyl]oxy}-3-pyridinyl)- 1-cyclopenten-1-yl]-2-fluorobenzoate | Rt = 3.86 min. [MH⁺] 514, 516. |
| 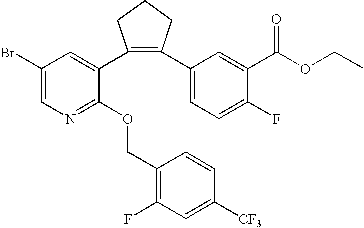 | Ethyl 5-{2-[5-bromo-2-({[2-fluoro-4- (trifluoro methyl)phenyl]methyl}oxy)-3- pyridinyl]-1-cyclopenten-1-yl}-2- fluorobenzoate | Rt = 4.61 min. [MH⁺] 582, 584. |
| 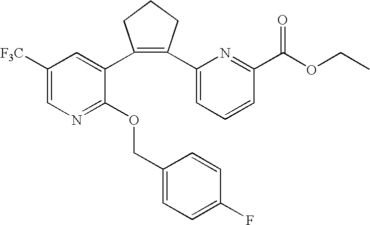 | Ethyl 6-(2-{2-[4- fluoro(phenylmethoxy)]-5- (trifluoromethyl)pyridin-3-yl}cyclopent- 1-en-1-yl)-pyridine-2-carboxylate | LC/MS Rt = 4.11 min [MH⁺] 487. |
| 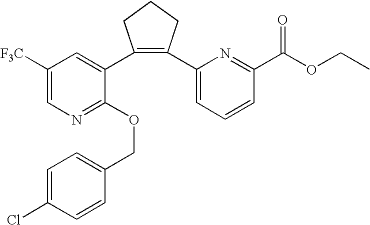 | Ethyl 6-{2-[2-{[(4- chlorophenyl)methyl]oxy}-5- (trifluoromethyl)-3-pyridinyl]-1- cyclopenten-1-yl}-2- pyridinecarboxylate | Rt = 4.24 min [MH⁺] 503 |
| 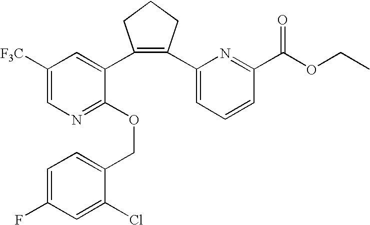 | Ethyl 6-{2-[2-{[(2-chloro-4- fluorophenyl)methyl]oxy}-5- (trifluoromethyl)-3-pyridinyl]-1- cyclopenten-1-yl}-2- pyridinecarboxylate | Rt = 4.28 min [MH⁺] 521 |
| 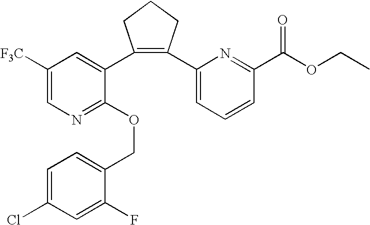 | Ethyl 6-{2-[2-{[(4-chloro-2- fluorophenyl)methyl]oxy}-5- (trifluoromethyl)-3-pyridinyl]-1- cyclopenten-1-yl}-2- pyridinecarboxylate | Rt = 4.28 min [MH⁺] 521 |
| 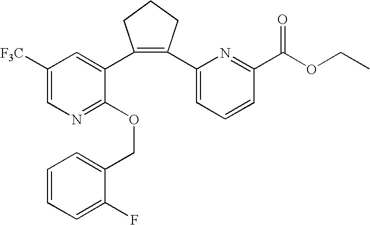 | Ethyl 6-{2-[2-{[(2- fluorophenyl)methyl]oxy}-5- (trifluoromethyl)-3-pyridinyl]-1- cyclopenten-1-yl}-2- pyridinecarboxylate | Rt = 4.11 min [MH⁺] 487 |
| 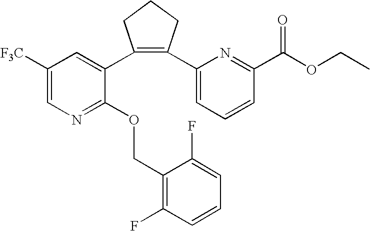 | Ethyl 6-{2-[2-{[(2,6- difluorophenyl)methyl]oxy}-5- (trifluoromethyl)-3-pyridinyl]-1- cyclopenten-1-yl}-2- pyridinecarboxylate | Rt = 4.08 min [MH⁺] 505 |
| 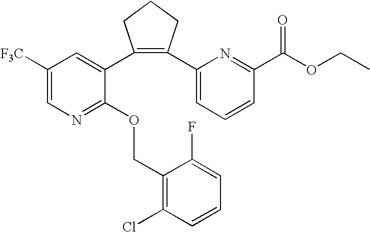 | Ethyl 6-{2-[2-{[(2-chloro-6- fluorophenyl)methyl]oxy}-5- (trifluoromethyl)-3-pyridinyl]-1- cyclopenten-1-yl}-2- pyridinecarboxylate | Rt = 4.20 min [MH⁺] 521 |
| 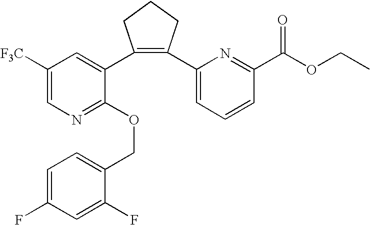 | Ethyl 6-{2-[2-{[(2,4- difluorophenyl)methyl]oxy}-5- (trifluoromethyl)-3-pyridinyl]-1- cyclopenten-1-yl}-2- pyridinecarboxylate | Rt = 4.13 min [MH⁺] 505 |
| 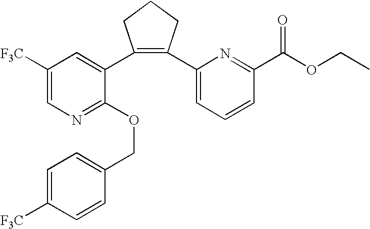 | Ethyl 6-{2-[5-(trifluoromethyl)-2-({[4- (trifluoromethyl)phenyl]methyl}oxy)-3- pyridinyl]-1-cyclopenten-1-yl}-2- pyrdininecarboxylate | Rt = 4.25 min [MH⁺] 537 |
| 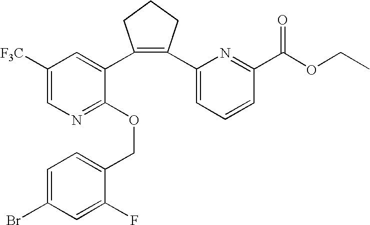 | Ethyl 6-{2-[2-{[(4-bromo-2- fluorophenyl)methyl]oxy}-5- (trifluoromethyl)-3-pyridinyl]-1- cyclopenten-1-yl}-2- pyridinecarboxylate | Rt = 4.31 min [MH⁺] 565, 567 |
| 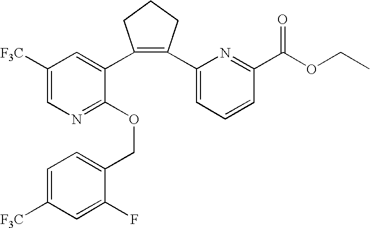 | Ethyl 6-{2-[2-({[2-fluoro-4- (trifluoromethyl)phenyl]methyl}oxy)-5- (trifluoromethyl)-3-pyridinyl]-1- cyclopenten-1-yl}-2- pyridinecarboxylate | Rt = 4.29 min [MH⁺] 555 |
| 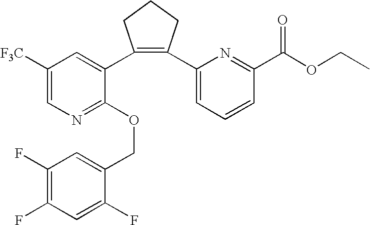 | Ethyl 6-[2-(5-(trifluoromethyl)-2- {[(2,4,5-trifluorophenyl)methyl]oxy}- 3-pyridinyl)-1-cyclopenten-1-yl]-2- pyridinecarboxylate | Rt = 4.17 min [MH⁺] 523 |
| 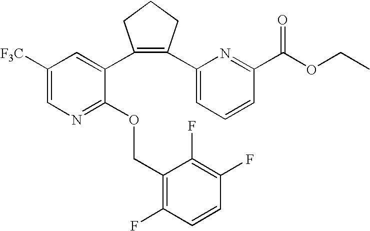 | Ethyl 6-[2-(5-(trifluoromethyl)-2- {[(2,3,6-trifluorophenyl)methyl]oxy}- 3-pyridinyl)-1-cyclopenten-1-yl]-2- pyridinecarboxylate | Rt = 4.10 min [MH⁺] 523 |

### (4-Fluorophenyl)methyl 2-fluoro-5-[2-(2-{[(4-fluorophenyl)methyl]oxy}-3-pyridinyl)-1-cyclopenten-1-yl]benzoate

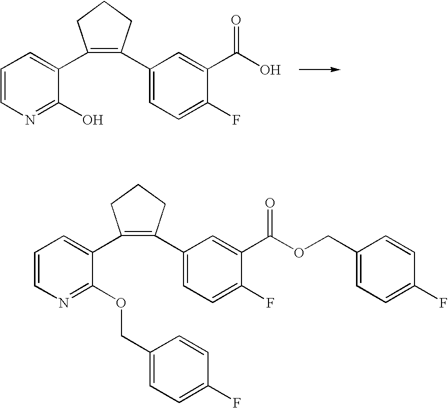

2-Fluoro-5-[2-(2-oxo-1,2-dihydro-3-pyridinyl)-1-cyclopenten-1-yl]benzoic acid (65 mg, 0.217 mmol) was dissolved in toluene (2 ml) and silver carbonate (1 32 mg, 0.478 mmol) and 4-fluorobenzyl bromide (60 μl, 0.478 mmol) added. The mixture was heated to reflux for 16 hours. After filtration and dilution with ethyl acetate, the solution was washed with water, dried (MgSO₄) and evaporated. The residue was flash chromatographed eluting with 3% ethyl acetate/isohexane to give the title compound (32 mg).

LC/MS Rt=4.40 min [MH⁺] 516.

The following intermediates were prepared by a similar route to (4-fluorophenyl)methyl 2-fluoro-5-[2-(2-{[(4-fluorophenyl)methyl]oxy}-3-pyridinyl)-1-cyclopenten-1-yl]benzoate from the appropriate intermediates.

| Structure | COMPOUND NAME | LCMS |
|---|---|---|
| 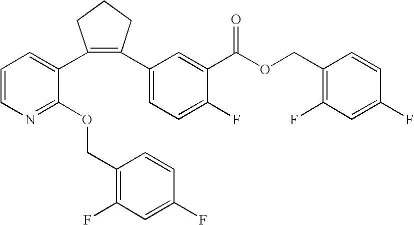 | (2,4-Difluorophenyl) methyl 5-[2-(2- {[(2,4-difluorophenyl)methyl]oxy}-3- pyridinyl)-1-cyclopenten-1-yl]-2- fluorobenzoate | Rt = 4.64 min [MH⁺] 552. |
| 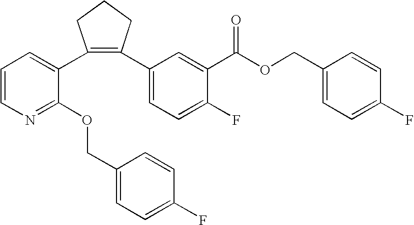 | (4-Fluorophenyl)methyl 2-fluoro-5- [2-(2-{[(4-fluorophenyl)methyl]oxy}- 3-pyridinyl)-1-cyclopenten-1- yl]benzoate | Rt = 4.37 min [MH⁺] 516. |
| 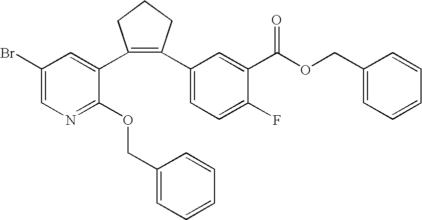 | Phenylmethyl 5-(2-{5-bromo-2- [(phenylmethyl)oxy]-3-pyridinyl}-1- cyclopenten-1-yl)-2-fluorobenzoate | Rt = 4.64 min [MH⁺] 558, 560. |
| 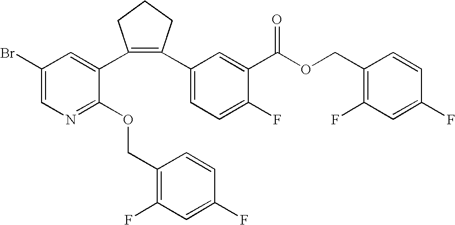 | (2,4-Difluorophenyl) methyl 5-[2-(5- bromo-2-{[(2,4-difluorophenyl) methyl]oxy}-3-pyridinyl)-1- cyclopenten-1-yl]-2-fluorobenzoate | Rt = 4.66 min [MH⁺] 630, 632. |
| 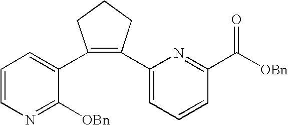 | Phenylmethyl 6-(2-{2- [(phenylmethyl)oxy]-3-pyridinyl}-1- cyclopenten-1-yl)-2- pyridinecarboxylate | Rt = 4.04 min [MH⁺] 463. |
| 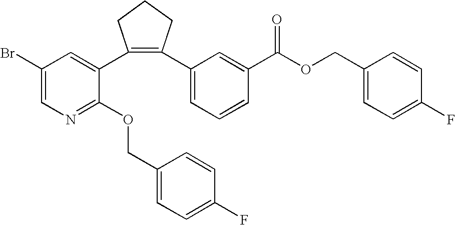 | (4-Fluorophenyl)methyl 3-[2-(5- bromo-2-{[(4-fluorophenyl) methyl]oxy}-3-pyridinyl)-1- cyclopenten-1-yl]benzoate | Rt = 4.63 min [MH⁺] 576, 578 |
| 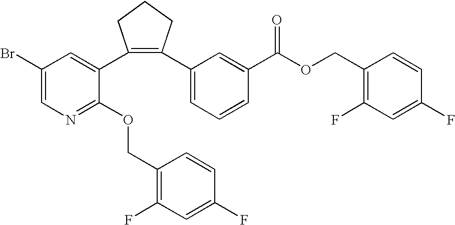 | (2,4-Difluorophenyl)methyl 3-[2-(5- bromo-2-{[(2,4-difluorophenyl) methyl]oxy}-3-pyridinyl)-1- cyclopenten-1-yl]benzoate | Rt = 4.46 min [MH⁺] 612, 614 |

### Ethyl 2-fluoro-5-{2-[2-(phenylmethoxy)-5-(trifluoromethyl)pyridin-3-yl]cyclopent-1-en-1-yl}benzoate

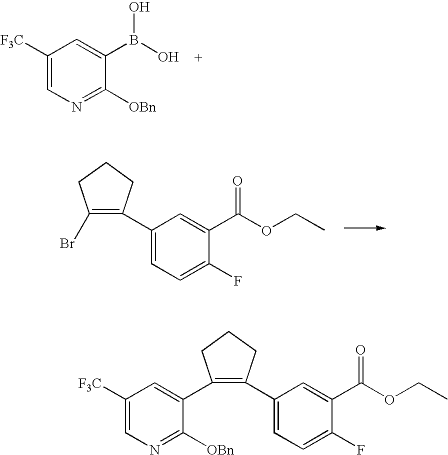

2-(Phenylmethoxy)-5-(trifluoromethyl)pyridine-3-boronic acid (10.32 g, 34.7 mmol) and ethyl 5-(2-bromocyclopent-1-enyl)-2-fluorobenzoate (5.44 g, 17.4 mmol) were dissolved in dimethoxyethane (120 mL) under nitrogen, and Pd(PPh₃)₄ (1.00 g, 0.87 mmol) and 2N aqueous sodium carbonate solution (60 ml) were added. The mixture was heated at 80° C. for 18 hours, but TLC analysis showed incomplete reaction. Further Pd(PPh₃)₄ was added and heating was continued for 3 hours. After cooling, the solvents were removed in vacuo, and the residue was partitioned between diethyl ether and water. The aqueous was extracted with further ether, and the combined organic extracts were dried (Na₂SO₄) and concentrated in vacuo. The resulting dark brown oil was purified by flash chromatography on silica (gradient elution, 0-6% ethyl acetate/cyclohexane) to give the title compound (7.02 g). LC/MS Rt=4.23 min [MH⁺] 485.

### Ethyl 2-fluoro-5-(2-{2-[(4-fluorophenyl)methoxy]-5-(trifluoromethyl)pyridin-3-yl}cyclopent-1-en-1-yl)-benzoate

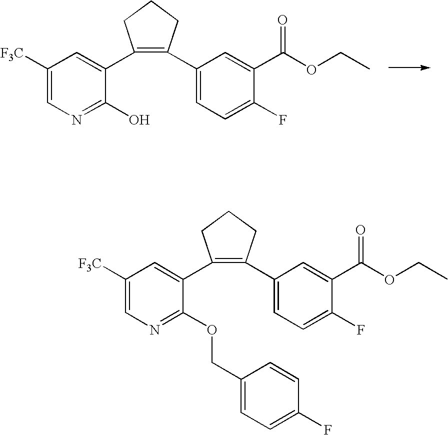

Ethyl 2-fluoro-5-{2-[2-hydroxy-5-(trifluoromethyl)pyridin-3-yl]cyclopent-1-en-1-yl}benzoate (250 mg, 0.633 mmol) was dissolved in toluene (4 ml), and silver carbonate (210 mg, 0.764 mmol) and 4-fluorobenzyl bromide (1 30 mg, 1.45 mmol) added. The mixture was heated to reflux for 5.5 hours. The mixture was concentrated in vacuo, and the residue was partitioned between water and dichloromethane. The organic extract was concentrated in vacuo. The residue was purified by flash chromatography on silica (gradient elution, 0-4% ethyl acetate/cyclohexane) to give the title compound.

LC/MS Rt=4.31 min [MH⁺] 504.

The following compounds (table) were prepared by the same method from ethyl 2-fluoro-5-{2-[2-hydroxy-5-(trifluoromethyl)pyridin-3-yl]cyclopent-1-en-1-yl}benzoate by reaction with appropriately substituted benzyl bromides.

| STRUCTURE | COMPOUND NAME | LCMS |
|---|---|---|
| 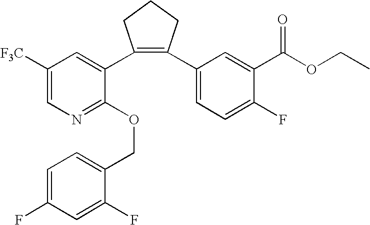 | Ethyl 5-(2-{2-[(2,4- difluorophenyl) methoxy]-5- (trifluoromethyl) pyridin-3- yl}cyclopent-1-en-1-yl)-2- fluorobenzoate | Rt = 4.33 min [MH⁺] 522 |
| 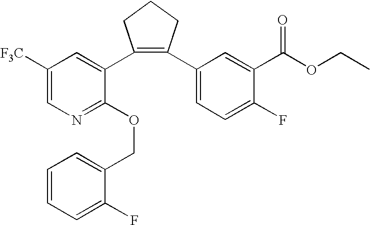 | Ethyl 2-fluoro-5-(2-{2-[(2- fluorophenyl)methoxy]-5- (trifluoromethyl)pyridin-3- yl}cyclopent-1-en-1-yl)- benzoate | Rt = 4.32 min [MH⁺] 504 |
| 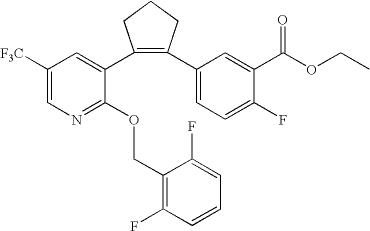 | Ethyl 5-(2-{2-[(2,6- difluorophenyl) methoxy]-5- (trifluoromethyl) pyridin-3- yl}cyclopent-1-en-1-yl)-2- fluorobenzoate | Rt = 4.30 min [MH⁺] 522 |
| 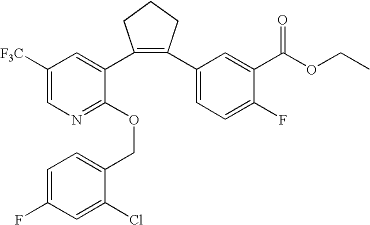 | Ethyl 5-(2-{2-[(2-chloro-4- fluorophenyl)methoxy]-5- (trifluoromethyl)pyridin-3- yl}cyclopent-1-en-1-yl)-2- fluorobenzoate | Rt = 4.45 min [MH⁺] 539 |

### Ethyl 3-fluoro-5-{2-[2-(phenylmethoxy)-5-(trifluoromethyl)pyridin-3-yl]cyclopent-1-en-1-yl}benzoate

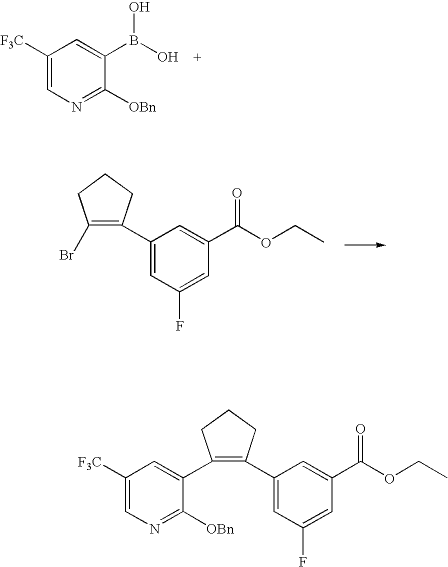

2-(Phenylmethoxy)-5-(trifluoromethyl)pyridine-3-boronic acid (10.53 g, 33.6 mmol) and ethyl 5-(2-bromocyclopent-1-enyl)-3-fluorobenzoate (5.93 g, 20.0 mmol) were dissolved in dimethoxyethane (120 mL) under nitrogen, and Pd(PPh₃)₄ (1.15 g, 1.0 mmol) and 2N aqueous sodium carbonate solution (60 ml) were added. The mixture was heated at 80° C. for 18 hours. After cooling, the solvents were removed in vacuo, and the residue was partitioned between diethyl ether and water. The aqueous was extracted with further ether, and the combined organic layers were dried (Na₂SO₄) and concentrated in vacuo. The resulting dark brown oil was purified by flash chromatography on silica (gradient elution, 0-4% ethyl acetate/cyclohexane) to give the title compound (7.42 g).

LC/MS Rt=4.32 min [MH⁺] 485.

### Ethyl 3-amino-5-{2-[2-(phenylmethoxy)-5-(trifluoromethyl)pyridin-3-yl]cyclopent-1-en-1-yl}benzoate

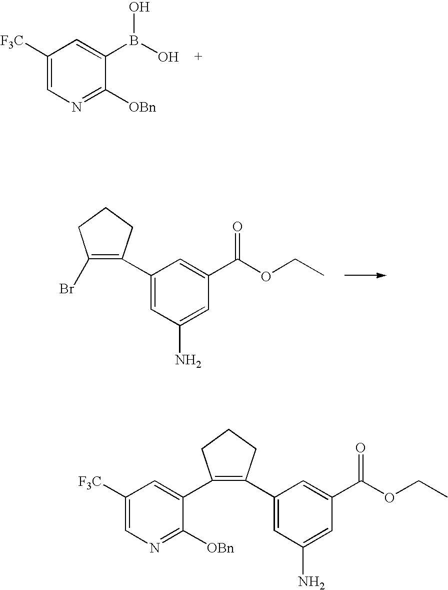

2-(Phenylmethoxy)-5-(trifluoromethyl)pyridine-3-boronic acid (6.0 g, 20.2 mmol) and ethyl 3-amino-5-(2-bromocyclopent-1-enyl)benzoate (3.16 g, 10.1 mmol) were dissolved in dimethoxyethane (50 mL) under nitrogen, and Pd(PPh₃)₄ (0.58 g, 0.5 mmol) and 2N aqueous sodium carbonate solution (10 ml) were added. The mixture was heated at 80° C. for 18 hours. After cooling, the solvents were removed in vacuo, and the residue was partitioned between diethyl ether and water. The aqueous was extracted with further ether (×2), and the combined organic layers were dried (Na₂SO₄) and concentrated in vacuo. The resulting dark brown oil was purified using an acidic solid phase cartridge (Isolute® Flash SCX-2, 50 g), loading the crude material as a methanol solution and eluting with 1 0% aqueous ammonia in methanol. Concentration of the relevant fractions in vacuo gave the title compound (4.01 g). LC/MS Rt=4.01 min [MH⁺] 483.

### General Procedure

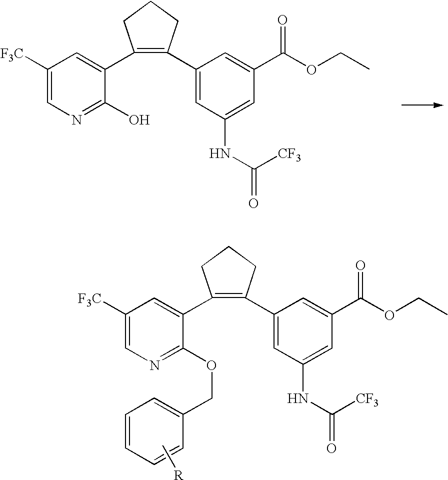

Ethyl 5-{2-[2-(hydroxy)-5-(trifluoromethyl)pyridin-3-yl]cyclopent-1-en-1-yl}-3-(trifluoroacetamido)benzoate (122 mg, 0.25 mmol) was dissolved in toluene (4 ml), together with silver carbonate (76 mg, 0.275 mmol) and a substituted benzyl bromide (1.1 equiv.), and this was heated to reflux for 18 hours. The mixture was filtered and concentrated in vacuo. The residue was-purified by flash chromatography on silica (gradient elution, 0-10% ethyl acetate/cyclohexane).

The following compounds were prepared by the above General Procedure from ethyl 2-fluoro-5-{2-[2-hydroxy-5-(trifluoromethyl)pyridin-3-yl]cyclopent-1-en-1-yl}benzoate by reaction with appropriately substituted benzyl bromides.

| | COMPOUND NAME | LCMS |
|---|---|---|
| 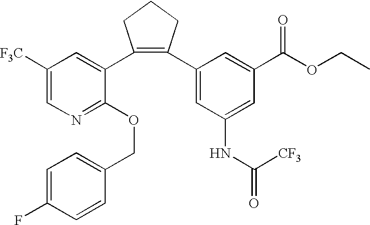 | Ethyl 5-(2-{2-[(4- fluorophenyl)methoxy]-5- (trifluoromethyl)pyridin-3- yl}cyclopent-1-en-1-yl)-3- (trifluoroacetamido)benzoate | Rt = 4.27 min [MH⁺] 597 |
| 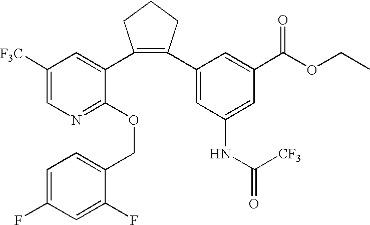 | Ethyl 5-(2-{2-[(2,4- difluorophenyl)methoxy]-5- (trifluoromethyl)pyridin-3- yl}cyclopent-1-en-1-yl)-3- (trifluoroacetamido)benzoate | Rt = 4.29 min [MH⁺] 615 |
| 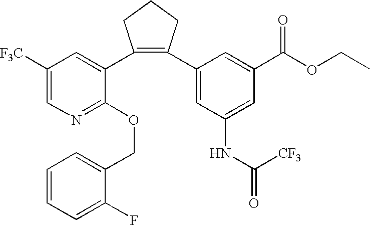 | Ethyl 5-(2-{2-[(2- fluorophenyl)methoxy]-5- (trifluoromethyl)pyridin-3- yl}cyclopent-1-en-1-yl)-3- (trifluoroacetamido)benzoate | Rt = 4.28 min [MH⁺] 597 |
| 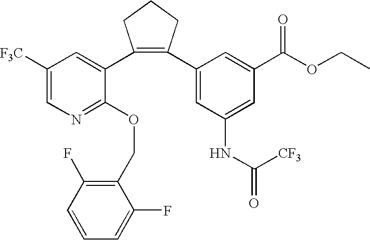 | Ethyl 5-(2-{2-[(2,6- difluorophenyl)methoxy]-5- (trifluoromethyl)pyridin-3- yl}cyclopent-1-en-1-yl)-3- (trifluoroacetamido)benzoate | Rt = 4.25 min [MH⁺] 615 |
| 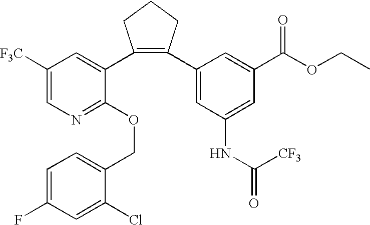 | Ethyl 5-(2-{2-[(2-chloro-4- fluorophenyl)methoxy]-5- (trifluoromethyl)pyridin-3- yl}cyclopent-1-en-1-yl-3- (trifluoroacetamido)benzoate | Rt = 4.30 min [MH⁺] 631 |
| 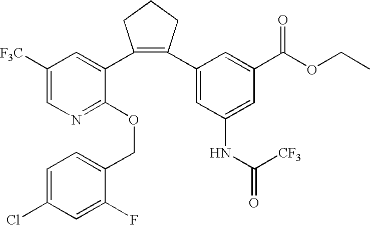 | Ethyl 5-(2-{2-[(4-chloro-2- fluorophenyl)methoxy]-5- (trifluoromethyl)pyridin-3- yl}cyclopent-1-en-1-yl)-3- (trifluoroacetamido)benzoate | Rt = 4.29 min [MH⁺] 631 |
| 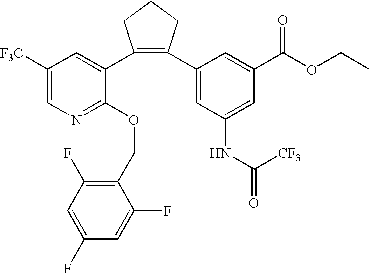 | Ethyl 3-(trifluoroacetamido)-5-(2- {5-(trifluoromethyl)-2-[(2,4,6- trifluorophenyl) methoxy]pyridin- 3-yl}cyclopent-1-en-1-yl)- benzoate | Rt = 4.17 min [MH⁺] 633 |
| 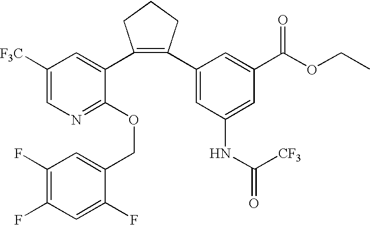 | Ethyl 3-(trifluoroacetamido)-5-(2- {5-(trifluoromethyl)-2-[(2,4,5- trifluorophenyl) methoxy]pyridin- 3-yl}cyclopent-1-en-1-yl)- benzoate | Rt = 4.30 min [MH⁺] 633 |
| 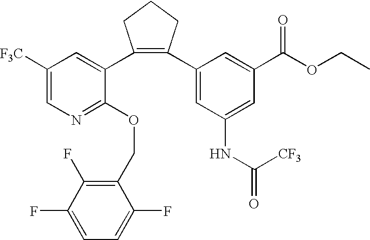 | Ethyl 3-(trifluoroacetamido)-5-(2- {5-(trifluoromethyl)-2-[(2,3,6- trifluorophenyl) methoxy]pyridin- 3-yl}cyclopent-1-en-1-yl)- benzoate | Rt = 4.26 min [MH⁺] 633 |
| 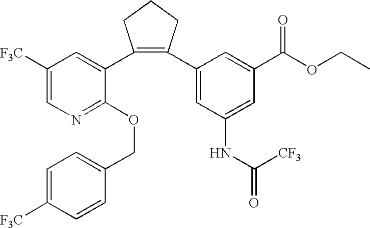 | Ethyl 3-(trifluoroacetamido)-5-[2- (5-[trifluoromethyl]-2-{[4- (trifluoromethyl)phenyl] methoxy}pyridin-3-yl)cyclopent- 1-en-1-yl]- benzoate | Rt = 4.37 min [MH⁺] 647 |
| 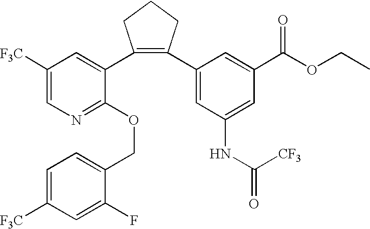 | Ethyl 5-[2-(2-{[2-fluoro-4- (trifluoromethyl)phenyl]methoxy}- 5-[trifluoromethyl]pyridin-3- yl)cyclopent-1-en-1-yl]-3- (trifluoroacetamido)benzoate | Rt = 4.40 min [MH⁺] 665 |
| 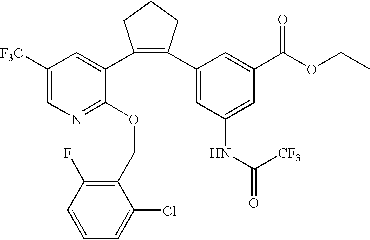 | Ethyl 5-(2-{2-[(2-chloro-6- fluorophenyl)methoxy}-5- (trifluoromethyl)pyridin-3- yl}cyclopent-1-en-1-yl)-3- (trifluoroacetamido)benzoate | Rt = 4.32 min [MH⁺] 631 |
| 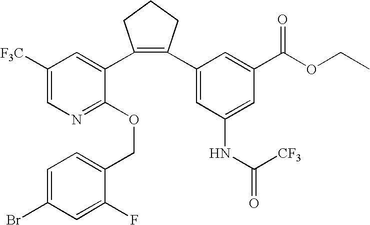 | Ethyl 5-(2-{2-[(4-bromo-2- fluorophenyl)methoxy]-5- (trifluoromethyl)pyridin-3- yl}cyclopent-1-en-1-yl)-3- (trifluoroacetamido)benzoate | Rt = 4.39 min [MH⁺] 675, 677 |

### PREPARATION OF EXAMPLES

### Example 1

### 6-{2-[2-{[(2,4-Dichlorophenyl)methyl]oxy}-5-(trifluoromethyl)phenyl]-1-cyclopenten-1-yl}-2-pyridinecarboxylic acid

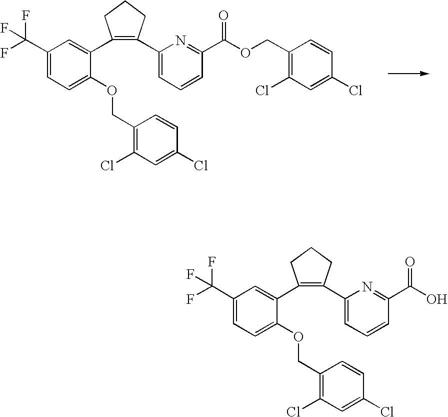

(2,4-Dichlorophenyl)methyl 6-{2-[2-{[(2,4-dichlorophenyl)methyl]oxy}-5-(trifluoromethyl)phenyl]-1-cyclopenten-1-yl}-2-pyridinecarboxylate (0.095 g), ethanol (2 ml) and 2M sodium hydroxide solution were heated in a Smithcreator® microwave to 120° C. for 3 minutes. After cooling the reaction was diluted with ethyl acetate and washed with dilute citric acid and brine, dried over MgSO₄, filtered and concentrated in vacuo to yield a yellow oil which was freeze-dried from acetonitrile/H₂O to give the title compound as an off-white solid.

¹H-NMR (CDCl₃) δ: 2.12-2.21 (2H, m), 2.91-2.98 (2H, m), 3.02-3.10 (2H, m), 5.03 (2H, s), 7.04 (1H, d), 7.08-7.16 (2H, m), 7.29 (1H, d), 7.35 (1H, d), 7.41 (1H, d), 7.58 (1H, dd), 7.72 (1H, t), 7.90 (1H, d). LC/MS Rt=4.50 min, [MH⁺] 508, 510, 512.

### Example 2

### 6-{2-[2-{[(2,6-Difluorophenyl)methyl]oxy}-5-(trifluoromethyl)phenyl]-1-cyclopenten-1-yl}-2-pyridinecarboxylic acid

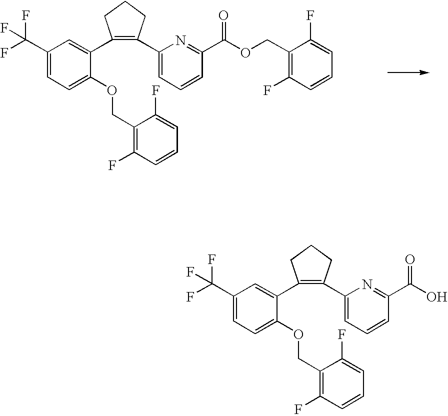

Procedure as for 6-{2-[2-{[(2,4-dichlorophenyl)methyl]oxy}-5-(trifluoromethyl)phenyl]-1-cyclopenten-1-yl}-2-pyridinecarboxylic acid.

LC/MS t=3.83, [MH⁺] 476.

### Example 3

### 6-[2-(5-(trifluoromethyl)-2-{[(2,4,6-trifluorophenyl)methyl]oxy}phenyl)-1-cyclopenten-1-yl]-2-pyrazinecarboxylic acid

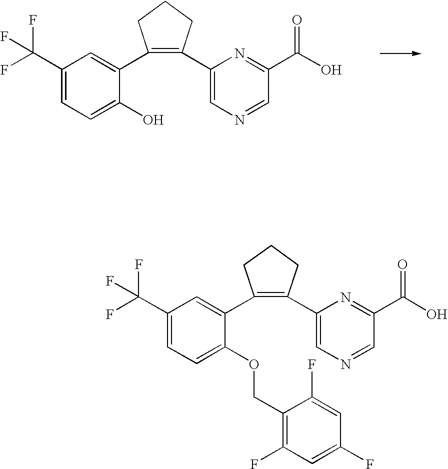

6-{2-[2-Hydroxy-5(trifluoromethyl)phenyl]-1-cyclopenten-1-yl}-2-pyrazinecarboxylic acid (0.15 g, 0.43 mmol), 2,4,6-trifluorobenzyl bromide (0.192 g, 0.86 mmol), potassium carbonate (0.13 g, 0.94 mmol) and potassium iodide (0.014 g, 0.086 mmol) were refluxed in methanol (10 ml) for 1 hour. The solvent was then removed in vacuo, the residue taken up in ethyl acetate and washed with acidified water (pH3). The aqueous layer was washed with ethyl acetate (×2). The combined organic layers were washed with brine, dried over MgSO₄, filtered and concentrated in vacuo to yield a yellow oil. This was purified by preparative HPLC to yield the title compound as an off-white solid (0.075 g).

¹H-NMR (MeOD) δ: 2.02-2.11 (2H, m), 2.85-2.93 (2H, m), 3.01-3.09 (2H, m), 5.04 (2H, s), 6.82 (2H, t), 7.35 (1H, d), 7.44 (1H, s), 7.64 (1H, d), 8.10 (1H, s), 8.86 (1H, s).

LC/MS Rt=3.90 min, [MH⁺] 495.

### Example 4

### 6-{2-[2-{[(2,6-Difluorophenyl)methyl]oxy}-5(trifluoromethyl)phenyl]-1-cyclopenten-1-yl}-2-pyrazinecarboxylic acid

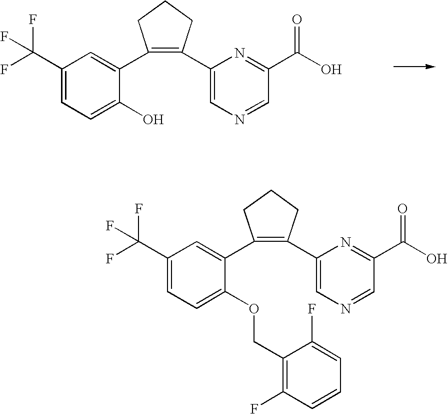

Procedure as for 6-[2-(5-(trifluoromethyl)-2-{[(2,4,6-trifluorophenyl)methyl]oxy}phenyl)-1-cyclopenten-1-yl]-2-pyrazinecarboxylic acid. LC/MS Rt=3.92 min, [MH⁺] 477.

### Standard Hydrolysis Procedure A

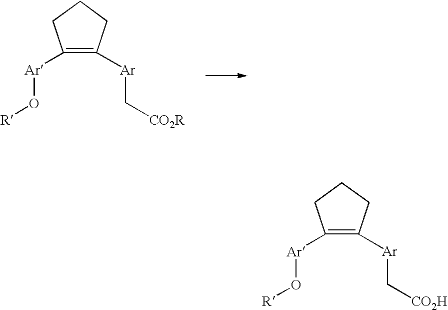

The ester (0.5 mmol) was dissolved in methanol or ethanol (2 ml) and 2M sodium hydroxide (1 ml) added. The mixture was either stirred at from room temperature to reflux for from 30 minutes to 20 hours until the reaction was complete by tic or heated at 120° C. in a Smithcreator® microwave for 3 minutes. The solution was diluted with water then extracted with isohexane or diethyl ether and acidified to pH4 with either hydrochloric acid, citric acid or acetic acid. The mixture was extracted with diethyl ether or dichloromethane. The organic solution was dried over magnesium sulphate and evaporated to give the title compound.

### Standard Hydrolysis Procedure B

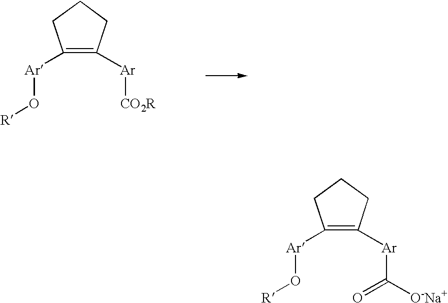

The ester (0.5 mmol) was dissolved in methanol or ethanol (2 ml) and 2M sodium hydroxide (1 ml) added. The mixture was stirred at from room temperature to reflux for from 30 minutes to 20 hours until the reaction was complete by tic or heated at 120° C. in a Smithcreator® microwave for 3 minutes then evaporated to dryness. The residue was dissolved in water/ethyl acetate or dichloromethane and the organic phase dried (magnesium sulphate), evaporated and the residue either dissolved in a small volume of ether and iso-hexane added to precipitate the salt or dissolved in dioxan and water and freeze-dried.

The following Examples were prepared by Standard Hydrolysis Procedure A:

| Example | Structure | Name | Data |
|---|---|---|---|
| 5 | 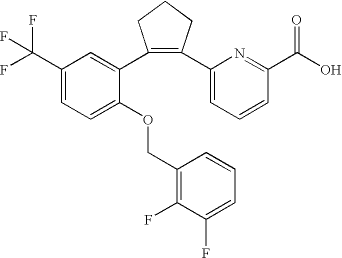 | 6-{2-[2-{[(2,3- Difluorophenyl)methyl] oxy}-5-(trifluoromethyl) phenyl]-1-cyclopenten-1- yl}-2-pyridinecarboxylic acid | LC/MS Rt = 3.91, [MH⁺] 476 |
| 6 | 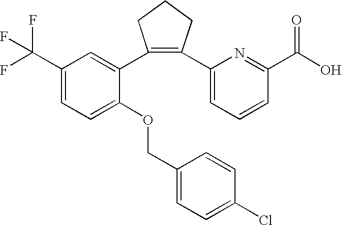 | 6-{2-[2-{[(4- Chlorophenyl)methyl] oxy}-5-(trifluoromethyl) phenyl]-1-cyclopenten-1-yl}- 2-pyridinecarboxylic acid | LC/MS Rt = 3.97, [MH+] 474, 476 |
| 7 | 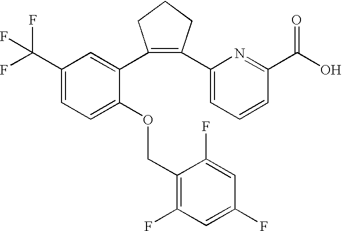 | 6-[2-(5-(Trifluoro- methyl)-2-{[(2,4,6- trifluorophenyl)methyl] oxy}phenyl)-1- cyclopenten-1-yl]-2- pyridinecarboxylic acid | LC/MS Rt = 3.82, [MH⁺] 494 |
| 8 | 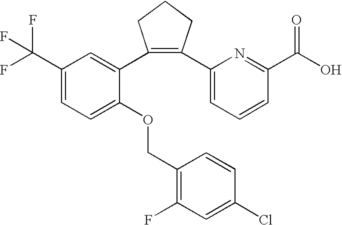 | 6-{2-[2-{[(4-Chloro-2- fluorophenyl)methyl] oxy}-5-(trifluoromethyl) phenyl]-1-cyclopenten-1- yl}-2-pyridinecarboxylic acid | LC/MS Rt = 4.05, [MH⁺] 492, 494 |
| 9 | 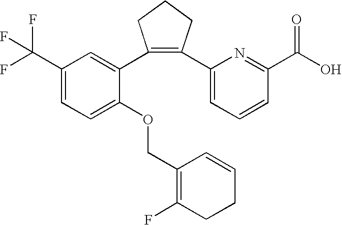 | 6-{2-[2-{[(2- Fluorophenyl)methyl) oxy}-5-(trifluoromethyl) phenyl]-1-cyclopenten-1-yl}- 2-pyridinecarboxylic acid | LC/MS Rt = 3.83, [MH⁺] 458 |
| 10 | 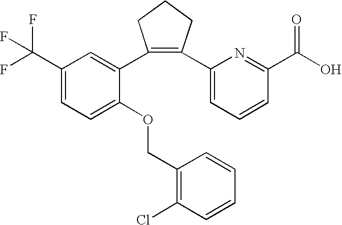 | 6-{2-[2-{[(2-Chloro- phenyl)methyl]oxy}-5- (trifluoromethyl)phenyl]- 1-cyclopenten-1-yl}- 2-pyridinecarboxylic acid | LC/MS Rt = 4.05, [MH⁺] 474, 476 |
| 11 | 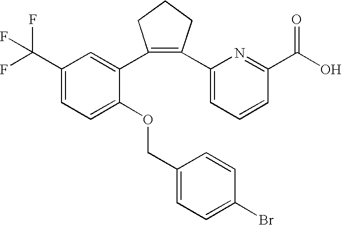 | 6-{2-[2-{[(4-Bromo- phenyl)methyl]oxy}-5- (trifluoromethyl)phenyl]- 1-cyclopenten-1-yl}- 2-pyridinecarboxylic acid | LC/MS Rt = 4.08, [MH⁺] 518, 520. |
| 12 | 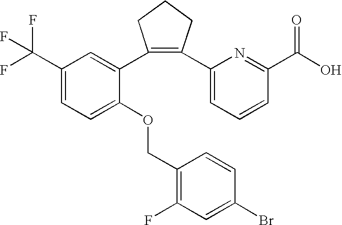 | 6-{2-[2-{[(4-Bromo-2- fluorophenyl)methyl] oxy}-5-(trifluoromethyl) phenyl]-1-cyclopenten-1-yl}- 2-pyridinecarboxylic acid | LC/MS Rt = 4.13, [MH⁺] 536, 538. |
| 13 | 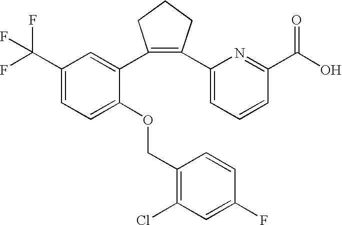 | 6-{2-[2-{[(2-Chloro-4- fluorophenyl)methyl] oxy}-5-(trifluoromethyl) phenyl]-1-cyclopenten-1-yl}- 2-pyridinecarboxylic acid | LC/MS Rt = 4.07 [MH⁺] 492, 494. |
| 14 | 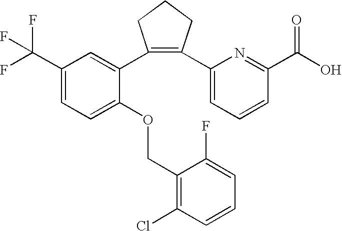 | 6-{2-[2-{[(2-Chloro-6- fluorophenyl)methyl] oxy}-5-(trifluoromethyl) phenyl]-1-cyclopenten-1-yl}- 2-pyridinecarboxylic acid | LC/MS Rt = 3.93 [MH⁺] 492, 494 |
| 15 | 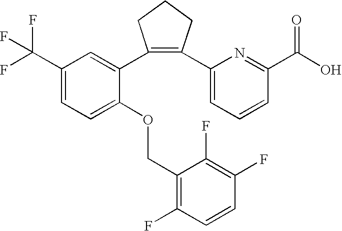 | 6-[2-(5-(Trifluoromethyl)- {[(2,3,6-trifluorophenyl) methyl]oxy}phenyl)-1- cyclopenten-1-yl]-2- pyridinecarboxylic acid | LC/MS Rt = 3.83 [MH⁺] 494 |
| 16 | 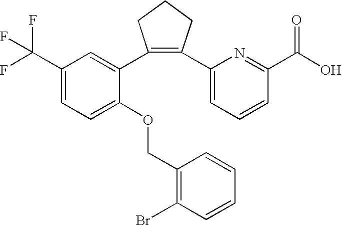 | 6-{2-[2-{[(2- Bromophenyl)methyl] oxy}-5-(trifluoromethyl) phenyl]-1-cyclopenten-1-yl}- 2-pyridinecarboxylic acid | LC/MS Rt = 4.10 [MH⁺] 518, 520 |
| 17 | 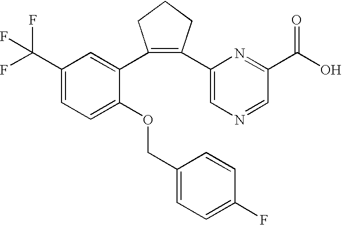 | 6-{2-[2-{[(4- Fluorophenyl)methyl] oxy}-5-(trifluoromethyl) phenyl]-1-cyclopenten]- 1-yl}-2-pyrazinecarboxylic acid | LC/MS Rt = 3.93 [MH⁺] 459. |
| 18 | 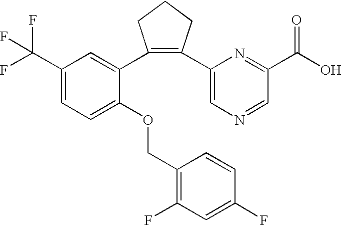 | 6-{2-[2-{[(2,4- Difluorophenyl)methyl] oxy}-5-(trifluoromethyl) phenyl]-1-cyclopenten-1-yl}- 2-pyrazinecarboxylic acid | LC/MS Rt = 4.00, [MH⁺] 477. |
| 19 | 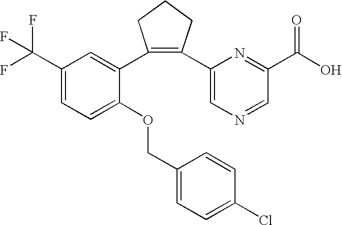 | 6-{2-[2-{[(4- Chlorophenyl)methyl] oxy}-5-fluoromethyl) phenyl]-1-cyclopenten-1- yl}-2-pyrazinecarboxylic acid | LC/MS Rt = 4.08, [MH⁺] 475, 477 |
| 20 | 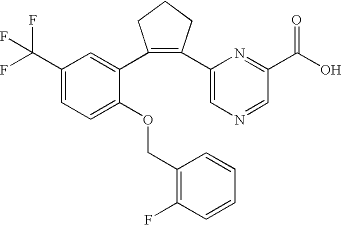 | 6-{2-[2-{[(2- Fluorophenyl)methyl] oxy}-5-(trifluoromethyl) phenyl]-1-cyclopenten-1-yl}- 2-pyrazinecarboxylic acid | LC/MS Rt = 3.89, [MH⁺] 459 |
| 21 | 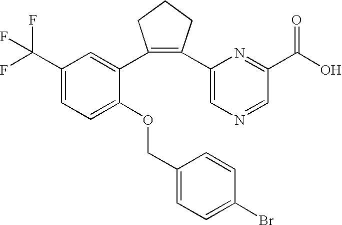 | 6-{2-[2-{[(4- Bromophenyl)methyl] oxy}-5-(trifluoromethyl) phenyl]-1-cyclopenten-1-yl}- 2-pyrazinecarboxylic acid | LC/MS Rt = 4.09, [MH⁺] 517, 519 |
| 22 | 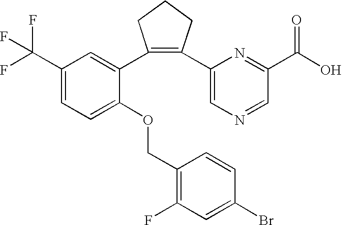 | 6-{2-[2-{[(4-Bromo-2- fluorophenyl)methyl] oxy}-5-(trifluoromethyl) phenyl]-1-cyclopenten-1-yl}- 2-pyrazinecarboxylic acid | LC/MS Rt = 4.15, [MH⁺] 537, 539 |
| 23 | 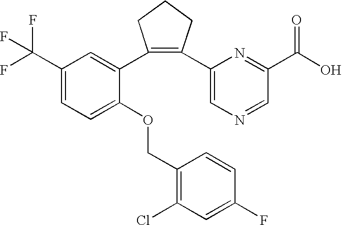 | 6-{2-[2-{[(2-Chloro-4- fluorophenyl)methyl] oxy}-5-(trifluoromethyl) phenyl]-1-cyctopenten-1-yl)- 2-pyrazinecarboxylic acid | LC/MS Rt = 4.04, [MH⁺] 493/495 |
| 24 | 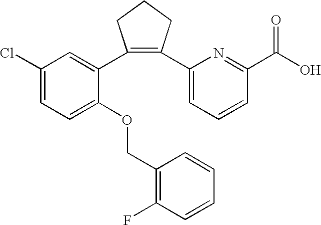 | 6-[2-(5-Chloro-2-{[(2- fluorophenyl)methyl] oxy}phenyl)-1- cyclopenten-1-yl]-2- pyridinecarboxylic acid | ¹H NMR (CDCl₃) δ: 2.09- 2.15 (2H, m), 2.86-2.92 (2H, m), 2.98-3.04 (2H, m), 4.97 (2H, s), 6.93-7.02 (3H, m), 7.05-7.11 (2H, m), 7.23-7.27 (3H, m), 7.61-7.72 (1H, bs), 7.86- 7.93 (1H, bs). LC/MS Rt = 3.60, [MH+] 424, 426, 427 [MH−] 422, 424 |
| 25 | 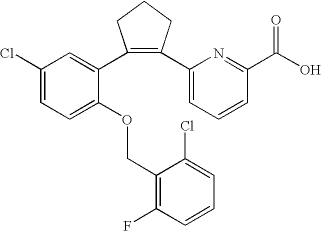 | 6-[2-(5-Chloro-2-{[(2- chloro-6- fluorophenyl)methyl] oxy}phenyl)-1- cyclopenten-1-yl]-2- pyridinecarboxylic acid | ¹H NMR (CDCl₃) δ: 2.02- 2.06 (2H, m), 2.78-2.84 (2H, m), 2.93-2.97 (2H, m), 5.05 (2H, s), 6.90 (1H, t), 7.07-7.09 (3H, m), 7.15-7.21 (2H, m), 7.21- 7.28 (1H, m), 7.63-7.67 (1H, m), 7.86 (1H, d). LC/MS Rt = 3.68, [MH+] 458, 461 [MH−] 456, 459 |
| 26 | 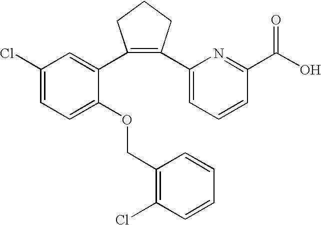 | 6-[2-(5-Chloro-2-{[(2- chlorophenyl)methyl] oxy}phenyl)-1- cyclopenten-1-yl]-2- pyridinecarboxylic acid | ¹H NMR (CDCl₃) δ: 2.11- 2.15 (2H, m), 2.90-2.94 (2H, m), 3.02-3.06 (2H, m), 5.00 (2H, s), 6.92 (1H, d), 7.12-7.18 (3H, m), 7.19-7.31 (4H, m), 7.69 (1H, t), 7.89 (1H, d). LC/MS Rt = 3.79, [MH+] 440, 443 [MH−] 438, 441 |
| 27 | 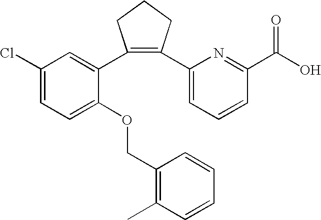 | 6-[2-(5-Chloro-2-{[(2- methylphenyl)methyl] oxy}phenyl)-1- cyclopenten-1-yl]-2- pyridinecarboxylic acid | ¹H NMR (CDCl₃) δ: 2.07- 2.1 (2H, m), 2.85-2.89 (2H, m), 2.98-3.02 (2H, m), 4.8 (2H, s), 6.94 (1H, d), 7.06-7.09 (4H, m), 7.14-7.18 (1H, m), 7.23- 7.26 (2H, m), 7.64-7.68 (1H, m), 7.87 (1H, d). LC/MS Rt = 3.68, [MH+] 420, 422 [MH−] 418, 420 |
| 28 | 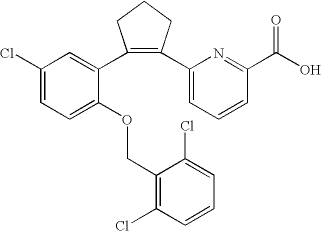 | 6-[2-(5-Chloro-2- {[(2,6-dichlorophenyl) methyl]oxy}phenyl)-1- cyclopenten-1-yl]-2- pyridinecarboxylic acid | ¹H NMR (CDCl₃) δ: 2.01- 2.05 (2H, m), 2.85-2.87 (2H, m), 2.91-2.95 (2H, m), 5.24 (2H, s), 7.09-7.32 (7H, m), 7.63-7.67 (1H, m), 7.86 (1H, d). LC/MS Rt = 3.81, [MH+] 476, 478 [MH−] 474, 476 |
| 29 | 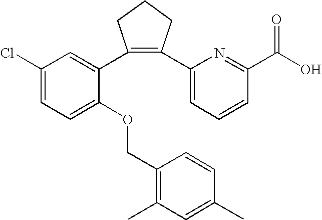 | 6-[2-(5-Chloro-2- {[(2,4-dimethylphenyl) methyl]oxy}phenyl)- 1-cyclopenten-1-yl]- 2-pyridinecarboxylic acid | ¹H NMR (CDCl₃) δ: 2.05- 2.11 (2H, m), 2.14 (3H, s), 2.26 (3H, s), 2.85-2.89 (2H, m), 2.97-3.01 (2H, m), 4.85 (2H, s), 6.88 (1H, s), 6.92-6.96 (2H, m), 7.08 (1H, s), 7.22-7.26 (3H, m), 7.66 (1H, t), 7.87 (1H, d). LC/MS Rt = 3.81, [MH+] 434, 436 [MH−] 432, 434 |
| 30 | 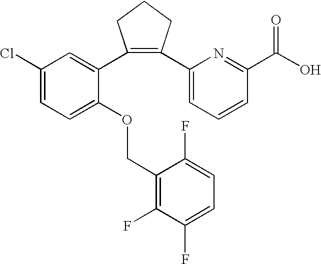 | 6-[2-(5-Chloro-2- {[(2,3,6- trifluorophenyl)methyl] oxy}phenyl)-1- cyclopenten-1-yl]-2- pyridinecarboxylic acid | ¹H NMR (CDCl₃) δ: 2.03- 2.11 (2H, m), 2.81-2.85 (2H, m), 2.95-2.99 (2H, m), 5.0 (2H, s), 6.73-6.75 (1H, m), 7.03-7.09 (3H, m), 7.25-7.29 (2H, m), 7.68 (1H, t), 7.88 (1H, d). LC/MS Rt = 3.60, [MH+] 460, 463 |
| 31 | 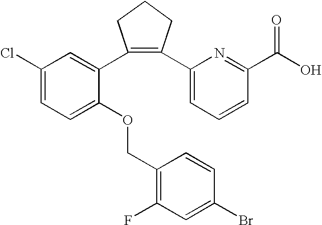 | 6-[2-(2-{[(4-Bromo-2- fluorophenyl)methyl] oxy}-5-chlorophenyl)-1- cyclopenten-1-yl]-2- pyridinecarboxylic acid | ¹H NMR (CDCl₃) δ: 2.08- 2.13 (2H, m), 2.86-2.89 (2H, m), 2.99-3.03 (2H, m), 4.93 (2H, s), 6.93 (1H, d), 6.99 (1H, t), 7.01 (1H, s), 7.14-7.18 (2H, m), 7.25-7.27 (2H, m), 7.71 (1H, t), 7.91 (1H, d). LC/MS Rt = 3.86, [MH+] 504, 506 [MH−] 502, 503 |
| 32 | 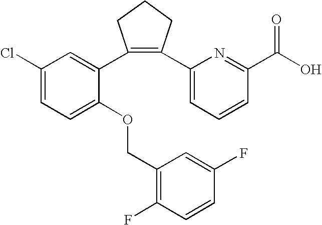 | 6-[2-(5-Chloro-2- {[(2,5- difluorophenyl)methyl] oxy}phenyl)-1- cyclopenten-1-yl]-2- pyridinecarboxylic acid | ¹H NMR (CDCl₃) δ: 2.13- 2.16 (2H, m), 2.89-2.93 (2H, m), 3.02-3.07 (2H, m), 4.94 (2H, s), 6.78-6.81 (1H, m), 6.90-6.96 (3H, m), 7.14 (1H, bs), 7.25- 7.27 (2H, m), 7.69-7.71 (1H, m), 7.86-7.89 (1H, m). LC/MS Rt = 3.86, [MH+] 504, 506 [MH−] 502, 503 |
| 33 | 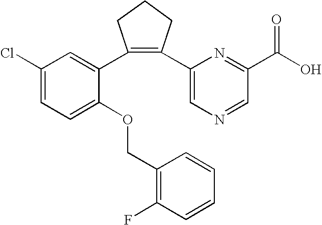 | 6-[2-(5-Chloro-2-{[(2- fluorophenyl)methyl] oxy}phenyl)-1- cyclopenten-1-yl]-2- pyrazinecarboxylic acid | ¹H NMR (CDCl₃) δ: 2.10- 2.18 (2H, m), 2.91-2.95 (2H, m), 3.02-3.06 (2H, m), 4.94 (2H, s), 6.96-7.29 (7H, m), 8.53 (1H, s), 9.04 (1H, s). LC/MS Rt = 4.32, [MH+] 425, 427 [MH−] 423, 425 |
| 34 | 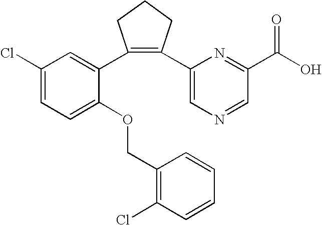 | 6-[2-(5-Chloro-2-{[(2- chlorophenyl)methyl] oxy}phenyl)-1- cyclopenten-1-yl]-2- pyrazinecarboxylic acid | ¹H NMR (CDCl₃) δ: 2.12- 2.20 (2H, m) 2.94-2.97 (2H, m), 3.05-3.08 (2H, m), 4.98 (2H, s), 6.95 (1H, d), 7.12-7.31 (6H, m), 8.55 (1H, s), 9.03 (1H, s). LC/MS Rt = 4.65, [MH+] 441, 444 [MH−] 439, 443 |
| 35 | 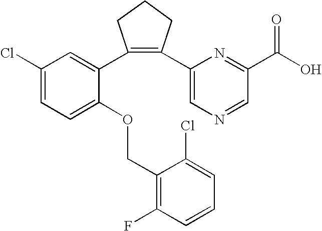 | 6-[2-(5-chloro-2-{[(2- chloro-6- fluorophenyl)methyl] oxy}phenyl)-1- cyclopenten-1-yl]-2- pyrazinecarboxylic acid | ¹H NMR (CDCl₃) δ: 2.06- 2.10 (2H, m), 2.85-2.88 (2H, m), 2.96-2.99 (2H, m), 5.02 (2H, s), 6.90 (1H, t, J = 8.9 Hz), 7.06- 7.12 (2H, m), 7.16-7.2 (2H, m), 7.30 (1H, dd, J = 8.8 J = 2.6 Hz), 8.48 (1H, s), 9.03 (1H, s). LC/MS Rt = 4.40, [MH+] 459, 462 [MH−] 457, 460 |
| 36 | 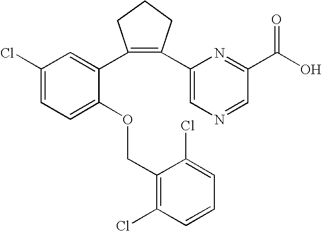 | 6-[2-(5-Chloro-2- [[(2,6- dichlorophenyl)methyl] oxy}phenyl)-1- cyclopenten-1-yl]-2- pyrazinecarboxylic acid | ¹H NMR (CDCl₃) δ: 2.05- 2.09 (2H, m), 2.85-2.89 (2H, m), 2.94-2.97 (2H, m), 5.11 (2H, s), 7.09 (1H, d, J = 8.8 Hz), 7.06- 7.12 (2H, m), 7.15-7.32 (5H, m), 8.48 (1H, s), 9.02 (1H, s). LC/MS Rt = 4.62, [MH+] 477, 479 [MH−] 475, 477 |
| 37 | 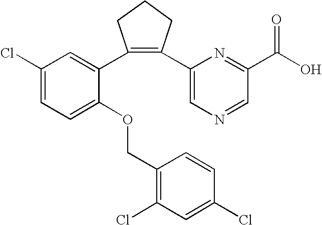 | 6-[2-(5-Chloro-2- {[(2,4- dichlorophenyl)methyl] oxy}phenyl)-1- cyclopenten-1-yl]-2- pyrazinecarboxylic acid | ¹H NMR (DMSO) δ: 1.97- 2.03 (2H, m), 2.81-2.85 (2H, m), 2.95-2.98 (2H, m), 5.10 (2H, s), 7.06 (1H, d), 7.17 (1H, d), 7.26 (1H, d), 7.33 (1H, dd), 7.43 (1H, dd), 7.63 (1H, d), 7.80 (1H, s), 8.58 (1H, s). LC/MS Rt = 4.92, [MH+] 477, 479 [MH−] 475, 477 |
| 38 | 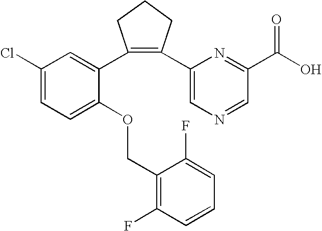 | 6-[2-(5-Chloro-2- {[(2,6- difluorophenyl)methyl] oxy}phenyl)-1- cyclopenten-1-yl]-2- pyrazinecarboxylic acid | ¹H NMR (DMSO) δ: 1.89- 1.97 (2H, m), 2.75-2.79 (2H, m), 2.89-2.93 (2H, m), 5.06 (2H, s), 7.03- 7.11 (3H, m), 7.30 (1H, d), 7.38-7.47 (2H, m), 7.96 (1H, s), 8.75 (1H, s). LC/MS Rt = 4.65, [MH+] 443, 445 [MH−] 441, 443 |
| 39 | 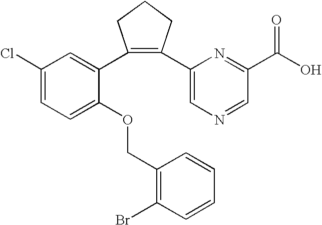 | 6-[2-(2-{[(2- Bromophenyl)methyl] oxy}-5-chlorophenyl)- 1-cyclopenten-1-yl]-2- pyrazinecarboxylic acid | ¹H NMR (CDCl₃) δ: 2.14- 2.18 (2H, m), 2.94-2.98 (2H, m), 3.05-3.09 (2H, m), 4.95 (2H, s), 6.94 (1H, d), 7.11-7.19 (4H, m), 7.27-7.29 (1H, m), 7.48 (1H, d), 8.54 (1H, s), 9.03 (1H, s). LC/MS Rt = 4.75, [MH+] 487, 489 [MH−] 485, 487 |
| 40 | 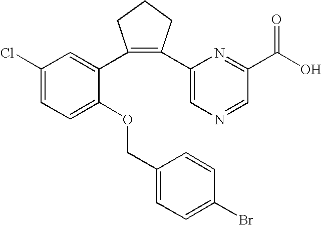 | 6-[2-(2-{[(4- Bromophenyl)methyl] oxy}-5-chlorophenyl)- 1-cyclopenten-1-yl]-2- pyrazinecarboxylic acid | ¹H NMR (CDCl₃) δ: 2.12- 2.16 (2H, m), 2.91-2.95 (2H, m), 3.05-3.09 (2H, m), 4.85 (2H, s), 6.88 (1H, d), 6.99 (2H, d), 7.11 (1H, bs), 7.23-7.25 (1H, m), 7.39 (2H), 8.51 (1H, s), 9.04 (1H, s). LC/MS Rt = 4.64, [MH+] 487, 488 [MH−] 485, 487 |
| 41 | 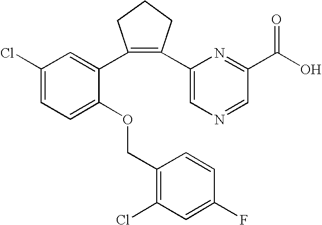 | 6-[2-(5-Chloro-2-{[(2- chloro-4- fluorophenyl)methyl] oxy}phenyl)-1- cyclopenten-1-yl]-2- pyrazinecarboxylic acid | ¹H NMR (CDCl₃) δ: 2.13- 2.17 (2H, m), 2.91-2.95 (2H, m), 3.05-3.08 (2H, m), 4.94 (2H, s), 6.89- 6.95 (2H, m), 7.07 (1H, dd), 7.11-7.15 (2H, m), 7.27-7.30 (1H, m), 8.55 (1H, s), 9.06 (1H, s). LC/MS Rt = 4.59, [MH+] 459, 462 [MH−] 457, 461 |
| 42 | 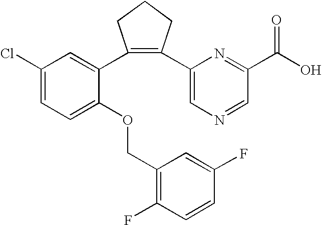 | 6-[2-(5-Chloro-2- {[(2,5- difluorophenyl)methyl] oxy}phenyl)-1- cyclopenten-1-yl]-2- pyrazinecarboxylic acid | ¹H NMR (CDCl₃) δ: 2.12- 2.21 (2H, m), 2.92-2.98 (2H, m), 3.03-3.11 (2H, m), 4.94 (2H, s), 6.78- 6.84 (1H, m), 6.91-6.98 (3H, m), 7.15 (1H, s), 7.26-7.31 (1H, m), 8.55 (1H, s), 9.06 (1H, s). LC/MS Rt = 4.29, [MH+] 443, 445 [MH−] 441, 443 |
| 43 | 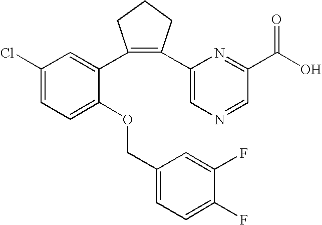 | 6-[2-(5-Chloro-2- {[(3,4- difluorophenyl)methyl] oxy}phenyl)-1- cyclopenten-1-yl]-2- pyrazinecarboxylic acid | ¹H NMR (CDCl₃) δ: 2.15- 2.21 (2H, m), 2.91-2.97 (2H, m), 3.08-3.11 (2H, m), 4.86 (2H, s), 6.81- 6.85 (1H, m), 6.89 (1H, d), 6.92-6.97 (1H, m), 7.03-7.12 (1H, m), 7.14 (1H, s), 7.26-7.31 (1H, m), 8.58 (1H, s), 9.08 (1H, s). LC/MS Rt = 4.29, [MH+] 443, 445 [MH−] 441, 443 |
| 44 | 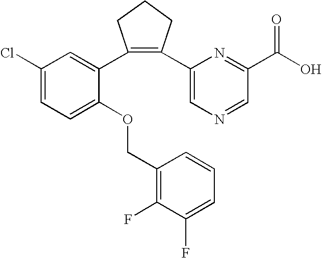 | 6-[2-(5-Chloro-2- {[(2,3- difluorophenyl)methyl] oxy}phenyl)-1- cyclopenten-1-yl]-2- pyrazinecarboxylic acid | ¹H NMR (CDCl₃) δ: 2.12- 2.19 (2H, m), 2.89-2.95 (2H, m), 3.03-3.10 (2H, m), 4.98 (2H, s), 6.81- 6.87 (1H, m), 6.92- 6.97 (2H, m), 7.04-7.14 (2H, m), 7.26-7.31 (1H, m), 8.56 (1H, s), 9.06 (1H, s). LC/MS Rt = 4.34, [MH+] 443, 445 [MH−] 441 |
| 45 | 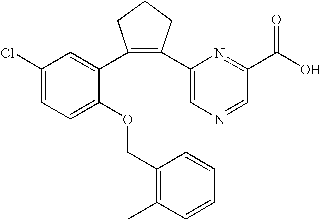 | 6-[2-(5-Chloro-2-{[(2- methylphenyl)methyl] oxy}phenyl)-1- cyclopenten-1-yl]-2- pyrazinecarboxylic acid | ¹H NMR (DMSO) δ: 1.95- 2.03 (2H, m), 2.19 (3H, s), 2.83-2.86 (2H, m), 2.96- 3.0 (2H, m), 5.01 (2H, s), 7.08-7.18 (5H, m), 7.23 (1H, d), 7.36 (1H, dd), 8.03 (1H, s), 8.73 (1H, s). LC/MS Rt = 4.42, [MH+] 421, 423 [MH−] 419, 421 |
| 46 | 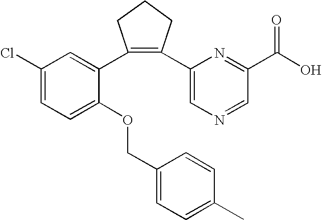 | 6-[2-(5-chloro-2-{[(4- methylphenyl)methyl] oxy}phenyl)-1- cyclopenten-1-yl]-2- pyrazinecarboxylic acid | ¹H NMR (DMSO) δ: 1.96- 2.03 (2H, m), 2.25 (3H, s), 2.84-2.86 (2H, m), 2.95- 2.99 (2H, m), 4.97 (2H, s), 7.02-7.14 (6H, m), 7.31 (1H, dd, J = 8.8, 2.8 Hz), 7.95 (1H, s), 8.69 (1H, s). LC/MS Rt = 4.42, [MH+] 421 [MH−] 419, 421 |
| 47 | 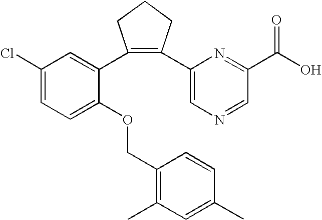 | 6-[2-(5-Chloro-2- {[(2,4-dimethylphenyl) methyl]oxy}phenyl)-1- cyclopenten-1-yl]-2- pyrazinecarboxylic acid | ¹H NMR (DMSO) δ: 1.91- 1.99 (2H, m), 2.13 (3H, s), 2.21 (3H, s), 2.83-2.90 (2H, m), 2.96-3.0 (2H, m), 4.94 (2H, s), 6.95- 7.04 (3H, m), 7.18-7.24 (2H, m), 7.36-7.40 (1H, m), 8.10 (1H, s), 8.77 (1H, s), 13.65 (1H, s). LC/MS Rt = 4.64, [MH+] 435 [MH−] 433, 436 |
| 48 | 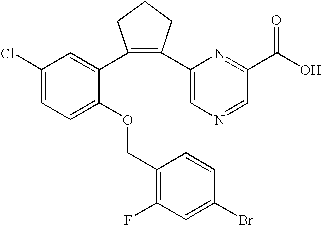 | 6-[2-(2-{[(4-Bromo-2- fluorophenyl)methyl] oxy}-5-chlorophenyl)-1- cyclopenten-1-yl]-2- pyrazinecarboxylic acid | ¹H NMR (CDCl₃) δ: 2.12- 2.16 (2H, m), 2.87- 2.91 (2H, m), 2.99-3.07 (2H, m), 4.91 (2H, s), 6.93-7.01 (2H, m), 7.12 (1H, bs), 7.18 (1H, d), 7.26-7.29 (1H, m), 8.53 (1H, s), 9.07 (1H, s). LC/MS Rt = 4.64, [MH+] 505, 507 [MH−] 502, 505 |
| 49 | 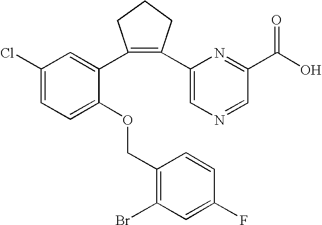 | 6-[2-(2-{[(2-Bromo-4- fluorophenyl)methyl] oxy}-5-chlorophenyl)-1- cyclopenten-1-yl]-2- pyrazinecarboxylic acid | ¹H NMR (CDCl₃) δ: 2.13- 2.17 (2H, m), 2.92- 2.96 (2H, m), 3.05-3.09 (2H, m), 4.92 (2H, s), 6.92-6.96 (2H, m), 7.10- 7.14 (2H, m), 7.24-7.30 (2H, m), 8.54 (1H, s), 9.06 (1H, s). LC/MS Rt = 4.67, [MH+] 505, 507 [MH−] 503, 505 |
| 50 | 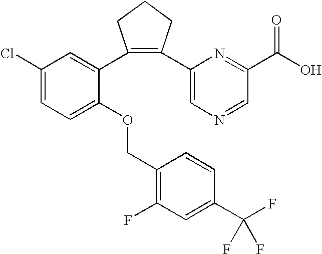 | 6-{2-[5-Chloro-2-({[2- fluoro-4- (trifluoromethyl)phenyl] methyl}oxy)phenyl]- 1-cyclopenten-1-yl}-2- pyrazinecarboxylic acid | ¹H NMR (DMSO) δ: 1.99- 2.03 (2H, m), 2.85- 2.88 (2H, m), 2.97-3.01 (2H, m), 5.15 (2H, s), 7.18 (1H, d), 7.23 (1H, d), 7.37 (1H, dd), 7.42 (1H, t), 7.54 (1H, d), 7.63 (1H, d), 8.06 (1H, s), 8.74 (1H, s). LC/MS Rt = 4.46, [MH+] 493, 495 [MH−] 491, 493 |
| 51 | 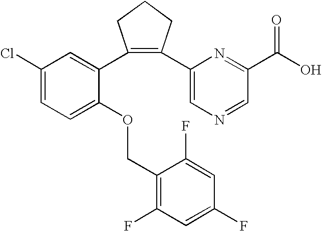 | 6-[2-(5-Chloro-2- {[(2,4,6- trifluorophenyl)methyl] oxy}phenyl)-1- cyclopenten-1-yl]-2- pyrazinecarboxylic acid | ¹H NMR (DMSO) δ: 1.91- 1.99 (2H, m), 2.78-2.81 (2H, m), 2.90-2.94 (2H, m), 4.96 (2H, s), 7.12 (2H, t), 7.21 (1H, d), 7.28 (1H, d), 7.39 (1H, dd), 8.07 (1H, s), 8.81 (1H, s), 13.65 (1H, s). LC/MS Rt = 4.20, [MH+] 461, 463 [MH−] 459, 461 |
| 52 | 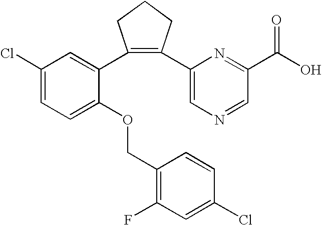 | 6-[2-(5-Chloro-2-{[(4- chloro-2- fluorophenyl)methyl] oxy}phenyl)-1- cyclopenten-1-yl]-2- pyrazinecarboxylic acid | ¹H NMR (DMSO) δ: 1.96- 2.04 (2H, m), 2.78-2.83 (2H, m), 2.90-2.98 (2H, m), 5.04 (2H, s), 7.12- 7.24 (4H, m), 7.32-7.40 (2H, m), 8.07 (1H, s), 8.79 (1H, s). LC/MS Rt = 4.55, [MH+] 459, 462 [MH−] 457, 460 |
| 53 | 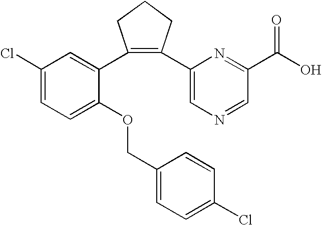 | 6-[2-(5-Chloro-2-{[(4- chlorophenyl)methyl] oxy)phenyl)-1- cyclopenten-1-yl]-2- pyrazinecarboxylic acid | ¹H NMR (DMSO) δ: 1.91- 2.05 (2H, m), 2.86-2.90 (2H, m), 2.98-3.01 (2H, m), 4.99 (2H, s), 7.12- 7.18 (4H, m), 7.33-7.46 (3H, m), 8.06 (1H, s), 8.74 (1H, s). LC/MS Rt = 4.51, [MH+] 441, 444 [MH−] 439, 442 |
| 54 | 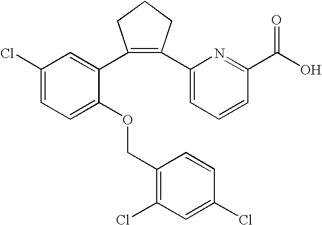 | 6-[2-(5-Chloro-2- {[(2,4-dichlorophenyl) methyl]oxy}phenyl)-1- cyclopenten-1-yl]-2- pyridinecarboxylic acid | LC/MS Rt = 4.01, [MH+] 476, 478 |
| 55 | 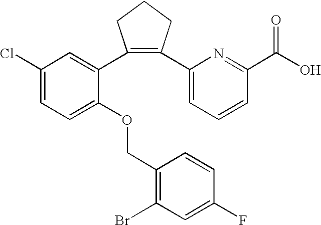 | 6-[2-(2-{[(2-Bromo-4- fluorophenyl)methyl] oxy}-5-chlorophenyl)-1- cyclopenten-1-yl]-2- pyridinecarboxylic acid | LC/MS Rt = 4.01, [MH+] 504, 506 [MH−] 502, 503 |
| 56 | 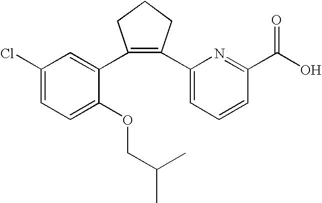 | 6-(2-{5-Chloro-2-[(2- methylpropyl)oxy]phenyl}- 1-cyclopenten-1-yl)-2- pyridinecarboxylic acid | LC/MS Rt = 3.96, [MH+] 372, 374 |
| 57 | 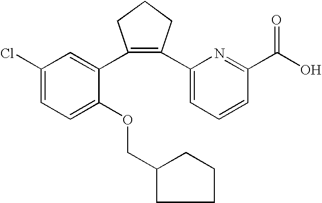 | 6-(2-{5-Chloro-2- [(cyclopentylmethyl)oxy] phenyl}-1-cyclopenten-1- yl)-2-pyridinecarboxylic acid | LC/MS Rt = 4.26, [MH+] 398, 400 [MH−] 396, 398 |
| 58 | 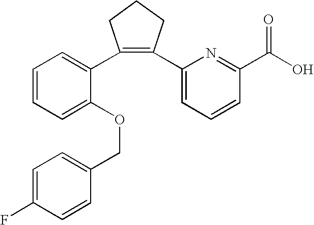 | 6-[2-(2-{[(4- Fluorophenyl)methyl] oxy}phenyl)-1- cyclopenten-1-yl]-2- pyridinecarboxylic acid | ¹H NMR (DMSO) δ: 1.95-2.03 (2H, m), 2.83- 2.87 (2H, m), 2.99-3.33 (2H, m), 5.01 (2H, s), 6.90-6.92 (1H, m), 6.99 (1H, d), 7.04 (1H, dd), 7.09-7.13 (3H, m), 7.22- 7.28 (3H, m), 7.58-7.62 (1H, m), 7.74 (1H, d), 12.55-12.95 (1H, brs). LC/MS: Rt = 3.39 min, [M − H] 388, 390. |
| 59 | 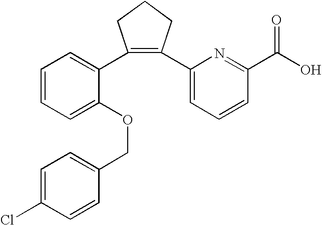 | 6-[2-(2-{[(4- Chlorophenyl)methyl] oxy}phenyl)-1- cyclopenten-1-yl]-2- pyridinecarboxylic acid | ¹H NMR (DMSO) δ: 1.95- 2.03 (2H, m), 2.83-2.87 (2H, m), 3.00-3.34 (2H, m), 5.03 (2H, s), 6.89-6.93 (1H, m), 7.00 (1H, d), 7.05 (1H, dd), 7.09 (1H, d), 7.20-7.22 (2H, m), 7.24-7.30 (1H, m), 7.34- 7.36 (2H, m), 7.55-7.59 (1H, m), 7.72 (1H, d). LC/MS: Rt = 3.68 min, [M + H] 406. |
| 60 | 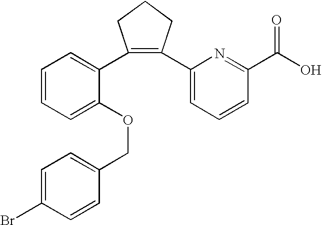 | 6-[2-(2-{[(4- Bromophenyl)methyl] oxy}phenyl)-1- cyclopenten-1-yl]-2- pyridinecarboxylic acid | ¹H NMR (DMSO) δ: 1.96- 2.03 (2H, m), 2.83-2.87 (2H, m), 3.00-3.03 (2H, m), 5.01 (2H, s), 6.89-6.93 (1H, m), 6.96 (1H, d), 7.05 (1H, dd), 7.08 (1H, d), 7.15 (2H, d), 7.24-7.28 (1H, m), 7.48 (2H, d), 7.56-7.60 (1H, m), 7.73 (1H), 12.55-12.95 (1H, brs). LC/MS: Rt = 3.77 min, [M + H] 452. |
| 61 | 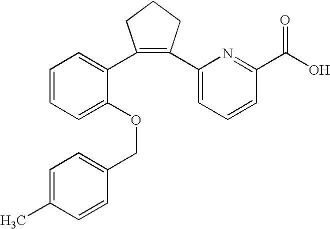 | 6-[2-(2-{[(4- Methylphenyl)methyl] oxy}phenyl)-1- cyclopenten-1-yl]-2- pyridinecarboxylic acid | ¹H NMR (DMSO) δ: 1.94- 2.01 (2H, m), 2.26 (3H, s), 2.83-2.87 (2H, m), 2.98-3.02 (2H, m), 5.00 (2H, s), 6.86- 6.89 (1H, m), 6.92 (1H, d), 7.00 (1H, dd) 7.09-7.11 (5H, m), 7.22-7.26 (1H, m), 7.52- 7.55 (1H, m), 7.69 (1H, d, J = 7.5 Hz). LC/MS: Rt = 3.56 min, [M + H] 386. |
| 62 | 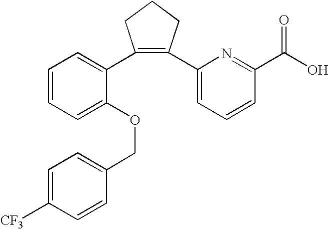 | 6-{2-[2-({[4- (Trifluoromethyl)phenyl] methyl}oxy)phenyl]-1- cyclopenten-1-yl}-2- pyridinecarboxylic acid | ¹H NMR (DMSO) δ: 1.97- 2.05 (2H, m), 2.86-2.89 (2H, m), 3.02-3.05 (2H, m), 5.14 (2H, s), 6.91-6.95 (1H, m), 6.98 (1H, d), 7.07-7.11 (2H, m), 7.25-7.28 (1H, m), 7.41 (2H, d), 7.56-7.60 (1H, m), 7.66 (2H, d), 7.71 (1H, d). LC/MS: Rt = 3.76 min, [M + H] 440. |
| 63 | 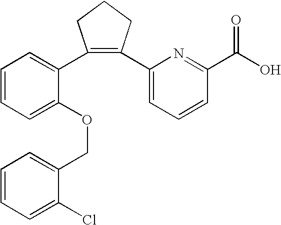 | 6-[2-(2-{[(2- Chlorophenyl)methyl] oxy}phenyl)-1- cyclopenten-1-yl]-2- pyridinecarboxylic acid | ¹H NMR (DMSO) δ: 1.95- 2.02 (2H, m), 2.84-2.88 (2H, m), 3.00-3.03 (2H, m), 5.10 (2H, s), 6.91-6.95 (1H, m), 6.99 (1H, d), 7.04 (1H, dd), 7.14 (1H, d), 7.25-7.32 (4H, m), 7.45 (1H, d), 7.58-7.62 (1H, m), 7.73 (1H, d). LC/MS: Rt = 3.83 min, [M + H] 406. |
| 64 | 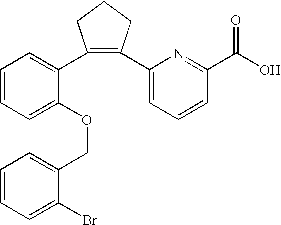 | 6-[2-(2-{[(2- Bromophenyl)methyl] oxy}phenyl)-1- cyclopenten-1-yl]-2- pridinecarboxylic acid | ¹H NMR (CDCl₃) δ: 2.11- 2.18 (2H, m), 2.95-2.88 (2H, m), 3.03-3.08 (2H, m), 5.00 (2H, s), 6.99-7.04 (2H, m), 7.09-7.21 (4H, m), 7.28-7.35 (2H, m), 7.49 (1H, dd), 7.64- 7.68 (1H, m), 7.86 (1H, d). LC/MS: Rt = 3.77 min, [M + H] 450. |
| 65 | 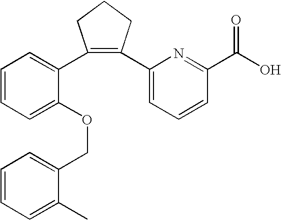 | 6-[2-(2-{[(2- Methylphenyl)methyl] oxy}phenyl)-1- cyclopenten-1-yl]-2- pyridinecarboxylic acid | ¹H NMR (DMSO) δ: 1.93- 2.01 (2H, m), 2.23 (3H, s), 2.81-2.84 (2H, m), 2.99-3.02 (2H, m), 5.05 (2H, s), 6.86- 6.90 (1H, m), 6.96 (1H, dd), 7.00 (1H, dd), 7.10-7.20 (5H, m), 7.56-7.60 (1H, m), 7.73 (1H, dd), 12.43-13.10 (1H, brs). LC/MS: Rt = 3.64 min, [M + H] 386. |
| 66 | 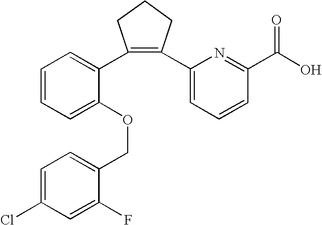 | 6-[2-(2-{[(4-Chloro-2- fluorophenyl)methyl] oxy}phenyl)-1- cyclopenten-1-yl]-2- pyridinecarboxylic acid | ¹H NMR (CDCl₃) δ: 2.08- 2.16 (2H, m), 2.90-2.94 (2H, m), 3.00-3.04 (2H, m), 4.98 (2H, s), 6.99-7.03 (3H, m), 7.08-7.13 (2H, m), 7.28-7.31 (3H, m), 7.66-7.70 (1H, m), 7.87 (1H, d, J = 7.6 Hz). LC/MS: Rt = 3.75 min, [M + H] 424. |
| 67 | 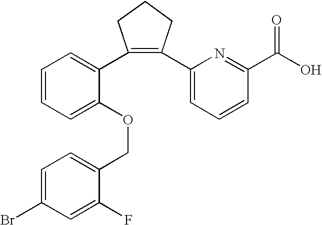 | 6-[2-(2-{[(4-Bromo-2- fluorophenyl)methyl] oxy}phenyl)-1- cyclopenten-1-yl]-2- pyridinecarboxylic acid | ¹H NMR (DMSO) δ: 1.93- 2.01 (2H, m), 2.80-2.84 (2H, m), 2.98-3.01 (2H, m), 5.05 (2H, s), 6.91-6.94 (2H, m), 7.03 (1H, dd), 7.18-7.23 (2H, m), 7.30-7.32 (1H, m), 7.38 (1H, dd), 7.53 (1H, dd), 7.56- 7.60 (1H, m), 7.72 (1H, d, J = 7.2 Hz), 12.56-13.05 (1H, br s). LC/MS: Rt = 3.96 min, [M + H] 470. |
| 68 | 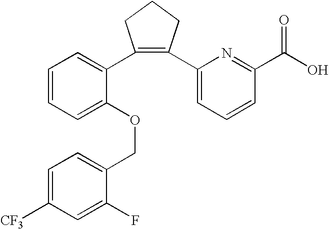 | 6-{2-[2-({[2-Fluoro-4- (trifluoromethyl)phenyl] methyl}oxy)phenyl]-1- cyclopenten-1-yl}-2- pyridinecarboxylic acid | ¹H NMR (DMSO) δ: 1.96- 2.03 (2H, m), 2.83-2.87 (2H, m), 3.00-3.04 (2H, m), 5.16 (2H, s), 6.93-6.97 (2H, m), 7.07 (1H, d), 7.18 (1H, d), 7.28-7.32 (1H, m), 7.44-7.48 (1H, m), 7.52-7.59 (2H, m), 7.65 (1H, d), 7.70 (1H, d). LC/MS: Rt = 3.98 min, [M + H] 458. |
| 69 | 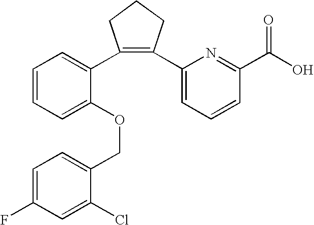 | 6-[2-(2-{[(2-Chloro-4- fluorophenyl)methyl] oxy}phenyl)-1- cyclopenten-1-yl]-2- pyridinecarboxylic acid | ¹H NMR (CDCl₃) δ: 2.10- 2.27 (2H, m), 2.92-2.96 (2H, m), 3.02-3.06 (2H, m), 5.00 (2H, s), 6.85-6.90 (1H, m), 6.99-7.07 (3H, m), 7.13 (1H, dd), 7.19-7.21 (1H, m), 7.28- 7.34 (2H, m), 7.66-7.70 (1H, m), 7.87 (1H, d). LC/MS: Rt = 3.76 min, [M + H] 424. |
| 70 | 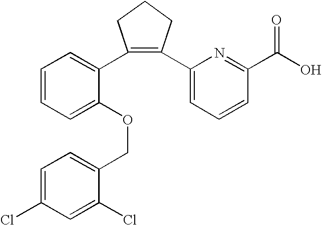 | 6-[2-(2-{[(2,4- Dichlorophenyl)methyl] oxy}phenyl)-1- cyclopenten-1-yl]-2- pyridinecarboxylic acid | ¹H NMR (CDCl₃) δ: 2.10- 2.18 (2H, m), 2.93-2.96 (2H, m), 3.02-3.06 (2H, m), 5.00 (2H, s), 6.98-7.04 (2H, m), 7.11-7.17 (3H, m), 7.28-7.34 (3H, m), 7.67-7.71 (1H, m), 7.87 (1H, d). LC/MS: Rt = 4.08 min, [M + H] 440. |
| 71 | 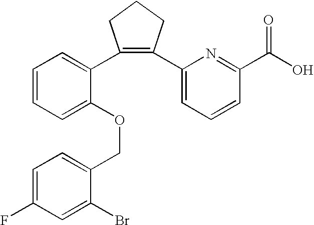 | 6-[2-(2-{[(2-Bromo-4- fluorophenyl)methyl] oxy}phenyl)-1- cyclopenten-1-yl]-2- pyridinecarboxylic acid | ¹H NMR (DMSO) δ: 1.94- 2.02 (2H, m), 2.83-2.87 (2H, m), 2.99-3.03 (2H, m), 5.02 (2H, s), 6.94-6.99 (2H, m), 7.05 (1H, dd), 7.14 (1H, d), 7.19-7.23 (1H, m), 7.30-7.37 (2H, m), 7.57-7.61 (2H, m), 7.72 (1H, d), 12.56-12.94 (1H, br s). LC/MS: Rt = 3.81 min, [M + H] 468. |
| 72 | 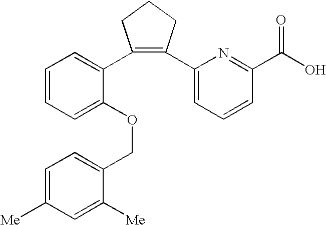 | 6-[2-(2-{[(2,4- Dimethylphenyl)methyl] oxy}phenyl)-1- cyclopenten-1-yl]-2- pyridinecarboxylic acid | ¹H NMR (DMSO) δ: 1.92- 2.00 (2H, m), 2.18 (3H, s), 2.22 (3H, s), 2.79-2.83 (2H, m), 2.97-3.01 (2H, m), 5.00 (2H, s), 6.87-6.94 (4H, m), 6.98 (1H, dd), 7.06 (1H, d), 7.19 (1H, d), 7.28-7.30 (1H, m), 7.55-7.59 (1H, m), 7.72 (1H, dd), 12.52-12.87 (1H, br s). LC/MS: Rt = 3.70 min, [M − H] 398, 400. |
| 73 | 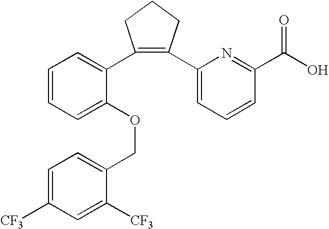 | 6-{2-[2-({[2,4- Bis(trifluoromethyl) phenyl]methyl}oxy)phenyl]- 1-cyclopenten-1-yl}-2- pyridinecarboxylic acid | ¹H NMR (DMSO) δ: 1.97- 2.05 (2H, m), 2.85-2.89 (2H, m), 3.02-3.06 (2H, m), 5.20 (2H, s), 6.97-7.02 (2H, m), 7.08 (1H, d), 7.14 (1H, dd), 7.27-7.31 (1H, m), 7.58-7.62 (1H, m), 7.66-7.71 (2H, m), 8.02 (1H, d), 12.61-13.05 (1H, br s). LC/MS: Rt = 4.10 min [M + H] 508. |
| 74 | 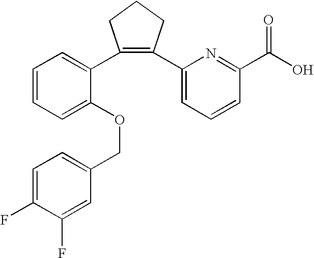 | 6-[2-(2-{[(3,4- Difluorophenyl)methyl] oxy}phenyl)-1- cyclopenten-1-yl]-2- pyridinecarboxylic acid | ¹H NMR (DMSO) δ: 1.96- 2.04 (2H, m), 2.84-2.88 (2H, m), 3.01-3.04 (2H, m), 5.00 (2H, s), 6.92-6.95 (1H, m), 6.99 (1H, d), 7.07-7.11 (3H, m), 7.15-7.20 (1H, m), 7.26- 7.40 (2H, m), 7.58-7.62 (1H, m), 7.73 (1H, d), 12.41-12.98 (1H, br s). LC/MS: Rt = 3.52 min, [M − H] 408, 408. |
| 75 | 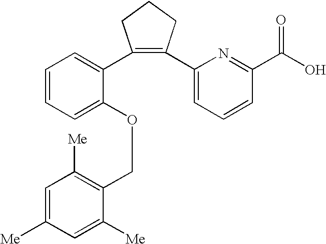 | 6-[2-(2-{[(2,4,6- Trimethylphenyl)methyl] oxy}phenyl)-1- cyclopenten-1-yl]-2- pyridinecarboxylic acid | ¹H NMR (DMSO) δ: 1.84- 1.92 (2H, m), 2.18 (9H, s), 2.70-2.74 (2H, m), 2.90-2.94 (2H, m), 4.98 (2H, s), 6.81 (2H, s), 6.90-6.96 (3H, m), 7.31-7.33 (2H, m), 7.56-7.60 (1H, m), 7.71 (1H, dd). LC/MS: Rt = 3.76 min, [M − H] 412, 414. |
| 76 | 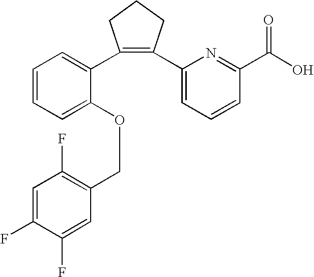 | 6-[2-(2-{[(2,4,5- Trifluorophenyl)methyl] oxy}phenyl)-1- cyclopenten-1-yl]-2- pyridinecarboxylic acid | ¹H NMR (DMSO) δ: 1.94- 2.02 (2H, m), 2.81-2.85 (2H, m), 2.98-3.02 (2H, m), 5.02 (2H, s), 6.95-6.97 (2H, m), 7.07 (1H, dd), 7.18 (1H, d), 7.24-7.33 (2H, m), 7.50-7.61 (2H, m), 7.72 (1H, dd), 12.57-12.87 (1H, br s). LC/MS: Rt = 3.58 min, [M + H] 426. |
| 77 | 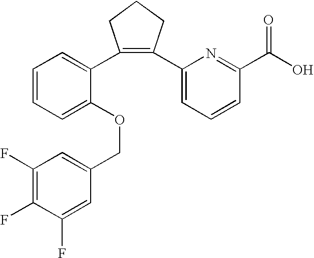 | 6-[2-(2-{[(3,4,5- Trifluorophenyl)methyl] oxy}phenyl)-1- cyclopenten-1-yl]-2- pyridinecarboxylic acid | ¹H NMR (DMSO) δ: 1.98- 2.05 (2H, m), 2.86-2.89 (2H, m), 3.02-3.06 (2H, m), 4.98 (2H, s), 6.97-7.00 (2H, m), 7.03-7.09 (3H, m), 7.13 (1H, dd), 7.27-7.29 (1H, m), 7.58- 7.61 (1H, m), 7.73 (1H, d). LC/MS: Rt = 3.68 min, [M − H] 424, 426. |
| 78 | 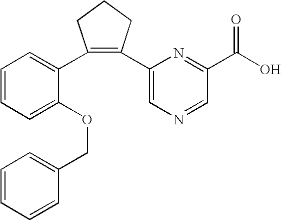 | 6-(2-{2- [(Phenylmethyl)oxy] phenyl}-1-cyclopenten-1- yl)-2-pyrazinecarboxylic acid | ¹H NMR (DMSO) δ: 1.99- 2.06 (2H, m), 2.90-2.94 (2H, m), 2.99-3.02 (2H, m), 5.03 (2H, s), 6.92-6.96 (1H, m), 7.11 (1H, dd), 7.14-7.19 (3H, m), 7.25-7.34 (4H, m), 8.11 (1H, s), 8.80 (1H, s), 13.28- 13.89 (1H, br s). LC/MS: Rt = 4.18 min, [M + H] 373. |
| 79 | 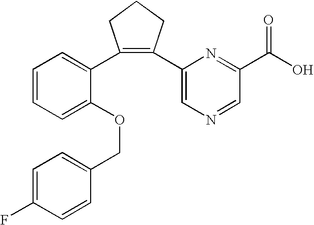 | 6-[2-(2-{[(4- Fluorophenyl)methyl] oxy}phenyl)-1- cyclopenten-1-yl]-2- pyrazinecarboxylic acid | ¹H NMR (DMSO) δ: 1.98- 2.06 (2H, m), 2.89-2.93 (2H, m), 2.98-3.02 (2H, m), 4.99 (2H, s), 6.93-6.97 (1H, m), 7.08-7.17 (4H, m), 7.20-7.24 (2H, m), 7.30-7.34 (1H, m), 8.09 (1H, s), 8.79 (1H, s), 13.20-13.95 (1H, br s). LC/MS: Rt = 4.16 min, [M − H] 389, 391. |
| 80 | 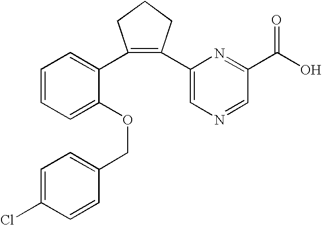 | 6-[2-(2-{[(4- Chlorophenyl)methyl] oxy}phenyl)-1- cyclopenten-1-yl]-2- pyrazinecarboxylic acid | ¹H NMR (DMSO) δ: 1.99- 2.06 (2H, m), 2.89-2.93 (2H, m), 2.93-3.02 (2H, m), 5.01 (2H, s), 6.94-6.97 (1H, m), 7.11-7.15 (2H, m), 7.19 (2H, d), 7.30-7.35 (3H, m), 8.09 (1H, s), 8.80 (1H, s), 13.21- 13.89 (1H, br s). LC/MS: Rt = 4.50 min, [M − H] 405, 407. |
| 81 | 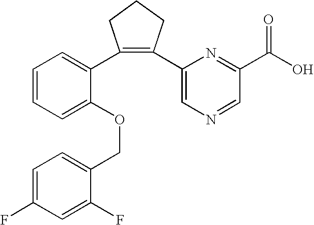 | 6-[2-(2-{[(2,4- Difluorophenyl)methyl]- oxy}phenyl)-1- cyclopenten-1-yl]-2- pyrazinecarboxylic acid | ¹H NMR (DMSO) δ: 1.96- 2.03 (2H, m), 2.85-2.89 (2H, m), 2.95-2.99 (2H, m), 5.02 (2H, s), 6.94-7.01 (2H, m), 7.11 (1H, dd), 7.16-77.23 (2H, m), 7.29-7.38 (2H, m), 8.05 (1H, s), 8.78 (1H, s), 13.19-13.78 (1H, br s). LC/MS: Rt = 4.20 min, [M − H] 407, 409. |
| 82 | 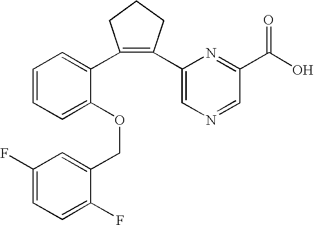 | 6-[2-(2-{[(2,5- Difluorophenyl)methyl] oxy}phenyl)-1- cyclopenten-1-yl]-2- pyrazinecarboxylic acid | ¹H NMR (DMSO) δ: 1.98- 2.05 (2H, m), 2.87-2.91 (2H, m), 2.98-3.01 (2H, m), 5.06 (2H, s), 6.97-7.02 (2H, m), 7.13 (1H, dd), 7.16-7.23 (3H, m), 7.33-7.37 (1H, m), 8.08 (1H, s), 8.78 (1H, s), 13.30- 13.78 (1H, br s). LC/MS: Rt = 4.22 min, [M + H] 409. |
| 83 | 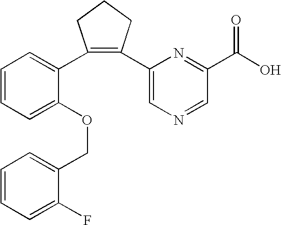 | 6-[2-(2-{[(2- Fluorophenyl)methyl] oxy}phenyl)-1- cyclopenten-1-yl]-2- pyrazinecarboxylic acid | ¹H NMR (DMSO) δ: 1.96- 2.04 (2H, m), 2.86-2.90 (2H, m), 2.96-3.00 (2H, m), 5.01 (2H, s), 6.94-6.98 (1H, m), 7.08-7.28 (5H, m), 7.32-7.34 (2H, m), 8.08 (1H, s), 8.79 (1H, s), 13.20-13.88 (1H, br s). LC/MS: Rt = 4.14 min, [M − H] 389, 391. |
| 84 | 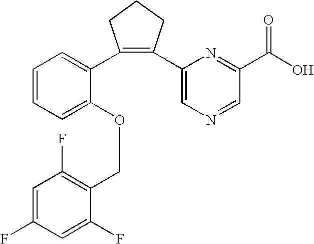 | 6-[2-(2-{[(2,4,6- Trifluorophenyl)methyl] oxy}phenyl)-1- cyclopenten-1-yl]-2- pyrazinecarboxylic acid | ¹H NMR (DMSO) δ: 1.91- 1.99 (2H, m), 2.79-2.83 (2H, m), 2.91-2.94 (2H, m), 5.01 (2H, s), 6.97-7.00 (1H, m), 7.09-7.15 (3H, m), 7.28 (1H, d), 7.34-7.40 (1H, m), 7.99 (1H, s), 8.78 (1H, s), 13.31- 13.79 (1H, br s). |
| 85 | 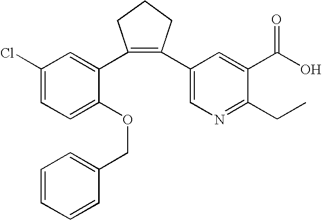 | 5-(2-{5-Chloro-2- [(phenylmethyl)oxy] phenyl)-1-cyclopenten-1- yl)-2-ethyl-3- pyridinecarboxylic acid | ¹H NMR (CDCl₃) δ: 1.27 (3H, t), 2.05-2.13 (2H, m), 2.86- 2.95 (4H, m), 3.15 (2H, q), 4.94 (2H, s), 6.84 (1H, d), 7.05 (1H, d), 7.14-7.19 (2H, m), 7.27-7.32 (4H, m), 8.00 (1H, d), 8.41 (1H, d). LC/MS: Rt = 3.95 min, [M − H] 432, 434. |
| 86 | 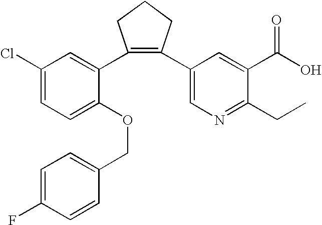 | 5-[2-(5-Chloro-2-{[(4- fluorophenyl)methyl] oxy}phenyl)-1- cyclopenten-1-yl]-2- ethyl-3- pyridinecarboxylic acid | ¹H NMR (CDCl₃) δ: 1.27 (3H, t), 2.05-2.12 (2H, m), 2.84-2.88 (2H, m), 2.90-2.94 (2H, m), 3.15 (2H, q), 4.87 (2H, s), 6.83 (1H, d), 6.96- 7.00 (2H, m), 7.07 (1H, d), 7.11-7.17 (3H, m), 7.99 (1H, d), 8.39 (1H, d). LC/MS: Rt = 3.99 min, [M − H] 450, 452. |
| 87 | 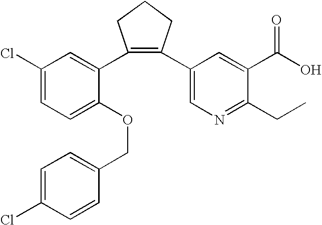 | 5-[2-(5-Chloro-2-{[(4- chlorophenyl)methyl] oxy}phenyl)-1- cyclopenten-1-yl]-2- ethyl-3- pyridinecarboxylic acid | ¹H NMR (CDCl₃) δ: 1.27 (3H, t), 2.06-2.13 (2H, m), 2.84-2.88 (2H, m), 2.91-2.95 (2H, m), 3.15 (2H, q), 4.87 (2H, s), 6.81 (1H, d), 7.08- 7.10 (2H, m), 7.15 (1H, dd), 7.26-7.28 (3H, m), 7.99 (1H, d), 8.39 (1H, d). LC/MS: Rt = 4.24 min, [M − H] 466, 468. |
| 88 | 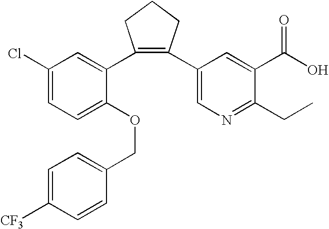 | 5-{2-[5-Chloro-2-({[4- (trifluoromethyl)phenyl] methyl}oxy)phenyl]-1- cyclopenten-1-yl}-2- ethyl-3- pyridinecarboxylic acid | ¹H NMR (CDCl₃) δ: 1.25 (3H, t), 2.07-2.14 (2H, m), 2.86-2.89 (2H, m), 2.92-2.95 (2H, m), 3.12 (2H, q), 4.96 (2H, s), 6.81 (1H, d), 7.10 (1H, d), 7.16 (1H, dd), 7.26- 7.29 (2H, m), 7.55-7.57 (2H, m), 7.97 (1H, d), 8.39 (1H, d). LC/MS: Rt = 4.28 min, [M − H] 500, 502. |
| 89 | 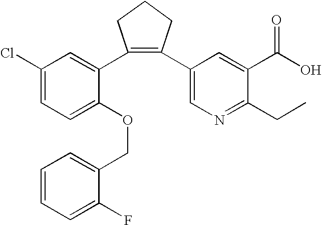 | 5-[2-(5-Chloro-2-{[(2- fluorophenyl)methyl] oxy}phenyl)-1- cyclopenten-1-yl]-2- ethyl-3- pyridinecarboxylic acid | ¹H NMR (CDCl₃) δ: 1.13 (3H, t), 1.90-1.97 (2H, m), 2.70-2.80 (2H, m), 2.97-3.02 (2H, m) 4.97 (2H, s), 6.81 (1H, d), 6.93 (1H, d), 6.96- 7.03 (2H, m), 7.08 (1H, dd), 7.16-7.21 (2H, m), 7.24 (1H, d), 8.23 (1H, d). LC/MS: Rt = 3.98 min, [M − H] 450, 452. |
| 90 | 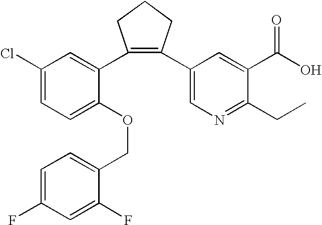 | 5-[2-(5-Chloro-2-{[(2,4- difluorophenyl)methyl] oxy}phenyl)-1- cyclopenten-1-yl]-2- ethyl-3- pyridinecarboxylic acid | ¹H NMR (CDCl₃) δ: 1.26 (3H, t), 2.05-2.12 (2H, m), 2.82-2.86 (2H, m), 2.90-2.94 (2H, m), 3.14 (2H, q), 4.93 (2H, s), 6.75-6.82 (2H, m), 6.87 (1H, d), 7.07 (1H, d), 7.11-7.15 (1H, m), 7.18 (1H, dd), 7.97 (1H, d), 8.38 (1H, d). LC/MS: Rt = 4.01 min, [M − H] 468, 470. |
| 91 | 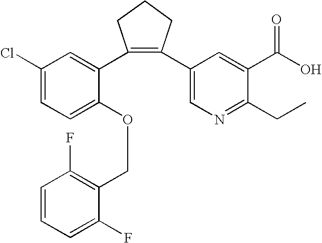 | 5-[2-(5-Chloro-2-{[(2,6- difluorophenyl)methyl] oxy}phenyl)-1- cyclopenten-1-yl-2- ethyl-3- pyridinecarboxylic acid | ¹H NMR (CDCl₃) δ: 1.28 (3H, t), 1.99-2.06 (2H, m), 2.76-2.80 (2H, m), 2.84-2.88 (2H, m), 3.16 (2H, q), 5.02 (2H, s), 6.83-6.87 (2H, m), 6.99-7.01 (2H, m), 7.20 (1H, dd), 7.24-7.28 (1H, m), 7.95 (1H, d), 8.34 (1H, d). LC/MS: Rt = 3.90 min, [M − H] 468, 470. |
| 92 | 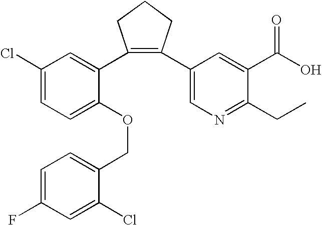 | 5-[2-(5-Chloro-2-{[(2- chloro-4- fluorophenyl)methyl] oxy}phenyl)-1- cyclopenten-1-yl]-2- ethyl-3- pyridinecarboxylic acid | ¹H NMR (CDCl₃) δ: 1.25 (3H, t), 2.07-2.14 (2H, m), 2.85-2.89 (2H, m), 2.93-2.96 (2H, m), 3.14 (2H, q), 4.95 (2H, s), 6.85 (1H, d), 6.90- 6.94 (1H, m), 7.07-7.10 (2H, m), 7.17-7.21 (2H, m), 7.99 (1H, d), 8.40 (1H, d). LC/MS: Rt = 4.30 min, [M − H] 484, 486. |
| 93 | 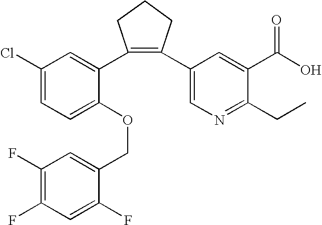 | 5-[2-(5-Chloro-2- {[(2,4,5- trifluorophenyl)methyl] oxy}phenyl)-1- cyclopenten-1-yl]-2- ethyl-3- pyridinecarboxylic acid | ¹H NMR (CDCl₃) δ: 1.25 (3H, t), 2.08-2.16 (2H, m), 2.84-2.88 (2H, m), 2.94-2.98 (2H, m), 3.14 (2H, q), 4.90 (2H, s), 6.84-6.92 (2H, m), 6.99-7.05 (1H, m), 7.11 (1H, d), 7.19 (1H, dd,), 7.99 (1H, d), 8.39 (1H, d). LC/MS: Rt = 4.13 min, [M − H] 486, 488. |
| 94 | 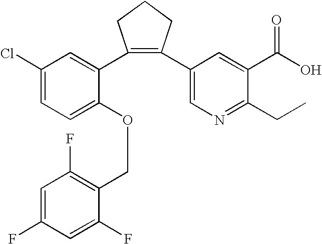 | 5-[2-(5-chloro-2- {[(2,4,6- trifluorophenyl)methyl] oxy}phenyl)-1- cyclopenten-1-yl]-2- ethyl-3- pyridinecarboxylic acid | ¹H NMR (CDCl₃) δ: 1.28 (3H, t), 2.00-2.07 (2H, m), 2.76-2.80 (2H, m), 2.85-2.89 (2H, m), 3.16 (2H, q), 4.95 (2H, s), 6.60-6.64 (2H, m), 6.97 (1H, d), 7.03 (1H, d), 7.20 (1H, dd), 7.95 (1H, d), 8.35 (1H, d). LC/MS: Rt = 3.98 min, [M − H] 486, 488. |
| 95 | 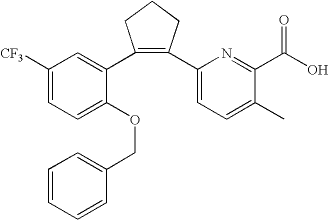 | 3-Methyl-6-{2-[2- [(phenylmethyl)oxy]-5- (trifluoromethyl)phenyl]- 1-cyclopenten-1-yl}-2- pyridinecarboxylic acid | ¹H NMR (CDCl₃) δ: 2.10- 2.17 (2H, m), 2.65 (3H, s), 2.91-2.95 (2H, m), 3.00-3.05 (2H, m), 5.00 (2H, s), 7.03 (1H, d), 7.11-7.16 (3H, m), 2.27-2.28 (3H, m), 7.38 (1H, d), 7.44 (1H, d), 7.53 (1H, dd), 1075-11.23 (1H, br s). LC/MS: Rt = 4.10 min, [M − H] 452, 454. |
| 96 | 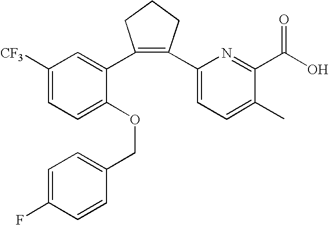 | 6-{2-[2-{[(4- Fluorophenyl)methyl] oxy}-5- (trifluoromethyl)phenyl]- 1-cyclopenten-1-yl}-3- methyl-2- pyridinecarboxylic acid | ¹H NMR (CDCl₃) δ: 2.09- 2.16 (2H, m), 2.66 (3H, s), 2.89-2.93 (2H, m), 2.99-3.03 (2H, m), 4.97 (2H, s), 6.94- 6.98 (2H, m), 7.03 (1H, d), 7.09-7.13 (2H, m), 7.15 (1H, d), 7.37 (1H, d), 7.46 (1H, d), 7.54 (1H, dd), 10.42-11.20 (1H, br s). LC/MS: Rt = 4.06 min, [M − H] 470, 472. |
| 97 | 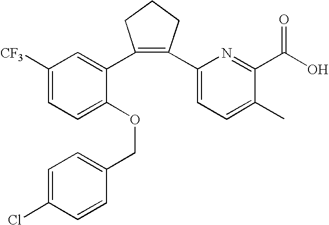 | 6-{2-[2-{[(4- Chlorophenyl)methyl] oxy}-5- (trifluoromethyl)phenyl]- 1-cyclopenten-1-yl}-3- methyl-2- pyridinecarboxylic acid | ¹H NMR (CDCl₃) δ: 2.10- 2.17 (2H, m), 2.66 (3H, s), 2.90-2.93 (2H, m), 3.00-3.04 (2H, m), 4.97 (2H, s), 7.02 (1H, d), 7.07 (2H, d), 7.16 (1H, d), 7.23-7.25 (2H, m), 7.37 (1H, d), 7.46 (1H, d), 7.54 (1H, dd), 10.50-10.98 (1H, br s). LC/MS: Rt = 4.22 min, [M − H] 486, 488. |
| 98 | 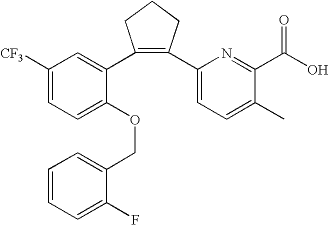 | 6-{2-[2-{[(2- Fluorophenyl)methyl] oxy}-5- (trifluoromethyl)phenyl]- 1-cyclopenten-1-yl}-3- methyl-2- pyridinecarboxylic acid | ¹H NMR (CDCl₃) δ: 2.09- 2.16 (2H, m), 2.65 (3H, s), 2.90-2.94 (2H, m), 3.00-3.04 (2H, m), 5.05 (2H, s), 6.97- 7.04 (2H, m), 7.07-7.09 (2H, m), 7.13 (1H, d), 7.25-7.28 (1H, m), 7.39 (1H, d), 7.43 (1H, d), 7.56 (1H, dd), 10.75- 11.09 (1H, br, s). LC/MS: Rt = 4.07 min, [M − H] 470, 472. |
| 99 | 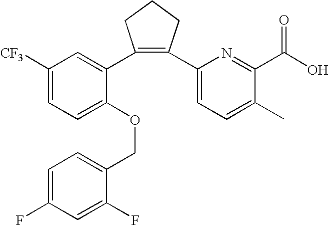 | 6-{2-[2-{[(2,4- Difluorophenyl)methyl] oxy}-5- (trifluoromethyl)phenyl]- 1-cyclopenten-1-yl)-3- methyl-2- pyridinecarboxylic acid | ¹H NMR (CDCl₃) δ: 2.08- 2.16 (2H, m), 2.66 (3H, s), 2.88-2.91 (2H, m), 2.98-3.03 (2H, m), 5.01 (2H, s), 6.74- 6.79 (2H, m), 7.07-7.11 (2H, m), 7.14 (1H, d), 7.38 (1H, d), 7.46 (1H, d), 7.57 (1H, dd), 10.59-11.05 (1H, br s). LC/MS: Rt = 4.10 min, [M − H] 488, 490. |
| 100 | 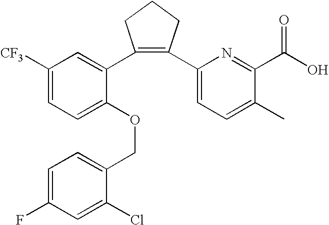 | 6-{2-[2-{[(2-Chloro-4- fluorophenyl)methyl] oxy}-5- (trifluoromethyl)phenyl]- 1-cyclopenten-1-yl}-3- methyl-2- pyridinecarboxylic acid | ¹H NMR (CDCl₃) δ: 2.10- 2.18 (2H, m), 2.65 (3H, s), 2.90-2.94 (2H, m), 3.01-3.05 (2H, m), 5.04 (2H, s), 6.87- 6.91 (1H, m), 7.05-7.09 (2H, m), 7.14-7.18 (2H, m), 7.39 (1H, d), 7.46 (1H, d), 7.57 (1H, dd), 10.56-11.02 (1H, br s). LC/MS: Rt = 4.27 min, [M − H] 504, 506. |
| 101 | 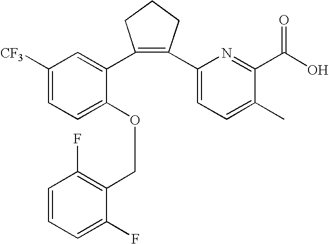 | 6-{2-[2-{[(2,6- Difluorophenyl)methyl] oxy}-5- (trifluoromethyl)phenyl]- 1-cyclopenten-1-yl}-3- methyl-2- pyridinecarboxylic acid | ¹H NMR (CDCl₃) δ: 2.03- 2.10 (2H, m), 2.65 (3H, s), 2.83-2.87 (2H, m), 2.93-2.97 (2H, m), 5.05 (2H, s), 6.79- 6.83 (2H, m), 7.08 (1H, d), 7.17 (1H, d), 7.24-7.28 (1H, m), 7.37 (1H, d), 7.41 (1H, d), 7.58 (1H, dd). LC/MS: Rt = 4.03 min, [M − H] 488, 490. |
| 102 | 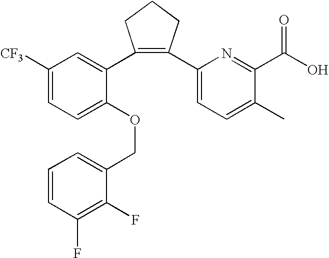 | 6-{2-[2-{[(2,3- Difluorophenyl)methyl] oxy}-5- (trifluoromethyl)phenyl]- 1-cyclopenten-1-yl)-3- methyl-2- pyridinecarboxylic acid | ¹H NMR (CDCl₃) δ: 2.09- 2.17 (2H, m), 2.65 (3H, s), 2.89-2.93 (2H, m), 3.00-3.04 (2H, m), 5.07 (2H, s), 6.88- 6.89 (1H, m), 6.95-7.0 (1H, m), 7.06-7.10 (2H, m), 7.14 (1H, d), 7.40 (1H, d), 7.44 (1H, d), 7.57 (1H, dd), 10.61- 10.99 (1H, br s). LC/MS: Rt = 4.10 min, [M − H] 488, 490. |
| 103 | 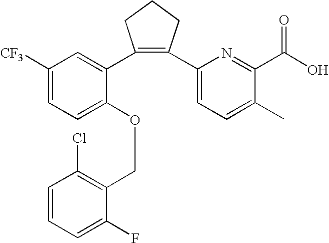 | 6-{2-[2-{[(2-Chloro-6- fluorophenyl)methyl] oxy}-5- (trifluoromethyl)phenyl]- 1-cyclopenten-1-yl}-3- methyl-2- pyridinecarboxylic acid | ¹H NMR (CDCl₃) δ: 2.01- 2.09 (2H, m), 2.65 (3H, s), 2.83-2.87 (2H, m), 2.92-2.96 (2H, m), 5.11 (1H, d), 6.91- 6.97 (1H, m), 7.07 (1H, d), 7.11 (1H, d), 7.19-7.24 (2H, m), 7.37-7.41 (2H, m), 7.59 (1H, dd). LC/MS: Rt = 4.14 min, [M − H] 504, 506. |
| 104 | 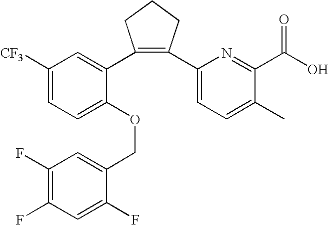 | 3-Methyl-6-[2-(5- (trifluoromethyl]-2- {[(2,4,5- trifluorophenyl)methyl] oxy}phenyl)-1- cyclopenten-1-yl]-2- pyridinecarboxylic acid | ¹H NMR (CDCl₃) δ: 2.12- 2.20 (2H, m), 2.65 (3H, s), 2.89-2.93 (2H, m), 3.02-3.06 (2H, m), 4.99 (2H, s), 6.86- 6.93 (2H, m), 7.05 (1H, d), 7.19 (1H, d), 7.41 (1H, d), 7.49 (1H, d), 7.58 (1H, dd), 10.56-10.90 (1H, br s). LC/MS: Rt = 4.15 min, [M − H] 506, 508. |
| 105 | 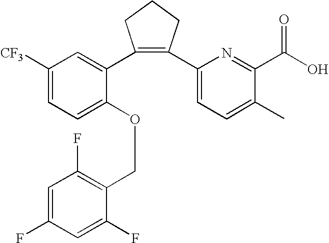 | 3-Methyl-6-[2-(5- (trifluoromethyl)-2- {[(2,4,6- trifluorophenyl)methyl] oxy}phenyl)-1- cyclopenten-1-yl]-2- pyridinecarboxylic acid | ¹H NMR (CDCl₃) δ: 2.03- 2.11 (2H, m), 2.66 (3H, s), 2.82-2.86 (2H, m), 2.93-2.98 (2H, m), 5.01 (2H, s), 6.56- 6.60 (2H, m), 7.10 (1H, d), 7.17 (1H, d), 7.36 (1H, d), 7.44 (1H, d), 7.58 (1H, dd), 10.56-11.00 (1H, br s). LC/MS: Rt = 4.07 min, [M − H] 506, 508. |
| 106 | 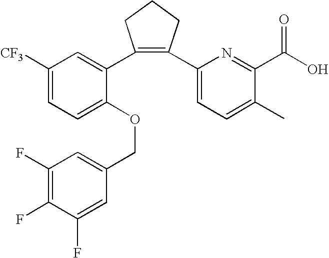 | 3-Methyl-6-[2-(5- (trifluoromethyl)-2- {[(3,4,5- trifluorophenyl)methyl] oxy}phenyl)-1- cyclopenten-1-yl]-2- pyridinecarboxylic acid | ¹H NMR (CDCl₃) δ: 2.13- 2.21 (2H, m), 2.66 (3H, s), 2.90-2.94 (2H, m), 3.03-3.07 (2H, m), 4.93 (2H, s), 6.73- 6.76 (2H, m), 6.97 (1H, d), 7.22 (1H, d), 7.42 (1H, d), 7.51 (1H, d), 7.56 (1H, dd), 10.53-10.80 (1H, br s). LC/MS: Rt = 4.18 min, [M − H] 506, 508. |
| 107 | 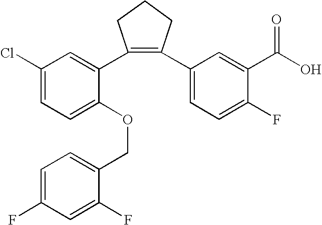 | 5-[2-(5-Chloro-2- {[(2,4- difluorophenyl)methyl] oxy}phenyl)-1- cyclopenten-1-yl]-2- fluorobenzoic acid | LC/MS: Rt = 4.00 min. [M + H] = 476 |
| 108 | 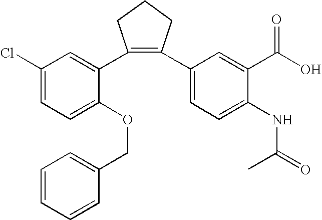 | 2-(Acetylamino)-5-(2- {5-chloro-2- [(phenylmethyl)oxy] phenyl}-1- cyclopenten-1- yl)benzoic acid | LC/MS: Rt = 4.05 min. [M + H]= 462 |
| 109 | 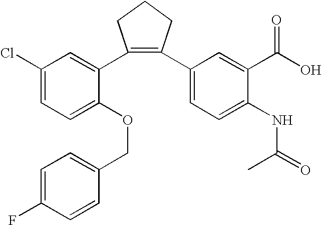 | 2-(Acetylamino)-5-[2- (5-chloro-2-{[(4- fluorophenyl)methyl] oxy}phenyl)-1-cyclopenten- 1-yl]benzoic acid | LC/MS: Rt = 4.04 min. [M + H] = 480 |
| 110 | 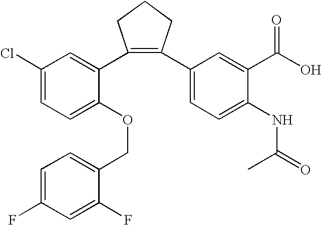 | 2-(Acetylamino)-5-[2- (5-chloro-2-{[(2,4- difluorophenyl)methyl] oxy}phenyl)-1-cyclopenten- 1-yl]benzoic acid | LC/MS: Rt = 4.06 min. [M + H] = 498 |
| 111 | 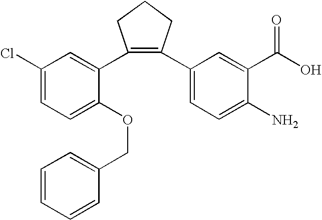 | 2-Amino-5-[2-(5- chloro-2- [(phenylmethyl)oxy] phenyl}-1-cyclopenten- 1-yl)benzoic acid | LC/MS: Rt = 3.87 min. [M + H] = 420 |
| 112 | 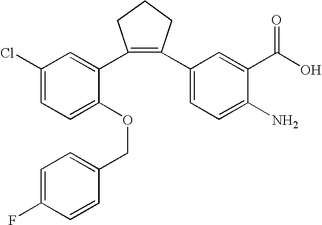 | 2-Amino-5-[2-(5- chloro-2-{[(4- fluorophenyl)methyl] oxy}phenyl)-1-cyclopenten- 1-yl]benzoic acid | LC/MS: Rt = 3.87 min. [M + H]= 438 |
| 113 | 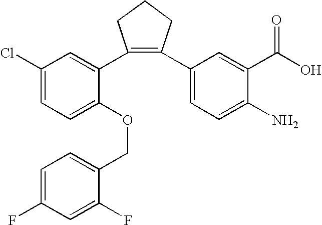 | 2-Amino-5-[2-(5- chloro-2-{[(2,4- difluorophenyl)methyl] oxy}phenyl)-1-cyclopenten- 1-yl]benzoic acid | LC/MS: Rt = 3.91 min. [M + H] = 456 |
| 114 | 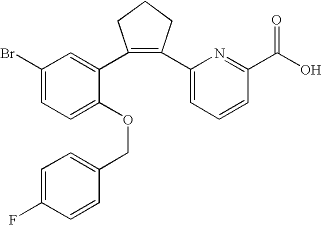 | 6[2-(5-Bromo-2-{[(4- fluorophenyl)methyl] oxy}phenyl)-1-cyclopenten- 1-yl]-2-pyridinecarboxylic acid | LC/MS: Rt = 3.66 min. [M + H] = 468, 470 |
| 115 | 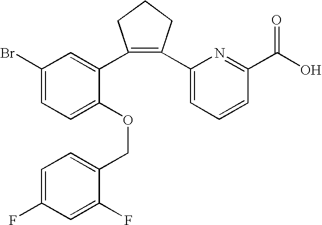 | 6-[2-(5-Bromo-2- {[(2,4- difluorophenyl)methyl] oxy}phenyl)-1-cyclopenten-1- yl]-2-pyridinecarboxylic acid | LC/MS: Rt = 3.69 min. [M + H] = 486, 488 |
| 116 | 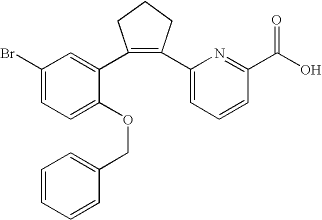 | 6-(2-{5-Bromo-2- [(phenylmethyl)oxy] phenyl}-1-cyclopenten-1- yl)-2-pyridinecarboxylic acid | LC/MS: Rt = 3.65 min. [M + H] = 450, 452 |
| 117 | 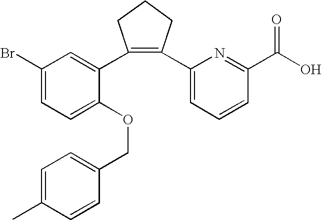 | 6-[2-(5-Bromo-2-{[(4- methylphenyl)methyl] oxy}phenyl)-1- cyclopenten-1-yl]-2- pyridinecarboxylic acid | LC/MS: Rt = 4.10 min. [M + H] = 464, 466 |
| 118 | 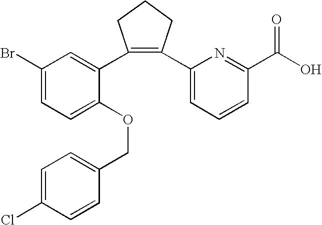 | 6-[2-(5-Bromo-2-{[(4- chlorophenyl)methyl] oxy}phenyl)-1-cyclopenten- 1-yl]-2-pyridinecarboxyic acid | LC/MS: Rt = 4.20 min. [M + H] = 484, 486 |
| 119 | 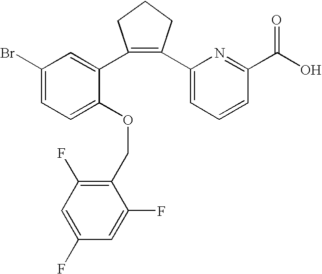 | 6-[2-(5-Bromo-2- {[(2,4,6- trifluorophenyl)methyl] oxy}phenyl)-1-cyclopenten- 1-yl]-2-pyridinecarboxylic acid | LC/MS: Rt = 4.07 min. [M + H] = 504, 506 |
| 120 | 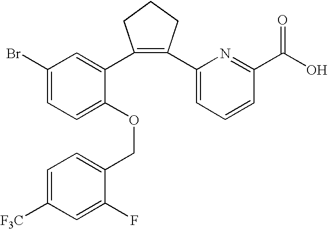 | 6-{2-[5-Bromo-2-({[2- fluoro-4-(trifluoromethyl) phenyl]methyl}oxy)phenyl]- 1-cyclopenten-1-yl}- 2-pyridinecarboxylic acid | LC/MS: Rt = 4.36 min. [M + H] = 536, 538 |
| 121 | 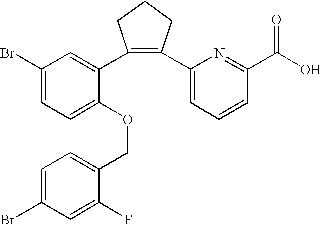 | 6-[2-(5-Bromo-2-{[(4- bromo-2-fluorophenyl) methyl]oxy}phenyl)-1- cyclopenten-1-yl]-2- pyridinecarboxylic acid | LC/MS: Rt = 4.32 min. [M + H] = 546, 548, 550 |
| 122 | 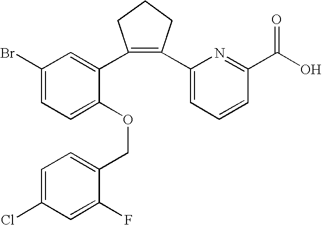 | 6-[2-(5-Bromo-2-{[(4- chloro-2-fluorophenyl) methyl]oxy}phenyl)-1- cyclopenten-1-yl]-2- pyridinecarboxylic acid | LC/MS: Rt = 4.25 min. [M + H] = 502, 504 |
| 123 | 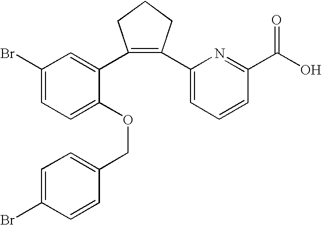 | 6-[2-(5-Bromo-2-{[(4- bromophenyl)methyl] oxy}phenyl)-1-cyclopenten- 1-yl]-2-pyridinecarboxylic acid | LC/MS: Rt = 4.26 min. [M + H] = 528, 530, 532 |
| 124 | 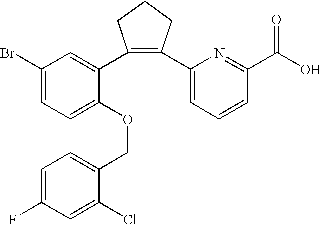 | 6-[2-(5-Bromo-2-{[(2- chloro-4-fluorophenyl) methyl]oxy}phenyl)-1- cyclopenten-1-yl]-2- pyridinecarboxylic acid | LC/MS: Rt = 4.35 min. [M + H] = 502, 504 |
| 125 | 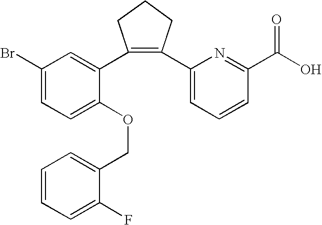 | 6-[2-(5-Bromo-2-{[(2- fluorophenyl)methyl] oxy}phenyl)-1- cyclopenten-1-yl]-2- pyridinecarboxylic acid | LC/MS: Rt = 4.08 min. [M + H] = 468, 470 |
| 126 | 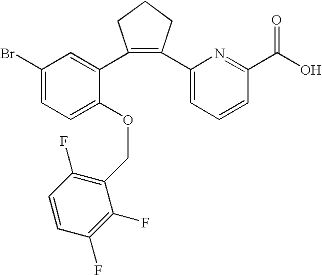 | 6-[2-(5-Bromo-2- {[(2,3,6- trifluorophenyl)methyl] oxy}phenyl)-1- cyclopenten-1-yl]-2- pyridinecarboxylic acid | LC/MS: Rt = 4.05 min. [M + H] = 504, 506 |
| 127 | 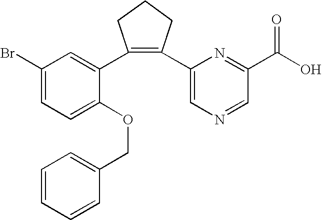 | 6-(2-{5-Bromo-2- [(phenylmethyl)oxy] phenyl}-1-cyclopenten- 1-yl)-2-pyrazinecarboxylic acid | LC/MS: Rt = 4.15 min. [M + H] = 451, 453 |
| 128 | 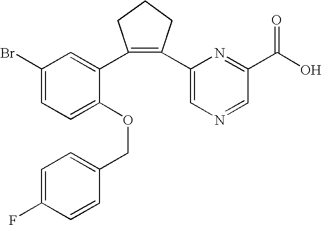 | 6-[2-(5-Bromo-2-{[(4- fluorophenyl)methyl] oxy}phenyl)-1-cyclopenten- 1-yl]-2-pyrazinecarboxylic acid | LC/MS: Rt = 4.54 min. [M + H] = 469, 471 |
| 129 | 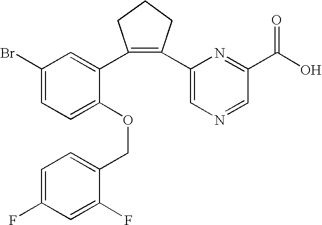 | 6-[2-(5-Bromo-2- {[(2,4-difluorophenyl) methyl]oxy}phenyl)-1- cyclopenten-1-yl]-2- pyrazinecarboxylic acid | LC/MS: Rt = 4.57 min. [M + H] 487, 489 |
| 130 | 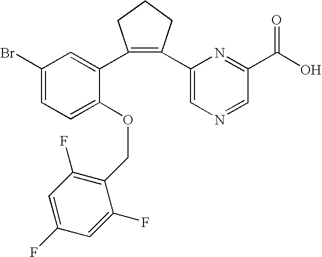 | 6-[2-(5-Bromo-2- {[(2,4,6-trifluorophenyl) methyl]oxy}phenyl)-1- cyclopenten-1-yl]-2- pyrazinecarboxylic acid | LC/MS: Rt = 4.47 min. [M + H] 505, 507 |
| 131 | 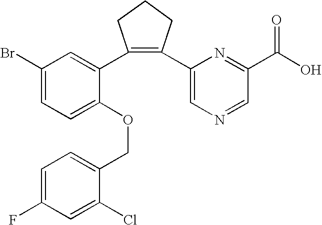 | 6-[2-(5-Bromo-2-{[(2- chloro-4-fluorophenyl) methyl]oxy}phenyl)-1- cyclopenten-1-yl]-2- pyrazinecarboxylic acid | LC/MS: Rt = 4.94 min. [M + H] = 503, 505 |
| 132 | 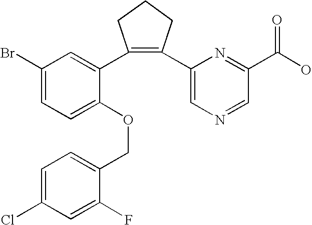 | 6-[2-(5-Bromo-2-{[(4- chloro-2-fluorophenyl) methyl]oxy}phenyl)-1- cyclopenten-1-yl]-2- pyrazinecarboxylic acid | LC/MS: Rt = 4.87 min. [M + H] = 503, 505 |
| 133 | 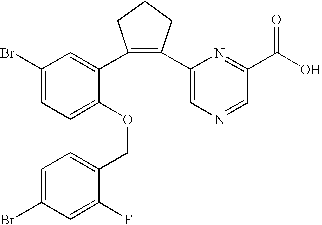 | 6-[2-(5-bromo-2-{[(4- bromo-2-fluorophenyl) methyl]oxy}phenyl)-1- cyclopenten-1-yl]-2- pyrazinecarboxylic acid | LC/MS: Rt = 4.91 min. [M + H] = 547, 549, 551 |
| 134 | 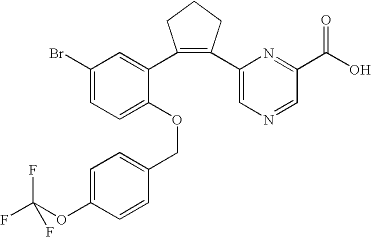 | 6-(2-{5-Bromo-2-[({4- [(trifluoromethyl)oxy] phenyl}methyl)oxy] phenyl}-1-cyclopenten-1- yl)-2-pyrazinecarboxylic acid | LC/MS: Rt = 4.67 min. [M + H] = 535, 537 |
| 135 | 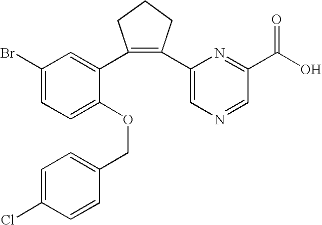 | 6-[2-(5-Bromo-2-{[(4- chlorophenyl)methyl] oxy}phenyl)-1-cyclopenten- 1-yl]-2-pyrazinecarboxylic acid | LC/MS: Rt = 4.77 min. [M + H] = 485, 487 |
| 136 | 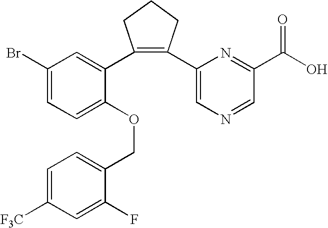 | 6-{2-[5-Bromo-2-({[2- fluoro-4-(trifluoromethyl) phenyl]methyl}oxy)phenyl]- 1-cyclopenten-1-yl}- 2-pyrazinecarboxylic acid | LC/MS: Rt = 4.69 min. [M + H] = 537, 539 |
| 137 | 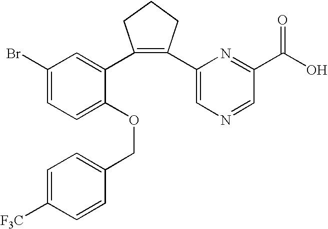 | 6-{2-[5-Bromo-2-({[4- (trifluoromethyl)phenyl] methyl}oxy)phenyl]-1- cyclopenten-1-yl}- 2-pyrazinecarboxylic acid | LC/MS: Rt = 4.64 min. [M + H] = 519, 521 |
| 138 | 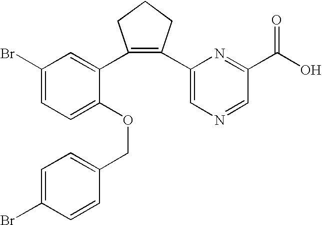 | 6-[2-(5-bromo-2-{[(4- bromophenyl)methyl] oxy}phenyl)-1-cyclopenten- 1-yl]-2-pyrazinecarboxylic acid | LC/MS: Rt = 4.90 min. [M + H] = 529, 531, 533 |
| 139 | 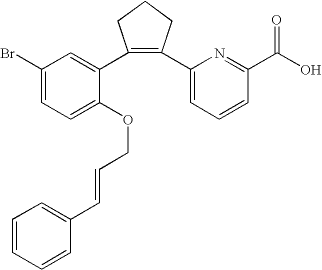 | 6-[2-(5-Bromo-2- {[(2E)-3-phenyl-2-propen- 1-yl]oxy}phenyl)-1- cyclopenten-1-yl]-2- pyridinecarboxylic acid | ¹H NMR (DMSO) δ: 2.00- 2.05 (2H, m), 2.67-3.02 (4H, br m), 4.50 (2H, s), 6.10-6.15 (1H, m), 6.5-6.7 (1H, m), 6.90-7.10 (3H, m), 7.15-7.35 (6H, m), 7.50-7.60 (1H, m), 7.65-7.70 (1H, m). |
| 140 | 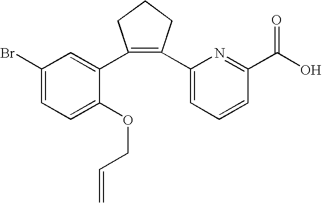 | 6-{2-[5-Bromo-2-(2- propen-1-yloxy)phenyl]-1- cyclopenten-1-yl}-2- pyridinecarboxylic acid | ¹H NMR (DMSO) δ: 1.98- 2.04 (2H, m), 2.70-2.75 (2H, m), 2.95-3.05 (2H, m), 4.40 (2H, m), 5.10-5.21 (2H, m), 5.73-5.76 (1H, m), 6.94-6.97 (1H, m), 7.05-7.15 (2H, m), 7.20-7.25 (1H, m), 7.60- 7.80 (2H, m). |
| 141 | 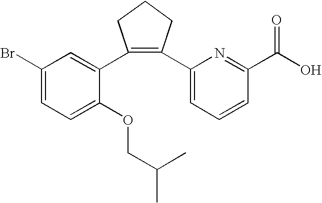 | 6-(2-{5-Bromo-2-[(2- methylpropyl)oxy] phenyl}-1-cyclopenten- 1-yl)-2-pyridinecarboxylic acid | LC/MS: Rt = 3.95 min. [M + H] = 416, 418 |
| 142 | 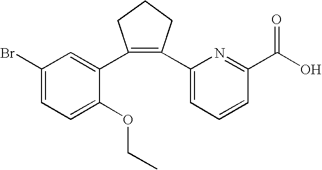 | 6-{2-[5-Bromo-2- (ethyloxy)phenyl]-1- cyclopenten-1-yl}-2- pyridinecarboxylic acid | LC/MS: Rt = 3.44 min. [M + H] = 388, 390 |
| 143 | 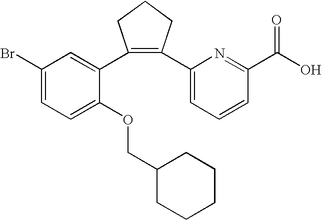 | 6-(2-{5-Bromo-2- [(cyclohexylmethyl) oxy]phenyl}-1-cyclopenten- 1-yl)-2-pyridinecarboxylic acid | LC/MS: Rt = 4.41 min. [M + H] = 456, 458 |
| 144 | 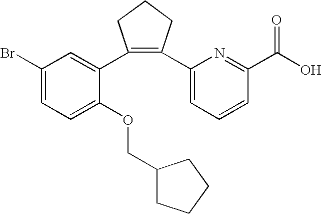 | 6-(2-{5-Bromo-2- [(cyclopentylmethyl) oxy]phenyl}-1-cyclopenten- 1-yl)-2-pyridinecarboxylic acid | LC/MS: Rt = 4.25 min. [M + H] = 442, 444 |
| 145 | 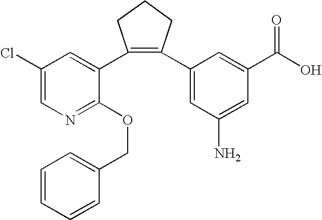 | 3-Amino-5-(2-{5- chloro-2-[(phenylmethyl) oxy]-3-pyridinyl)-1- cyclopenten-1-yl)benzoic acid | LC/MS: Rt = 3.74 min. [M + H] = 421 |
| 146 | 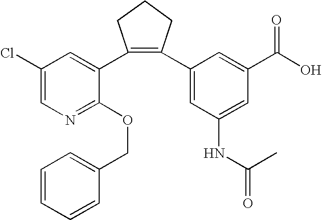 | 3-(Acetylamino)-5-(2- {5-chloro-2-[(phenylmethyl) oxy]-3-pyridinyl}-1- cyclopenten-1-yl)benzoic acid | LC/MS: Rt = 3.74 min. [M + H] = 463 |
| 147 | 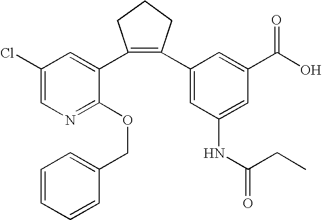 | 3-(2-{5-Chloro-2- [(phenylmethyl)oxy]- 3-pyridinyl}-1-cyclopenten- 1-yl)-5-(propanoylamino) benzoic acid | LC/MS: Rt = 3.90 min. [M + H] = 477 |
| 148 | 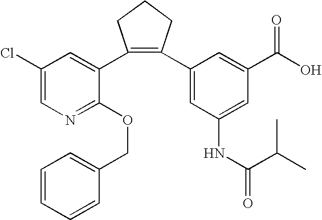 | 3-(2-{5-Chloro-2- [(phenylmethyl)oxy]- 3-pyridinyl}-1-cyclopenten- 1-yl)-5-[(2-methylpropanoyl) amino]benzoic acid | LC/MS: Rt = 4.02 min. [M + H] = 491 |
| 149 | 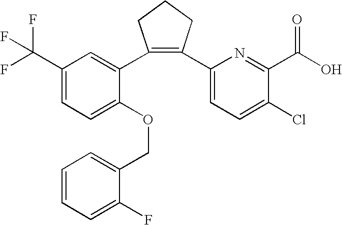 | 3-Chloro-6-{2-[2-{[(2- fluorophenyl)methyl] oxy}-5-(trifluoromethyl) phenyl]-1-cyclopenten-1- yl}-2-pyridinecarboxylic acid | LC/MS: Rt = 4.33 min. [M + H] = 492 |
| 150 | 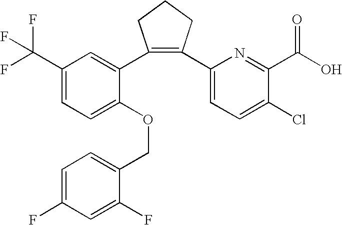 | 3-Chloro-6-{2-[2- {[(2,4-difluorophenyl) methyl]oxy}-5- (trifluoromethyl)phenyl]-1- cyclopenten-1-yl}-2- pyridinecarboxylic acid | LC/MS: Rt = 4.34 min. [M + H] = 510 |
| 151 | 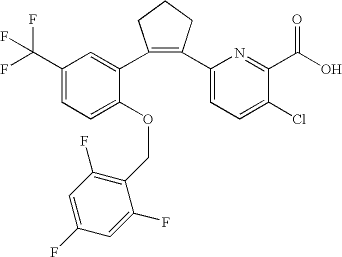 | 3-Chloro-6-[2-(5- (trifluoromethyl)-2- {[(2,4,6-trifluorophenyl) methyl]oxy}phenyl)-1- cyclopenten-1-yl]-2- pyridinecarboxylic acid | LC/MS: Rt = 4.30 min. [M + H] = 528 |
| 152 | 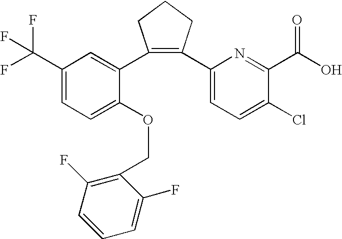 | 3-Chloro-6-{2-[2- {[(2,6-difluorophenyl) methyl]oxy}-5-(trifluoro- methyl)phenyl]-1- cyclopenten-1-yl}-2- pyridinecarboxylic acid | LC/MS: Rt = 4.31 min. [M + H] = 510 |
| 153 | 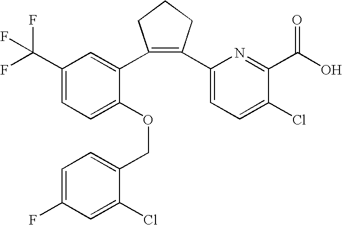 | 3-chloro-6-{2-[2-{[(2- chloro-4-fluorophenyl) methyl]oxy}-5-(trifluoro- methyl)phenyl]-1- cyclopenten-1-yl}-2- pyridinecarboxylic acid | LC/MS: Rt = 4.54 min. [M + H] = 526 |
| 154 | 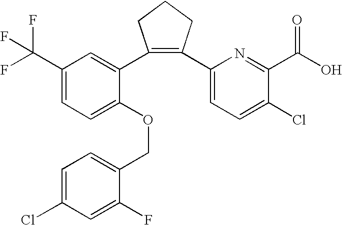 | 3-Chloro-6-{2-[2-{[(4- chloro-2-fluorophenyl) methyl]oxy}-5-(trifluoro- methyl)phenyl]-1- cyclopenten-1-yl}-2- pyridinecarboxylic acid | LC/MS: Rt = 4.55 min. [M + H] = 526 |
| 155 | 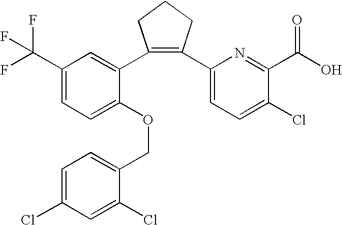 | 3-Chloro-6-{2-[2- {[(2,4-dichlorophenyl) methyl]oxy}-5- (trifluoromethyl)phenyl]-1- cyclopenten-1-yl}-2- pyridinecarboxylic acid | LC/MS: Rt = 4.77 min. [M + H] = 542, 544 |
| 156 | 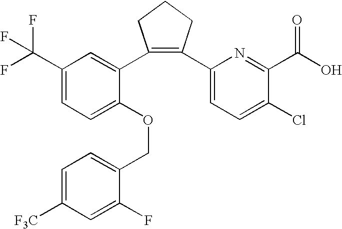 | 3-Chloro-6-{2-[2-({[2- fluoro-4-(trifluoromethyl) phenyl]methyl}oxy)-5- (trifluoromethyl)phenyl]-1- cyclopenten-1-yl}-2- pyridinecarboxylic acid | LC/MS: Rt = 4.41 min. [M + H] = 560 |
| 157 | 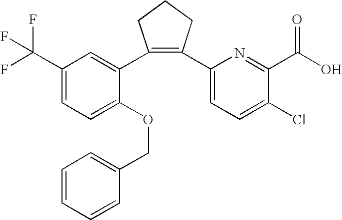 | 3-Chloro-6-{2-[2- [(phenylmethyl)oxy]-5- (trifluoromethyl)phenyl]-1- cyclopenten-1-yl}-2- pyridinecarboxylic acid | LC/MS: Rt = 4.31 min. [M + H] = 474 |
| 158 | 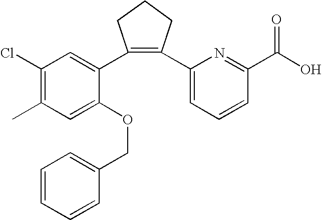 | 6-(2-{5-Chloro-4- methyl-2-[(phenylmethyl) oxy]phenyl}-1- cyclopenten-1-yl)-2- pyridinecarboxylic acid | LC/MS: Rt = 3.89 min. [M + H] = 420 |
| 159 | 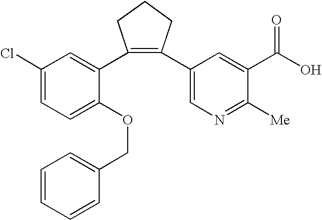 | 5-(2-{5-Chloro-2- [(phenylmethyl)oxy]phenyl}- 1-cyclopenten-1-yl)-2- methyl-3-pyridinecarboxylic acid | ¹H NMR (DMSO-d₆) δ: 1.98-2.05 (2H, m), 2.62 (3H, s), 2.79-2.83 (2H, m), 2.87- 2.90 (2H, m), 5.03 (2H, s), 7.09-7.33 (8H, m), 7.84 (1H, d), 8.21 (1H, d), 13.1 (1H, s) LC/MS: Rt = 3.81 [MH+] 420.4, 422.4 |
| 160 | 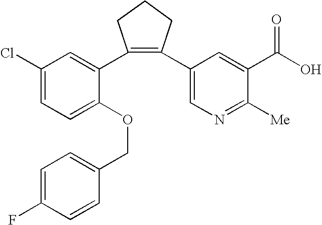 | 5-[2-(5-Chloro-2-{[(4- fluorophenyl)methyl]oxy} phenyl)-1-cyclopenten-1- yl]-2-methyl-3- pyridinecarboxylic acid | ¹H NMR (DMSO-d₆) δ: 1.99-2.06 (2H, m), 2.62 (3H, s), 2.78-2.82 (2H, m), 2.86- 2.90 (2H, m), 5.00 (2H, s), 7.10-7.14 (4H, m), 7.20-7.23 (2H, m), 7.31 (1H, dd), 7.81 (1H, d), 8.19 (1H, d), 13.1 (1H, s) LC/MS: Rt = 3.85 [MH+] 438.4, 440.4 |
| 161 | 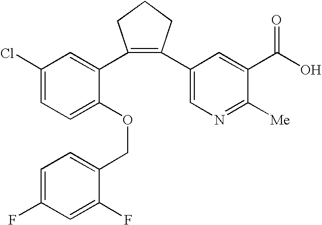 | 5-[2-(5-Chloro-2-{[(2,4- difluorophenyl)methyl] oxy}phenyl)-1- cyclopenten-1-yl]-2-methyl- 3-pyridinecarboxylic acid | ¹H NMR (DMSO-d₆) δ: 1.94-2.02 (2H, m), 2.61 (3H, s), 2.74-2.78 (2H, m), 2.83- 2.87 (2H, m), 5.01 (2H, s), 7.02 (1H, dt) 7.10 (1H, d), 7.18-7.35 (4H, m), 7.77 (1H, d), 8.14 (1H, d), 13.1 (1H, s) LC/MS: Rt = 3.89 [MH+] 456.3, 458.3 |
| 162 | 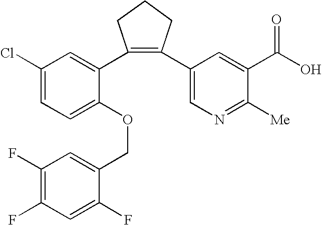 | 5-[2-(5-Chloro-2-{[(2,4,5- trifluorophenyl)methyl] oxy}phenyl)-1- cyclopenten-1-yl]-2-methyl- 3-pyridinecarboxylic acid | ¹H NMR (DMSO-d₆) δ: 1.96-2.02 (2H, m), 2.60 (3H, s), 2.77-2.80 (2H, m), 2.85- 2.88 (2H, m), 4.98 (2H, s), 7.16 (1H, d), 7.21 (1H, d) 7.21-7.26 (1H, m), 7.35 (1H, dd), 7.47-7.53 (1H, m), 7.75 (1H, d), 8.12 (1H, d), 13.0 (1H, s) LC/MS: Rt = 3.86 [MH+] 474.4, 476.4 |
| 163 | 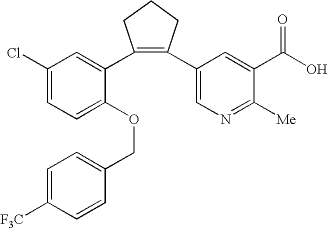 | 5-{2-[5-Chloro-2-({[4- (trifluoromethyl)phenyl] methyl}oxy)phenyl]- 1-cyclopenten-1-yl}- 2-methyl-3- pyridinecarboxylic acid | ¹H NMR (DMSO-d₆) δ: 1.99-2.06 (2H, m), 2.60 (3H, s), 2.81-2.85 (2H, m), 2.89- 2.93 (2H, m), 5.13 (2H, s), 7.10 (1H, d), 7.14 (1H, d), 7.31-7.38 (3H, m), 7.83 (2H, d), 7.83 (1H, d), 8.21 (1H, d), 13.1 (1H, s) LC/MS: Rt = 4.02 [MH+] 488.4, 490.4 |
| 164 | 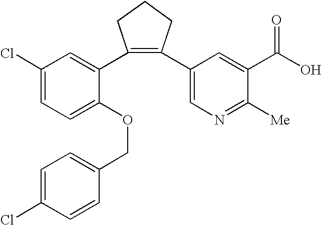 | 5-[2-(5-Chloro-2-{[(4- chlorophenyl)methyl] oxy}phenyl)-1- cyclopenten-1-yl]-2- methyl-3- pyridinecarboxylic acid | ¹H NMR (DMSO-d₆) δ: 1.96-2.04 (2H, m), 2.62 (3H, s), 2.79-2.82 (2H, m), 2.87-2.91 (2H, m), 5.01 (2H, s), 7.08-7.11 (2H, m), 7.18 (2H, d), 7.29-7.37 (3H, m), 7.82 (1H, d), 8.19 (1H, d), 13.1 (1H, s) LC/MS: Rt = 4.02 [MH+] 454.4 |
| 165 | 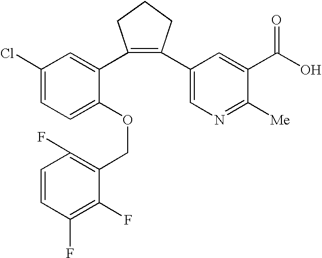 | 5-[2-(5-Chloro-2- {[(2,3,6- trifluorophenyl)methyl] oxy}phenyl)-1- cyclopenten-1-yl]-2- methyl-3- pyridinecarboxylic acid | ¹H NMR (DMSO-d₆) δ: 1.91-2.01 (2H, m), 2.61 (3H, s), 2.68-2.72 (2H, m), 2.87- 2.91 (2H, m), 5.06 (2H, s), 7.07-7.12 (2H, m), 7.27 (1H, d), 7.37 (1H, dd), 7.44-7.53 (1H, m), 7.71 (1H, d), 8.07 (1H, d), 13.1 (1H, s) LC/MS: Rt = 3.68 [MH+] 474.6, 476.4 |
| 166 | 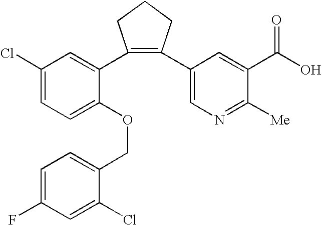 | 5-[2-(5-chloro-2-{[(2- chloro-4-fluorophenyl) methyl]oxy}phenyl)-1- cyclopenten-1-yl]-2-methyl-3- pyridinecarboxylic acid | ¹H NMR (DMSO-d₆) δ: 1.96-2.03 (2H, m), 2.60 (3H, s), 2.77-2.80 (2H, m), 2.85- 2.89 (2H, m), 5.01 (2H, s), 7.12-7.20 (3H, m), 7.31-7.35 (2H, m), 7.42 (1H, dd), 7.77 (1H, d), 8.14 (1H, d), 13.1 (1H, s) LC/MS: Rt = 4.05 [MH+] 472.4 |
| 167 | 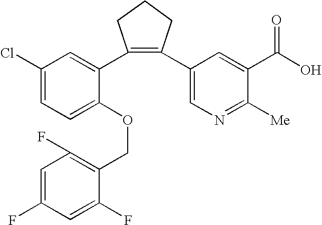 | 5-[2-(5-Chloro-2- {[(2,4,6-trifluorophenyl) methyl]oxy}phenyl)-1- cyclopenten-1-yl]-2-methyl- 3-pyridinecarboxylic acid | ¹H NMR (DMSO-d₆) δ: 1.90-1.97 (2H, m), 2.61 (3H, s), 2.68-2.72 (2H, m), 2.79- 2.82 (2H, m), 4.99 (2H, s), 7.09-7.17 (3H, m), 7.26 (1H, d), 7.36 (1H, dd), 7.71 (1H, d), 8.07 (1H, d), 13.1 (1H, s) LC/MS: Rt = 3.72 [MH+] 474.4, 476.4 |
| 168 | 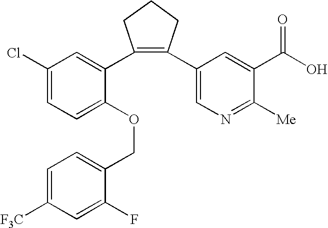 | 5-{2-[5-chloro-2-({[2- fluoro-4-(trifluoromethyl) phenyl]methyl}oxy)phenyl]- 1-cyclopenten-1-yl)-2-methyl- 3-pyridinecarboxylic acid | ¹H NMR (DMSO-d₆) δ: 1.97-2.05 (2H, m), 2.59 (3H, s), 2.78-2.81 (2H, m), 2.87- 2.91 (2H, m), 5.14 (2H, s), 7.15 (1H, d), 7.20 (1H, d), 7.34 (1H, dd), 7.42 (1H, t), 7.53 (1H, d), 7.65 (1H, d), 7.78 (1H, d), 8.15 (1H, d), 13.1 (1H, s) LC/MS: Rt = 4.05 [MH+] 506.5, 508.4 |
| 169 | 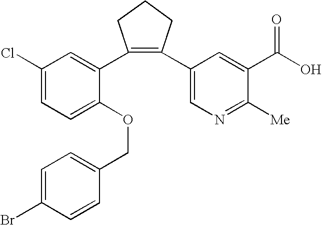 | 5-[2-(5-Chloro-2-{[(4- bromophenyl)methyl] oxy}phenyl)-1- cyclopenten-1-yl]-2- methyl-3-pyridinecarboxylic acid | ¹H NMR (DMSO-d₆) δ: 1.97-2.05 (2H, m), 2.62 (3H, s), 2.79-2.82 (2H, m), 2.87-2.91 (2H, m), 5.00 (2H, s), 7.08-7.13 (4H, m), 7.31 (1H, dd), 7.49 (2H, d), 7.82 (1H, d), 8.19 (1H, d), 13.1 (1H, s) LC/MS: Rt = 4.09 [MH+] 500.3, 502.3 |
| 170 | 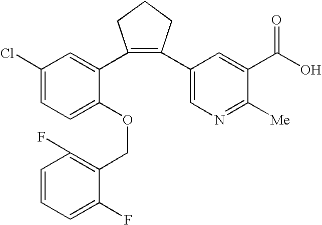 | 5-[2-(5-Chloro-2-{[(2,6- difluorophenyl)methyl] oxy}phenyl)-1-cyclopenten- 1-yl]-2-methyl-3- pyridinecarboxylic acid | ¹H NMR (DMSO-d₆) δ: 1.88-1.96 (2H, m), 2.62 (3H, s), 2.66-2.70 (2H, m), 2.78- 2.81 (2H, m), 5.05 (2H, s), 7.05-7.11 (3H, m), 7.26 (1H, d), 7.35 (1H, dd), 7.41-7.48 (1H, m), 7.74 (1H, d), 8.09 (1H, d), 13.1 (1H, s) LC/MS: Rt = 3.63 [MH+] 456.5, 458.4 |
| 171 | 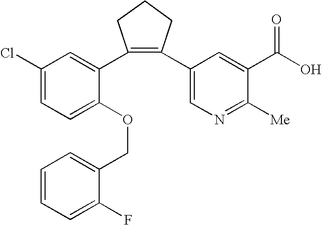 | 5-[2-(5-Chloro-2-{[(2- fluorophenyl)methyl] oxy}phenyl)-1- cyclopenten-1-yl]-2- methyl-3- pyridinecarboxylic acid | ¹H NMR (DMSO-d₆) δ: 1.95-2.02 (2H, m), 2.61 (3H, s), 2.75-2.79 (2H, m), 2.84- 2.88 (2H, m), 5.07 (2H, s), 7.08-7.24 (5H, m), 7.31-7.37 (2H, m), 7.80 (1H, d), 8.16 (1H, d), 13.1 (1H, s) LC/MS: Rt = 3.73 [MH+] 438.5, 440.4 |
| 172 | 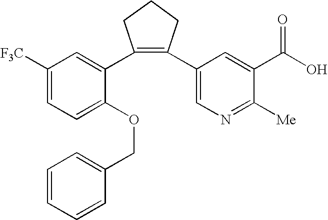 | 2-Methyl-5-{2-[2- [(phenylmethyl)oxy]-5- (trifluoromethyl)phenyl]- 1-cyclopenten-1-yl}-3- pyridinecarboxylic acid | ¹H NMR (DMSO-d₆) δ: 1.99-2.08 (2H, m), 2.61 (3H, s), 2.83-2.87 (2H, m), 2.89- 2.92 (2H, m), 5.14 (2H, s), 7.19-7.21 (2H, m), 7.26-7.34 (4H, m), 7.37 (1H, d), 7.63 (1H, dd), 7.82 (1H, d), 8.20 (1H, d), 13.0 (1H, s) LC/MS: Rt = 3.72 [MH+] 454.4 |
| 173 | 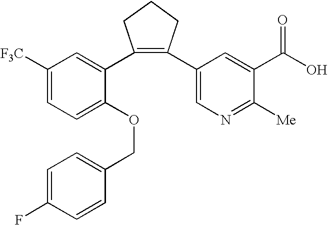 | 5-{2-[2-{[(4- Fluorophenyl)methyl] oxy}-5-(trifluoromethyl) phenyl]-1-cyclopenten-1- yl}-2-methyl-3- pyridinecarboxylic acid | ¹H NMR (DMSO-d₆) δ: 1.99-2.07 (2H, m), 2.61 (3H, s), 2.82-2.86 (2H, m), 2.88- 2.91 (2H, m), 5.10 (2H, s), 7.11-7.16 (2H, m), 7.22-7.30 (3H, m), 7.38 (1H, d), 7.64 (1H, dd), 7.79 (1H, d), 8.18 (1H, d), 13.0 (1H, s) LC/MS: Rt = 3.74 [MH+] 472.4 |
| 174 | 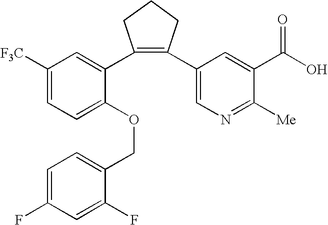 | 5-{2-[2-{[(2,4- Difluorophenyl)methyl] oxy}-5-(trifluoromethyl) phenyl]-1-cyclopenten-1- yl}-2-methyl-3- pyridinecarboxylic acid | ¹H NMR (DMSO-d₆) δ: 1.96-2.03 (2H, m), 2.60 (3H, s), 2.78-2.82 (2H, m), 2.85- 2.89 (2H, m), 5.12 (2H, s), 7.04 (1H, dt), 7.22 (1H, dt), 7.32-7.38 (3H, m), 7.65 (1H, dd), 7.74 (1H, d), 8.13 (1H, d), 13.1 (1H, s). LC/MS: Rt = 3.78 [MH+] 490.4 |
| 175 | 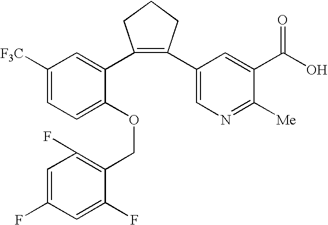 | 5-{2-[2-{[(2,4,6- Trifluorophenyl)methyl] yl]oxy}-5-(trifluoromethyl) phenyl]-1-cyclopenten-1- yl}-2-methyl-3- pyridinecarboxylic acid | ¹H NMR (DMSO-d₆) δ: 1.92-1.99 (2H, m), 2.61 (3H, s), 2.72-2.76 (2H, m), 2.80- 2.84 (2H, m), 5.09 (2H, s), 7.14-7.19 (2H, m), 7.37 (1H, d), 7.43 (1H, d), 7.67-7.69 (2H, m), 8.07 (1H, d), 13.1 (1H, s) LC/MS: Rt = 3.74 [MH+] 508.4 |
| 176 | 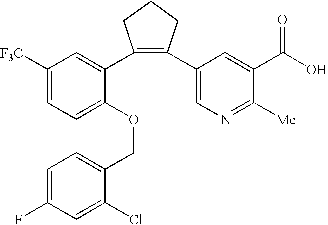 | 5-{2-[2-{[(2-Chloro-4- fluorophenyl)methyl] oxy}-5-(trifluoromethyl) phenyl]-1-cyclopenten-1- yl}-2-methyl-3- pyridinecarboxylic acid | ¹H NMR (DMSO-d₆) δ: 1.97-2.04 (2H, m), 2.59 (3H, s), 2.80-2.84(2H, m), 2.87- 2.90 (2H, m), 5.12 (2H, s), 7.19 (1H, dt), 7.35-7.40 (3H, m), 7.45 (1H, dd), 7.66 (1H, dd), 7.75 (1H, d), 8.14 (1H, d), 13.1 (1H, s) LC/MS: Rt = 4.00 [MH+] 506.4, 508.4 |
| 177 | 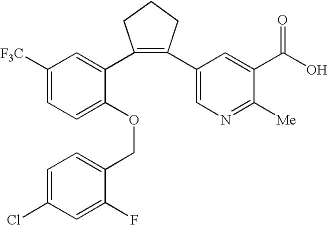 | 5-{2-[2-{[(4-Chloro-2- fluorophenyl)methyl] oxy}-5-(trifluoromethyl) phenyl]-1-cyclopenten-1- yl}-2-methyl-3- pyridinecarboxylic acid | ¹H NMR (DMSO-d₆) δ: 1.97-2.04 (2H, m), 2.60 (3H, s), 2.79-2.82 (2H, m), 2.86- 2.90 (2H, m), 5.14 (2H, s), 7.23-7.43 (5H, m), 7.66 (1H, dd), 7.75 (1H, d), 8.14 (1H, d), 13.1 (1H, br s) LC/MS: Rt = 3.99 [MH+] 506.4, 508.4 |
| 178 | 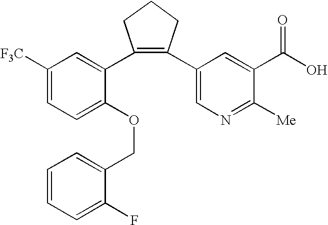 | 5-{2-[2-{[(2- Fluorophenyl)methyl]oxy}-5- (trifluoromethyl)phenyl]- 1-cyclopenten-1- yl}-2-methyl-3- pyridinecarboxylic acid | ¹H NMR (DMSO-d₆) δ: 1.97-2.04 (2H, m), 2.61 (3H, s), 2.79-2.83 (2H, m), 2.86- 2.90 (2H, m), 5.18 (2H, s), 7.13-7.27 (3H, m), 7.35-7.39 (3H, m), 7.65 (1H, dd), 7.78 (1H, d), 8.16 (1H, d), 13.1 (1H, br s) LC/MS: Rt = 3.84 [MH+] 472.5 |
| 179 | 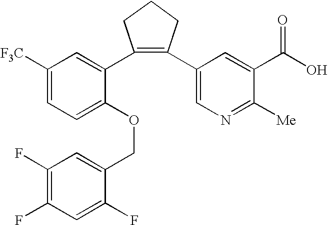 | 2-Methyl-5-[2-(5- (trifluoromethyl)-2-{[(2,4,5- trifluorophenyl)methyl] oxy}phenyl)-1- cyclopenten-1-yl]-3- pyridinecarboxylic acid | ¹H NMR (DMSO-d₆) δ: 1.98-2.05 (2H, m), 2.59 (3H, s), 2.80-2.84 (2H, m), 2.87- 2.90 (2H, m), 5.09 (2H, s), 7.28-7.31 (1H, dt, m), 7.37 (1H, d), 7.43 (1H, d), 7.51- 7.54 (1H, m), 7.67 (1H, dd), 7.73 (1H, d), 8.12 (1H, d), 13.1 (1H, s) LC/MS: Rt = 3.95 [MH+] 508.4 |
| 180 | 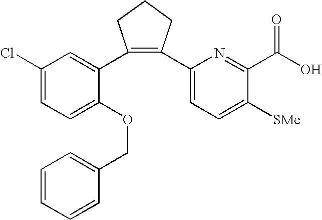 | 6-(2-{5-Chloro-2- [(phenylmethyl)oxy] phenyl}-1-cyclopenten-1- yl)-3-(methylthio)-2- pyridinecarboxylic acid | ¹H NMR (DMSO-d₆) δ: 1.95-2.02 (2H, m), 2.36 (3H, s), 2.82-2.85 (2H, m), 2.97- 3.01 (2H, m), 5.02 (2H, s), 7.03 (1H, d), 7.11-7.16 (4H, m), 7.25-7.33 (4H, m), 7.60 (1H, d), 12.6 (1H, s) LC/MS: Rt = 4.08 [MH+] 452.4, 454.4 |
| 181 | 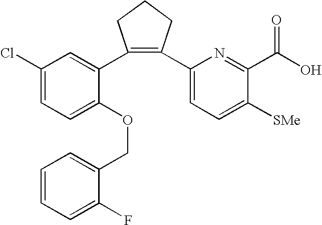 | 6-[2-(5-Chloro-2-{[(2- fluorophenyl)methyl] oxy}phenyl)-1- cyclopenten-1-yl]-3- (methylthio)-2- pyridinecarboxylic acid | ¹H NMR (DMSO-d₆) δ: 1.92- 2.00 (2H, m), 2.35 (3H, s), 2.78-2.82 (2H, m), 2.95-2.99 (2H, m), 5.07 (2H, s), 7.00 (1H, d), 7.09-7.21 (4H, m), 7.32-7.37 (2H, m), 7.58 (1H, d), 7.79 (1H, d), 12.5 (1H, s) LC/MS: Rt = 4.08 [MH+] 470.4, 472.4 |
| 182 | 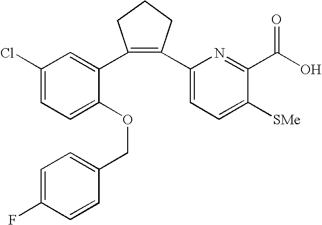 | 6-[2-(5-Chloro-2-{[(4- fluorophenyl)methyl] oxy}phenyl)-1-cyclopenten- 1-yl]-3-(methylthio)-2- pyridinecarboxylic acid | ¹H NMR (DMSO-d₆) δ: 1.92- 2.00 (2H, m), 2.19 (3H, s), 2.74-2.80 (2H, m), 2.85-2.92 (2H, m), 5.08 (2H, s), 6.65 (1H, d), 7.08 (1H, d), 7.18- 7.23 (4H, m), 7.25-7.41 (3H, m). LC/MS: Rt = 4.05 [MH+] 470.4, 472.4 |
| 183 | 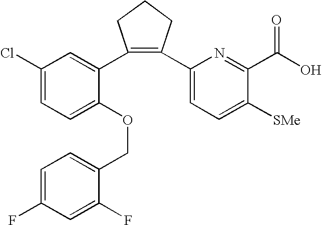 | 6-[2-(5-Chloro-2-{[(2,4- difluorophenyl)methyl] oxy}phenyl)-1-cyclopenten- 1-yl]-3-(methylthio)-2- pyridinecarboxylic acid | ¹H NMR (DMSO-d₆) δ: 1.92- 1.99 (2H, m), 2.35 (3H, s), 2.77-2.81 (2H, m), 2.94-2.98 (2H, m), 5.02 (2H, s), 6.96- 7.01 (2H, m), 7.15-7.24 (4H, m), 7.34 (1H, dd), 7.57 (1H, d), 7.79 (1H, d), 12.5 (1H, s) LC/MS: Rt = 4.09 [MH+] 488.4, 490.4 |
| 184 | 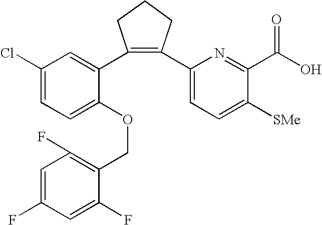 | 6-[2-(5-Chloro-2-{[(2,4,6- trifluorophenyl)methyl] oxy}phenyl)-1-cyclopenten- 1-yl]-3-(methylthio)-2- pyridinecarboxylic acid | ¹H NMR (DMSO-d₆) δ: 1.87- 1.95 (2H, m), 2.36 (3H, s), 2.71-2.74 (2H, m), 2.89-2.93 (2H, m), 5.02 (2H, s), 6.91 (1H, d), 7.10-7.14 (3H, m), 7.28 (1H, d), 7.37 (1H, dd), 7.56 (1H, d), 12.5 (1H, s) LC/MS: Rt = 4.06 [MH+] 506.3, 508.3 |
| 185 | 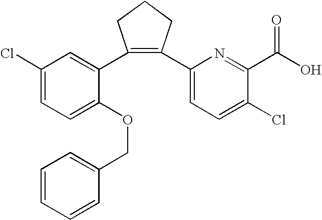 | 3-Chloro-6-(2-{5-chloro-2- [(phenylmethyl)oxy] phenyl}-1-cyclopenten-1-yl)- 2-pyridinecarboxylic acid | ¹H NMR (DMSO-d₆) δ: 1.94- 2.01 (2H, m), 2.82-2.86 (2H, m), 2.92-2.96 (2H, m), 5.01 (2H, s), 6.95 (1H, d), 7.10- 7.15 (4H, m), 7.26-7.33 (4H, m), 7.74 (1H, d), 13.6 (1H, s) LC/MS: Rt = 4.51 [MH+] 440.4 |
| 186 | 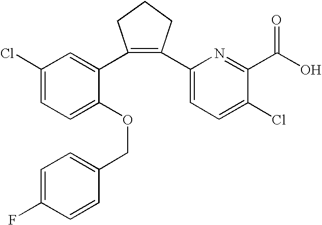 | 3-Chloro-6-[2-(5- chloro-2-{[(4- fluorophenyl)methyl] oxy}phenyl)-1- cyclopenten-1-yl]-2- pyridinecarboxylic acid | ¹H NMR (DMSO-d₆) δ: 1.93- 2.01 (2H, m), 2.81-2.84 (2H, m), 2.91-2.95 (2H, m), 4.97 (2H, s), 6.94 (1H, d), 7.09- 7.17 (6H, m), 7.32 (1H, dd), 7.74 (1H, d), 13.6 (1H, s) LC/MS: Rt = 4.50 [MH+] 458.4 |
| 187 | 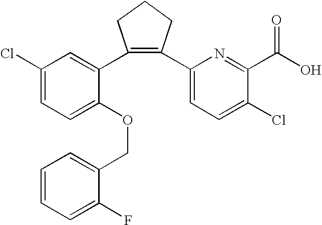 | 3-Chloro-6-[2-(5- chloro-2-{[(2-fluorophenyl) methyl]oxy}phenyl)-1- cyclopenten-1-yl]-2- pyridinecarboxylic acid | ¹H NMR (DMSO-d₆) δ: 1.94- 1.99 (2H, m), 2.78-2.82 (2H, m), 2.90-2.94 (2H, m), 5.07 (2H, s), 6.92 (1H, d), 7.11- 7.20 (5H, m), 7.32-7.38 (2H, m), 7.73 (1H, d), 13.6 (1H, s) LC/MS: Rt = 4.51 [MH+] 458.4 |
| 188 | 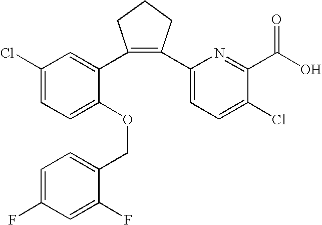 | 3-Chloro-6-[2-(5-chloro-2- {[(2,4-difluorophenyl) methyl]oxy}phenyl)-1- cyclopenten-1-yl]-2- pyridinecarboxylic acid | ¹H NMR (DMSO-d₆) δ: 1.93- 1.99 (2H, m), 2.77-2.81 (2H, m), 2.89-2.93 2H, m), 5.02 (2H, s), 6.90 (1H, d), 7.01 (1H, dt), 7.14-7.26 (4H, m), 7.34 (1H, dd), 7.72 (1H, d), 13.6 (1H, br s) LC/MS: Rt = 4.55 [MH+] 476.4 |
| 189 | 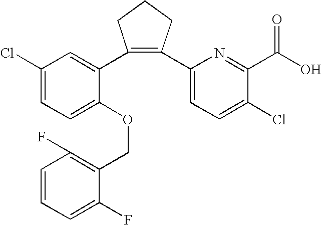 | 3-Chloro-6-[2-(5-chloro- 2-{[(2,6-difluorophenyl) methyl]oxy}phenyl)-1- cyclopenten-1-yl]-2- pyridinecarboxylic acid | ¹H NMR (DMSO-d₆) δ: 1.85- 1.93 (2H, m), 2.70-2.73 (2H, m), 2.83-2.87 (2H, m), 5.07 (2H, s), 6.83 (1H, d), 7.05- 7.11 (3H, m), 7.27 (1H, d), 7.36-7.47 (2H, m), 7.70 (1H, d), 13.6 (1H, s) LC/MS: Rt = 4.43 [MH+] 476.4 |
| 190 | 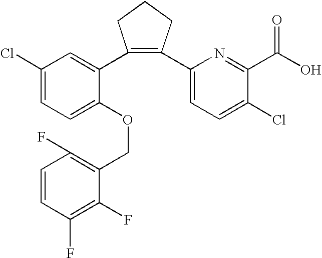 | 3-Chloro-6-[2-(5-chloro-2- {[(2,3,6-trifluorophenyl) methyl]oxy}phenyl)-1- cyclopenten-1-yl]-2- pyridinecarboxylic acid | ¹H NMR (DMSO-d₆) δ: 1.87- 1.94 (2H, m), 2.70-2.74 (2H, m), 2.84-2.88 (2H, m), 5.09 (2H, s), 6.84 (1H, d), 7.10- 7.15 (2H, m), 7.27 (1H, d), 7.37 (1H, dd), 7.49 (1H, m), 7.71 (1H, d), 13.6 (1H, s) LC/MS: Rt = 4.38 [MH+] 494.4 |
| 191 | 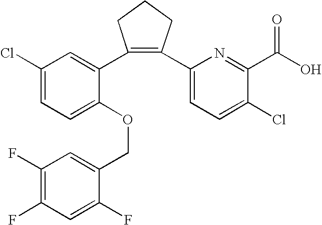 | 3-Chloro-6-[2-(5- chloro-2-{[(2,4,5- trifluorophenyl)methyl] oxy}phenyl)-1- cyclopenten-1-yl]-2- pyridinecarboxylic acid | ¹H NMR (DMSO-d₆) δ: 1.93- 2.01 (2H, m), 2.80-2.83 (2H, m), 2.91-2.94 (2H, m), 4.98 (2H, s), 6.92 (1H, d), 7.18- 7.28 (3H, m), 7.35 (1H, dd), 7.50 (1H, m), 7.72 (1H, d), 13.6 (1H, s) LC/MS: Rt = 4.48 [MH+] 494.4 |
| 192 | 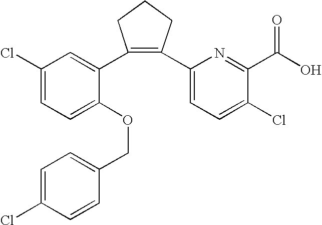 | 3-Chloro-6-[2-(5- chloro-2-{[(4- chlorophenyl)methyl] oxy}phenyl)-1- cyclopenten-1-yl]-2- pyridinecarboxylic acid | ¹H NMR (DMSO-d₆) δ: 1.94- 2.01 (2H, m), 2.81-2.85 (2H, m), 2.92-2.96 (2H, m), 4.99 (2H, s), 6.94 (1H, d), 7.08- 7.17 (4H, m), 7.30-7.36 (3H, m), 7.72 (1H, d), 13.7 (1H, s) LC/MS: Rt =0 4.78 [MH+] 476.4 |
| 193 | 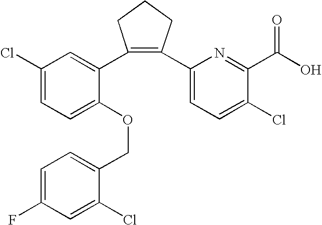 | 3-Chloro-6-[2-(5- chloro-2-{[(2-Chloro- 4-fluorophenyl)methyl] oxy}phenyl}-1- cyclopenten-1-yl]-2- pyridinecarboxylic acid | ¹H NMR (DMSO-d₆) δ: 1.93- 2.00 (2H, m), 2.80-2.83 (2H, m), 2.91-2.95 (2H, m), 5.03 (2H, s), 6.92 (1H, d), 7.15- 7.19 (3H, m), 7.25 (1H, dd), 7.34 (1H, dd), 7.43 (1H, dd), 7.73 (1H, d), 13.6 (1H, s) LC/MS: Rt = 4.86 [MH+] 494.3 |
| 194 | 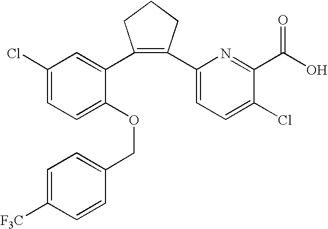 | 3-Chloro-6-{2-[5-chloro-2- ({[4-(trifluoromethyl) phenyl]methyl}oxy)phenyl]- 1-cyclopenten-1-yl}- 2-pyridinecarboxylic acid | ¹H NMR (DMSO-d₆) δ: 1.94- 2.03 (2H, m), 2.83-2.87 (2H, m), 2.94-2.98 (2H, m), 5.10 (2H, s), 6.97 (1H, d), 7.09 (1H, d), 7.20 (1H, d), 7.29- 7.35 (4H, m), 7.66 (1H, d), 7.73 (1H, d), 13.6 (1H, s) LC/MS: Rt = 4.58 [MH+] 508.4 |
| 195 | 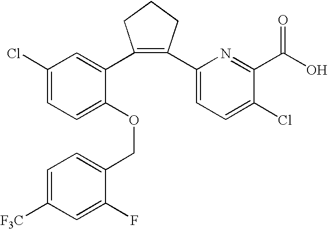 | 3-Chloro-6-{2-[5- chloro-2-({[2-fluoro- 4-(trifluoromethyl)phenyl] methyl}oxy)phenyl]- 1-cyclopenten-1-yl}- 2-pyridinecarboxylic acid | ¹H NMR (DMSO-d₆) δ: 1.94- 2.02 (2H, m), 2.81-2.84 (2H, m), 2.92-2.96 (2H, m), 5.14 (2H, s), 6.94 (1H, d), 7.17- 7.20 (2H, m), 7.32-7.36 (2H, m), 7.53 (1H, d), 7.65 (1H, d), 7.71 (1H, d), 13.6 (1H, s) LC/MS: Rt = 4.39 [MH+] 526.3 |
| 196 | 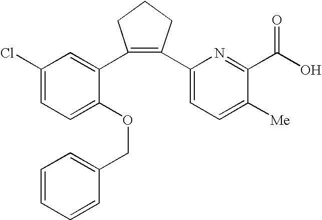 | 6-(2-{5-Chloro-2- [(phenylmethyl)oxy]phenyl}- 1-cyclopenten-1-yl)-3-methyl- 2-pyridinecarboxylic acid | ¹H NMR (DMSO-d₆) δ: 1.94- 2.02 (2H, m), 2.38 (3H, s), 2.82-2.85 (2H, m), 2.93-2.99 (2H, m), 5.02 (2H, s), 6.95 (1H, d), 7.10-7.16 (4H, m), 7.26-7.32 (4H, m), 7.50 (1H, d), 12.6 (1H, s) LC/MS: Rt = 4.02 [MH+] 420.4, 422.5 |
| 197 | 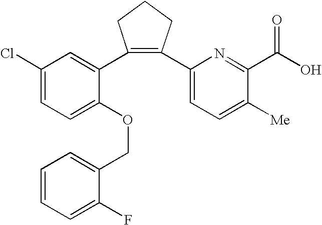 | 6-[2-(5-Chloro-2-{[(2- fluorophenyl)methyl] oxy}phenyl)-1-cyclopenten- 1-yl]-3-methyl-2- pyridinecarboxylic acid | ¹H NMR (DMSO-d₆) δ: 1.92- 1.99 (2H, m), 2.37 (3H, s), 2.78-2.82 (2H, m), 2.93-2.97 (2H, m), 5.07 (2H, s), 6.92 (1H, d), 7.08-7.22 (5H, m), 7.31-7.36 (2H, m), 7.48 (1H, d), 12.6 (1H, s) LC/MS: Rt = 4.04 [MH+] 438.4, 440.4 |
| 198 | 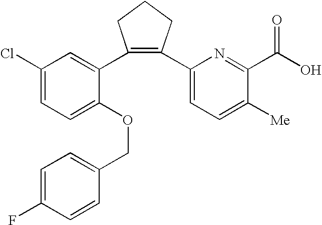 | 6-[2-(5-Chloro-2-{[(4- fluorophenyl)methyl]oxy} phenyl)-1-cyclopenten-1- yl]-3-methyl-2- pyridinecarboxylic acid | ¹H NMR (DMSO-d₆) δ: 1.94- 2.01 (2H, m), 2.37 (3H, s), 2.80-2.84 (2H, m), 2.94-2.98 (2H, m), 4.99 (2H, s), 6.93 (1H, d), 7.08-7.19 (6H, m), 7.31, (1H, dd), 7.49 (1H, d), 12.6 (1H, s) LC/MS: Rt = 4.04 [MH+] 438.4, 440.4 |
| 199 | 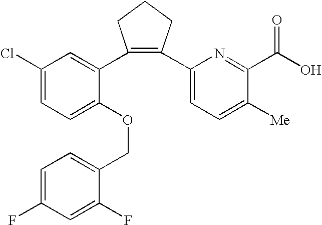 | 6-[2-(5-Chloro-2-{[(2,4- difluorophenyl)methyl] oxy}phenyl)-1-cyclopenten- 1-yl]-3-methyl-2- pyridinecarboxylic acid | ¹H NMR (DMSO-d₆) δ: 1.92- 1.99 (2H, m), 2.37 (3H, s), 2.77-2.80 (2H, m), 2.92-2.96 (2H, m), 5.02 (2H, s), 6.91 (1H, d), 7.00 (1H, dt), 7.10 (1H, d), 7.19-7.26 (4H, m), 7.33, (1H, dd), 7.48 (1H, d), 12.6 (1H, s) LC/MS: Rt = 4.09 [MH+] 456.4, 458.4 |
| 200 | 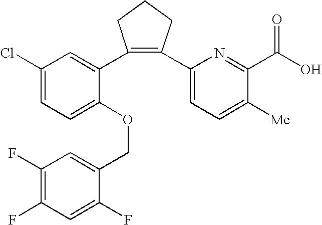 | 6-[2-(5-Chloro-2-{[(2,4,5- trifluorophenyl)methyl] oxy}phenyl)-1-cyclopenten- 1-yl]-3-methyl-2- pyridinecarboxylic acid | ¹H NMR (DMSO-d₆) δ: 1.93- 2.00 (2H, m), 2.36 (3H, s), 2.79-2.82 (2H, m), 2.93-2.97 (2H, m), 4.99 (2H, s), 6.92 (1H, d), 7.15 (1H, d), 7.19- 7.24 (2H, m), 7.34, (1H, dd), 7.47-7.52 (2H, m), 12.6 (1H, s) LC/MS: Rt = 4.17 [MH+] 474.4, 476.4 |
| 201 | 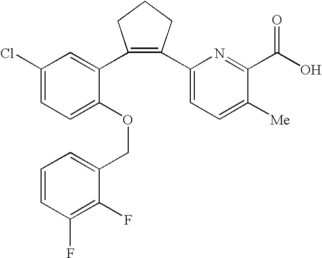 | 6-[2-(5-Chloro-2-{[(2,3- difluorophenyl)methyl] oxy}phenyl)-1-cyclopenten- 1-yl]-3-methyl-2- pyridinecarboxylic acid | ¹H NMR (DMSO-d₆) δ: 1.93- 2.00 (2H, m), 2.37 (3H, s), 2.78-2.82 (2H, m), 2.93-2.97 (2H, m), 5.11 (2H, s), 6.92 (1H, d), 7.01 (1H, t), 7.10- 7.15 (2H, m), 7.20 (1H, d), 7.32-7.37 (2H, m), 7.48 (1H, d), 12.6 (1H, s) LC/MS: Rt = 4.09 [MH+] 456.4, 458.4 |
| 202 | 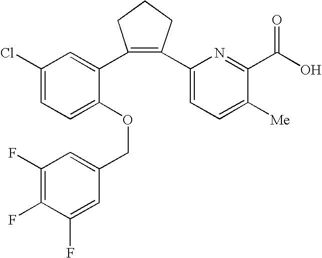 | 6-[2-(5-Chloro-2-{[(3,4,5- trifluorophenyl)methyl] oxy}phenyl)-1-cyclopenten- 1-yl]-3-methyl-2- pyridinecarboxylic acid | ¹H NMR (DMSO-d₆) δ: 1.97- 2.04 (2H, m), 2.36 (3H, s), 2.83-2.86 (2H, m), 2.97-3.00 (2H, m), 4.96 (2H, s), 6.95- 7.09 (4H, m), 7.19 (1H, d), 7.33, (1H, dd), 7.50 (1H, d), 12.6 (1H, s) LC/MS: Rt = 4.24 [MH+] 474.4, 476.4 |
| 203 | 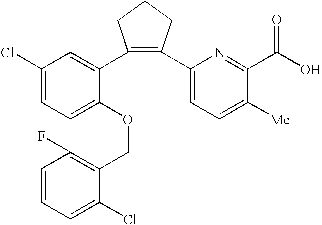 | 6-[2-(5-Chloro-2-{[(2- chloro-6-fluorophenyl) methyl]oxy}phenyl)-1- cyclopenten-1-yl]-3-methyl-2- pyridinecarboxylic acid | ¹H NMR (DMSO-d₆) δ: 1.85- 1.92 (2H, m), 2.38 (3H, s), 2.70-2.73 (2H, m), 2.86-2.89 (2H, m), 5.12 (2H, s), 6.85 (1H, d), 7.02 (1H, d), 7.21, (1H, t), 7.30-7.48 (5H, m), 12.5 (1H, s) LC/MS: Rt = 4.24 [MH+] 472.4 |
| 204 | 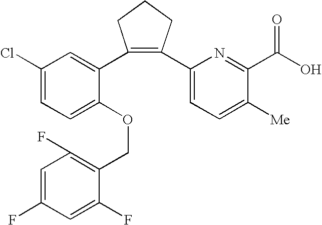 | 6-[2-(5-Chloro-2-{[(2,4,6- trifluorophenyl)methyl] oxy}phenyl)-1-cyclopenten- 1-yl]-3-methyl-2- pyridinecarboxylic acid | ¹H NMR (DMSO-d₆) δ: 1.87- 1.95 (2H, m), 2.38 (3H, s), 2.70-2.74 (2H, m), 2.87-2.91 (2H, m), 5.02 (2H, s), 6.85 (1H, d), 7.07 (1H, d), 7.10- 7.16 (2H, m), 7.27 (1H, d), 7.36 (1H, dd), 7.47 (1H, d), 12.6 (1H, s) LC/MS: Rt = 4.06 [MH+] 474.4, 476.4 |
| 205 | 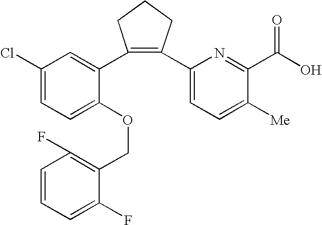 | 6-[2-(5-Chloro-2-{[(2,6- difluorophenyl)methyl] oxy}phenyl)-1-cyclopenten- 1-yl]-3-methyl-2- pyridinecarboxylic acid | ¹H NMR (DMSO-d₆) δ: 1.86- 1.93 (2H, m), 2.38 (3H, s), 2.70-2.73 (2H, m), 2.87-2.90 (2H, m), 5.08 (2H, s), 6.85 (1H, d), 7.03-7.09 (3H, m), 7.28 (1H, d), 7.34-7.48 (3H, m), 12.6 (1H, s) LC/MS: Rt = 3.99 [MH+] 456.4, 458.4 |
| 206 | 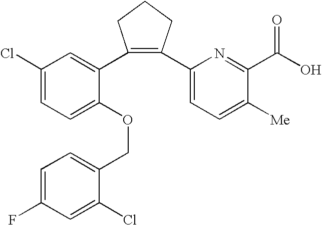 | 6-[2-(5-Chloro-2-{[(2- chloro-4-fluorophenyl) methyl]oxy}phenyl)-1- cyclopenten-1-yl]-3-methyl- 2-pyridinecarboxylic acid | ¹H NMR (DMSO-d₆) δ: 1.94- 2.00 (2H, m), 2.36 (3H, s), 2.79-2.83 (2H, m), 2.94-2.97 (2H, m), 5.03 (2H, s), 6.91 (1H, d), 7.12-7.20 (3H, m), 7.27-7.34 (2H, m), 7.41 (1H, dd), 7.48 (1H, d), 12.6 (1H, s) LC/MS: Rt = 4.31 [MH+] 472.4 |
| 207 | 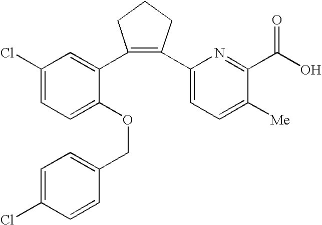 | 6-[2-(5-Chloro-2-{[(4- chlorophenyl)methyl] oxy}phenyl)-1-cyclopenten- 1-yl]-3-methyl-2- pyridinecarboxylic acid | ¹H NMR (DMSO-d₆) δ: 1.95- 2.01 (2H, m), 2.37 (3H, s), 2.81-2.84 (2H, m), 2.95-2.99 (2H, m), 5.00 (2H, s), 6.94 (1H, d), 7.10-7.15 (4H, m), 7.29-7.35 (3H, m), 7.49 (1H, d), 12.6 (1H, s) t = 4.26 [MH+] 454.4 |
| 208 | 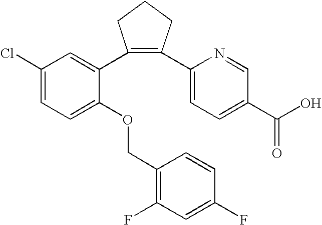 | 6-[2-(5-Chloro-2-{[(2,4- difluorophenyl)methyl] oxy}phenyl)-1- cyclopenten-1-yl]-3- pyridinecarboxylic acid | ¹H NMR (DMSO-d₆) δ: 1.92- 2.00 (2H, m), 2.79-2.82 (2H, m), 2.94-2.98 (2H, m), 5.00 (2H, s), 6.94 (1H, d), 7.01 (1H, dt), 7.10 (1H, d), 7.15- 7.27 (3H, m), 7.34 (1H, dd), 7.92 (1H, dd), 8.86 (1H), 13.2 (1H, s) LC/MS: Rt = 4.25 [MH+] 442.3, 444.3 |
| 209 | 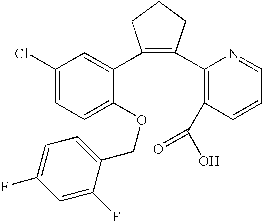 | 2-[2-(5-Chloro-2-{[(2,4- difluorophenyl)methyl] oxy}phenyl)-1- cyclopenten-1-yl]-3- pyridinecarboxylic acid | ¹H NMR (DMSO-d₆) δ: 1.94- 2.02 (2H, m), 2.75-2.79 (4H, m), 5.01 (2H, s), 6.73 (1H, d), 6.97 (1H, d), 7.10-7.17 (2H, m), 7.28-7.33 (2H, m), 7.52 (1H, q), 7.98 (1H, dd), 8.56 (1H, dd), 13.0 (1H, s) LC/MS: Rt = 3.54 [MH+] 442.3, 444.3 |
| 210 | 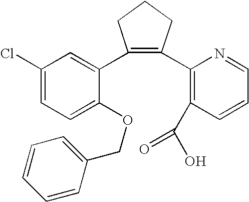 | 2-(2-{5-Chloro-2- [(phenylmethyl)oxy] phenyl}-1-cyclopenten- 1-yl)-3-pyridinecarboxylic acid | LC/MS: Rt = 3.41 [MH+] 406.4, 408.4 |
| 211 | 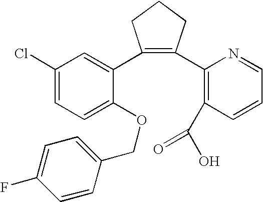 | 2-[2-(5-Chloro-2-{[(4- fluorophenyl)methyl] oxy}phenyl)-1-cyclopenten- 1-yl]-3-pyridinecarboxylic acid | LC/MS: Rt = 3.46 [MH+] 424.3, 426.3 |
| 212 | 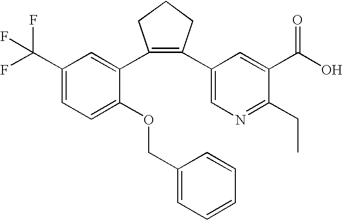 | 2-Ethyl-5-{2-[2- [(phenylmethyl)oxy]- 5-(trifluoromethyl)phenyl]- yl]-1-cyclopenten-1-yl}-3- pyridinecarboxylic acid | ¹H NMR (DMSO-d₆) δ: 1.12 (3H, t), 2.00-2.07 (2H, m), 2.83-2.92 (4H, m), 2.98 (2H, q), 5.13 (2H, s), 7.19 (2H, m), 7.27-7.36 (5H, m), 7.63 (1H, dd), 7.79 (1H, d), 8.24 (1H, d), 13.2 (1H, s) LC/MS: Rt = 3.87 [MH+] 468.4 |
| 213 | 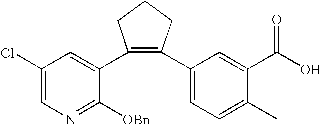 | 5-(2-{5-Chloro-2- [(phenylmethyl)oxy]- 3-pyridinyl}-1- cyclopenten-1-yl)-2- methylbenzoic acid | LC/MS: Rt = 4.04 min. [MH⁺] 420, 422. |
| 214 | 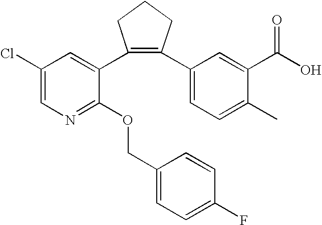 | 5-[2-(5-Chloro-2-{[(4- fluorophenyl)methyl] oxy}-3-pyridinyl)-1- cyclopenten-1-yl]-2- methylbenzoic acid | LC/MS: Rt = 4.04 min. [MH⁺] 438, 440. |
| 215 | 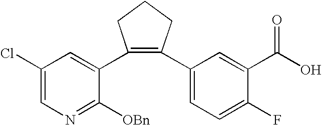 | 5-(2-{5-Chloro-2- [(phenylmethyl)oxy]- 3-pyridinyl}-1-cyclopenten- 1-yl)-2-fluorobenzoic acid | LC/MS: Rt = 4.44 min. [MH⁺] 424, 426. |
| 216 | 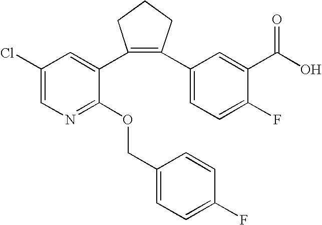 | 5-[2-(5-Chloro-2-{[(4- fluorophenyl)methyl] oxy}-3-pyridinyl)-1- cyclopenten-1-yl]-2- fluorobenzoic acid | LC/MS: Rt = 4.39 min. [MH⁺] 442, 444. |
| 217 | 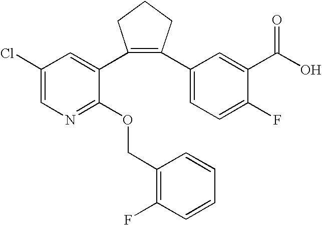 | 5-[2-(5-chloro-2-{[(2- fluorophenyl)methyl] oxy}-3-pyridinyl)-1- cyclopenten-1-yl]-2- fluorobenzoic acid | LC/MS: Rt = 4.32 min. [MH⁺] 442, 444. |
| 218 | 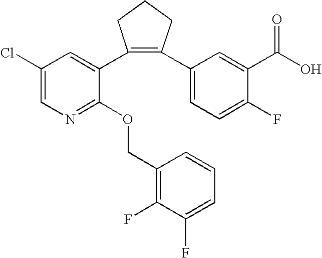 | 5-[2-(5-Chloro-2- {[(2,3-difluorophenyl) methyl]oxy}-3-pyridinyl)-1- cyclopenten-1-yl]-2- fluorobenzoic acid | LC/MS: Rt = 4.26 min. [MH⁺] 460, 462. |
| 219 | 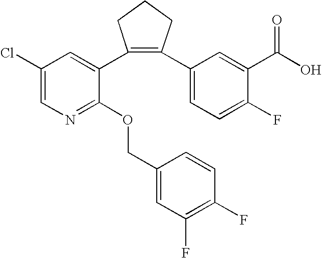 | 5-[2-(5-Chloro-2- {[(3,4-difluorophenyl) methyl]oxy}-3-pyridinyl)-1- cyclopenten-1-yl]-2- fluorobenzoic acid | LC/MS: Rt = 4.31 min. [MH⁺] 460, 462. |
| 220 | 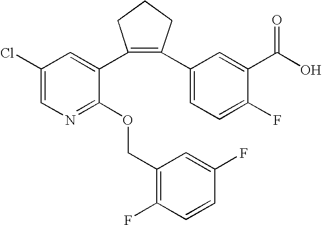 | 5-[2-(5-Chloro-2- {[(2,5-difluorophenyl)methyl] oxy}-3-pyridinyl)-1- cyclopenten-1-yl]-2- fluorobenzolc acid | LC/MS: Rt = 4.32 min. [MH⁺] 460, 462. |
| 221 | 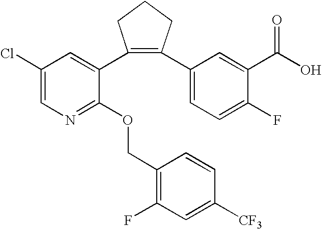 | 5-{2-[5-Chloro-2-({[2- fluoro-4-(trifluoromethyl) phenyl]methyl}oxy)-3- pyridinyl]-1-cyclopenten-1- yl}-2-fluorobenzoic acid | LC/MS: Rt = 4.46 min. [MH⁺] 510, 512. |
| 222 | 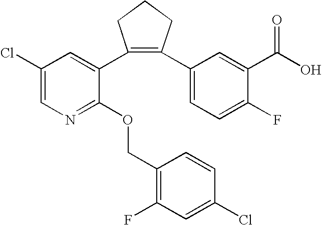 | 5-[2-(5-Chloro-2-{[(4- chloro-2-fluorophenyl) methyl]oxy}-3-pyridinyl)-1- cyclopenten-1-yl]-2- fluorobenzoic acid | LC/MS: Rt = 4.53 min. [MH⁺] 476, 477, 478, 479. |
| 223 | 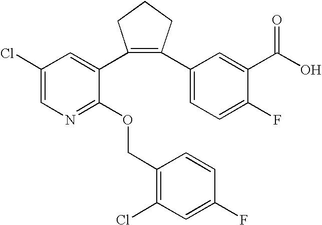 | 5-[2-(5-chloro-2-{[(2- chloro-4-fluorophenyl) methyl]oxy}-3-pyridinyl)-1- cyclopenten-1-yl]-2- fluorobenzoic acid | LC/MS: Rt = 4.54 min. [MH⁺] 476, 477, 478, 479. |
| 224 | 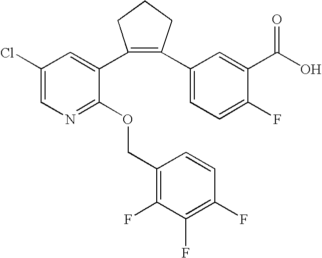 | 5-[2-(5-Chloro-2- {[(2,3,4-trifluorophenyl) methyl]oxy}-3-pyridinyl)-1- cyclopenten-1-yl]-2- fluorobenzoic acid | LC/MS: Rt = 4.34 min. [MH⁺] 478, 480. |
| 225 | 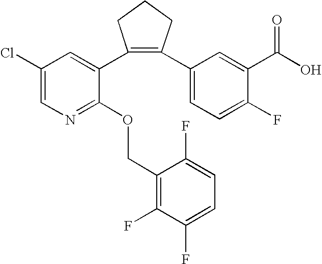 | 5-[2-(5-Chloro-2- {[(2,3,6-trifluorophenyl) methyl]oxy}-3-pyridinyl)-1- cyclopenten-1-yl]-2- fluorobenzoic acid | LC/MS: Rt = 4.26 min. [MH⁺] 478, 480. |
| 226 | 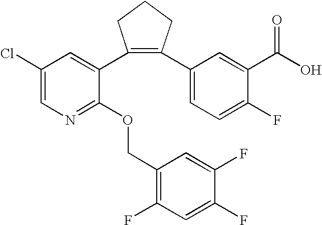 | 5-[2-(5-Chloro-2- {[(2,4,5-trifluorophenyl) methyl]oxy}-3-pyridinyl)-1- cyclopenten-1-yl-2- fluorobenzoic acid | LC/MS: Rt = 4.34 min. [MH⁺] 478, 480. |
| 227 | 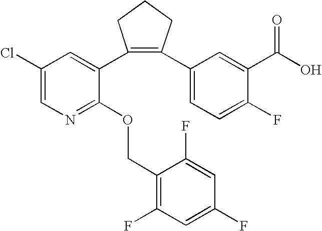 | 5-[2-(5-Chloro-2- {[(2,4,6-trifluorophenyl) methyl]oxy}-3-pyridinyl)-1- cyclopenten-1-yl]-2- fluorobenzoic acid | LC/MS: Rt = 4.28 min. [MH⁺] 478, 480. |
| 228 | 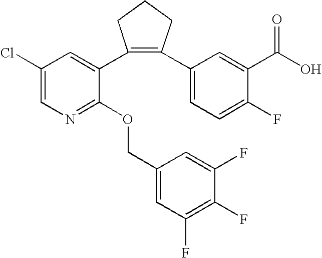 | 5-[2-(5-chloro-2- {[(3,4,5-trifluorophenyl) methyl]oxy}-3-pyridinyl)-1- cyclopenten-1-yl]-2- fluorobenzoic acid | LC/MS: Rt = 4.29 min. [MH⁺] 478, 480. |
| 229 | 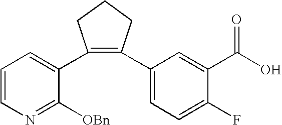 | 2-Fluoro-5-(2-{2- [(phenylmethyl)oxy]- 3-pyridinyl}-1-cyclopenten- 1-yl)benzoic acid | LC/MS: Rt = 4.04 min. [MH⁺] 390. |
| 230 | 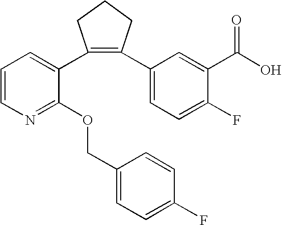 | 2-Fluoro-5-[2-(2-{[(4- fluorophenyl)methyl] oxy}-3-pyridinyl)-1- cyclopenten-1-yl]benzoic acid | LC/MS: Rt = 4.06 min [MH⁺] 408. |
| 231 | 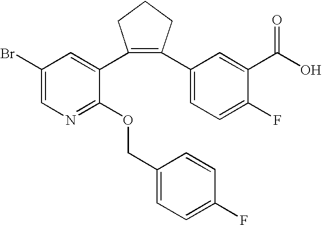 | 5-[2-(5-Bromo-2-{[(4- fluorophenyl)methyl]oxy}- 3-pyridinyl)-1-cyclopenten-1- yl]-2-fluorobenzoic acid | LC/MS: Rt = 4.26 min. MH⁺] 486, 488. |
| 232 | 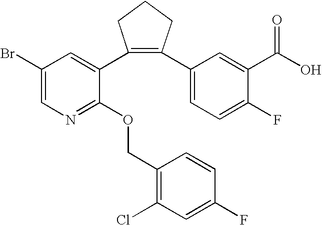 | 5-[2-(5-Bromo-2-{[(2- chloro-4-fluorophenyl) methyl]oxy}-3-pyridinyl)-1- cyclopenten-1-yl]-2- fluorobenzoic acid | LC/MS: Rt = 4.25 min. [MH⁺] 520, 522. |
| 233 | 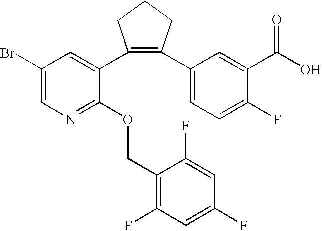 | 5-[2-(5-Bromo-2-{[(2,4,6- trifluorophenyl)methyl] oxy}-3-pyridinyl)-1- cyclopenten-1-yl]-2- fluorobenzoic acid | LC/MS: Rt = 4.23 min. [MH⁺] 522, 524. |
| 234 | 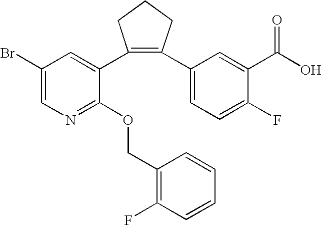 | 5-[2-(5-Bromo-2-{[(2- fluorophenyl)methyl] oxy}-3-pyridinyl)-1- cyclopenten-1-yl]-2- fluorobenzoic acid | LC/MS: Rt = 4.38 min. [MH⁺] 486, 488. |
| 235 | 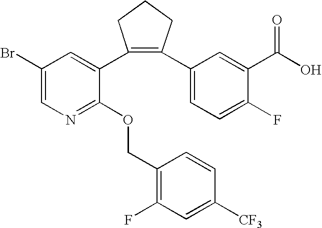 | 5-{2-[5-Bromo-2-({[2- fluoro-4-(trifluoromethyl) phenyl]methyl}oxy)-3- pyridinyl]-1-cyclopenten-1- yl}-2-fluorobenzoic acid | LC/MS: Rt = 4.54 min. [MH⁺] 554, 556. |
| 236 | 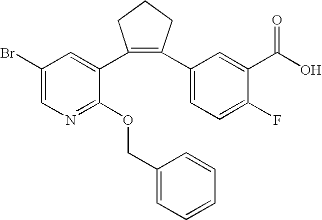 | 5-(2-{5-Bromo-2- [(phenylmethyl)oxy]- 3-pyridinyl}-1-cyclopenten- 1-yl)-2-fluorobenzoic acid | LC/MS: Rt = 4.42 min [MH⁺] 468, 470. |
| 237 | 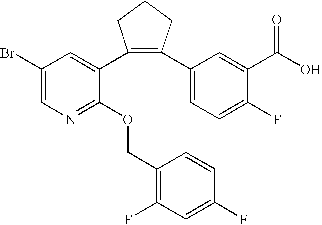 | 5-[2-(5-Bromo-2- {[(2,4-difluorophenyl) methyl]oxy}-3-pyridinyl)-1- cyclopenten-1-yl]-2- fluorobenzoic acid | LC/MS: Rt = 4.50 min [MH⁺] 504, 506. |
| 238 | 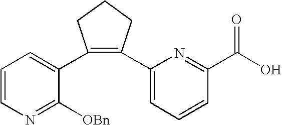 | 6-(2-{2-[(Phenylmethyl)oxy]- 3-pyridinyl}-1-cyclopenten- 1-yl)-2-pyridinecarboxylic acid | LC/MS: Rt = 3.24 min [MH⁺] 373. |
| 239 | 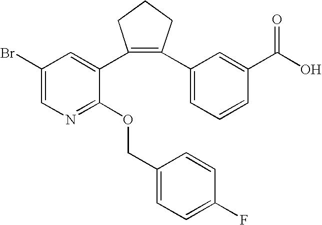 | 3-[2-(5-Bromo-2-{[(4- fluorophenyl)methyl]oxy}-3- pyridinyl)-1-cyclopenten-1- yl]benzoic acid | LC/MS: Rt = 4.26 min [MH⁺] 468, 470 |
| 240 | 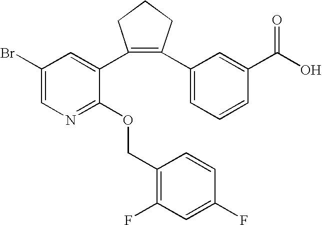 | 3-[2-(5-Bromo-2-{[(2,4- difluorophenyl)methyl] oxy}-3-pyridinyl)-1- cyclopenten-1-yl]benzoic acid | LC/MS: Rt = 3.93 min [MH+] 486, 488 |
| 241 | 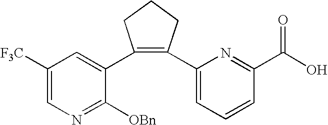 | 6-{2-[2-(Phenylmethoxy)- 5-(trifluoromethyl)pyridin- 3-yl]cyclopent-1-en-1-yl}- pyridine-2-carboxylic acid | LC/MS Rt = 3.92 min [MH⁺] 441. |
| 242 | 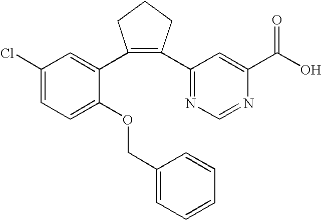 | 6-(2-{5-Chloro-2- [(phenylmethyl)oxy]phenyl}- 1-cyclopenten-1-yl)-4- pyrimidinecarboxylic acid | LC/MS: Rt = 4.88 [MH+] 407.3, 409.3 |
| 243 | 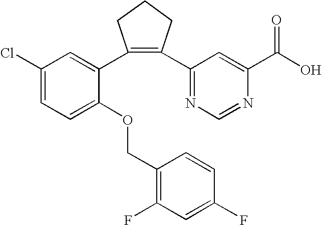 | 6-[2-(5-Chloro-2-{[(2,4- difluorophenyl)methyl]oxy} phenyl)-1-cyclopenten-1-yl]- 4-pyrimidinecarboxylic acid | LC/MS: Rt = 5.13 [MH+] 443.3, 445.3 |

### Example 244

### 6-[2-(2-{[(4-Bromo-2-fluorophenyl)methyl]oxy}-5-fluorophenyl)-1-cyclopenten-1-yl]-2-pyridinecarboxylic acid

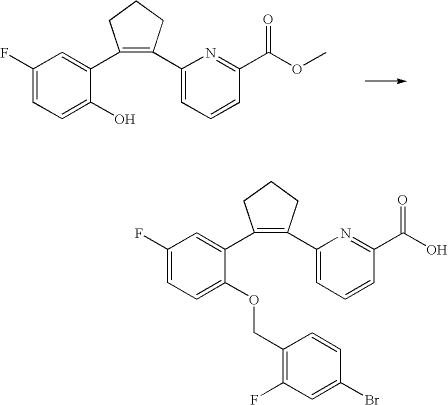

Methyl 6-[2-(5-fluoro-2-hydroxyphenyl)1-cyclopenten-1-yl]-2-pyridinecarboxylate (104 mg, 0.333 mmol) was treated with 4-bromo-2-fluorobenzyl bromide (96 mg, 0.358 mmol) and potassium carbonate (140 mg, 1.0 mmol) in 2-butanone (4 ml). The reaction mixture was then refluxed overnight under nitrogen, filtered through celite and reduced under vacuum to an oil. The oil was dissolved in methanol (3 ml), 2M sodium hydroxide (2 ml) was added and the reaction mixture stirred at 65° C. for one hour. The reaction mixture was then reduced down to −1 ml under vacuum, diluted to 20 ml with water and 2M hydrochloric acid (1.6 ml) added as well as a couple of drops of acetic acid to pH-6, extracted with ethyl acetate (2×20 ml). The organic extract was then dried over magnesium sulphate, filtered and evaporated down to a solid (69 mg, 42%)

LC/MS Rt=3.94 min [MH⁺] 488.

The following Examples were prepared by the procedure used for 6-[2-(2-{[(4bromo-2-fluorophenyl)methyl]oxy}-fluorophenyl)-cyclopenten-1-yl]-2-pyridinecarboxylic acid:

| Example | | Name | LC/MS |
|---|---|---|---|
| 245 | 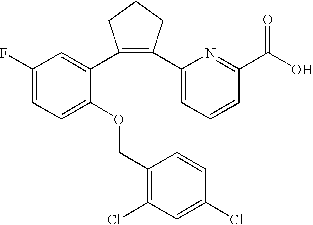 | 6-[2-(2-{[(2,4- Dichlorophenyl)methyl]oxy}- 5-fluorophenyl)-1- cyclopenten-1-yl]-2- pyridinecarboxylic acid | Rt = 4.16, [MH⁺] 458 |
| 246 | 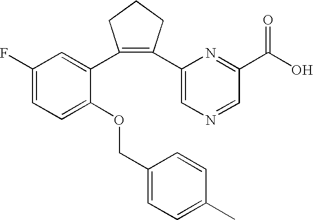 | 6-[2-(5-Fluoro-2-{[(4- methylphenyl)methyl]oxy} phenyl)-1-cyclopenten-1-yl]-2- pyrazinecarboxylic acid | Rt = 3.75, [MH⁺] 405 |
| 247 | 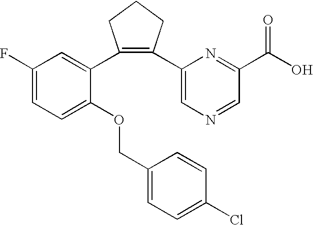 | 6-[2-(2-{[(4- Chlorophenyl)methyl]oxy}- 5-fluorophenyl)-1-cyclopenten- 1-yl]-2-pyrazinecarboxylic acid | Rt = 4.43, [MH⁺] 425 |
| 248 | 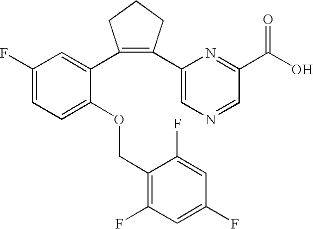 | 6-[2-(5-Fluoro-2-{[(2,4,6- trifluorophenyl)methyl]oxy} phenyl)-1-cyclopenten-1-yl]-2- pyrazinecarboxylic acid | Rt = 4.08, [MH⁺] 445 |
| 249 | 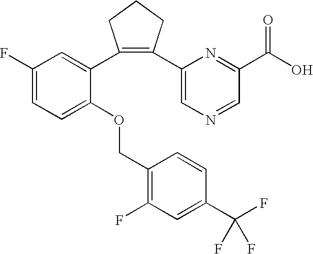 | 6-{2-[5-Fluoro-2-({[2-fluoro-4- (trifluoromethyl)phenyl]methyl} oxy)phenyl]-1-cyclopenten-1- yl}-2-pyrazinecarboxylic acid | Rt = 4.27, [MH⁺] 475 |
| 250 | 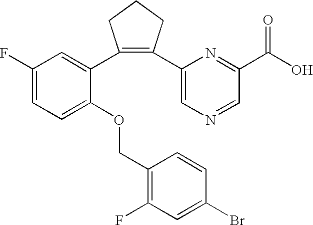 | 6-[2-(2-{[(4-Bromo-2- fluorophenyl)methyl]oxy}-5- fluorophenyl)-1-cyclopenten- 1-yl]-2-pyrazinecarboxylic acid | Rt = 4.57, [MH⁺] 489 |
| 251 | 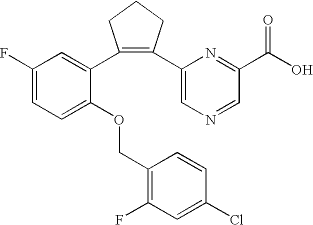 | 6-[2-(2-{[(4-Chloro-2- fluorophenyl)methyl]oxy}-5- fluorophenyl)-1-cyclopenten- 1-yl]-2-pyrazinecarboxylic acid | Rt = 4.47, [MH⁺] 443 |
| 252 | 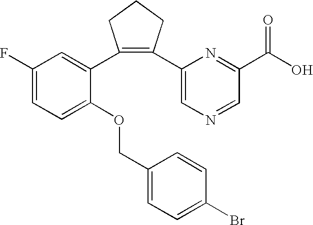 | 6-[2-(2-{[(4- Bromophenyl)methyl]oxy}-5- fluorophenyl)-1-cyclopenten- 1-yl]-2-pyrazinecarboxylic acid | Rt = 4.52, [MH⁺] 471 |
| 253 | 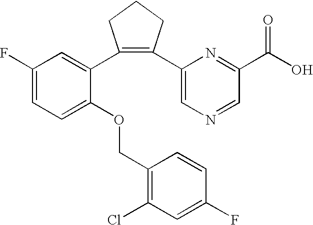 | 6-[2-(2-{[(2-Chloro-4- fluorophenyl)methyl]oxy}-5- fluorophenyl)-1-cyclopenten- 1-yl]-2-pyrazinecarboxylic acid | Rt = 4.51, [MH⁺] 443 |
| 254 | 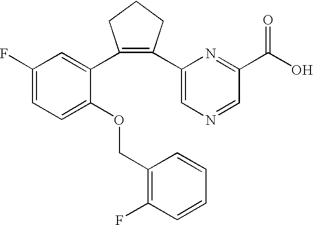 | 6-[2-(5-Fluoro-2-{[(2- fluorophenyl)methyl]oxy}phenyl)- 1-cyclopenten-1-yl]-2- pyrazinecarboxylic acid | Rt = 4.20, [MH⁺] 409 |
| 255 | 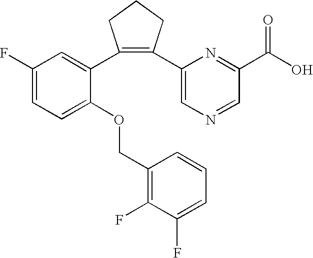 | 6-[2-(2-{[(2,3- Difluorophenyl)methyl]oxy)-5- fluorophenyl)-1-cyclopenten- 1-yl]-2-pyrazinecarboxylic acid | Rt = 4.21, [MH⁺] 427 |
| 256 | 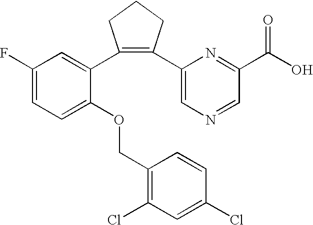 | 6-[2-(2-{[(2,4- Dichlorophenyl)methyl]oxy}- 5-fluorophenyl)-1- cyclopenten-1-yl]-2- pyrazinecarboxylic acid | Rt = 4.83, [MH⁺] 459 |
| 257 | 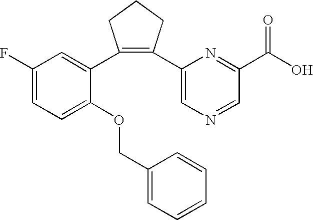 | 6-(2-{5-Fluoro-2- [(phenylmethyl)oxy]phenyl}-1- cyclopenten-1-yl)-2- pyrazinecarboxylic acid | Rt = 4.14, [MH⁺] 391 |
| 258 | 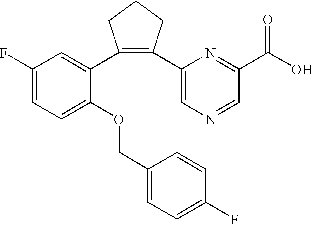 | 6-[2-(5-Fluoro-2-{[(4- fluorophenyl)methyl]oxy} phenyl}-1-cyclopenten-1-yl]-2- pyrazinecarboxylic acid | Rt = 4.12, [MH⁺] 409 |
| 259 | 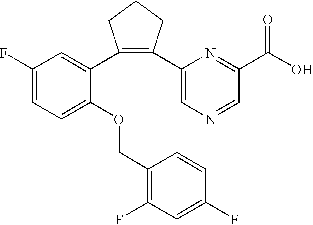 | 6-[2-(2-{[(2,4- Difluorophenyl)methyl]oxy}-5- fluorophenyl)-1-cyclopenten- 1-yl]-2-pyrazinecarboxylic acid | Rt = 4.15, [MH⁺] 427 |
| 260 | 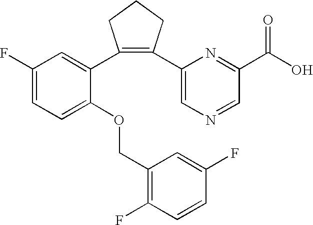 | 6-[2-(2-{[(2,5- Difluorophenyl)methyl]oxy}-5- fluorophenyl)-1-cyclopenten- 1-yl]-2-pyrazinecarboxylic acid | Rt = 4.13, [MH⁺] 427 |
| 261 | 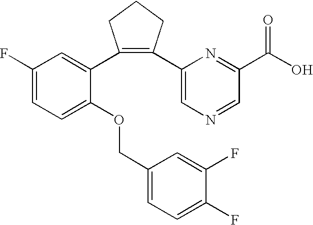 | 6-[2-(2-{[(3,4- Difluorophenyl)methyl]oxy}-5- fluorophenyl)-1-cyclopenten- 1-yl]-2-pyrazinecarboxylic acid | Rt = 4.14, [MH⁺] 427 |
| 262 | 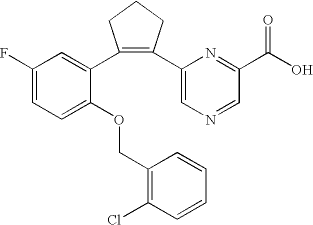 | 6-[2-(2-{[(2- Chlorophenyl)methyl]oxy}-5- fluorophenyl)-1-cyclopenten- 1-yl]-2-pyrazinecarboxylic acid | Rt = 4.15, [MH⁺] 425 |
| 263 | 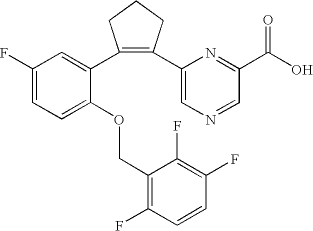 | 6-[2-(5-Fluoro-2-{[(2,3,6- trifluorophenyl)methyl]oxy} phenyl)-1-cyclopenten-1-yl]-2- pyrazinecarboxylic acid | Rt = 4.03, [MH⁺] 445 |
| 264 | 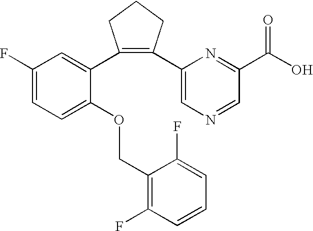 | 6-[2-(2-{[(2,6- Difluorophenyl)methyl]oxy}-5- fluorophenyl)-1-cyclopenten- 1-yl]-2-pyrazinecarboxylic acid | Rt = 5.15, [MH⁺] 427 |
| 265 | 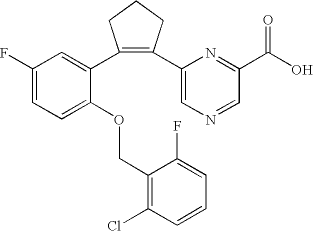 | 6-[2-(2-{[(2-Chloro-6- fluorophenyl)methyl]oxy}-5- fluorophenyl)-1-cyclopenten- 1-yl]-2-pyrazinecarboxylic acid | Rt = 4.19, [MH⁺] 443 |
| 266 | 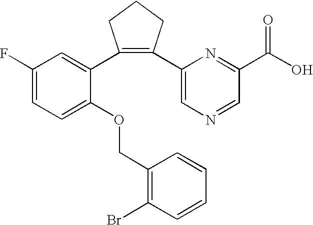 | 6-[2-(2-{[(2- Bromophenyl)methyl]oxy}-5- fluorophenyl)-1-cyclopenten- 1-yl]-2-pyrazinecarboxylic acid | Rt = 4.51, [MH⁺] 471 |
| 267 | 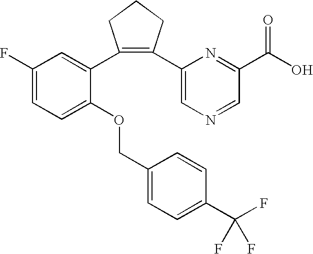 | 6-{2-[5-Fluoro-2-({[4- (trifluoromethyl)phenyl]methyl} oxy)phenyl]-1-cyclopenten-1- yl}-2-pyrazinecarboxylic acid | Rt = 4.25, [MH⁺] 459 |

### Example 268

### 5-[2-(5-Bromo-2{[(2,4-difluorophenyl)methyl]oxy}phenyl)-1-cyclopenten-1-yl]-3-pyridazinecarboxylic acid

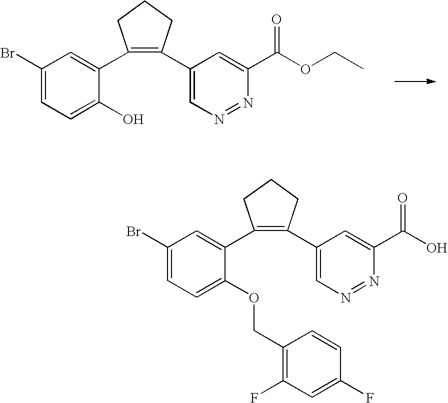

Ethyl 5-[2-(5-bromo-2-hydroxyphenyl)-1-cyclopenten-1-yl]-3-pyridazinecarboxylate (130 mg. 0.333 mmol) in dimethylformamide (4 ml) was treated with 2,4-difluorobenzyl bromide (80 mg, 0.386 mmol) and potassium carbonate (200 mg, 1.45 mmol). The reaction mixture was then stirred at room temperature for 5 hours, filtered through celite, and washed with ethyl acetate (3×15 ml). The filtrate was then washed with brine (2×50 ml), dried over magnesium sulphate and chromatographed, eluting with 1:1 diethyl ether/isohexane. The product was dissolved in 2M sodium hydroxide (2 ml) and methanol (3 ml) and heated with stirring for one hour at 70° C. The mixture was evaporated to ˜1 ml, diluted to 10 ml with water and treated with 2M hydrochloric acid (1.8 ml) and a couple of drops of acetic acid. The mixture was extracted with ethyl acetate (3×10 ml), dried over magnesium sulphate, filtered and evaporated to give the title compound (120 mg, 75% yield)

LC/MS Rt=4.25 min [MH⁺] 489

The following Examples were prepared by the procedure used for 5-[2-(5-bromo-2-{[(2,4-difluorophenyl)methyl]oxy}phenyl)-1-cyclopenten-1-yl]-3-pyridazinecarboxylic acid:

| Example | Structure | Name | ¹H NMR/LCMS |
|---|---|---|---|
| 269 | 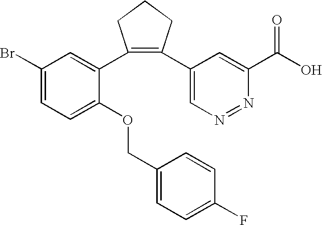 | 5-[2-(5-Bromo-2-{[(4- fluorophenyl)methyl]oxy} phenyl)-1-cyclopenten-1- yl]-3-pyridazinecarboxylic acid | ¹H NMR (CD₃OD) 2.05-2.15(2H, m) 2.85- 2.97(4H, m) 4.84(2H, s) 6.93-7.04(3H, m) 7.06- 7.13(2H, m) 7.25-7.29(1H, d) 7.40-7.45(1H, dd) 7.83(1H, s) 8.69(1H, s) |
| 270 | 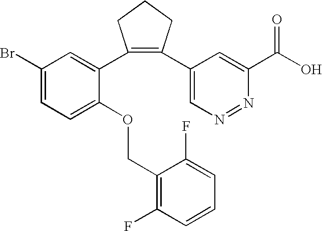 | 5-[2-(5-Bromo-2-{[(2-6- difluorophenyl)methyl]oxy} phenyl)-1-cyclopenten-1- yl]-3-pyridazinecarboxylic acid | Rt = 4.19, [MH⁺] 489 |
| 271 | 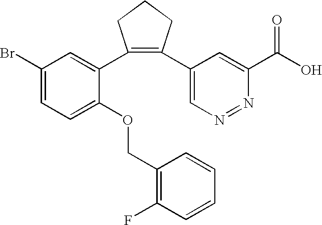 | 5-[2-(5-Bromo-2-{[(2- fluorophenyl)methyl]oxy} phenyl-1-cyclopenten-1-yl]- 3-pyridazinecarboxylic acid | Rt = 4.29, [MH⁺] 471 |
| 272 | 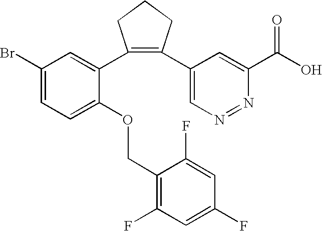 | 5-[2-(5-Bromo-2-{[(2,4,6- trifluorophenyl)methyl]oxy} phenyl)-1-cyclopenten-1- yl]-3-pyridazinecarboxylic acid | Rt = 4.19, [MH⁺] 535 |
| 273 | 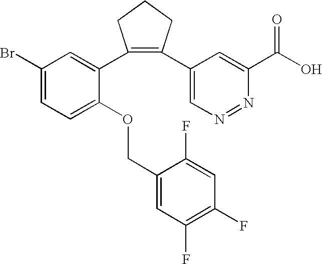 | 5-[2-(5-Bromo-2-{[(2,4,5- trifluorophenyl)methyl]oxy} phenyl)-1-cyclopenten-1- yl]-3-pyridazinecarboxylic acid | Rt = 4.30, [MH⁺] 507 |
| 274 | 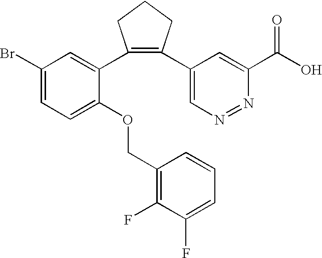 | 5-[2-(5-Bromo-2-{[2,3-di- fluorophenyl)methyl[oxy} phenyl)-1-cyclopenten-1- yl]-3-pyridazinecaboxylic acid | Rt = 4.27, [MH⁺] 489 |
| 275 | 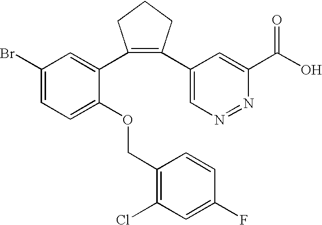 | 5-[2-(5-Bromo-2-{[(2- chloro-4-fluorophenyl) methyl]oxy}phenyl)-1- cyclopenten-1-yl]-3- pyridazinecarboxylic acid | Rt = 4.61, [MH⁺] 505 |
| 276 | 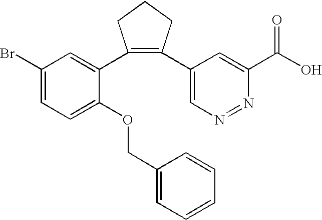 | 5-[2-(5-Bromo-2- [(phenylmethyl)oxy]phenyl)- 1-cyclopenten-1-yl]-3- pyridazinecarboxylic acid | Rt = 4.39, [MH⁺] 453 |

The following Examples were prepared by Standard Hydrolysis Procedure B:

| Example | Structure | Name | Data |
|---|---|---|---|
| 277 | 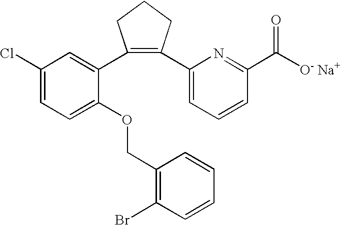 | Sodium 6-[2-(2-{[(2- bromophenyl)methyl] oxy}-5-chlorophenyl)-1- cyclopenten-1-yl]-2- pyridinecarboxylate | ¹H NMR (DMSO) δ: 1.91-1.97 (2H, m), 2.79- 2.83 (2H, m), 2.93-2.98 (2H, m), 5.12 (2H, s), 6.63 (1H, d), 6.96 (1H, s), 7.12 (1H, d), 7.26- 7.38 (5H, m), 7.50 (1H, d), 7.64 (1H, d). |
| 278 | 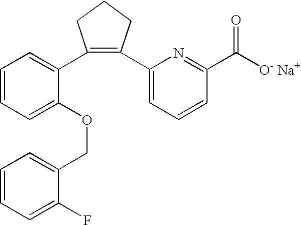 | Sodium 6-[2-(2-{[(2- fluorophenyl)methyl] oxy}phenyl)-1- cyclopenten-1-yl]-2- pyridinecarboxylate | ¹H NMR (CDCl₃) δ: 1.91-1.98 (2H, m), 2.95- 2.98 (2H, m), 5.10 (2H, s), 6.66-6.72 (2H, m), 6.78- 6.80 (1H, m), 7.03 (1H, d), 7.08-7.16 (3H, m), 7.29- 7.32 (2H, m), 7.38-7.41 (1H, m), 7.60 (1H, d). LC/MS: Rt = 3.38 min, [M + H] 390. |
| 279 | 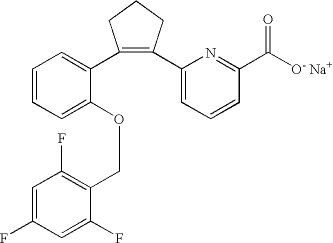 | Sodium 6-[2-(2- {[(2,4,6- trifluorophenyl)methyl] oxy}phenyl)-1- cyclopenten-1-yl]-2- pyridinecarboxylate | ¹H NMR (DMSO, 50° C.) δ: 1.85-1.96 (2H, m), 2.67-2.93 (4H, m), 5.05 (2H, s), 6.69-6.75 (3H, m), 6.95- 7.00 (2H, m), 7.09 (1H, d), 7.14-7.18 (1H, m), 7.35- 7.40 (1H, m), 7.64 (1H, d). LC/MS: Rt = 3.40 min, [M + H] 426. |
| 280 | 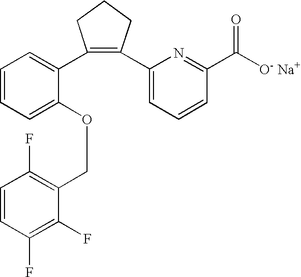 | Sodium 6-[2-(2- {[(2,3,6- trifluorophenyl)methyl] oxy}phenyl)-1- cyclopenten-1-yl]-2- pyridinecarboxylate | ¹H NMR (DMSO) δ: 1.86-1.94 (2H, m), 2.70- 2.73 (2H, m), 2.88-2.92 (2H, m), 5.24 (2H, s), 6.57 (1H, d), 6.85-6.86 (2H, m), 7.20-7.29 (3H, m), 7.32- 7.35 (1H, m), 7.51-7.58 (1H, m), 7.62 (1H, d). LC/MS: Rt = 3.38 min, [M + H] 426. |
| 281 | 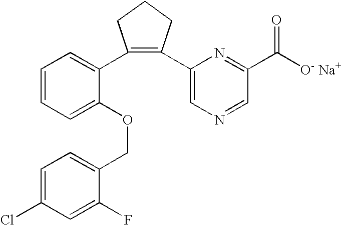 | Sodium 6-[2-(2-{[(4- chloro-2- fluorophenyl)methyl] oxy}phenyl)-1- cyclopenten-1-yl]-2- pyrazinecarboxylate | ¹H NMR (DMSO) δ: 1.94-2.01 (2H, m), 2.80- 2.85 (2H, m), 2.95-2.98 (2H, m), 5.12 (2H, s), 6.89- 6.94 (1H, m), 6.98 (1H, dd), 7.18 (1H, d), 7.27-7.30 (2H, m), 7.44 (1H, dd), 7.74 (1H, s), 8.55 (1H, s). LC/MS: Rt = 4.48 min, [M − H] 423, 425. |
| 282 | 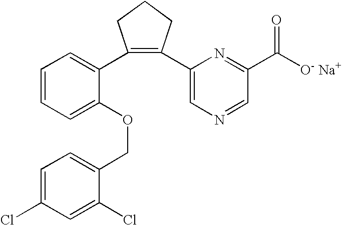 | Sodium 6-[2-(2-{[(2,4- dichlorophenyl)methyl] oxy}phenyl)-1- cyclopenten-1-yl]-2- pyrazinecarboxylate | ¹H NMR (DMSO) δ: 1.95-2.03 (2H, m), 2.83- 2.87 (2H, m), 2.95-2.99 (2H, m), 5.12 (2H, s), 6.91- 6.94 (1H, m), 7.01 (1H, dd), 7.15 (1H, d), 7.28-7.33 (2H, m), 7.43 (1H, dd), 7.63 (1H, d), 7.77 (1H, s), 8.56 (1H, s). LC/MS: Rt = 4.98 min, [M − H] 439, 441. |
| 283 | 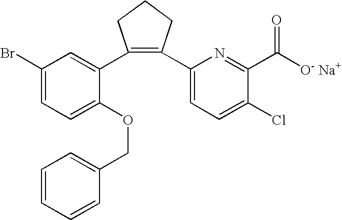 | 6-(2-{5-Bromo-2- [(phenylmethyl)oxy] phenyl}-1-cyclopenten-1- yl)-3-chloro-2- pyridinecarboxylic acid sodium salt | LC/MS: Rt = 4.43, [MH+]486.3 |
| 284 | 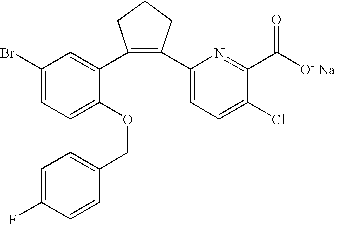 | 6-[2-(5-Bromo-2-{[(4- fluorophenyl)methyl] oxy}phenyl)-1- cyclopenten-1-yl]-3- chloro-2- pyridinecarboxylic acid sodium salt | LC/MS: Rt = 4.38 [MH+] 504.3 |
| 285 | 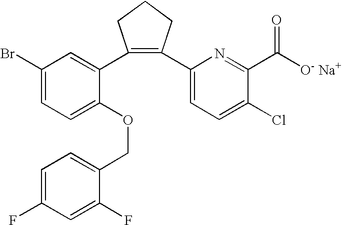 | Sodium 6-[2-(5-bromo- 2-{[(2-4- difluorophenyl)methyl] oxy}phenyl)-1- cyclopenten-1-yl]-3- chloro-2- pyridinecarboxylate | LC/MS: Rt = 4.42 [MH+] 522.3 |
| 286 | 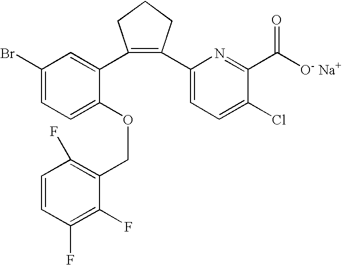 | Sodium 6-[2-(5-bromo- 2-{[(2,3,6- trifluorophenyl)methyl] oxy}phenyl)-1- cyclopenten-1-yl]-3- chloro-2- pyridinecarboxylate | LC/MS: Rt = 4.27 [MH+] 540.3 |
| 287 | 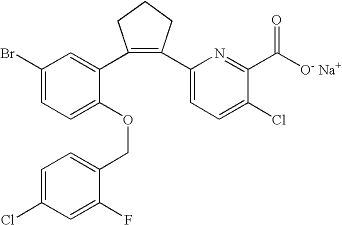 | Sodium 6-[2-(5-bromo- 2-{[(4-chloro-2- fluorophenyl)methyl] oxy}phenyl)-1- cyclopenten-1-yl]-3- chloro-2- pyridinecarboxylate | LC/MS: Rt = 4.65 [MH+] 538.3 |
| 288 | 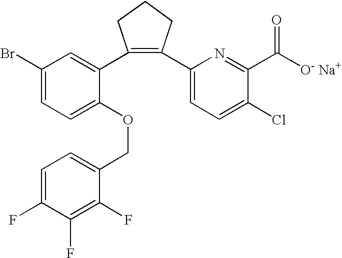 | Sodium 6-[2-(5-bromo- 2-{[(2,3,4- trifluorophenyl)methyl] oxy}phenyl)-1- cyclopenten-1-yl]-3- chloro-2- pyridinecarboxylate | LC/MS: Rt = 4.44 [MH+] 540.3 |
| 289 | 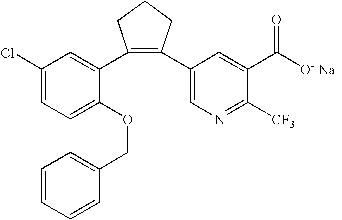 | Sodium 5-(2-{5-chloro- 2-[(phenylmethyl)oxy] phenyl}-1-cyclopenten-1- yl)-2-(trifluoromethyl)- 3-pyridinecarboxylate | LC/MS: Rt = 3.37, [MH+] 474.4, 476.3 |
| 290 | 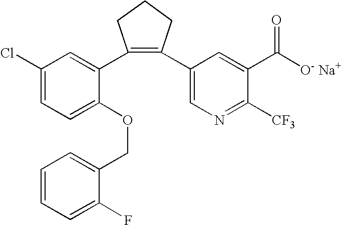 | Sodium 5-[2-(5-chloro- 2-{[(2-fluorophenyl) methyl]oxy}phenyl)-1- cyclopenten-1-yl]-2- (trifluoromethyl)-3- pyridinecarboxylate | LC/MS: Rt = 4.30 [MH+] 492.3, 494.3 |
| 291 | 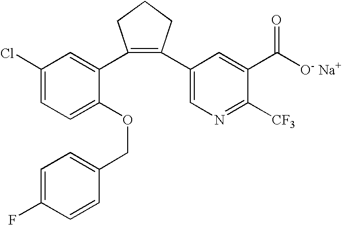 | Sodium 5-[2-(5-chloro- 2-{[(4-fluorophenyl) methyl]oxy}phenyl)-1- cyclopenten-1-yl]-2- (trifluoromethyl)-3- pyridinecarboxylate | LC/MS: Rt = 3.83 [MH+] 492.3, 494.3 |
| 292 | 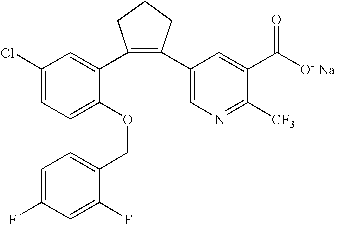 | Sodium 5-[2-(5-chloro- 2-{[(2,4- difluorophenyl)methyl] oxy}phenyl)-1- cyclopenten-1-yl]-2- (trifluoromethyl)-3- pyridinecarboxylate | LC/MS: Rt = 4.02 [MH+] 510.3, 512.3 |
| 293 | 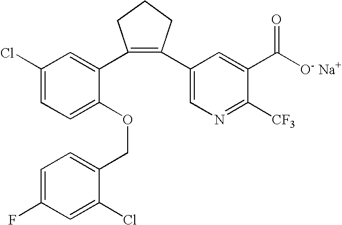 | Sodium 5-[2-(5-chloro- 2-{[(2-chloro-4- fluorophenyl)methyl] oxy}phenyl)-1- cyclopenten-1-yl]-2- (trifluoromethyl)-3- pyridinecarboxylate | LC/MS: Rt = 4.25 [MH+] 526.3 |
| 294 | 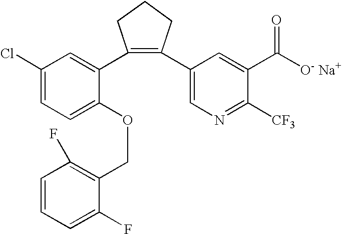 | Sodium 5-[2-(5-chloro- 2-{[(2,6- difluorophenyl)methyl] oxy}phenyl)-1- cyclopenten-1-yl]-2- (trifluoromethyl)-3- pyridinecarboxylate | LC/MS: Rt = 4.08 [MH+] 510.3, 512.3 |
| 295 | 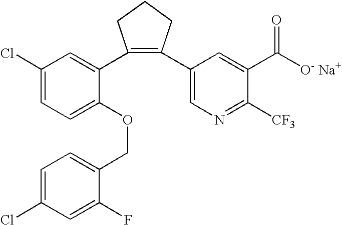 | Sodium 5-[2-(5-chloro- 2-{[(4-chloro-2- fluorophenyl)methyl] oxy}phenyl)-1- cyclopenten-1-yl]-2- (trifluoromethyl)-3- pyridinecarboxylate | LC/MS: Rt = 4.37 [MH+] 526.3 |
| 296 | 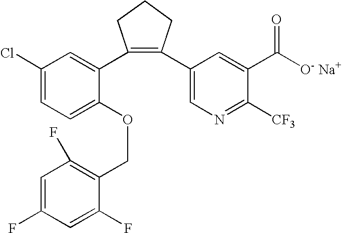 | Sodium 5-[2-(5-chloro- 2-{[(2,4,6- trifluorophenyl)methyl] oxy}phenyl)-1- cyclopenten-1-yl]-2- (trifluoromethyl)-3- pyridinecarboxylate | LC/MS: Rt = 4.15 [MH+] 528.3, 530.3 |
| 297 | 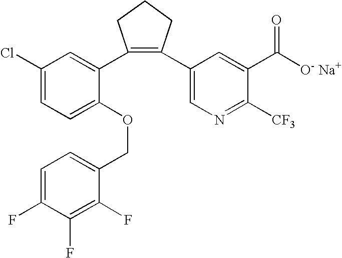 | Sodium 5-[2-(5-chloro- 2-{[(2,4,5- trifluorophenyl)methyl] oxy}phenyl)-1- cyclopenten-1-yl]-2- (trifluoromethyl)-3- pyridinecarboxylate | LC/MS: Rt = 4.24 [MH+] 528.3, 530.3 |
| 298 | 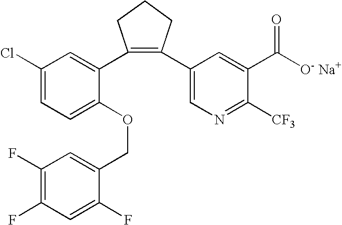 | 5-[2-(5-Chloro-2- {[(2,4,5-trifluorophenyl) methyl]oxy}phenyl)-1- cyclopenten-1-yl]-2- (trifluoromethyl)-3- pyridinecarboxylic acid sodium salt | LC/MS: Rt = 4.17 [MH+] 528.3, 530.3 |
| 299 | 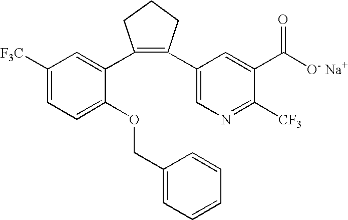 | Sodium 5-{2-[2- [(phenylmethyl)oxy]-5- (trifluoromethyl)phenyl]- 1-cyclopenten-1-yl}-2- (trifluoromethyl)-3- pyridinecarboxylate. | LC/MS: Rt = 3.84 min. [M + H] = 508 |
| 300 | 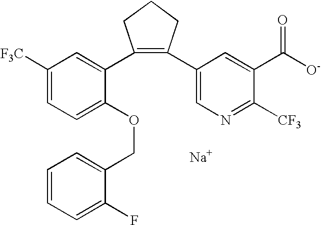 | 5-{2-[2-{[(2- fluorophenyl)methyl] oxy}-5-(trifluoromethyl) phenyl]-1-cyclopenten-1- yl}-2-(trifluoromethyl)- 3-pyridinecarboxylic acid Sodium salt | Rt = 4.23 min [M + H]526 |
| 301 | 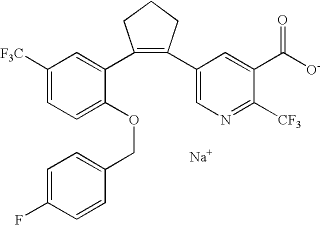 | 5-{2-[2-{[(4- fluorophenyl)methyl] oxy}-5- (trifluoromethyl)phenyl]- 1-cyclopenten-1-yl}-2- (trifluoromethyl)-3- pyridinecarboxylic acid sodium salt | Rt = 3.77 min [M + H] 526 |
| 302 | 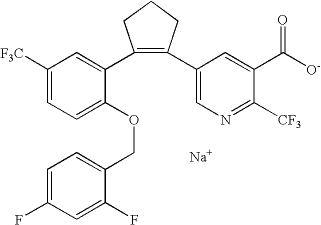 | 5-{2-[2-{[(2,4- difluorophenyl)methyl] oxy}-5- (trifluoromethyl)phenyl]- 1-cyclopenten-1-yl}-2- (trifluoromethyl)-3- pyridinecarboxylic acid sodium salt | Rt = 4.24 min [M + H] 544 |
| 303 | 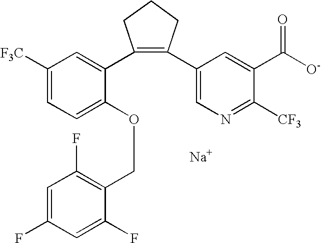 | 2-(trifluoromethyl)-5-[2- (5-(trifluoromethyl)-2- {[(2,4,6- trifluorophenyl)methyl] oxy}phenyl)-1- cyclopenten-1-yl]-3- pyridinecarboxylic acid sodium salt | Rt = 4.16 min [M + H] 562 |
| 304 | 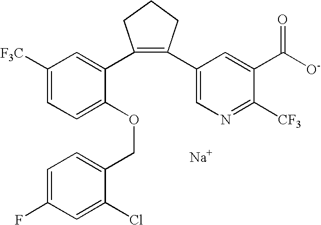 | 5-{2-[2-{[(2-chloro-4- fluorophenyl)methyl] oxy}-5- (trifluoromethyl)phenyl]- 1-cyclopenten-1-yl}-2- (trifluoromethyl)-3- pyridinecarboxylic acid sodium salt | Rt = 4.51 min [M + H] 560 (1Cl) |
| 305 | 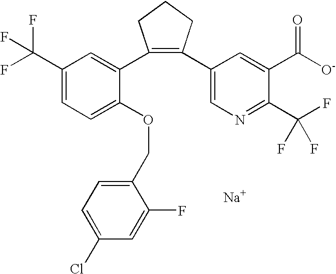 | Sodium 5-{2-[2-{[(4- chloro-2- fluorophenyl)methyl] oxy}-5- (trifluoromethyl)phenyl]- 1-cyclopenten-1-yl]-2- (trifluoromethyl)-3- pyridinecarboxylate | Rt = 4.53 min [M + H] 560 (1Cl) |
| 306 | 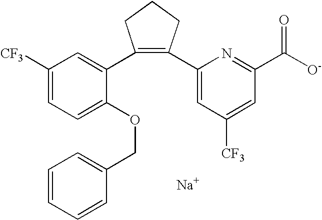 | sodium 6-{2-[2- [(phenylmethyl)oxy]-5- (trifluoromethyl)phenyl]- 1-cyclopenten-1-yl}-4- (trifluoromethyl)-2- pyridinecarboxylate | Rt = 3.77 min (M + H] 508 |
| 307 | 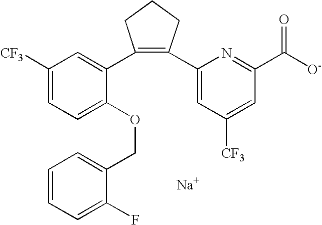 | sodium 6-{2-[2-{[(2- fluorophenyl)methyl] oxy}-5- (trifluoromethyl)phenyl]- 1-cyclopenten-1-yl}-4- (trifluoromethyl)-2- pyridinecarboxylate | Rt = 4.46 min (M + H] 526 |
| 308 | 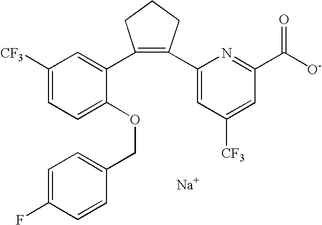 | sodium 6-{2-[2-{[(4- fluorophenyl)methyl] oxy}-5- (trifluoromethyl)phenyl]- 1-cyclopenten-1-yl)-4- (trifluoromethyl)-2- pyridinecarboxylate | Rt = 3.74 min (M + H] 526 |
| 309 | 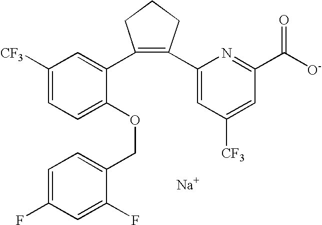 | sodium 6-{2-[2-{[(2,4- difluorophenyl)methyl] oxy}-5- (trifluoromethyl)phenyl]- 1-cyclopenten-1-yl}-4- (trifluoromethyl)-2- pyridinecarboxylate | Rt = 4.43 min [M + H] 544 |
| 310 | 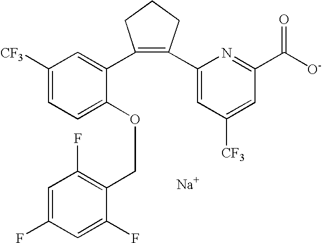 | sodium 4- (trifluoromethyl)-6-[2- (5-(trifluoromethyl)-2- {[(2,4,6- trifluorophenyl)methyl] oxy}phenyl)-1- cyclopenten-1-yl]-2- pyridinecarboxylate | Rt = 4.36 min [M + H] 562 |
| 311 | 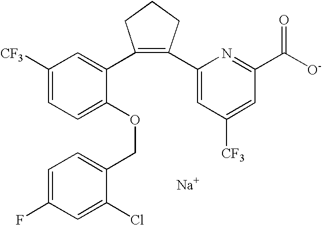 | sodium 6-{2-[2-{[(2- chloro-4-fluorophenyl) methyl]oxy}-5- (trifluoromethyl)phenyl]- 1-cyclopenten-1-yl}-4- (trifluoromethyl)-2- pyridinecarboxylate | Rt = 4.65 min [M + H] 560 (1Cl) |
| 312 | 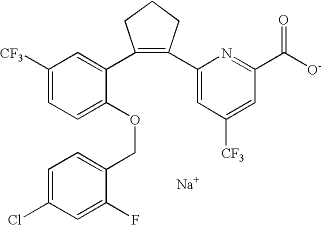 | sodium 6-{2-[2-{[(4- chloro-2- fluorophenyl)methyl] oxy}-5- (trifluoromethyl)phenyl]- 1-cyclopenten-1-yl}-4- (trifluoromethyl)-2- pyridinecarboxylate | Rt = 4.23 min [M + H] 560 (1Cl) |
| 313 | 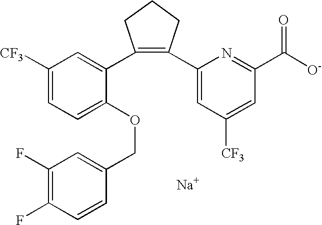 | sodium 6-{2-[2-{[(3,4- difluorophenyl)methyl] oxy}-5- (trifluoromethyl)phenyl]- 1-cyclopenten-1-yl}-4- (trifluoromethyl)-2- pyridinecarboxylate | Rt = 4.38 min [M + H] 544 |
| 314 | 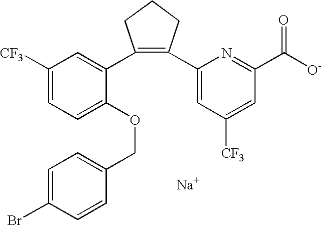 | sodium 6-{2-[2-{[(4- bromophenyl)methyl] oxy}-5- (trifluoromethyl)phenyl]- 1-cyclopenten-1-yl}-4- (trifluoromethyl)-2- pyridinecarboxylate | Rt = 4.50 min [M + H] 586, 588 (1Br) |
| 315 | 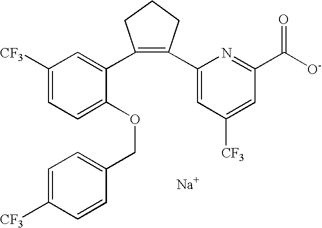 | sodium 4-(trifluoro- methyl)-6-{2-[5- (trifluoromethyl)-2- ({[4-(trifluoromethyl) phenyl]methyl}oxy) phenyl]-1-cyclopenten- 1-yl}-2- pyridinecarboxylate | Rt = 4.55 min [M + H] 576 |
| 316 | 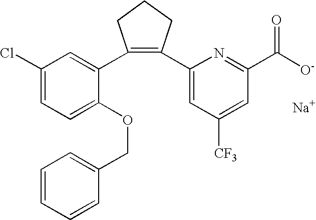 | Sodium 6-(2-{5-chloro- 2-[(phenylmethyl)oxy] phenyl}-1-cyclopenten-1- yl)-4-(trifluoromethyl)- 2-pyridinecarboxylate | [M + H] 474.4, 476.4 Rt = 4.26 min |
| 317 | 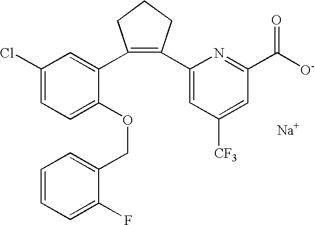 | Sodium 6-[2-(5-chloro- 2-{[(2-fluorophenyl) methyl]oxy}phenyl)-1- cyclopenten-1-yl]-4- (trifluoromethyl)-2- pyridinecarboxylate | [M + H] 492.4, 494.4 Rt = 3.85 min |
| 318 | 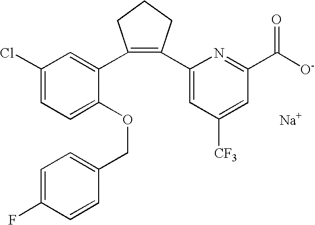 | Sodium 6-[2-(5-chloro- 2-{[(4-fluorophenyl) methyl]oxy}phenyl)-1- cyclopenten-1-yl]-4- (trifluoromethyl)-2- pyridinecarboxylate | [M + H] 492.3, 494.3 Rt = 4.53 min |
| 319 | 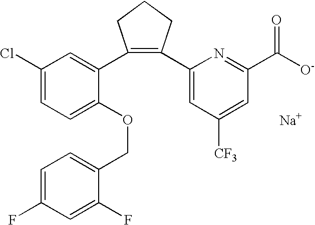 | Sodium 6-[2-(5-chloro- 2-{[(2,4-difluorophenyl) methyl]oxy}phenyl)-1- cyclopenten-1-yl]-4- (trifluoromethyl)-2- pyridinecarboxylate | [M + H] 510.3, 512.3 Rt = 4.50 min |
| 320 | 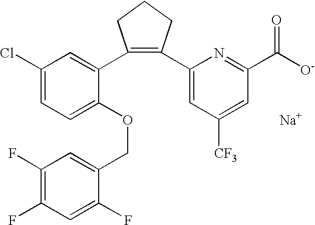 | Sodium 6-[2-(5-chloro- 2-{[(2,4,5- trifluorophenyl)methyl] oxy}phenyl)-1- cyclopenten-1-yl]-4- (trifluoromethyl)-2- pyridinecarboxylate | [M + H] 528.3, 530.3 Rt = 4.54 min |
| 321 | 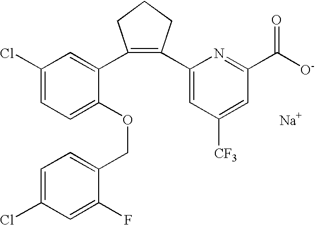 | Sodium 6-[2-(5-chloro- 2-{[(4-chloro-2- fluorophenyl)methyl] oxy}phenyl)-1- cyclopenten-1-yl]-4- (trifluoromethyl)-2- pyridinecarboxylate | [M + H] 526.3, 528.3 Rt = 4.78 min |
| 322 | 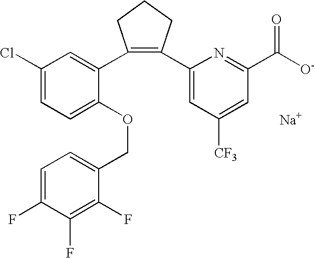 | Sodium 6-[2-(5-chloro- 2-{[(2,3,4- trifluorophenyl)methyl] oxy}phenyl)-1- cyclopenten-1-yl]-4- (trifluoromethyl)-2- pyridinecarboxylate | [M + H] 528.3, 530.3 Rt = 4.58 min |
| 323 | 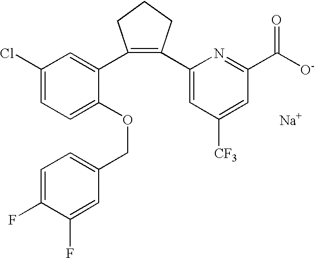 | Sodium 6-[2-(5-chloro- 2-{[(3,4-difluorophenyl) methyl]oxy}phenyl)-1- cyclopenten-1-yl]-4- (trifluoromethyl)-2- pyridinecarboxylate | [M + H] 510.3, 512.3 Rt = 4.49 min |
| 324 | 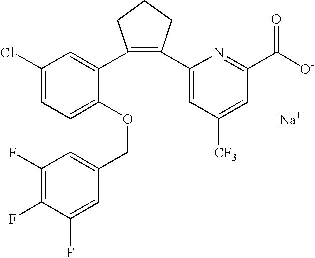 | Sodium 6-[2-(5-chloro- 2-{[(3,4,5-trifluoro- phenyl)methyl] oxy}phenyl)-1- cyclopenten-1-yl]-4- (trifluoromethyl)-2- pyridinecarboxylate | [M + H] 528.3, 530.3 Rt = 3.74 min |
| 325 | 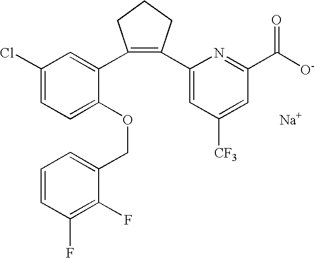 | Sodium 6-[2-(5-chloro- 2-{[(2,3-difluorophenyl) methyl]oxy}phenyl)-1- cyclopenten-1-yl]-4- (trifluoromethyl)-2- pyridinecarboxylate | [M + H] 510.3, 512.3 Rt = 4.55 min |
| 326 | 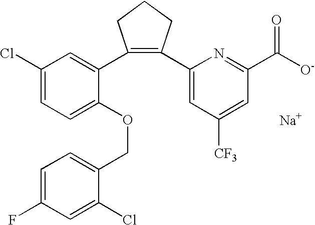 | Sodium 6-[2-(5-chloro- 2-{[(2-chloro-4- fluorophenyl)methyl] oxy}phenyl)-1- cyclopenten-1-yl]-4- (trifluoromethyl)-2- pyridinecarboxylate | [M + H] 526.3, 528.3 Rt = 4.82 min |
| 327 | 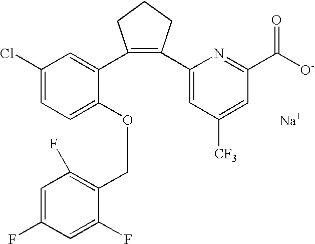 | Sodium 6-[2-(5-chloro- 2-{[(2,4,6-trifluoro- phenyl)methyl]oxy} phenyl)-1-cyclopenten- 1-yl]-4-(trifluoro- methyl)-2- pyridinecarboxylate | [M + H] 528.3, 530.3 Rt = 3.87 min |
| 328 | 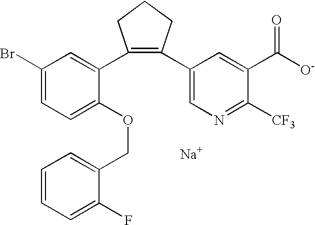 | sodium 5-[2-(5-bromo- 2-{[(2-fluorophenyl) methyl]oxy}phenyl)-1- cyclopenten-1-yl]-2- (trifluoromethyl)-3- pyridinecarboxylate | Rt = 4.0 min [M + H] 536, 539 (1Br) |
| 329 | 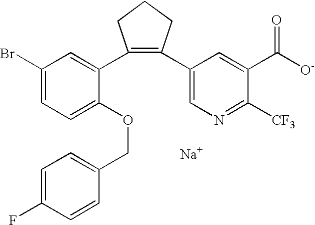 | sodium 5-[2-(5-bromo- 2-{[(4-fluorophenyl) methyl]oxy}phenyl)-1- cyclopenten-1-yl]-2- (trifluoromethyl)-3- pyridinecarboxylate | Rt = 4.0 min [M + H] 536, 539 (1Br) |
| 330 | 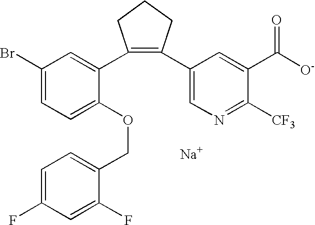 | sodium 5-[2-(5-bromo- 2-{[(2,4-difluorophenyl) methyl]oxy}phenyl)-1- cyclopenten-1-yl]-2- (trifluoromethyl)-3- pyridinecarboxylate | Rt = 4.0 min [M + H] 556, 557 (1Br) |
| 331 | 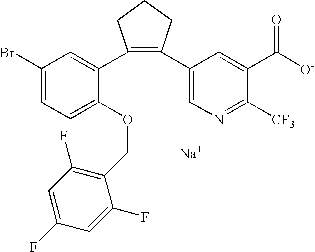 | sodium 5-[2-(5-bromo- 2-{[2,4,6-trifluoro- phenyl)methyl]oxy} phenyl)-1-cyclopenten-1- yl]-2-(trifluoromethyl)-3- pyridinecarboxylate | Rt = 3.9 min [M + H] 574, 575 (1Br) |
| 332 | 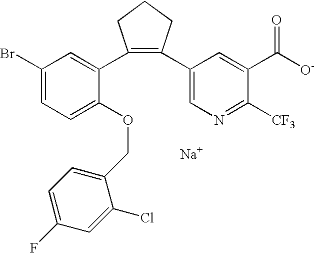 | sodium 5-[2-(5-bromo- 2-{[(2-chloro-4- fluorophenyl)methyl] oxy}phenyl)-1- cyclopenten-1-yl]-2- (trifluoromethyl)-3- pyridinecarboxylate | Rt = 4.3 min [M + H] 572, 573 (1Br) |
| 333 | 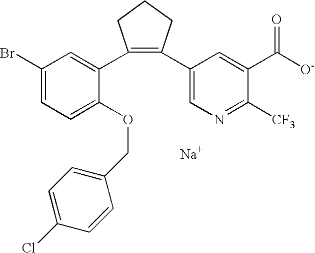 | sodium 5-[2-(5-bromo- 2-{[(4-chlorophenyl) methyl]oxy}phenyl)-1- cyclopenten-1-yl]-2- (trifluoromethyl)-3- pyridinecarboxylate | Rt = 4.2 min [M + H] 554, 556 (1 Br) |
| 334 | 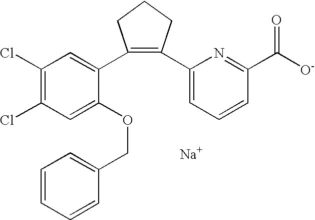 | sodium 6-(2-{4,5- dichloro-2-[(phenylmethyl) oxy]phenyl}-1-cyclo- penten-1-yl)-2- pyridinecarboxylate | Rt = 4.09 [MH+] 440.4, 442.4 |
| 335 | 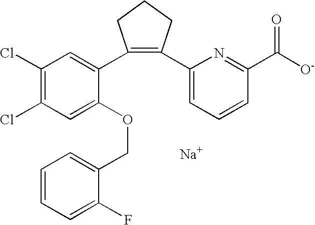 | sodium 6-[2-(4,5- dichloro-2-{[(2- fluorophenyl)methyl] oxy}phenyl)-1- cyclopenten-1-yl]-2- pyridinecarboxylate | Rt = 4.11 [MH+] 458.4, 460.4 |
| 336 | 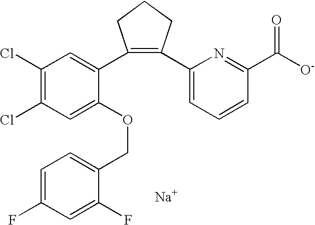 | sodium 6-[2-(4,5- dichloro-2-{[(2,4- difluorophenyl)methyl] oxy}phenyl)-1- cyclopenten-1-yl]-2- pyridinecarboxylate | Rt = 4.12 [MH+] 476.4, 478.4 |
| 337 | 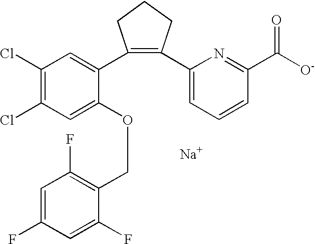 | sodium 6-[2-(4,5- dichloro-2-{[(2,4,6- trifluorophenyl)methyl] oxy)phenyl)-1- cyclopenten-1-yl]-2- pyridinecarboxylate | Rt = 4.14 [MH+] 494.4, 496.4 |
| 338 | 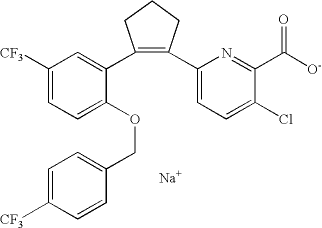 | sodium 3-chloro-6-{2- [5-(trifluoromethyl)-2-({[4- (trifluoromethyl)phenyl] methyl}oxy)phenyl]-1- cyclopenten-1-yl}-2- pyridinecarboxylate | Rt = 4.41 min [M + H] 542 (1Cl) |
| 339 | 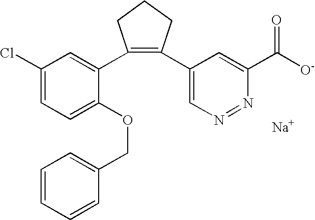 | 5-(2-{5-Chloro-2- [(phenylmethyl)oxy] phenyl]-1-cyclopenten-1- yl)-3-pyridazinecarboxylic acid | [M + H] 407.2, 409.2 Rt = 4.42 min |
| 340 | 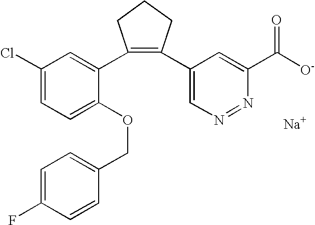 | 5-[2-(5-Chloro-2-{[(4- fluorophenyl)methyl] oxy}phenyl)-1- cyclopenten-1-yl]-3- pyridazinecarboxylic acid | [M + H] 425.1, 427.1 Rt = 4.30 min |
| 341 | 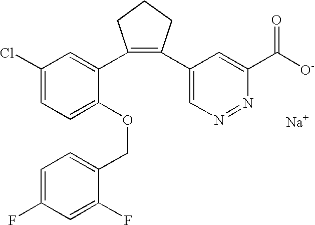 | 5-[2-(5-Chloro-2-{[(2,4- difluorophenyl)methyl] oxy}phenyl)-1- cyclopenten-1-yl]-3- pyridazinecarboxylic acid | [M + H] 443.1, 445.1 Rt = 4.30 min |
| 342 | 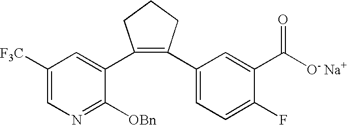 | Sodium 2-fluoro-5-{2- [2-[(phenylmethyl)oxy]- 5-(trifluoromethyl)-3- pyridinyl]-1- cyclopenten-1- yl}benzoate | LC/MS: Rt = 4.41 min. [MH⁺] 458 |
| 343 | 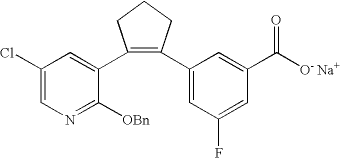 | Sodium 3-(2-{5-chloro- 2-[(phenylmethyl)oxy]- 3-pyridinyl}-1- cyclopenten-1-yl)-5- fluorobenzoate | LC/MS: Rt = 4.35 min. [MH⁺] 424, 426. |
| 344 | 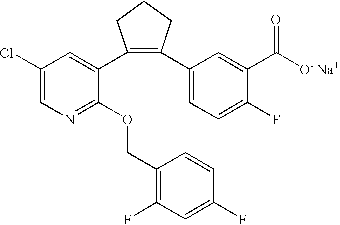 | Sodium 5-[2-(5-chloro- 2-{[(2,4-difluorophenyl) methyl]oxy}-3-pyridinyl)- 1-cyclopenten-1-yl]-2- fluorobenzoate | LC/MS: Rt = 4.42 min. [MH⁺] 460, 462. |
| 345 | 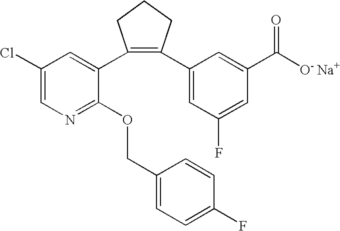 | Sodium 3-[2-(5-chloro- 2-{[(4-fluorophenyl) methyl]oxy}-3-pyridinyl)- 1-cyclopenten-1-yl]-5- fluorobenzoate | LC/MS: Rt = 4.36 min. [MH⁺] 442, 444. |
| 346 | 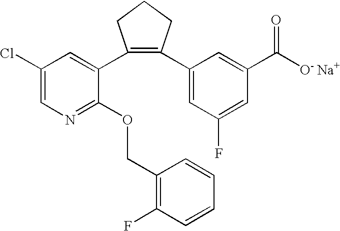 | Sodium 3-[2-(5-chloro- 2-{[(2-fluorophenyl) methyl]oxy)-3-pyridinyl)- 1-cyclopenten-1-yl]-5- fluorobenzoate | LC/MS: Rt = 4.41 min. [MH⁺] 442, 444. |
| 347 | 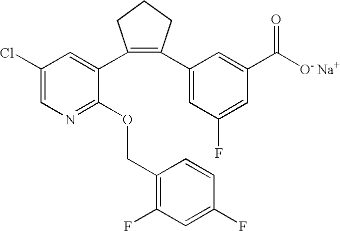 | Sodium 3-[2-(5-chloro- 2-{[(2,4-difluorophenyl) methyl]oxy}-3-pyridinyl)- 1-cyclopenten-1-yl]-5- fluorobenzoate | LC/MS: Rt = 4.42 min. [MH⁺] 460, 462. |
| 348 | 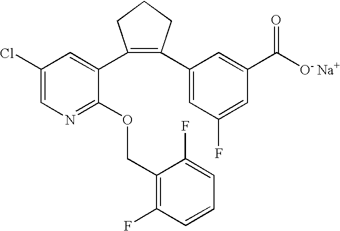 | Sodium 3-[2-(5-chloro- 2-{[(2,6-difluorophenyl) methyl]oxy}-3- pyridinyl)-1- cyclopenten-1-yl]-5- fluorobenzoate | LC/MS: Rt = 4.35 min. [MH⁺] 460, 462. |
| 349 | 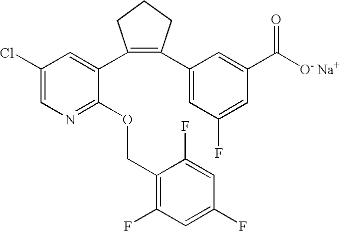 | Sodium 3-[2-(5-chloro- 2-{[(2,4,6- trifluorophenyl)methyl] oxy}-3-pyridinyl)-1- cyclopenten-1-yl]-5- fluorobenzoate | LC/MS: Rt = 4.39 min. [MH⁺] 478, 480. |
| 350 | 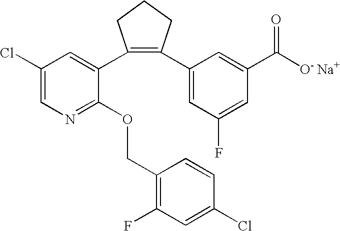 | Sodium 3-[2-(5-chloro- 2-{[(4-chloro-2- fluorophenyl)methyl] oxy}-3-pyridinyl)-1- cyclopenten-1-yl]-5- fluorobenzoate | LC/MS: Rt = 4.62 min. [MH⁺] 476, 477, 478, 479. |
| 351 | 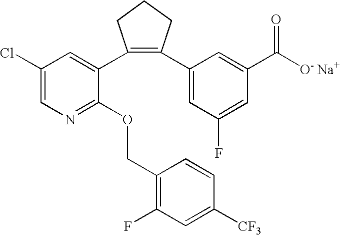 | Sodium 3-{2-[5-chloro- 2-({[2-fluoro-4- (trifluoromethyl)phenyl] methyl}oxy)-3-pyridinyl]- 1-cyclopenten-1-yl}-5- fluorobenzoate | LC/MS: Rt = 4.56 min. [MH⁺] 508, 510. |
| 352 | 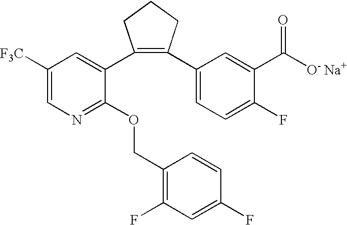 | Sodium 5-{2-[2-{[(2,4- difluorophenyl)methyl] oxy}-5-(trifluoromethyl)-3- pyridinyl]-1- cyclopenten-1-yl}-2- fluorobenzoate | LC/MS: Rt = 4.43 min. [MH⁺] 494. |
| 353 | 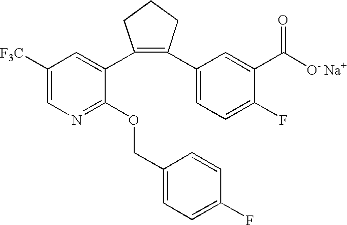 | Sodium 2-fluoro-5-{2- [2-{[(4-fluorophenyl) methyl]oxy}-5-(trifluoro- methyl)-3-pyridinyl]-1- cyclopenten-1-yl}benzoate | LC/MS: Rt = 4.30 min. [MH⁺] 476. |
| 354 | 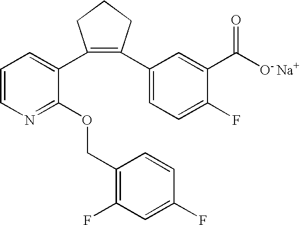 | Sodium 5-[2-(2-{[(2,4- difluorophenyl)methyl] oxy}-3-pyridinyl)-1- cyclopenten-1-yl]-2- fluorobenzoate | LC/MS: Rt = 4.20 min [MH⁺] 426. |
| 355 | 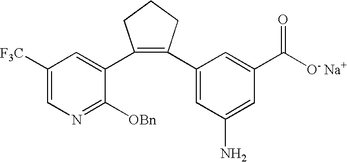 | Sodium 3-amino-5-{2- [2-[(phenylmethyl)oxy]- 5-(trifluoromethyl)-3- pyridinyl]-1- cyclopenten-1- yl}benzoate | ¹H NMR (MeOD) δ: 1.99-2.08 (2H, m), 2.80- 2.92 (4H, m), 5.35 (2H, s), 6.44 (1H, t), 7.15 (2H, dt), 7.23-7.35 (5H, m), 7.51 (1H, d), 8.27-8.29 (1H, m). LC/MS Rt = 3.71 min [MH⁺] 455. |
| 356 | 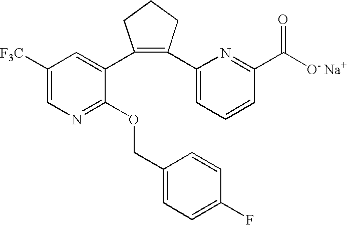 | Sodium 6-{2-[2-{[(4- fluorophenyl)methyl] oxy}-5-(trifluoromethyl)-3- pyridinyl]-1- cyclopenten-1-yl}-2- pyridinecarboxylate | ¹H NMR (MeOD) δ: 2.03-2.11 (2H, m), 2.86- 2.91 (2H, m), 3.08-3.13 (2H, m), 5.31 (2H, s), 6.79 (1H, d), 7.03 (2H, t), 7.26- 7.30 (2H, m), 7.44 (1H, t), 7.61 (1H, d), 7.72 (1H, d), 8.34 (1H, s). LC/MS Rt = 3.88 min [MH⁺] 459. |
| 357 | 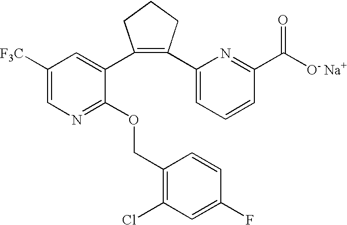 | Sodium 6-{2-[2-{[(2- chloro-4-fluorophenyl) methyl]oxy}-5-(trifluoro- methyl)-3-pyridinyl]-1- cyclopenten-1-yl}-2- pyridinecarboxylate | ¹H NMR (MeOD) δ: 2.03-2.11 (2H, m), 2.85- 2.90 (2H, m), 3.08-3.13 (2H, m), 5.38 (2H, s), 6.81 (1H, d), 7.15-7.20 (2H, m), 7.25 (1H, t), 7.46 (1H, t), 7.63 (1H, d), 7.72 (1H, d), 8.35 (1H, s). LC/MS Rt = 4.07 min [MH⁺] 493. |
| 358 | 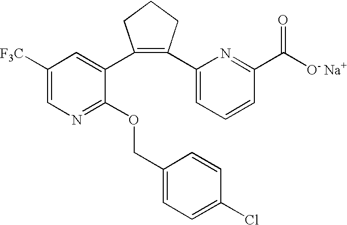 | Sodium 6-{2-[2-{[(4- chlorophenyl)methyl] oxy}-5-(trifluoromethyl)- 3-pyridinyl]-1- cyclopenten-1-yl}-2- pyridinecarboxylate | ¹H NMR (MeOD) δ: 2.04-2.12 (2H, m), 2.87- 2.92 (2H, m), 3.09-3.14 (2H, m), 5.31 (2H, s), 6.80 (1H, d), 7.23 (2H, d), 7.29-7.34 (2H, m), 7.45 (1H, t), 7.63 (1H, d), 7.72 (1H, d), 8.34 (1H, s). LC/MS Rt = 4.04 min [MH⁺] 475. |
| 359 | 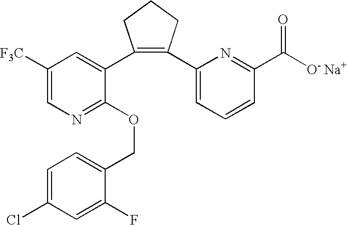 | Sodium 6-{2-[2-{[(4- chloro-2-fluorophenyl) methyl]oxy}-5-(trifluoro- methyl)-3-pyridinyl]-1- cyclopenten-1-yl}-2- pyridinecarboxylate | LC/MS: Rt = 4.07 min [MH⁺] 493 |
| 360 | 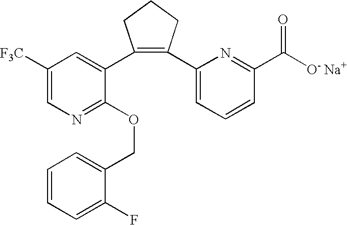 | Sodium 6-{2-[2-{[(2- fluorophenyl)methyl] oxy}-5-(trifluoromethyl)-3- pyridinyl]-1- cyclopenten-1-yl}-2- pyridinecarboxylate | LC/MS: Rt = 3.88 min [MH⁺] 459 |
| 361 | 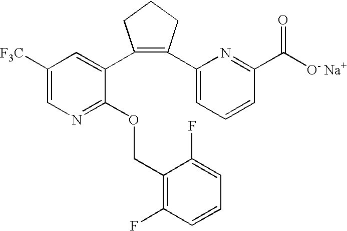 | Sodium 6-{2-[2-{[(2,6- difluorophenyl)methyl] oxy}-5-(trifluoromethyl)-3- pyridinyl]-1- cyclopenten-1-yl}-2- pyridinecarboxylate | LC/MS: Rt = 3.85 min [MH⁺] 477 |
| 362 | 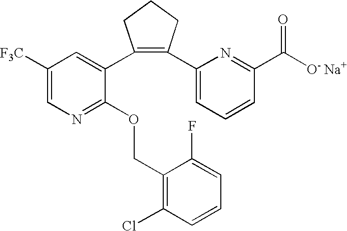 | Sodium 6-{2-[2-{[(2- chloro-6-fluorophenyl) methyl]oxy}-5-(trifluoro- methyl)-3-pyridinyl]-1- cyclopenten-1-yl}-2- pyridinecarboxylate | LC/MS: Rt = 3.98 min [MH⁺] 493 |
| 363 | 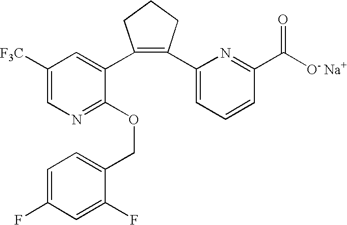 | Sodium 6-{2-[2-{[(2,4- difluorophenyl)methyl] oxy}-5-(trifluoromethyl)- 3-pyridinyl]-1- cyclopenten-1-yl}-2- pyridinecarboxylate | LC/MS: Rt = 3.91 min [MH⁺] 477 |
| 364 | 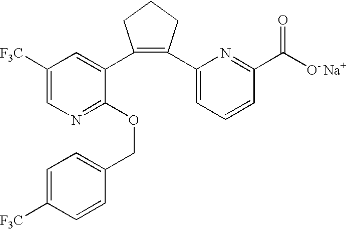 | Sodium 6-{2-[5- (trifluoromethyl)-2-({[4- (trifluoromethyl)phenyl] methyl}oxy)-3-pyridinyl]- 1-cyclopenten-1-yl}-2- pyridinecarboxylate | LC/MS: Rt = 4.04 min [MH⁺] 509 |
| 365 | 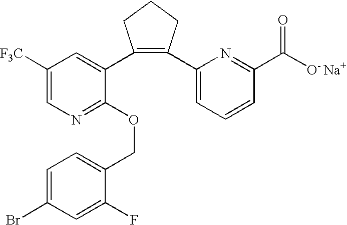 | Sodium 6-{2-[2-{[(4- bromo-2-fluorophenyl) methyl]oxy}-5-(trifluoro- methyl)-3-pyridinyl]-1- cyclopenten-1-yl}-2- pyridinecarboxylate | LC/MS: Rt = 4.11 min [MH⁺] 537, 539 |
| 366 | 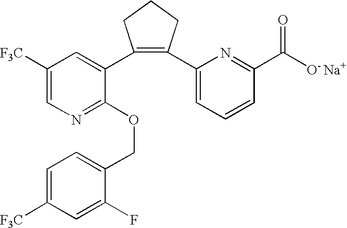 | Sodium 6-{2-[2-({[2- fluoro-4-(trifluoromethyl) phenyl]methyl}oxy)-5- (trifluoromethyl)-3- pyridinyl]-1- cyclopenten-1-yl}-2- pyridinecarboxylate | LC/MS: Rt = 4.07 min [MH⁺] 527 |
| 367 | 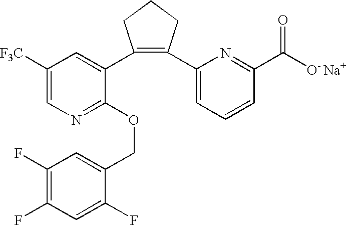 | Sodium 6-[2-(5- (trifluoromethyl)-2- {[(2,4,5-trifluorophenyl) methyl]oxy}-3-pyridinyl)- 1-cyclopenten-1-yl]-2- pyridinecarboxylate | LC/MS: Rt = 3.94 min [MH⁺] 495 |
| 368 | 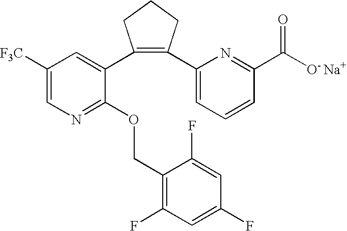 | Sodium 6-[2-(5- (trifluoromethyl)-2- {[(2,3,6-trifluorophenyl) methyl]oxy}-3-pyridinyl)- 1-cyclopenten-1-yl]-2- pyridinecarboxylate | LC/MS: Rt = 3.87 min [MH⁺] 495 |

### Example 369

### Sodium 6-[2-(5-fluoro-2-{[(4-methylphenyl)methyl]oxy}phenyl)-1-cyclopenten-1-yl]-2-pyridazinecarboxylate

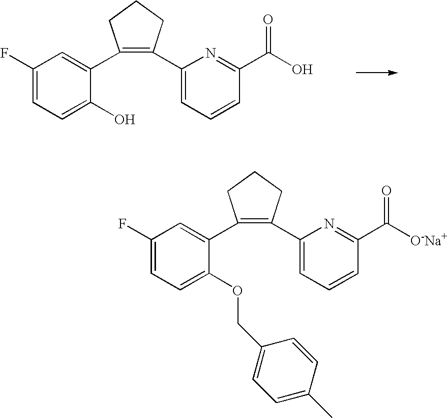

6-{2-[5-Fluoro-2-hydroxyphenyl]-1-cyclopenten-1-yl}-2-pyridinecarboxylic acid methyl ester (104 mg, 0.333 mmol) in dimethyl/formamide (4 ml) was treated with 4-methylbenzyl bromide (66 mg, 0.356 mmol) and potassium carbonate (140 mg, 1.0 mmol). The reaction mixture was then refluxed overnight under nitrogen, filtered through celite and reduced under vacuum to an oil. The oil was dissolved in methanol (3 ml), 2M sodium hydroxide (2 ml) was added and the reaction mixture stirred at 65° C. for one hour. The reaction mixture was then reduced down to ˜0.1 ml under vacuum, diluted to 20 ml with water and extracted with ethyl acetate (2×20 ml). The organic extract was then washed with brine (20 ml), dried over sodium sulphate and evaporated down under reduced pressure to the required product (52 mg, 36%). LC/MS Rt=3.73 min [MH⁺] 404.

The following Examples were prepared by the procedure used for sodium 6-[2-(5-fluoro-2-{[(4-methylphenyl)methyl]oxy}phenyl)-1-cyclopenten-1-yl]-2-pyridinecarboxylate:

| Example | Structure | Name | LC/MS |
|---|---|---|---|
| 370 | 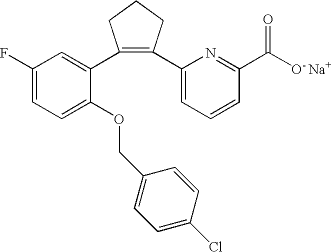 | Sodium 6-[2-(2-{[(4- chlorophenyl)methyl]oxy}- 5-fluorophenyl)-1- cyclopenten-1-yl]-2- pyridinecarboxylate | Rt = 3.83, [MH⁺] 424 |
| 371 | 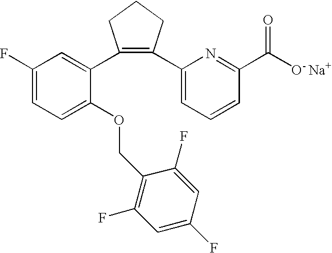 | Sodium 6-[2-(5-fluoro-2- {[(2,4,6- trifluorophenyl)methyl]oxy} phenyl)-1-cyclopenten-1- yl]-2-pyridinecarboxylate | Rt = 3.58, [MH⁺] 444 |
| 372 | 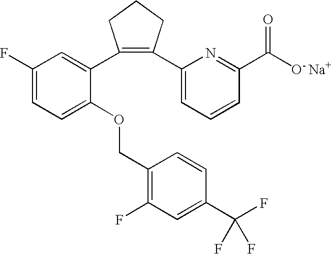 | Sodium 6-{2-[5-fluoro-2- ({[2-fluoro-4- (trifluoromethyl)phenyl] methyl}oxy)phenyl]-1- cyclopenten-1-yl}-2- pyridinecarboxylate | Rt = 3.94, [MH⁺] 476 |
| 373 | 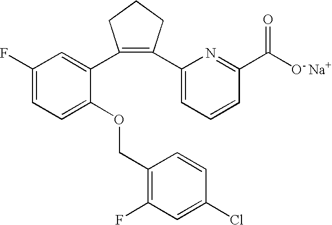 | Sodium 6-[2-(2-{[(4- chloro-2-fluorophenyl) methyl]oxy}-5- fluorophenyl)-1- cyclopenten-1-yl]-2- pyridinecarboxylate | Rt = 3.89, [MH⁺] 442 |
| 374 | 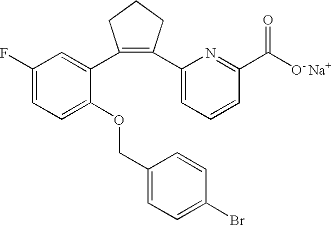 | Sodium 6-[2-(2-{[(4- bromophenyl)methyl]oxy}- 5-fluorophenyl)-1- cyclopenten-1-yl]-2- pyridinecarboxylate | Rt = 3.90, [MH⁺] 470 |
| 375 | 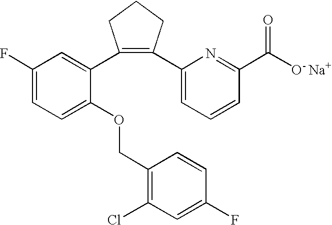 | Sodium 6-[2-(2-{[(2- chloro-4-fluorophenyl) methyl]oxy}- 5-fluorophenyl)-1- cyclopenten-1-yl]-2- pyridinecarboxylate | Rt = 3.88, [MH⁺] 442 |
| 376 | 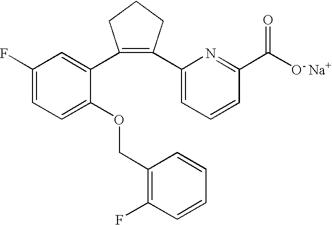 | Sodium 6-[2-(5-fluoro-2- {[(2-fluorophenyl) methyl]oxy}phenyl)- 1-cyclopenten-1- yl]-2-pyridinecarboxylate | Rt = 3.57, [MH⁺] 408 |
| 377 | 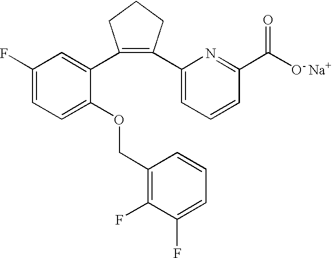 | Sodium 6-[2-(2-{[(2,3- difluorophenyl)methyl]oxy}- 5-fluorophenyl)-1- cyclopenten-1-yl]-2- pyridinecarboxylate | Rt = 3.64, [MH⁺] 426 |
| 378 | 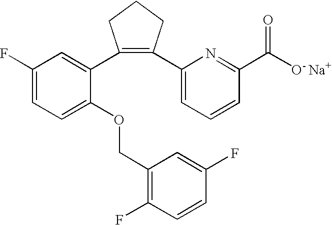 | Sodium 6-[2-(2-{[(2,5- difluorophenyl)methyl]oxy}- 5-fluorophenyl)-1- cyclopenten-1-yl]-2- pyridinecarboxylate | Rt = 3.67, [MH⁺] 426 |
| 379 | 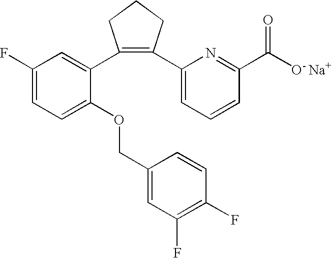 | Sodium 6-[2-(2-{[(3,4- difluorophenyl)methyl]oxy}- 5-fluorophenyl)-1- cyclopenten-1-yl]-2- pyridinecarboxylate | Rt = 3.68, [MH⁺] 426 |
| 380 | 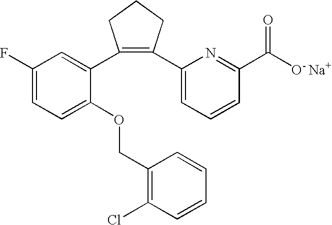 | Sodium 6-[2-(2-{[(2- chlorophenyl)methyl]oxy}- 5-fluorophenyl)-1- cyclopenten-1-yl]-2- pyridinecarboxylate | Rt = 3.84, [MH⁺] 424 |
| 381 | 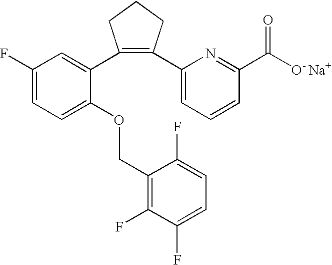 | Sodium 6-[2-(5-fluoro-2- {[(2,3,6-trifluoro- phenyl)methyl]oxy} phenyl)-1-cyctopenten-1- yl]-2-pyridinecarboxylate | Rt = 3.57, [MH⁺] 444 |
| 382 | 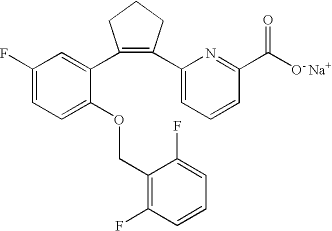 | Sodium 6-[2-(2-{[(2,6- difluorophenyl)methyl]oxy}- 5-fluorophenyl)-1- cyclopenten-1-yl]-2- pyridinecarboxylate | Rt = 3.49, [MH⁺] 426 |
| 383 | 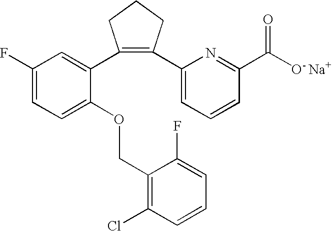 | Sodium 6-[2-(2-{[(2- chloro-6-fluorophenyl) methyl]oxy}-5- fluorophenyl)-1- cyclopenten-1-yl]-2- pyridinecarboxylate | Rt = 3.66, [MH⁺] 442 |
| 384 | 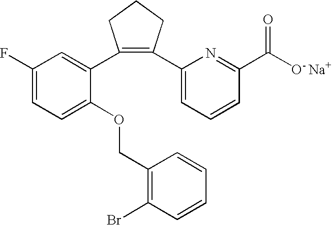 | Sodium 6-[2-(2-{[(2- bromophenyl)methyl]oxy}- 5-fluorophenyl)-1- cyclopenten-1-yl]-2- pyridinecarboxylate | Rt = 3.90, [MH⁺] 470 |
| 385 | 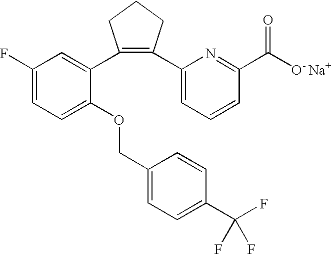 | Sodium 6-{2-[5-fluoro-2- ({[4-(trifluoromethyl) phenyl]methyl}oxy)phenyl]-1- cyclopenten-1-yl}-2- pyridinecarboxylate | Rt = 3.88, [MH⁺] 458 |

### Example 386

### 6-[2-(5-Chloro-2-{[(2,4-difluorophenyl)methyl]oxy}phenyl)-1-cyclopenten-1-yl]-N-(phenylsulfonyl)-2-pyridinecarboxamide

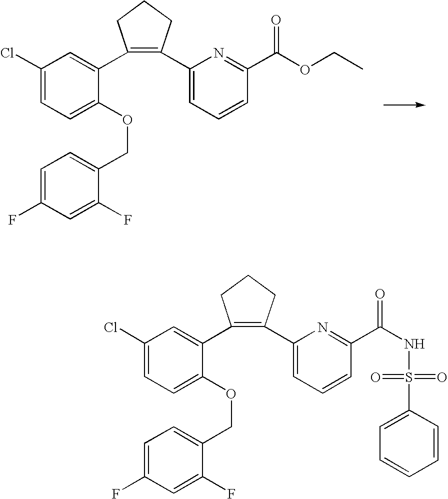

a) Ethyl 6-[2-(5-chloro-2-{[(2,4-difluorophenyl)methyl]oxy}phenyl)-1-cyclopenten-1-yl]-2-pyridinecarboxylate (140 mg, 0.30 mmol) was dissolved in ethanol (5 ml) and 2M sodium hydroxide (1 ml) and heated to reflux then left to cool for 60 minutes. The solution was diluted with water then extracted with isohexane and acidified to pH4 with hydrochloric acid. The mixture was extracted with diethyl ether. The organic solution was dried over magnesium sulphate and evaporated to give 6-[2-(5-chloro-2-{[(2,-difluorophenyl)methyl]oxy}phenyl)-1-cyclopenten-1-yl]-2-pyridinecarboxylic acid (110 mg).

LC/MS Rt=3.88 [MH⁺] 442.3, 444.3.

b) A mixture of 6-[2-(5-chloro-2-{[(2,4-difluorophenyl)methyl]oxy}phenyl)-1-cyclopenten-1-yl]-2-pyridinecarboxylic acid (110 mg, 0.25 mmol), benzenesulphonamide (58 mg, 0.3 mmol), 1-[3-(dimethylamino)propyl]-3-ethylcarbodiimide hydrochloride (58 mg, 0.3 mmol) and 4-dimethylaminopyridine (3 mg, 0.025 mmol) in 1:1 dichloromethane/tetrahydrofuran (4 ml) was stirred at room temperature for 2 hours and more benzenesulphonamide (16 mg, 0.1 mmol), 1-[3(dimethylamino)propyl]-3-ethylcarbodiimide hydrochloride (19 mg, 0.1 mmol) and 4-dimethylaminopyridine (1 mg) was added. After a further 2 hours the mixture was diluted with ether/water and the organic layer dried (magnesium sulphate), evaporated and purified by chromatography on silica eluting with ethyl acetate/iso-hexane to give a white solid (85 mg).

¹H NMR (CDCl₃) δ 2.07-2.14 (2H, m), 2.84-2.88 (2H, m), 2.98-3.01 (2H, m), 5.04 (2H, s), 6.74-6.79 (2H, m), 6.99 (1H, d), 7.06 (1H, d), 7.16-7.32 (3H, m), 7.54 (2H, t), 7.63 (2H, q), 7.81 (1H, d), 8.08-8.10 (2H, m), 9.55 (1H, s). LC/MS t=4.30, [MH+] 581.3, 583.3.

### Example 387

### 6-[2-(5-bromo-2-{[(2,4-difluorophenyl)methyl]oxy}phenyl)-1-cyclopenten-1-yl]-N-phenylsulfonyl)-2-pyridinecarboxamide

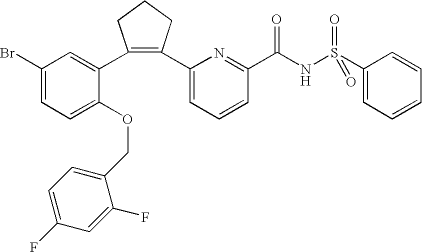

A mixture of 6-[2-(5-bromo-2-{[(2,4-difluorophenyl)methyl]oxy}phenyl)-1cyclopenten-1-yl]-2-pyridinecarboxylic acid (10 mg, 0.23 mmol), benzenesulphonamide (45 mg, 0.29 mmol), 1-[3-(dimethylamino)propyl]-3-ethylcarbodiimide hydrochloride (55 mg, 0.29 mmol), and 4 dimethylaminopyridine (5 mg) in 1:1 dichloromethane/tetrahydrofuran (5 ml) was stirred at RT for 24 hours. The reaction mixture was diluted with diethyl ether (25 ml) and washed with saturated sodium bicarbonate solution, water and brine. The organic phase was separated, dried and evaporated. Chromatography of the residue eluting with 1:9 ethyl acetate/hexane gave the title compound as a colourless solid (52 mg).

LC/MS: Rt=4.33 min. [M+H]=625, 627.

### Example 388

### 2-Fluoro-5-(2-{2-[(2-fluorophenyl)methoxy]-5-trifluoromethyl)pyridin-3-yl}cyclopent-1-en-1-yl)-benzoic acid, sodium salt

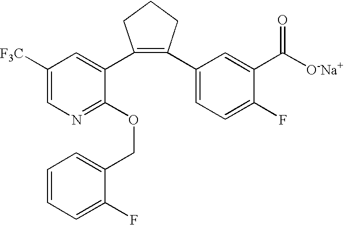

The corresponding ethyl ester was dissolved in ethanol (1 ml) and 2M aqueous sodium hydroxide (1 ml) was added. The mixture was heated to 120° C., by microwave, for 3mins. The reaction mixture was concentrated in vacuo, and the residue partitioned between ethyl acetate and water. The organic extract was dried (Na₂SO₄) and concentrated in vacuo to give the title compound as the sodium salt. LC/MS Rt=4.07 min [MH⁺] 477.

The following compounds were prepared as their sodium salts by the same method, starting from the appropriate ethyl esters.

| Example | Structure | COMPOUND NAME | LCMS |
|---|---|---|---|
| 389 | 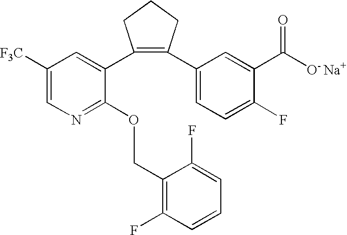 | 5-(2-{2-[(2,6-Difluorophenyl) methoxy]-5-(trifluoromethyl) pyridin-3-yl}cyclopent-1-en-1- yl)-2-fluorobenzoic acid, sodium salt | Rt = 4.03 min [MH⁺] 495 |
| 390 | 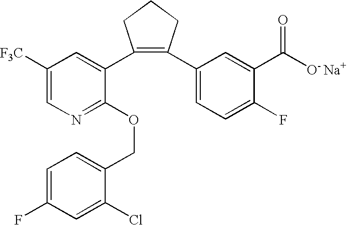 | 5-(2-{2-[(2-Chloro-4- fluorophenyl)methoxy]-5- (trifluoromethyl)pyridin-3- yl}cyclopent-1-en-1-yl)-2- fluorobenzoic acid, sodium salt | Rt = 4.25 min [MH⁺] 512 |

### General Procedure C

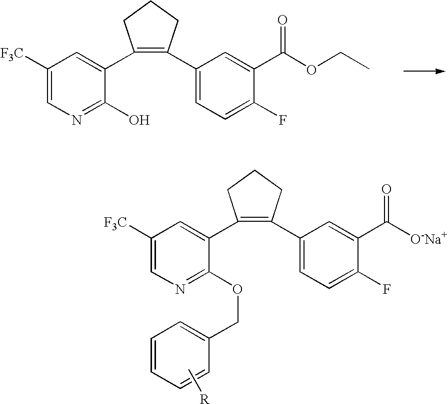

Ethyl 2-fluoro-5-{2-[2-hydroxy-5(trifluoromethyl)pyridin-3-yl]cyclopent-1-en-1-y}benzoate (250 mg, 0.63mmol) was dissolved in toluene (3 ml), together with silver carbonate (192 mg, 0.70 mmol) and a substituted benzyl bromide (1.1 equiv.). The mixture was heated to reflux for 4 hours, then concentrated in vacuo, and the product taken on without further purification.

Each residue was dissolved in a mixture of ethanol (2 ml) and 2N aqueous sodium hydroxide (2 ml), and this mixture was heated to 120° C., by microwave, for 3mins. The reaction mixture was filtered and concentrated in vacuo. The residue was dissolved in dichloromethane and treated with acetic acid, and then again concentrated in vacuo. The resulting material was purified using a basic solid phase extraction cartridge (Isolute® Flash NH2), loading the crude material as a methanol solution, and eluting with 10% aqueous HCl in methanol. The resulting adds were redissolved in dichloromethane and treated with aqueous 2N sodium hydroxide. The layers were separated, and the organic layer was concentrated in vacuo. The resulting sodium salt was redissolved in dioxane, which was removed by freeze-drying to give the product (sodium salt) as a solid.

The following compounds were prepared by General Procedure C:

| Examples | Structure | Compound Name | LCMS |
|---|---|---|---|
| 391 | 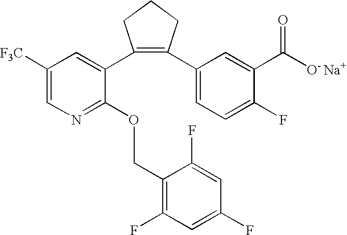 | 2-Fluoro-5-(2-{5- (trifluoromethyl)-2- [(2,4,6-trifluorophenyl) methoxy] pyridin-3- yl}cyclopent-1-en-1-yl)- benzoic acid, sodium salt | Rt = 4.07 min [MH⁺] 512 |
| 392 | 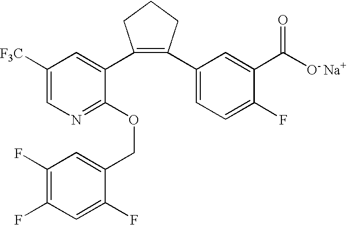 | 2-Fluoro-5-(2-{5- (trifluoromethyl)-2- [(2,4,5-trifluorophenyl) methoxy] pyridin-3- yl}cyclopent-1-en-1-yl)- benzoic acid, sodium salt | Rt = 4.09 min [MH⁺] 512 |
| 393 | 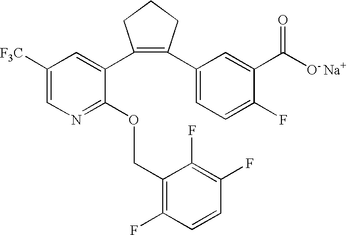 | 2-Fluoro-5-(2-{5- (trifluoromethyl)-2- [(2,3,6-trifluorophenyl) methoxy] pyridin-3- yl}cyclopent-1-en-1-yl)- benzoic acid, sodium salt | Rt = 4.11 min [MH⁺] 512 |
| 394 | 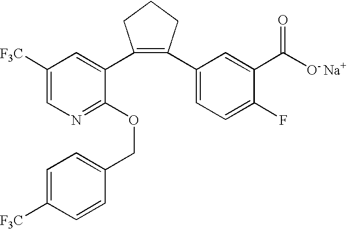 | 2-Fluoro-5-[2-(5- (trifluoromethyl)-2-{[4- (trifluoromethyl)phenyl] methoxy}pyridin-3- yl)cyclopent-1-en-1-yl]- benzoic acid, sodium salt | Rt = 4.19 min [MH⁺] 526 |
| 395 | 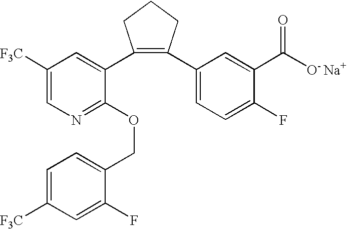 | 2-Fluoro-5-[2-(2-{(2- fluoro-4-(trifluoromethyl) phenyl]methoxy}-5- [trifluoromethyl] pyridin-3-yl)cyclopent- 1-en-1-yl]-benzoic acid, sodium salt | Rt = 4.28 min [MH⁺] 544 |
| 396 | 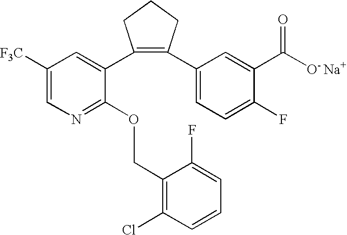 | 5-(2-{2-[(2-Chloro-6- fluorophenyl)methoxy]- 5-(trifluoromethyl)pyridin- 3-yl}cyclopent-1-en-1- yl)-2-fluorobenzoic acid, sodium salt | Rt = 4.16 min [MH⁺] 510 |
| 397 | 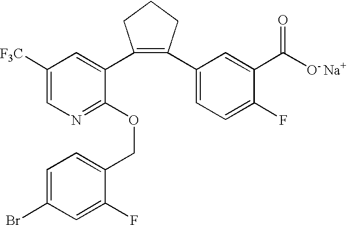 | 5-(2-{2-[(4-Bromo-2- fluorophenyl)methoxy]- 5-(trifluoromethyl)pyridin- 3-yl}cyclopent-1-en-1- yl)-2-fluorobenzoic acid, sodium salt | Rt = 4.28 min [MH⁺] 554, 556 |

### Example 398

### 3-Fluoro-5-{2-[2-(phenylmethoxy)-5-(trifluoromethyl)pyridin-3-yl]cyclopent-1en-1-yl}benzoic acid, sodium salt

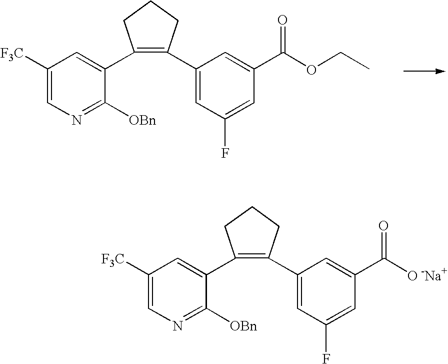

Ethyl 2-fluoro-5-{2-[2-(phenylmethoxy)-5-(trifluoromethyl)pyridin-3-yl]cyclopent-1-en-1-yl}benzoate (150 mg, 0.31 mmol) was dissolved in ethanol (2 ml) and 2M sodium hydroxide (1.0 ml) was added. The mixture was heated to reflux for 1 hour, by which time TLC analysis indicated that the reaction was complete. The cooled reaction mixture was diluted with water, acidified to pH5 with acetic acid, and then extracted with diethyl ether (×2). The combined organic extracts were washed with water, dried (Na₂SO₄) and concentrated in vacuo to give the crude acid, which was further purified by HPLC. The acid was treated with 2M aqueous sodium hydroxide, and this mixture extracted with dichloromethane. The organic extracts were concentrated in vacuo to give the title compound as the sodium salt.

LC/MS Rt=4.22 min [MH⁺] 458.

¹H NMR (MeOD) δ: 2.06-2.14 (2H, m), 2.86-2.97 (4H, m), 5.31 (2H, s), 6.93 (1H, ddd), 7.17-7.21 (2H, m), 7.24-7.30 (3H, m), 7.43 (1H, ddd), 7.51 (1H, t), 7.68 (1H, d), 8.38 (1H, dd).

### General Procedure D

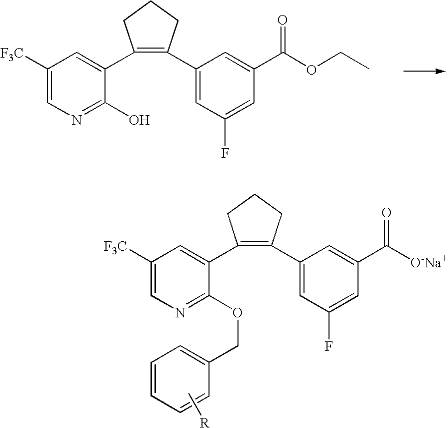

Ethyl 3-fluoro-5-{2-[2-hydroxy-5-(trifluoromethyl)pyridin-3-yl]cyclopent-1-en-1-yl}benzoate (250 mg, 0.63 mmol) was dissolved in toluene (3 ml), together with silver carbonate (192 mg, 0.70 mmol) and a substituted benzyl bromide (1.1 equiv.). The mixture was heated to reflux for 4 hours, then concentrated in vacuo, and the product taken on without further purification.

Each residue was dissolved in a mixture of ethanol (2 ml) and 2N aqueous sodium hydroxide (2 ml), and this mixture was heated to 120° C., by microwave, for 3mins. The reaction mixture was filtered and concentrated in vacuo. The residue was dissolved in dichloromethane and treated with acetic acid, and then again concentrated in vacuo. The resulting material was purified using a basic solid phase extraction cartridge (Isolute® Flash NH2), loading the crude material as a methanol solution, and eluting with 10% aqueous HCl in methanol. The resulting acid was redissolved in dichloromethane and treated with aqueous 2N sodium hydroxide. The layers were separated, and the organic layer was concentrated in vacuo. This was followed by further purification by HPLC. The pure acid was treated with 2M aqueous sodium hydroxide, and the mixture extracted with dichloromethane. The organic extracts were concentrated in vacuo to give the title compound as the sodium salt.

The following Examples were prepared by General Procedure D:

| Example | Structure | Compound Name | LCMS |
|---|---|---|---|
| 399 | 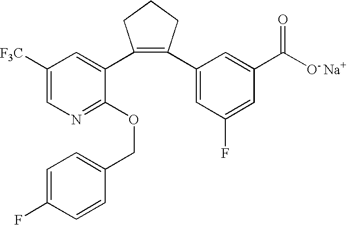 | 3-Fluoro-5-(2-{2-[(4- fluorophenyl)methoxy]-5- (trifluoromethyl)pyridin-3- yl}cyclopent-1-en-1-yl)- benzoic acid, sodium salt | Rt = 4.15 min [MH⁺] 476 |
| 400 | 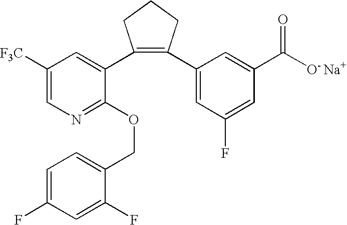 | 5-(2-{2-[(2,4-Difluoro- phenyl)methoxy]-5- (trifluoromethyl) pyridin- 3-yl}cyclopent-1-en-1-yl)- 3-fluorobenzoic acid, sodium salt | Rt = 4.17 min [MH⁺] 494 |
| 401 | 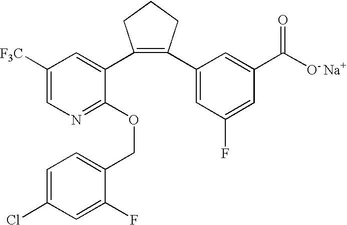 | 5-(2-{2-[(4-Chloro-2- fluorophenyl)methoxy]-5- (trifluoromethyl)pyridin-3- yl}cyclopent-1-en-1-yl)-3- fluorobenzoic acid, sodium salt | Rt = 4.31 min [MH⁺] 510 |
| 402 | 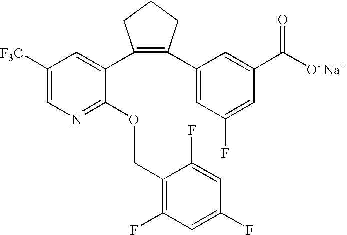 | 3-Fluoro-5-(2-{5- (trifluoromethyl)-2- [(2,4,6-trifluorophenyl) methoxy]pyridin-3-yl) cyclopent-1-en-1-yl)- benzoic acid, sodium salt | Rt = 4.12 min [MH⁺] 512 |
| 403 | 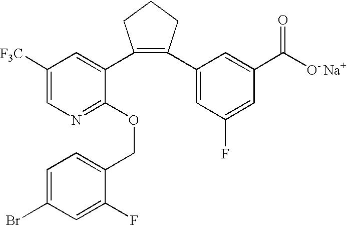 | 5-(2-{2-[(4-Bromo-2- fluorophenyl)methoxy]-5- (trifluoromethyl)pyridin-3- yl}cyclopent-1-en-1-yl)-3- fluorobenzoic acid, sodium salt | Rt = 4.40 min [MH⁺] 554, 556 |
| 404 | 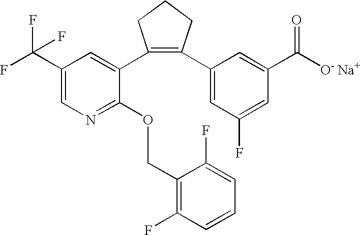 | Sodium 3-{2-[2-{[(2,6- difluorophenyl)methyl] oxy}-5-(trifluoromethyl)-3- pyridinyl]-1-cyclopenten- 1-yl)-5-fluorobenzoate | Rt = 4.12 min [MH⁺] 493 |

### General Procedure E

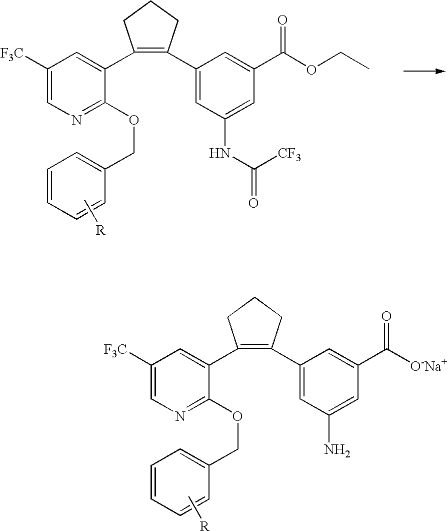

The ester was dissolved in ethanol (2 ml) and 2M aqueous sodium hydroxide (1 ml) was added. The mixture was heated to reflux for 2 hours. The reaction mixture was concentrated in vacuo, and treated according to procedure A or B.

Procedure A: The residue was triturated with aqueous sodium hydroxide to give the sodium salt as a solid, which was collected by filtration and washed with water.
Procedure B: The residue was partitioned between ethyl acetate and water. The organic layer was dried (Na₂SO₄), and concentrated in vacuo, to give the sodium salt as a glassy solid.

The following Examples were prepared as their sodium salts by General Procedure E, starting from the appropriate ethyl esters

| Example | Structure | Compound Name | LCMS |
|---|---|---|---|
| 405 | 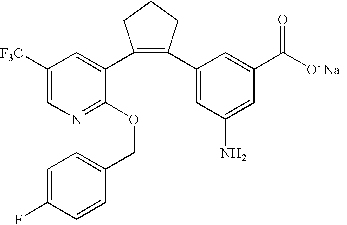 | 3-Amino-5-(2-{2-[(4- fluorophenyl)methoxy]-5- (trifluoromethyl)pyridin-3- yl}cyclopent-1-en-1-yl)- benzoic acid, sodium salt | Rt = 3.80 min [MH⁺] 473 |
| 406 | 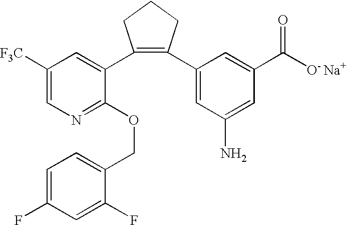 | 3-Amino-5-(2-{2-[(2,4- difluorophenyl)methoxy]-5- (trifluoromethyl)pyridin-3- yl}cyclopent-1-en-1-yl)- benzoic acid, sodium salt | Rt = 3.84 min [MH⁺] 491 |
| 407 | 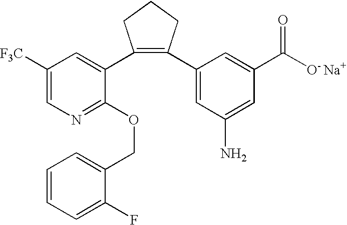 | 3-Amino-5-(2-{2-[(2- fluorophenyl)methoxy]-5- (trifluoromethyl)pyridin-3- yl}cyclopent-1-en-1-yl)- benzoic acid, sodium salt | Rt = 3.80 min [MH⁺] 473 |
| 408 | 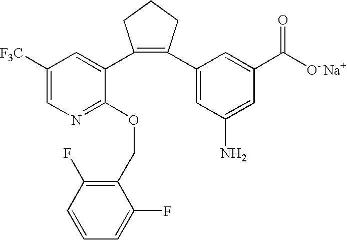 | 3-Amino-5-(2-{2-[(2,6- difluorophenyl)methoxy]-5- (trifluoromethyl)pyridin-3- yl}cyclopent-1-en-1-yl)- benzoic acid, sodium salt | Rt = 3.77 min [MH⁺] 491 |
| 409 | 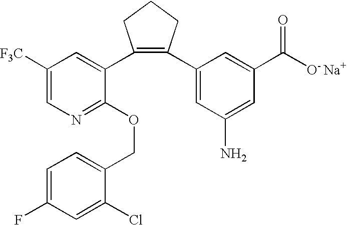 | 3-Amino-5-(2-{2-[(2-chloro- 4-fluorophenyl)methoxy]-5- (trifluoromethyl)pyridin-3- yl}cyclopent-1-en-1-yl)- benzoic acid, sodium salt | Rt = 3.98 min [MH⁺] 507 |
| 410 | 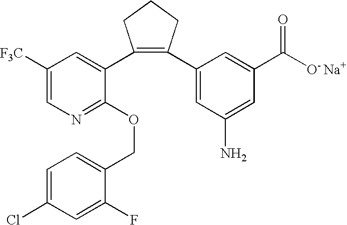 | 3-Amino-5-(2-{2-[(4-chloro- 2-fluorophenyl)methoxy]-5- (trifluoromethyl)pyridin-3- yl}cyclopent-1-en-1-yl)- benzoic acid, sodium salt | Rt = 3.98 min [MH⁺] 507 |
| 411 | 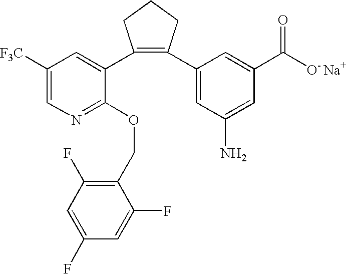 | 3-Amino-5-(2-{5- (trifluoromethyl)-2-[(2,4,6- trifluorophenyl)methoxy]- pyridin-3-yl}cyclopent-1-en- 1-yl)-benzoic acid, sodium salt | Rt = 3.84 min [MH⁺] 509 |
| 412 | 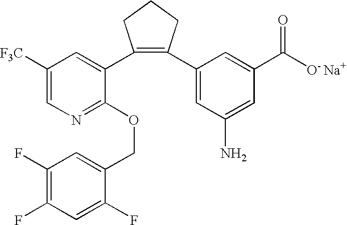 | 3-Amino-5-(2-{5- (trifluoromethyl)-2-[(2,4,5- trifluorophenyl)methoxy]- pyridin-3-yl}cyclopent-1-en- 1-yl)-benzoic acid, sodium salt | Rt = 3.87 min [MH⁺] 509 |
| 413 | 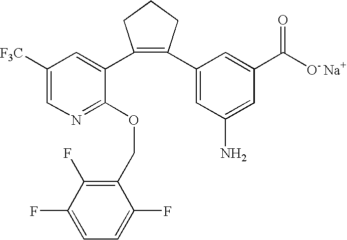 | 3-Amino-5-(2-{5- (trifluoromethyl)-2-[(2,3,6- trifluorophenyl)methoxy]- pyridin-3-yl}cyclopent-1-en- 1-yl)-benzoic acid, sodium salt | Rt = 3.85 min [MH⁺] 509 |
| 414 | 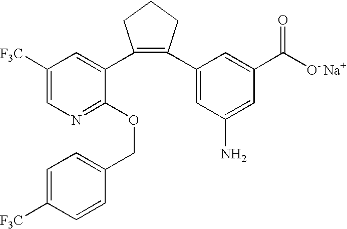 | 3-Amino-5-[2-(5- {trifluoromethyl)-2-{[4- (trifluoromethyl)phenyl] methoxy}-pyridin-3-yl) cyclopent-1-en-1-yl]- benzoic acid, sodium salt | Rt = 4.01 min [MH⁺] 523 |
| 415 | 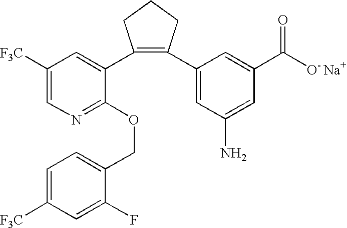 | 3-Amino-5-[2-(2-{[2-fluoro-4- (trifluoromethyl)phenyl] methoxy}-5-{trifluoro- methyl}pyridin-3- yl)cyclopent-1-en-1-yl]- benzoic acid, sodium salt | Rt = 4.01 min [MH⁺] 541 |
| 416 | 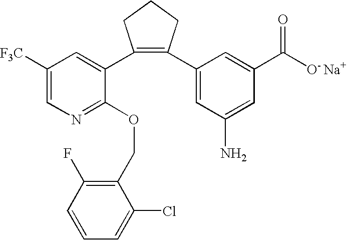 | 3-Amino-5-(2-{2-[(2-chloro- 6-fluorophenyl)methoxy]-5- (trifluoromethyl)pyridin-3- yl}cyclopent-1-en-1-yl)- benzoic acid, sodium salt | Rt = 3.89 min [MH⁺] 507 |
| 417 | 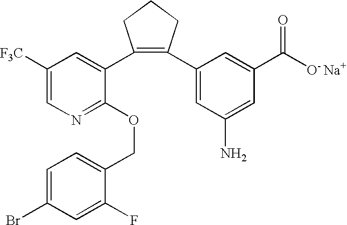 | 3-Amino-5-(2-{2-[(4-bromo- 2-fluorophenyl)methoxy]-5- (trifluoromethyl)pyridin-3- yl}cyclopent-1-en-1-yl)- benzoic acid, sodium salt | Rt = 4.03 min [MH⁺] 551, 553 |

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. The prostaglandin receptors investigated are DP, EP₁, EP₂, EP₃, EP₄, FP, IP and TP.

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]₁) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-3, AM and a suitable instrument such as a Fluorimetric Imaging Plate Reader (FLIPR). Increasing amounts of [Ca²⁺] produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing fluo-3 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine.

The concentration of compound that elicits a half-maximal inhibition of the calcium mobilisation induced by PGE₂ (pIC₅₀) may then be estimated.

### Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM Indomethacin for 5 min. The cells are isolated by centrifugation at 250×g for 5mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10s burst at full setting), centrifuged at 48,000×g for 20mins and the pellet containing the membrane fraction is washed three times by suspension and centrifugation at 48,000×g for 20mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at 80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques (GraphPad Prism 3) to determine the concentration of compound producing 50% inhibition of specific binding (IC₅₀).

By application of the binding assay technique, compounds of the examples had an antagonist pIC₅₀ value of 6.0 to 9.5 at EP₁ receptors. Compounds of the examples had a pIC50 value of <6.0 at EP₃ receptors when measured by the calcium mobilisation assay.

No toxicological effects are indicated/expected when a compound (of the invention) is administered in the above mentioned dosage range.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 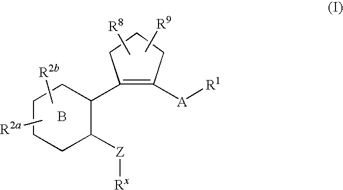
wherein:
A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂H, CN, CONR⁵R⁶, CH₂CO₂H, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2; optionally substituted alkenyl; or optionally substituted alkynyl: or R^{x} represents optionally substituted alkenyl, optionally substituted CQ^{a}Q^{b}-heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ and R⁹ each independently represents hydrogen, chloro, fluoro, CF₃, C₁₋₃alkoxy or C₁₋₃alkyl;
Q^{a} and Q^{b} are each independently selected from hydrogen and CH₃;
wherein when A is a 6-membered ring the R¹ substituent and cyclopentene ring are attached to carbon atoms 1,2-, 1,3- or 1,4-relative to each other, and when A is a five-membered ring or bicyclic heterocyclyl group the R¹ substituent and cyclopentene ring are attached to substitutable carbon atoms 1,2- or 1,3-relative to each other;
and derivatives thereof.

2. A compound according to claim 1 wherein B is pyridyl.

3. A compound according to claim 1 which is a compound of formula (IA): 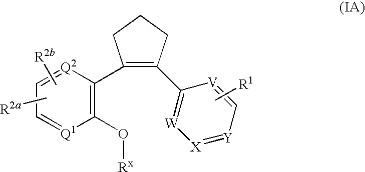
wherein:
W, X, and Y each represent CR¹² or N;
V represents CR¹, CR¹² or N;
wherein at least two of W, X, Y and V is CR¹², and R¹² is independently selected from hydrogen, halogen, CF₃, CH₃, NH₂, NHC₁₋₆alkyl, NHCOC₁₋₆alkyl, and SCH₃;
Q¹ and Q² each represents CH, or one of Q¹ and Q² is N and the other is CH;
R¹ is CO₂H, CONR⁵R⁶, CH₂CO₂H, SO₂C₁₋₆alkyl, SO₂NR⁵R⁶, NR⁵CONR⁵R⁶, tetrazolyl or COSO₂NR⁵R⁶;
R^{2a} and R^{2b} are selected from hydrogen, halogen, optionally substituted C₁₋₆alkyl, and optionally substituted C₁₋₆alkoxy;
R^{x} represents optionally substituted C₃₋₈alkyl, optionally substituted C₃₋₈alkenyl, and optionally substituted CH₂phenyl;
R⁵ is hydrogen or C₁₋₄alkyl;
R⁶ is hydrogen, C₁₋₄alkyl or SO₂phenyl;
R¹² is selected from hydrogen, halogen, NR⁵R⁶, NR⁵COC₁₋₆alkyl, NR⁵SO₂C₁₋₆alkyl, OR⁵, SR⁵, and optionally substituted C₁₋₆alkyl;
or derivatives thereof.

4. A compound according to claim 3 wherein one of Q¹ and Q² is N and the other is CH.

5.-6. (canceled)

7. A pharmaceutical composition comprising a compound according to claim 1 or a pharmaceutically acceptable derivative thereof together with a pharmaceutical carrier and/or excipient.

8.-9. (canceled)

10. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

11. A method of treating a human or animal subject suffering from a pain, inflammatory, immunological, bone, neurodegenerative or renal disorder, which method comprises administering to said subject an effective amount of a compound according to claim 1 or a pharmaceutically acceptable derivative thereof.

12. A method of treating a human or animal subject suffering from inflammatory pain, neuropathic pain or visceral pain which method comprises administering to said subject an effective amount of a compound according to claim 1 or a pharmaceutically acceptable derivative thereof.

13.-15. (canceled)

16. The method of claim 10 wherein the subject is human.

17. The method of claim 11 wherein the subject is human.

18. The method of claim 12 wherein the subject is human.

19. A method of mediating EP₁ receptors, comprising the step of administering an effective amount of a compound according to claim 1 or a pharmaceutically acceptable derivative thereof.
